(19) United States
(12) Patent Application Publication
(10) Pub. No.: US 2024/0130341 A1
(43) Pub. Date: Apr. 25, 2024

(54) CORRECTION OF HEPATOSTEATOSIS IN HUMANIZED LIVER ANIMALS THROUGH RESTORATION OF IL6/IL6R/GP130 SIGNALING IN HUMAN HEPATOCYTES

(71) Applicant: Regeneron Pharmaceuticals, Inc., Tarrytown, NY (US)

(72) Inventors: Joseph Zhe Li, Northvale, NJ (US); Marisa Carbonaro, Eastchester, NY (US); Gavin Thurston, Millerton, NY (US); Andrew J. Murphy, Croton-on-Hudson, NY (US)

(21) Appl. No.: 18/478,273

(22) Filed: Sep. 28, 2023

Related U.S. Application Data
(60) Provisional application No. 63/487,134, filed on Feb. 27, 2023.
(60) Provisional application No. 63/377,651, filed on Sep. 29, 2022.

(51) Int. Cl. A01K 67/0278; A61K 38/17; A61P 1/16; C07K 14/54; C07K 14/715; C12N 5/071; G01N 33/50

(52) U.S. Cl. CPC A01K 67/0278; A61K 38/1793; A61P 1/16; C07K 14/5412; C07K 14/7155; C12N 5/067; G01N 33/5067; A01K 2207/15; A01K 2227/105; A01K 2267/0362

(57) ABSTRACT

Genetically modified non-human animals that are immunodeficient and comprise xenotransplanted hepatocytes such as human hepatocytes, wherein the genetically modified non-human animal and/or the transplanted hepatocytes are modified to restore interleukin-6 (IL-6)/interleukin-6 receptor (IL-6R) signaling pathway activity or interleukin-6 receptor subunit beta (GP130) signaling pathway activity in the transplanted hepatocytes, are provided. Also provided are methods of assessing the activity of human-liver-targeting reagents in such non-human animals and methods of making animals with a humanized liver (e.g., with reduced steatosis). Also provided are genetically modified non-human animals comprising an inactivated endogenous Rag2 gene, an inactivated endogenous Il2rg gene, an inactivated endogenous Fah gene, a humanized IL6 gene, and optionally an inactivated endogenous Rag1 gene and methods of using and making such animals. Also provided are methods of reducing or ameliorating hepatosteatosis in non-human animals with humanized livers.

### CROSS-REFERENCE TO RELATED APPLICATIONS

This application claims the benefit of U.S. Application No. 63/377,651, filed Sep. 29, 2022, and U.S. Application No. 63/487,134, filed Feb. 27, 2023, each of which is herein incorporated by reference in its entirety for all purposes.

### REFERENCE TO A SEQUENCE LISTING SUBMITTED AS AN XML FILE VIA EFS WEB

The Sequence Listing written in file 602755SEQLIST.xml is 67.9 kilobytes, was created on Sep. 29, 2023, and is hereby incorporated by reference.

### BACKGROUND

Fatty liver disease is an increasing health issue in the developed world. However, there are few therapeutic options, in part because of a paucity of experimental models. Humanized liver mouse and rat models, in which donor human hepatocytes repopulate recipient rodent livers, have been used in studying human liver biology, diseases, and therapeutics. However, engrafted human hepatocytes in both humanized liver mice and rats show defects, including increased lipid droplet accumulation. The mechanisms underlying this phenomenon have not been well characterized. Ameliorating such imperfections would improve the accuracy of the models to recapitulate normal human liver biology.

### SUMMARY

Provided herein are genetically modified non-human animals that are immunodeficient and comprise xenotransplanted hepatocytes such as human hepatocytes, wherein the genetically modified non-human animal and/or the transplanted hepatocytes are modified to restore interleukin-6 (IL-6)/interleukin-6 receptor (IL-6R) signaling pathway activity or interleukin-6 receptor subunit beta (GP130) signaling pathway activity in the transplanted hepatocytes. Also provided are methods of assessing the activity of human-liver-targeting reagents in such non-human animals and methods of making animals with a humanized liver (e.g., with reduced steatosis). Also provided are genetically modified non-human animals comprising an inactivated endogenous Rag2 gene, an inactivated endogenous Il2rg gene, an inactivated endogenous Fah gene, and a humanized IL6 gene and methods of using and making such animals. Also provided are genetically modified non-human animals comprising an inactivated endogenous Rag1 gene, an inactivated endogenous Rag2 gene, an inactivated endogenous Il2rg gene, an inactivated endogenous Fah gene, and a humanized IL6 gene and methods of using and making such animals. Also provided are methods of reducing or ameliorating hepatosteatosis in non-human animals with humanized livers.

In one aspect, provided are genetically modified non-human animals. Some such non-human animals comprise transplanted hepatocytes from a different species than the non-human animal, optionally wherein the hepatocyte are human hepatocytes, wherein the genetically modified non-human animal and/or the transplanted hepatocytes are modified to restore interleukin-6 (IL-6)/interleukin-6 receptor (IL-6R) signaling pathway activity or to restore interleukin-6 receptor subunit beta (GP130) signaling pathway activity in the transplanted hepatocytes. In some such non-human animals, the genetically modified non-human animal is immunodeficient. In some such non-human animals, the non-human animal comprises an inactivated endogenous Il2rg gene. In some such non-human animals, the non-human animal comprises an inactivated endogenous Rag1 gene and/or an inactivated endogenous Rag2 gene. In some such non-human animals, the non-human animal comprises an inactivated endogenous Rag2 gene. In some such non-human animals, the non-human animal comprises an inactivated endogenous Rag1 gene and an inactivated endogenous Rag2 gene. In some such non-human animals, the genetically modified non-human animal comprises an inactivated endogenous Rag2 gene and an inactivated endogenous Il2rg gene. In some such non-human animals, the genetically modified non-human animal comprises an inactivated endogenous Rag1 gene, an inactivated endogenous Rag2 gene, and an inactivated endogenous Il2rg gene. In some such non-human animals, the non-human animal comprises a severe combined immunodeficiency (SCID) mutation in a Prkdc gene (Prkdc^{scid}). In some such non-human animals, the non-human animal is genetically modified so that endogenous non-human animal hepatocytes in the liver can be selectively and conditionally ablated. In some such non-human animals, the genetically modified non-human animal comprises a urokinase type plasminogen activator gene operably linked to a liver-specific promoter or a herpes simplex virus type 1 thymidine kinase (HSVtk) gene operably linked to a liver-specific promoter. In some such non-human animals, the genetically modified non-human animal comprises an inactivated endogenous Fah gene. In some such non-human animals, the transplanted hepatocytes are human hepatocytes. In some such non-human animals, the genetically modified non-human animal comprises an inactivated endogenous Rag2 gene, an inactivated endogenous Il2rg gene, and an inactivated endogenous Fah gene, wherein the genetically modified non-human animal comprises transplanted hepatocytes, wherein the transplanted hepatocytes are human hepatocytes, and wherein the genetically modified non-human animal and/or the transplanted hepatocytes are modified to restore interleukin-6 (IL-6)/interleukin-6 receptor (IL-6R) signaling pathway activity or interleukin-6 receptor subunit beta (GP130) signaling pathway activity in the transplanted hepatocytes. In some such non-human animals, the genetically modified non-human animal comprises an inactivated endogenous Rag1 gene, an inactivated endogenous Rag2 gene, an inactivated endogenous Il2rg gene, and an inactivated endogenous Fah gene, wherein the genetically modified non-human animal comprises transplanted hepatocytes, wherein the transplanted hepatocytes are human hepatocytes, and wherein the genetically modified non-human animal and/or the transplanted hepatocytes are modified to restore interleukin-6 (IL-6)/interleukin-6 receptor (IL-6R) signaling pathway activity or interleukin-6 receptor subunit beta (GP130) signaling pathway activity in the transplanted hepatocytes.

In some such non-human animals, the transplanted hepatocytes express non-human animal IL-6R (i.e., wherein the non-human animal IL-6R is from the same species as the genetically modified non-human animal, optionally wherein the genetically modified non-human animal is a mouse and the non-human animal IL-6R is mouse IL-6R, or optionally wherein the genetically modified non-human animal is a rat and the non-human animal IL-6R is rat IL-6R). In some such non-human animals, the transplanted hepatocytes express non-human animal oncostatin-M-specific receptor subunit beta (OSMR) (i.e., wherein the non-human animal OSMR is from the same species as the genetically modified non-human animal, optionally wherein the genetically modified non-human animal is a mouse and the non-human animal OSMR is mouse OSMR, or optionally wherein the genetically modified non-human animal is a rat and the non-human animal OSMR is rat OSMR). In some such non-human animals, the transplanted hepatocytes express non-human animal IL-6R and non-human animal OSMR (i.e., wherein the non-human animal IL-6R and non-human animal OSMR are from the same species as the genetically modified non-human animal, optionally wherein the genetically modified non-human animal is a mouse, the non-human animal IL-6R is mouse IL-6R, and the non-human animal OSMR is mouse OSMR, or optionally wherein the genetically modified non-human animal is a rat, the non-human animal IL-6R is rat IL-6R, and the non-human animal OSMR is rat OSMR). In some such non-human animals, the transplanted hepatocytes comprise a vector comprising an expression construct for the non-human animal IL-6R comprising a nucleic acid encoding the non-human animal IL-6R operably linked to a promoter. In some such non-human animals, the vector is a viral vector. In some such non-human animals, the viral vector is a lentivirus vector or an adeno-associated virus (AAV) vector. In some such non-human animals, the viral vector is the lentivirus vector. In some such non-human animals, the transplanted hepatocytes comprise in their genome a non-human animal IL-6R expression construct comprising a nucleic acid encoding the non-human animal IL-6R operably linked to a promoter. In some such non-human animals, the promoter is a liver-specific promoter. In some such non-human animals, the promoter is a constitutive promoter. In some such non-human animals, the transplanted hepatocytes comprise a vector comprising an expression construct for the non-human animal OSMR comprising a nucleic acid encoding the non-human animal OSMR operably linked to a promoter. In some such non-human animals, the vector is a viral vector. In some such non-human animals, the viral vector is a lentivirus vector or an adeno-associated virus (AAV) vector. In some such non-human animals, the viral vector is the lentivirus vector. In some such non-human animals, the transplanted hepatocytes comprise in their genome a non-human animal OSMR expression construct comprising a nucleic acid encoding the non-human animal OSMR operably linked to a promoter. In some such non-human animals, the promoter is a liver-specific promoter. In some such non-human animals, the promoter is a constitutive promoter.

In some such non-human animals, the transplanted hepatocytes express a ligand-independent, constitutively active form of interleukin-6 receptor subunit beta (GP130). In some such non-human animals, the transplanted hepatocytes comprise a vector comprising an expression construct for the constitutively active GP130 comprising a nucleic acid encoding the constitutively active GP130 operably linked to a promoter. In some such non-human animals, the vector is a viral vector. In some such non-human animals, the viral vector is a lentivirus vector or an AAV vector. In some such non-human animals, the viral vector is the lentivirus vector. In some such non-human animals, the transplanted hepatocytes comprise in their genome an expression construct for the constitutively active GP130 comprising a nucleic acid encoding the constitutively active GP130 operably linked to a promoter. In some such non-human animals, the promoter is a liver-specific promoter. In some such non-human animals, the promoter is a constitutive promoter. In some such non-human animals, the constitutively active GP130 is a constitutively active human GP130. In some such non-human animals, the constitutively active human GP130 comprises a deletion of the region of GP130 from Tyr186 to Tyr190 (GP130^{Y186-Y190del}).

In some such non-human animals, the non-human animal further comprises a GP130-activating ligand. In some such non-human animals, the GP130-activating ligand comprises human IL-6 or human-IL-6R-compatible ligand (e.g., human-IL-6R-compatible IL-6). In some such non-human animals, the GP130-activating ligand comprises human IL-6 or human-IL-6R-compatible IL-6. In some such non-human animals, the GP130-activating ligand comprises human oncostatin-M (OSM) or human-OSMR-compatible ligand (e.g., human-OSMR-compatible OSM). In some such non-human animals, the non-human animal further comprises one or more additional GP130-activating ligands. In some such non-human animals, the GP130-activating ligands comprise: (1) human IL-6 or human-IL-6R-compatible ligand (e.g., human-IL-6R-compatible IL-6); and (2) human OSM or human-OSMR-compatible ligand (e.g., human-OSMR-compatible OSM). In some such non-human animals, the non-human animal comprises a vector comprising an expression construct for the GP130-activating ligand comprising a nucleic acid encoding the GP130-activating ligand operably linked to a promoter. In some such non-human animals, the non-human animal comprises the vector in muscle cells. In some such non-human animals, the promoter is a tissue-specific promoter, optionally wherein the tissue-specific promoter is a muscle-specific promoter, optionally wherein the muscle-specific promoter is a hybrid mouse alpha-myosin heavy-chain (MH) and muscle creatine kinase (CK) promoter (MHCK7). In some such non-human animals, the promoter is a constitutive promoter. In some such non-human animals, the vector is a viral vector. In some such non-human animals, the viral vector is a lentivirus vector or an adeno-associated virus (AAV) vector. In some such non-human animals, the viral vector is the AAV vector, optionally wherein the AAV vector is a recombinant AAV9 vector. In some such non-human animals, comprising a nucleic acid encoding the GP130-activating ligand operably linked to a promoter. In some such non-human animals, the GP130-activating ligand comprises human IL-6 or human-IL-6R-compatible ligand (e.g., human-IL-6R-compatible IL-6). In some such non-human animals, the GP130-activating ligand comprises human IL-6 or human-IL-6R-compatible IL-6. In some such non-human animals, the GP130-activating ligand comprises human OSM or human-OSMR-compatible ligand (e.g., human-OSMR-compatible OSM). In some such non-human animals, the promoter is a tissue-specific promoter, optionally wherein the tissue-specific promoter is a muscle-specific promoter, optionally wherein the muscle-specific promoter is a hybrid mouse alpha-myosin heavy-chain (MI-1) and muscle creatine kinase (CK) promoter (MHCK7). In some such non-human animals, the promoter is a constitutive promoter. In some such non-human animals, the promoter is an exogenous promoter. In some such non-human animals, the promoter is an endogenous promoter.

In some such non-human animals, the non-human animal comprises a humanized non-human animal IL6 gene comprising a human IL6 nucleic acid encoding a human IL-6 protein. In some such non-human animals, the human nucleic acid comprises a region of human IL6 genomic sequence from the start codon to the stop codon. In some such non-human animals, the human nucleic acid comprises a human IL6 complementary DNA (cDNA). In some such non-human animals, the human nucleic acid replaces a corresponding region of the non-human animal IL6 gene. In some such non-human animals, the human nucleic acid is inserted into the non-human animal IL6 gene. In some such non-human animals, the human nucleic acid is operably linked to an endogenous non-human animal IL6 promoter. In some such non-human animals, the non-human animal is heterozygous for the humanized IL6 gene. In some such non-human animals, the non-human animal is homozygous for the humanized IL6 gene. In some such non-human animals, the non-human animal comprises the humanized IL6 gene in its germline. In some such non-human animals, the non-human animal comprises a humanized non-human animal OSM gene comprising a human OSM nucleic acid encoding a human OSM protein. In some such non-human animals, the human nucleic acid comprises a region of human OSM genomic sequence from the start codon to the stop codon. In some such non-human animals, the human nucleic acid comprises a human OSM complementary DNA (cDNA). In some such non-human animals, the human nucleic acid replaces a corresponding region of the non-human animal OSM gene. In some such non-human animals, the human nucleic acid is inserted into the non-human animal OSM gene. In some such non-human animals, the human nucleic acid is operably linked to an endogenous non-human animal OSM promoter. In some such non-human animals, the non-human animal is heterozygous for the humanized OSM gene. In some such non-human animals, the non-human animal is homozygous for the humanized OSM gene. In some such non-human animals, the non-human animal comprises the humanized OSM gene in its germline. In some such non-human animals, the non-human animal comprises a humanized non-human animal IL6 gene comprising a human IL6 nucleic acid encoding a human IL-6 protein, and wherein the non-human animal comprises a humanized non-human animal OSM gene comprising a human OSM nucleic acid encoding a human OSM protein.

In some such non-human animals, the genetically modified non-human animal does not comprise human Kupffer cells in the liver. In some such non-human animals, the genetically modified non-human animal does not comprise a reconstituted human immune system.

In some such non-human animals, the genetically modified non-human animal comprises a humanized non-human animal SIRPA gene. In some such non-human animals, the humanized non-human animal SIRPA gene comprises a replacement of exons 2-4 of the non-human animal SIRPA gene with exons 2-4 of human SIRPA, wherein the humanized non-human animal SIRPA gene encodes a chimeric SIRPA protein comprising an extracellular portion of a human SIRPA protein and an intracellular portion of a non-human animal SIRPA protein. In some such non-human animals, the humanized non-human animal SIRPA gene is operably linked to an endogenous non-human animal SIRPA promoter. In some such non-human animals, the non-human animal is heterozygous for the humanized SIRPA gene. In some such non-human animals, the non-human animal is homozygous for the humanized SIRPA gene. In some such non-human animals, the non-human animal comprises the humanized SIRPA gene in its germline.

In some such non-human animals, the non-human animal is a male. In some such non-human animals, the non-human animal is a female. In some such non-human animals, the non-human animal is a mammal. In some such non-human animals, the mammal is a rodent. In some such non-human animals, the rodent is a rat or a mouse. In some such non-human animals, the rodent is the rat. In some such non-human animals, the rodent is the mouse.

In some such non-human animals, the transplanted hepatocytes have reduced lipid droplet accumulation compared to transplanted hepatocytes in genetically modified non-human animals in which the genetically modified non-human animal and the transplanted hepatocytes are not modified to restore IL-6/IL-6R signaling pathway activity or interleukin-6 receptor subunit beta (GP130) signaling pathway activity in the transplanted hepatocytes.

In another aspect, provided are methods of assessing the activity of a human-liver-targeting reagent in vivo. Some such methods comprise: (a) administering the human-liver-targeting reagent to any of the above genetically modified non-human animals; and (b) assessing the activity of the human-liver-targeting reagent in the liver of the genetically modified non-human animal. In some such methods, step (a) comprises AAV-mediated delivery, lipid nanoparticle (LNP)-mediated delivery, hydrodynamic delivery (HDD), or injection. In some such methods, the human-liver-targeting reagent targets a target gene expressed in the human liver. In some such methods, step (b) comprises measuring expression of a messenger RNA or a protein encoded by the target gene. In some such methods, step (b) comprises assessing modification of a genomic locus comprising the target gene, optionally wherein step (b) comprises measuring the frequency of insertions or deletions within the genomic locus comprising the target gene. In some such methods, the human-liver-targeting reagent comprises a nuclease agent designed to target a region of the target gene. In some such methods, the nuclease agent comprises a Cas protein and a guide RNA designed to target a guide RNA target sequence in the target gene, optionally wherein the Cas protein is a Cas9 protein. In some such methods, the human-liver-targeting reagent comprises an exogenous donor nucleic acid, wherein the exogenous donor nucleic acid is designed to target the target gene, and optionally wherein the exogenous donor nucleic acid is delivered via AAV. In some such methods, the human-liver-targeting reagent targets a target RNA expressed in the human liver. In some such methods, the human-liver-targeting reagent is an RNAi agent or an antisense oligonucleotide. In some such methods, the human-liver-targeting reagent targets a target protein expressed in the human liver. In some such methods, the human-liver-targeting reagent is an antigen-binding protein or a small molecule.

In another aspect, provided are methods of making a non-human animal with a humanized liver. Some such methods comprise: (a) transplanting human hepatocytes or human hepatocyte progenitors into a genetically modified non-human animal; and (b) allowing the human hepatocytes or human hepatocyte progenitors to expand, wherein the genetically modified non-human animal and/or the transplanted human hepatocytes or human hepatocyte progenitors are modified to restore interleukin-6 (IL-6)/interleukin-6 receptor (IL-6R) signaling pathway activity or interleukin-6 receptor subunit beta (GP130) signaling pathway activity in the transplanted human hepatocytes or human hepatocyte progenitors.

In some such methods, the genetically modified non-human animal is immunodeficient. In some such methods, the non-human animal comprises an inactivated endogenous Il2rg gene. In some such methods, the non-human animal comprises an inactivated endogenous Rag1 gene and/or an inactivated endogenous Rag2 gene. In some such methods, the non-human animal comprises an inactivated endogenous Rag2 gene. In some such methods, the non-human animal comprises an inactivated endogenous Rag1 gene and an inactivated endogenous Rag2 gene. In some such methods, the genetically modified non-human animal comprises an inactivated endogenous Rag2 gene and an inactivated endogenous Il2rg gene. In some such methods, the genetically modified non-human animal comprises an inactivated endogenous Rag1 gene, an inactivated endogenous Rag2 gene, and an inactivated endogenous Il2rg gene. In some such methods, the non-human animal comprises a severe combined immunodeficiency (SCID) mutation in a Prkdc gene (Prkd^{scid}). In some such methods, the non-human animal is genetically modified so that endogenous non-human animal hepatocytes in the liver can be selectively and conditionally ablated. In some such methods, the genetically modified non-human animal comprises a urokinase type plasminogen activator gene operably linked to a liver-specific promoter or a herpes simplex virus type 1 thymidine kinase (HSVtk) gene operably linked to a liver-specific promoter. In some such methods, the genetically modified non-human animal comprises an inactivated endogenous Fah gene. Some such methods comprise: (a) transplanting human hepatocytes or human hepatocyte progenitors into a genetically modified non-human animal comprising an inactivated endogenous Rag2 gene, an inactivated endogenous Il2rg gene, and an inactivated endogenous Fah gene; and (b) allowing the human hepatocytes or human hepatocyte progenitors to expand, wherein the genetically modified non-human animal and/or the transplanted human hepatocytes or human hepatocyte progenitors are modified to restore interleukin-6 (IL-6)/interleukin-6 receptor (IL-6R) signaling pathway activity or interleukin-6 receptor subunit beta (GP130) signaling pathway activity in the transplanted human hepatocytes or human hepatocyte progenitors. Some such methods comprise: (a) transplanting human hepatocytes or human hepatocyte progenitors into a genetically modified non-human animal comprising an inactivated endogenous Rag1 gene, an inactivated endogenous Rag2 gene, an inactivated endogenous Il2rg gene, and an inactivated endogenous Fah gene; and (b) allowing the human hepatocytes or human hepatocyte progenitors to expand, wherein the genetically modified non-human animal and/or the transplanted human hepatocytes or human hepatocyte progenitors are modified to restore interleukin-6 (IL-6)/interleukin-6 receptor (IL-6R) signaling pathway activity or interleukin-6 receptor subunit beta (GP130) signaling pathway activity in the transplanted human hepatocytes or human hepatocyte progenitors.

In some such methods, the transplanted human hepatocytes or human hepatocyte progenitors ectopically express non-human animal IL-6R (i.e., wherein the non-human animal IL-6R is from the same species as the genetically modified non-human animal, optionally wherein the genetically modified non-human animal is a mouse and the non-human animal IL-6R is mouse IL-6R, or optionally wherein the genetically modified non-human animal is a rat and the non-human animal IL-6R is rat IL-6R). In some such methods, the transplanted human hepatocytes or human hepatocyte progenitors ectopically express non-human animal OSMR (i.e., wherein the non-human animal OSMR are from the same species as the genetically modified non-human animal, optionally wherein the genetically modified non-human animal is a mouse and the non-human animal OSMR is mouse OSMR, or optionally wherein the genetically modified non-human animal is a rat and the non-human animal OSMR is rat OSMR). In some such methods, the transplanted human hepatocytes or human hepatocyte progenitors ectopically express non-human animal IL-6R, and wherein the transplanted human hepatocytes or human hepatocyte progenitors ectopically express non-human animal OSMR (i.e., wherein the non-human animal IL-6R and non-human animal OSMR are from the same species as the genetically modified non-human animal, optionally wherein the genetically modified non-human animal is a mouse, the non-human animal IL-6R is mouse IL-6R, and the non-human animal OSMR is mouse OSMR, or optionally wherein the genetically modified non-human animal is a rat, the non-human animal IL-6R is rat IL-6R, and the non-human animal OSMR is rat OSMR). In some such methods, the human hepatocytes or human hepatocyte progenitors comprise a vector comprising an expression construct for the non-human animal IL-6R comprising a nucleic acid encoding the non-human animal IL-6R operably linked to a promoter. In some such methods, the vector is a viral vector. In some such methods, the viral vector is a lentivirus vector or an adeno-associated virus (AAV) vector. In some such methods, the viral vector is a lentivirus vector. In some such methods, the human hepatocytes or human hepatocyte progenitors comprise in their genome a non-human animal IL-6R expression construct comprising a nucleic acid encoding the non-human animal IL-6R operably linked to a promoter. In some such methods, the promoter is a liver-specific promoter. In some such methods, the promoter is a constitutive promoter. In some such methods, the human hepatocytes or human hepatocyte progenitors comprise a vector comprising an expression construct for the non-human animal OSMR comprising a nucleic acid encoding the non-human animal OSMR operably linked to a promoter. In some such methods, the vector is a viral vector. In some such methods, the viral vector is a lentivirus vector or an adeno-associated virus (AAV) vector. In some such methods, the viral vector is a lentivirus vector. In some such methods, the human hepatocytes or human hepatocyte progenitors comprise in their genome a non-human animal OSMR expression construct comprising a nucleic acid encoding the non-human animal OSMR operably linked to a promoter. In some such methods, the promoter is a liver-specific promoter. In some such methods, the promoter is a constitutive promoter.

In some such methods, the transplanted human hepatocytes or human hepatocyte progenitors express a ligand-independent, constitutively active form of interleukin-6 receptor subunit beta (GP130). In some such methods, the human hepatocytes or human hepatocyte progenitors comprise a vector comprising an expression construct for the constitutively active GP130 comprising a nucleic acid encoding the constitutively active GP130 operably linked to a promoter. In some such methods, the vector is a viral vector. In some such methods, the viral vector is a lentivirus vector or an AAV vector. In some such methods, the viral vector is the lentivirus vector. In some such methods, the human hepatocytes or human hepatocyte progenitors comprise in their genome an expression construct for the constitutively active GP130 comprising a nucleic acid encoding the constitutively active GP130 operably linked to a promoter. In some such methods, the promoter is a liver-specific promoter. In some such methods, the promoter is a constitutive promoter. In some such methods, the constitutively active GP130 is a constitutively active human GP130. In some such methods, the constitutively active human GP130 comprises a deletion of the region of GP130 from Tyr186 to Tyr190 (GP130^{Y186-Y190del}).

In some such methods, the non-human animal further comprises a GP130-activating ligand. In some such methods, the GP130-activating ligand comprises human IL-6 or human-IL-6R-compatible ligand (e.g., human-IL-6R-compatible IL-6). In some such methods, the GP130-activating ligand comprises human IL-6 or human-IL-6R-compatible IL-6. In some such methods, the GP130-activating ligand comprises human OSM or human-OSMR-compatible ligand (e.g., human-OSMR-compatible OSM). In some such methods, the non-human animal further comprises one or more additional GP130-activating ligands. In some such methods, the GP130-activating ligands comprise: (1) human IL-6 or human-IL-6R-compatible ligand (e.g., human-IL-6R-compatible IL-6); and (2) human OSM or human-OSMR-compatible ligand (e.g., human-OSMR-compatible OSM). In some such methods, the non-human animal comprises a vector comprising an expression construct for GP130-activating ligand comprising a nucleic acid encoding the GP130-activating ligand operably linked to a promoter. In some such methods, the non-human animal comprises the vector in muscle cells. In some such methods, the promoter is a tissue-specific promoter, optionally wherein the tissue-specific promoter is a muscle-specific promoter, optionally wherein the muscle-specific promoter is a hybrid mouse alpha-myosin heavy-chain (MH) and muscle creatine kinase (CK) promoter (MHCK7). In some such methods, the promoter is a constitutive promoter. In some such methods, the vector is a viral vector. In some such methods, the viral vector is a lentivirus vector or an adeno-associated virus (AAV) vector. In some such methods, the viral vector is the AAV vector, optionally wherein the AAV vector is a recombinant AAV9 vector. In some such methods, the non-human animal comprises in its genome the GP130-activating ligand expression construct comprising a nucleic acid encoding the GP130-activating ligand operably linked to a promoter. In some such methods, the GP130-activating ligand comprises human IL-6 or human-IL-6R-compatible ligand (e.g., human-IL-6R-compatible IL-6). In some such methods, the GP130-activating ligand comprises human IL-6 or human-IL-6R-compatible IL-6. In some such methods, the GP130-activating ligand comprises human OSM or human-OSMR-compatible ligand (e.g., human-OSMR-compatible OSM). In some such methods, the promoter is a tissue-specific promoter, optionally wherein the tissue-specific promoter is a muscle-specific promoter, optionally wherein the muscle-specific promoter is a hybrid mouse alpha-myosin heavy-chain (MH) and muscle creatine kinase (CK) promoter (MHCK7). In some such methods, the promoter is a constitutive promoter. In some such methods, the promoter is an exogenous promoter. In some such methods, the promoter is an endogenous promoter.

In some such methods, the non-human animal comprises a humanized non-human animal IL6 gene comprising a human IL6 nucleic acid encoding a human IL-6 protein. In some such methods, the human nucleic acid comprises a region of human IL6 genomic sequence from the start codon to the stop codon. In some such methods, the human nucleic acid comprises a human IL6 complementary DNA (cDNA). In some such methods, the human nucleic acid replaces a corresponding region of the non-human animal IL6 gene. In some such methods, the human nucleic acid is inserted into the non-human animal IL6 gene. In some such methods, the human nucleic acid is operably linked to an endogenous non-human animal IL6 promoter. In some such methods, the non-human animal is heterozygous for the humanized IL6 gene. In some such methods, the non-human animal is homozygous for the humanized IL6 gene. In some such methods, the non-human animal comprises the humanized IL6 gene in its germline. In some such methods, the non-human animal comprises a humanized non-human animal OSM gene comprising a human OSM nucleic acid encoding a human OSM protein. In some such methods, the human nucleic acid comprises a region of human OSM genomic sequence from the start codon to the stop codon. In some such methods, the human nucleic acid comprises a human OSM complementary DNA (cDNA). In some such methods, the human nucleic acid replaces a corresponding region of the non-human animal OSM gene. In some such methods, the human nucleic acid is inserted into the non-human animal OSM gene. In some such methods, the human nucleic acid is operably linked to an endogenous non-human animal OSM promoter. In some such methods, the non-human animal is heterozygous for the humanized OSM gene. In some such methods, the non-human animal is homozygous for the humanized OSM gene. In some such methods, the non-human animal comprises the humanized OSM gene in its germline. In some such methods, the non-human animal comprises a humanized non-human animal OSM gene comprising a human OSM nucleic acid encoding a human OSM protein and a humanized non-human animal IL6 gene comprising a human IL6 nucleic acid encoding a human IL-6 protein.

In some such methods, the non-human animal does not comprise human Kupffer cells in the liver. In some such methods, the non-human animal does not comprise a reconstituted human immune system. In some such methods, the method does not comprise administering human Kupffer cells to the non-human animal. In some such methods, the method does not comprise reconstituting a human immune system in the non-human animal.

In some such methods, the method further comprises modifying the human hepatocytes or human hepatocyte progenitors and/or modifying the non-human animal to restore interleukin-6 (IL-6)/interleukin-6 receptor (IL-6R) signaling pathway activity or interleukin-6 receptor subunit beta (GP130) signaling pathway activity in the transplanted human hepatocytes or human hepatocyte progenitors. In some such methods, the method comprises modifying the human hepatocytes or human hepatocyte progenitors prior to step (a) to ectopically express non-human animal IL-6R (i.e., wherein the non-human animal IL-6R is from the same species as the genetically modified non-human animal, optionally wherein the genetically modified non-human animal is a mouse and the non-human animal IL-6R is mouse IL-6R, or optionally wherein the genetically modified non-human animal is a rat and the non-human animal IL-6R is rat IL-6R). In some such methods, the method comprises modifying the human hepatocytes or human hepatocyte progenitors prior to step (a) to ectopically express non-human animal OSMR (i.e., wherein the non-human animal OSMR is from the same species as the genetically modified non-human animal, optionally wherein the genetically modified non-human animal is a mouse and the non-human animal OSMR is mouse OSMR, or optionally wherein the genetically modified non-human animal is a rat and the non-human animal OSMR is rat OSMR). In some such methods, the method comprises modifying the human hepatocytes or human hepatocyte progenitors prior to step (a) to ectopically express non-human animal OSMR and modifying the human hepatocytes or human hepatocyte progenitors prior to step (a) to ectopically express non-human animal IL-6R (i.e., wherein the non-human animal IL-6R and non-human animal OSMR are from the same species as the genetically modified non-human animal, optionally wherein the genetically modified non-human animal is a mouse, the non-human animal IL-6R is mouse IL-6R, and the non-human animal OSMR is mouse OSMR, or optionally wherein the genetically modified non-human animal is a rat, the non-human animal IL-6R is rat IL-6R, and the non-human animal OSMR is rat OSMR). In some such methods, the method comprises modifying the human hepatocytes or human hepatocyte progenitors prior to step (a) to comprise a vector comprising an expression construct for the non-human animal IL-6R comprising a nucleic acid encoding the non-human animal IL-6R operably linked to a promoter. In some such methods, the vector is a viral vector. In some such methods, the viral vector is a lentivirus vector or an adeno-associated virus (AAV) vector. In some such methods, the viral vector is a lentivirus vector. In some such methods, the method comprises modifying the human hepatocytes or human hepatocyte progenitors prior to step (a) to comprise in their genome a non-human animal IL-6R expression construct comprising a nucleic acid encoding the non-human animal IL-6R operably linked to a promoter. In some such methods, the promoter is a liver-specific promoter. In some such methods, the promoter is a constitutive promoter. In some such methods, the method comprises modifying the human hepatocytes or human hepatocyte progenitors prior to step (a) to comprise a vector comprising an expression construct for the non-human animal OSMR comprising a nucleic acid encoding the non-human animal OSMR operably linked to a promoter. In some such methods, the vector is a viral vector. In some such methods, the viral vector is a lentivirus vector or an adeno-associated virus (AAV) vector. In some such methods, the viral vector is a lentivirus vector. In some such methods, the method comprises modifying the human hepatocytes or human hepatocyte progenitors prior to step (a) to comprise in their genome a non-human animal OSMR expression construct comprising a nucleic acid encoding the non-human animal OSMR operably linked to a promoter. In some such methods, the promoter is a liver-specific promoter. In some such methods, the promoter is a constitutive promoter.

In some such methods, the method comprises modifying the human hepatocytes or human hepatocyte progenitors prior to step (a) to express a ligand-independent, constitutively active form of interleukin-6 receptor subunit beta (GP130). In some such methods, the method comprises modifying the human hepatocytes or human hepatocyte progenitors prior to step (a) to comprise a vector comprising an expression construct for the constitutively active GP130 comprising a nucleic acid encoding the constitutively active GP130 operably linked to a promoter. In some such methods, the vector is a viral vector. In some such methods, the viral vector is a lentivirus vector or an AAV vector. In some such methods, the viral vector is the lentivirus vector. In some such methods, the method comprises modifying the human hepatocytes or human hepatocyte progenitors prior to step (a) to comprise in their genome an expression construct for the constitutively active GP130 comprising a nucleic acid encoding the constitutively active GP130 operably linked to a promoter. In some such methods, the promoter is a liver-specific promoter. In some such methods, the promoter is a constitutive promoter. In some such methods, the constitutively active GP130 is a constitutively active human GP130. In some such methods, the constitutively active human GP130 comprises a deletion of the region of GP130 from Tyr186 to Tyr190 (GP130^{Y186-Y190del}).

In some such methods, the method further comprises modifying the non-human animal to comprise or express a GP130-activating ligand. In some such methods, the GP130-activating ligand comprises human IL-6 or human-IL-6R-compatible ligand (e.g., human-IL-6R-compatible IL-6). In some such methods, the GP130-activating ligand comprises human IL-6 or human-IL-6R-compatible IL-6. In some such methods, the GP130-activating ligand comprises human OSM or human-OSMR-compatible ligand (e.g., human-OSMR-compatible OSM). In some such methods, the method further comprises modifying the non-human animal to comprise or express one or more additional GP130-activating ligands. In some such methods, the GP130-activating ligands comprise: (1) human IL-6 or human-IL-6R-compatible ligand (e.g., human-IL-6R-compatible IL-6); and (2) human OSM or human-OSMR-compatible ligand (e.g., human-OSMR-compatible OSM). In some such methods, the modifying the non-human animal to comprise or express the GP130-activating ligand occurs prior to step (a). In some such methods, the modifying the non-human animal to comprise or express the GP130-activating ligand occurs after step (a). In some such methods, the non-human animal is modified to comprise a vector comprising an expression construct for the GP130-activating ligand comprising a nucleic acid encoding the GP130-activating ligand operably linked to a promoter. In some such methods, the GP130-activating ligand comprises human IL-6 or human-IL-6R-compatible ligand (e.g., human-IL-6R-compatible IL-6). In some such methods, the GP130-activating ligand comprises human IL-6 or human-IL-6R-compatible IL-6. In some such methods, the GP130-activating ligand comprises human OSM or human-OSMR-compatible ligand (e.g., human-OSMR-compatible OSM). In some such methods, the non-human animal is modified to comprise the vector in muscle cells. In some such methods, the promoter is a tissue-specific promoter, optionally wherein the tissue-specific promoter is a muscle-specific promoter, optionally wherein the muscle-specific promoter is a hybrid mouse alpha-myosin heavy-chain (MH) and muscle creatine kinase (CK) promoter (MHCK7). In some such methods, the promoter is a constitutive promoter. In some such methods, the vector is a viral vector. In some such methods, the viral vector is a lentivirus vector or an adeno-associated virus (AAV) vector. In some such methods, the viral vector is the AAV vector, optionally wherein the AAV vector is a recombinant AAV9 vector. In some such methods, the non-human animal is modified to comprise in its genome an expression construct for the GP130-activating ligand comprising a nucleic acid encoding the GP130-activating ligand operably linked to a promoter. In some such methods, the promoter is a tissue-specific promoter, optionally wherein the tissue-specific promoter is a muscle-specific promoter, optionally wherein the muscle-specific promoter is a hybrid mouse alpha-myosin heavy-chain (MH) and muscle creatine kinase (CK) promoter (MHCK7). In some such methods, the promoter is a constitutive promoter. In some such methods, the promoter is an exogenous promoter. In some such methods, the promoter is an endogenous promoter.

In some such methods, the non-human animal further comprises a humanized non-human animal SIRPA gene. In some such methods, the humanized non-human animal SIRPA gene comprises a replacement of exons 2-4 of the non-human animal SIRPA gene with exons 2-4 of human SIRPA, wherein the humanized non-human animal SIRPA gene encodes a chimeric SIRPA protein comprising an extracellular portion of a human SIRPA protein and an intracellular portion of a non-human animal SIRPA protein. In some such methods, the humanized non-human animal SIRPA gene is operably linked to an endogenous non-human animal SIRPA promoter. In some such methods, the non-human animal is heterozygous for the humanized SIRPA gene. In some such methods, the non-human animal is homozygous for the humanized SIRPA gene. In some such methods, the non-human animal comprises the humanized SIRPA gene in its germline.

In some such methods, the non-human animal is a male. In some such methods, the non-human animal is a female. In some such methods, the non-human animal is a mammal. In some such methods, the mammal is a rodent. In some such methods, the rodent is a rat or a mouse. In some such methods, the rodent is the rat. In some such methods, the rodent is the mouse.

In some such methods, exogenous urokinase plasminogen activator or an exogenous nucleic acid encoding urokinase plasminogen activator is administered to the genetically modified non-human animal prior to step (a) to prime the liver for improved repopulation by human hepatocytes, optionally wherein the exogenous nucleic acid is an adenovirus or adeno-associated virus (AAV) encoding urokinase plasminogen activator. In some such methods, exogenous herpes simplex virus type 1 thymidine kinase (HSVtk) or an exogenous nucleic acid encoding HSVtk is administered to the genetically modified non-human animal prior to step (a) to prime the liver for improved repopulation by human hepatocytes, optionally wherein the exogenous nucleic acid is an adenovirus or adeno-associated virus (AAV) encoding HSVtk. In some such methods, the human hepatocytes or human hepatocyte progenitors are injected into the genetically modified non-human animal intrasplenically in step (a). In some such methods, at least about ten million human hepatocytes or human hepatocyte progenitors are transplanted into the genetically modified non-human animal in step (a). In some such methods, step (a) is done in the absence of nitisinone or any other compound that ameliorates toxicity caused by Fah deficiency. In some such methods, some or all of step (b) is done in the absence of nitisinone or any other compound that ameliorates toxicity caused by Fah deficiency. In some such methods, nitisinone or any other compound that ameliorates toxicity caused by Fah deficiencies is administered to the non-human animals in step (b) in an on/off cycle to promote human hepatocyte repopulation. In some such methods, the on/off cycle comprises about 5 to about 7 days off and about 3 days on.

In some such methods, the humanized liver generated by the method has reduced lipid droplet accumulation or reduced steatosis compared to methods in which the genetically modified non-human animal and the transplanted human hepatocytes or human hepatocyte progenitors are not modified to restore IL-6/IL-6R signaling pathway activity or interleukin-6 receptor subunit beta (GP130) signaling pathway activity in the transplanted human hepatocytes or human hepatocyte progenitors.

In another aspect, provided is a genetically modified non-human animal, non-human animal cell, or non-human animal genome comprising an inactivated endogenous Rag2 gene, an inactivated endogenous Il2rg gene, an inactivated endogenous Fah gene, and a humanized IL6 gene comprising a human IL6 nucleic acid encoding a human IL-6 protein. In another aspect, provided is a genetically modified non-human animal, non-human animal cell, or non-human animal genome comprising an inactivated endogenous Rag1 gene, an inactivated endogenous Rag2 gene, an inactivated endogenous Il2rg gene, an inactivated endogenous Fah gene, and a humanized IL6 gene comprising a human IL6 nucleic acid encoding a human IL-6 protein. In some such animals, cells, or genomes, the human nucleic acid comprises a region of human IL6 genomic sequence from the start codon to the stop codon. In some such animals, cells, or genomes, the human nucleic acid comprises a human IL6 complementary DNA (cDNA). In some such animals, cells, or genomes, the human nucleic acid replaces a corresponding region of the non-human animal IL6 gene. In some such animals, cells, or genomes, the human nucleic acid is inserted into the non-human animal IL6 gene. In some such animals, cells, or genomes, the human nucleic acid is operably linked to an endogenous non-human animal IL6 promoter. In some such animals, cells, or genomes, the non-human animal, cell, or genome is heterozygous for the humanized IL6 gene. In some such animals, cells, or genomes, the non-human animal, cell, or genome is homozygous for the humanized IL6 gene. In some such animals, cells, or genomes, the non-human animal comprises the humanized IL6 gene in its germline. In another aspect, provided is a genetically modified non-human animal, non-human animal cell, or non-human animal genome comprising an inactivated endogenous Rag2 gene, an inactivated endogenous Il2rg gene, an inactivated endogenous Fah gene, and a humanized OSM gene comprising a human OSM nucleic acid encoding a human OSM protein. In another aspect, provided is a genetically modified non-human animal, non-human animal cell, or non-human animal genome comprising an inactivated endogenous Rag1 gene, an inactivated endogenous Rag2 gene, an inactivated endogenous Il2rg gene, an inactivated endogenous Fah gene, and a humanized OSM gene comprising a human OSM nucleic acid encoding a human OSM protein. In some such animals, cells, or genomes, the human nucleic acid comprises a region of human OSM genomic sequence from the start codon to the stop codon. In some such animals, cells, or genomes, the human nucleic acid comprises a human OSM complementary DNA (cDNA). In some such animals, cells, or genomes, the human nucleic acid replaces a corresponding region of the non-human animal OSM gene. In some such animals, cells, or genomes, the human nucleic acid is inserted into the non-human animal OSM gene. In some such animals, cells, or genomes, the human nucleic acid is operably linked to an endogenous non-human animal OSM promoter. In some such animals, cells, or genomes, the non-human animal is heterozygous for the humanized OSM gene. In some such animals, cells, or genomes, the non-human animal is homozygous for the humanized OSM gene. In some such animals, cells, or genomes, the non-human animal comprises the humanized OSM gene in its germline. In another aspect, provided is a genetically modified non-human animal, non-human animal cell, or non-human animal genome comprising an inactivated endogenous Rag2 gene, an inactivated endogenous Il2rg gene, an inactivated endogenous Fah gene, a humanized IL6 gene comprising a human IL6 nucleic acid encoding a human IL-6 protein, and a humanized OSM gene comprising a human OSM nucleic acid encoding a human OSM protein. In another aspect, provided is a genetically modified non-human animal, non-human animal cell, or non-human animal genome comprising an inactivated endogenous Rag1 gene, an inactivated endogenous Rag2 gene, an inactivated endogenous Il2rg gene, an inactivated endogenous Fah gene, a humanized IL6 gene comprising a human IL6 nucleic acid encoding a human IL-6 protein, and a humanized OSM gene comprising a human OSM nucleic acid encoding a human OSM protein.

In some such animals, cells, or genomes, the animal, cell, or genome further comprises a humanized non-human animal SIRPA gene. In some such animals, cells, or genomes, the humanized non-human animal SIRPA gene comprises a replacement of exons 2-4 of the non-human animal SIRPA gene with exons 2-4 of human SIRPA, wherein the humanized non-human animal SIRPA gene encodes a chimeric SIRPA protein comprising an extracellular portion of a human SIRPA protein and an intracellular portion of a non-human animal SIRPA protein. In some such animals, cells, or genomes, the humanized non-human animal SIRPA gene is operably linked to an endogenous non-human animal SIRPA promoter. In some such animals, cells, or genomes, the non-human animal is heterozygous for the humanized SIRPA gene. In some such animals, cells, or genomes, the non-human animal is homozygous for the humanized SIRPA gene. In some such animals, cells, or genomes, the non-human animal comprises the humanized SIRPA gene in its germline.

In some such animals, cells, or genomes, the non-human animal is a male. In some such animals, cells, or genomes, the non-human animal is a female. In some such animals, cells, or genomes, the non-human animal is a mammal. In some such animals, cells, or genomes, the mammal is a rodent. In some such animals, cells, or genomes, the rodent is a rat or a mouse. In some such animals, cells, or genomes, the rodent is the rat. In some such animals, cells, or genomes, the rodent is the mouse.

In another aspect, provided are methods of making any of the above genetically modified non-human animals. Some such methods comprise: (a) introducing a genetically modified non-human animal embryonic stem (ES) cell comprising an inactivated endogenous Rag2 gene, an inactivated endogenous Il2rg gene, an inactivated endogenous Fah gene, and a humanized IL6 gene comprising a human IL6 nucleic acid encoding a human IL-6 protein into a non-human animal host embryo; and (b) implanting and gestating the non-human animal host embryo in a non-human animal surrogate mother, wherein the non-human animal surrogate mother produces an F0 progeny genetically modified non-human animal comprising the inactivated endogenous Rag2 gene, the inactivated endogenous Il2rg gene, the inactivated endogenous Fah gene, and the humanized IL6 gene. Some such methods further comprise modifying a non-human animal ES cell to generate the genetically modified non-human animal ES cell comprising the inactivated endogenous Rag2 gene, the inactivated endogenous Il2rg gene, the inactivated endogenous Fah gene, and the humanized IL6 gene prior to step (a). Some such methods comprise implanting and gestating a genetically modified non-human animal one-cell stage embryo comprising an inactivated endogenous Rag2 gene, an inactivated endogenous Il2rg gene, an inactivated endogenous Fah gene, and a humanized IL6 gene comprising a human IL6 nucleic acid encoding a human IL-6 protein in a non-human animal surrogate mother, wherein the non-human animal surrogate mother produces an F0 progeny genetically modified non-human animal comprising the inactivated endogenous Rag2 gene, the inactivated endogenous Il2rg gene, the inactivated endogenous Fah gene, and the humanized IL6 gene. Some such methods further comprise modifying a non-human animal one-cell stage embryo to generate the genetically modified non-human animal one-cell stage embryo comprising the inactivated endogenous Rag2 gene, the inactivated endogenous Il2rg gene, the inactivated endogenous Fah gene, and the humanized IL6 gene prior to gestating the genetically modified non-human animal one-cell stage embryo in the non-human animal surrogate mother. Some such methods comprise: (a) introducing a genetically modified non-human animal embryonic stem (ES) cell comprising an inactivated endogenous Rag2 gene, an inactivated endogenous Il2rg gene, an inactivated endogenous Fah gene, and a humanized OSM gene comprising a human OSM nucleic acid encoding a human OSM protein into a non-human animal host embryo; and (b) implanting and gestating the non-human animal host embryo in a non-human animal surrogate mother, wherein the non-human animal surrogate mother produces an F0 progeny genetically modified non-human animal comprising the inactivated endogenous Rag2 gene, the inactivated endogenous Il2rg gene, the inactivated endogenous Fah gene, and the humanized OSM gene. Some such methods further comprise modifying a non-human animal ES cell to generate the genetically modified non-human animal ES cell comprising the inactivated endogenous Rag2 gene, the inactivated endogenous Il2rg gene, the inactivated endogenous Fah gene, and the humanized OSM gene prior to step (a). Some such methods comprise implanting and gestating a genetically modified non-human animal one-cell stage embryo comprising an inactivated endogenous Rag2 gene, an inactivated endogenous Il2rg gene, an inactivated endogenous Fah gene, and a humanized OSM gene comprising a human OSM nucleic acid encoding a human OSM protein in a non-human animal surrogate mother, wherein the non-human animal surrogate mother produces an F0 progeny genetically modified non-human animal comprising the inactivated endogenous Rag2 gene, the inactivated endogenous Il2rg gene, the inactivated endogenous Fah gene, and the humanized IL6 gene. Some such methods further comprise modifying a non-human animal one-cell stage embryo to generate the genetically modified non-human animal one-cell stage embryo comprising the inactivated endogenous Rag2 gene, the inactivated endogenous Il2rg gene, the inactivated endogenous Fah gene, and the humanized OSM gene prior to gestating the genetically modified non-human animal one-cell stage embryo in the non-human animal surrogate mother. Some such methods comprise: (a) introducing a genetically modified non-human animal embryonic stem (ES) cell comprising an inactivated endogenous Rag2 gene, an inactivated endogenous Il2rg gene, an inactivated endogenous Fah gene, a humanized IL6 gene comprising a human IL6 nucleic acid encoding a human IL-6 protein, and a humanized OSM gene comprising a human OSM nucleic acid encoding a human OSM protein into a non-human animal host embryo; and (b) implanting and gestating the non-human animal host embryo in a non-human animal surrogate mother, wherein the non-human animal surrogate mother produces an F0 progeny genetically modified non-human animal comprising the inactivated endogenous Rag2 gene, the inactivated endogenous Il2rg gene, the inactivated endogenous Fah gene, the humanized IL6 gene, and the humanized OSM gene. Some such methods further comprise modifying a non-human animal ES cell to generate the genetically modified non-human animal ES cell comprising the inactivated endogenous Rag2 gene, the inactivated endogenous Il2rg gene, the inactivated endogenous Fah gene, the humanized IL6 gene, and the humanized OSM gene prior to step (a). Some such methods comprise implanting and gestating a genetically modified non-human animal one-cell stage embryo comprising an inactivated endogenous Rag2 gene, an inactivated endogenous Il2rg gene, an inactivated endogenous Fah gene, a humanized IL6 gene comprising a human IL6 nucleic acid encoding a human IL-6 protein, and a humanized OSM gene comprising a human OSM nucleic acid encoding a human OSM protein in a non-human animal surrogate mother, wherein the non-human animal surrogate mother produces an F0 progeny genetically modified non-human animal comprising the inactivated endogenous Rag2 gene, the inactivated endogenous Il2rg gene, the inactivated endogenous Fah gene, the humanized IL6 gene, and the humanized OSM gene. Some such methods further comprise modifying a non-human animal one-cell stage embryo to generate the genetically modified non-human animal one-cell stage embryo comprising the inactivated endogenous Rag2 gene, the inactivated endogenous Il2rg gene, the inactivated endogenous Fah gene, the humanized IL6 gene, and the humanized OSM gene prior to gestating the genetically modified non-human animal one-cell stage embryo in the non-human animal surrogate mother. In another aspect, provided are methods of making any of the above genetically modified non-human animals. Some such methods comprise: (a) introducing a genetically modified non-human animal embryonic stem (ES) cell comprising an inactivated endogenous Rag1 gene, an inactivated endogenous Rag2 gene, an inactivated endogenous Il2rg gene, an inactivated endogenous Fah gene, and a humanized IL6 gene comprising a human IL6 nucleic acid encoding a human IL-6 protein into a non-human animal host embryo; and (b) implanting and gestating the non-human animal host embryo in a non-human animal surrogate mother, wherein the non-human animal surrogate mother produces an F0 progeny genetically modified non-human animal comprising the inactivated endogenous Rag1 gene, the inactivated endogenous Rag2 gene, the inactivated endogenous Il2rg gene, the inactivated endogenous Fah gene, and the humanized IL6 gene. Some such methods further comprise modifying a non-human animal ES cell to generate the genetically modified non-human animal ES cell comprising the inactivated endogenous Rag1 gene, the inactivated endogenous Rag2 gene, the inactivated endogenous Il2rg gene, the inactivated endogenous Fah gene, and the humanized IL6 gene prior to step (a). Some such methods comprise implanting and gestating a genetically modified non-human animal one-cell stage embryo comprising an inactivated endogenous Rag1 gene, an inactivated endogenous Rag2 gene, an inactivated endogenous Il2rg gene, an inactivated endogenous Fah gene, and a humanized IL6 gene comprising a human IL6 nucleic acid encoding a human IL-6 protein in a non-human animal surrogate mother, wherein the non-human animal surrogate mother produces an F0 progeny genetically modified non-human animal comprising the inactivated endogenous Rag1 gene, the inactivated endogenous Rag2 gene, the inactivated endogenous Il2rg gene, the inactivated endogenous Fah gene, and the humanized IL6 gene. Some such methods further comprise modifying a non-human animal one-cell stage embryo to generate the genetically modified non-human animal one-cell stage embryo comprising the inactivated endogenous Rag1 gene, the inactivated endogenous Rag2 gene, the inactivated endogenous Il2rg gene, the inactivated endogenous Fah gene, and the humanized IL6 gene prior to gestating the genetically modified non-human animal one-cell stage embryo in the non-human animal surrogate mother. Some such methods comprise: (a) introducing a genetically modified non-human animal embryonic stem (ES) cell comprising an inactivated endogenous Rag1 gene, an inactivated endogenous Rag2 gene, an inactivated endogenous Il2rg gene, an inactivated endogenous Fah gene, and a humanized OSM gene comprising a human OSM nucleic acid encoding a human OSM protein into a non-human animal host embryo; and (b) implanting and gestating the non-human animal host embryo in a non-human animal surrogate mother, wherein the non-human animal surrogate mother produces an F0 progeny genetically modified non-human animal comprising the inactivated endogenous Rag1 gene, the inactivated endogenous Rag2 gene, the inactivated endogenous Il2rg gene, the inactivated endogenous Fah gene, and the humanized OSM gene. Some such methods further comprise modifying a non-human animal ES cell to generate the genetically modified non-human animal ES cell comprising the inactivated endogenous Rag1 gene, the inactivated endogenous Rag2 gene, the inactivated endogenous Il2rg gene, the inactivated endogenous Fah gene, and the humanized OSM gene prior to step (a). Some such methods comprise implanting and gestating a genetically modified non-human animal one-cell stage embryo comprising an inactivated endogenous Rag1 gene, an inactivated endogenous Rag2 gene, an inactivated endogenous Il2rg gene, an inactivated endogenous Fah gene, and a humanized OSM gene comprising a human OSM nucleic acid encoding a human OSM protein in a non-human animal surrogate mother, wherein the non-human animal surrogate mother produces an F0 progeny genetically modified non-human animal comprising the inactivated endogenous Rag1 gene, the inactivated endogenous Rag2 gene, the inactivated endogenous Il2rg gene, the inactivated endogenous Fah gene, and the humanized IL6 gene. Some such methods further comprise modifying a non-human animal one-cell stage embryo to generate the genetically modified non-human animal one-cell stage embryo comprising the inactivated endogenous Rag1 gene, the inactivated endogenous Rag2 gene, the inactivated endogenous Il2rg gene, the inactivated endogenous Fah gene, and the humanized OSM gene prior to gestating the genetically modified non-human animal one-cell stage embryo in the non-human animal surrogate mother. Some such methods comprise: (a) introducing a genetically modified non-human animal embryonic stem (ES) cell comprising an inactivated endogenous Rag1 gene, an inactivated endogenous Rag2 gene, an inactivated endogenous Il2rg gene, an inactivated endogenous Fah gene, a humanized IL6 gene comprising a human IL6 nucleic acid encoding a human IL-6 protein, and a humanized OSM gene comprising a human OSM nucleic acid encoding a human OSM protein into a non-human animal host embryo; and (b) implanting and gestating the non-human animal host embryo in a non-human animal surrogate mother, wherein the non-human animal surrogate mother produces an F0 progeny genetically modified non-human animal comprising the inactivated endogenous Rag1 gene, the inactivated endogenous Rag2 gene, the inactivated endogenous Il2rg gene, the inactivated endogenous Fah gene, the humanized IL6 gene, and the humanized OSM gene. Some such methods further comprise modifying a non-human animal ES cell to generate the genetically modified non-human animal ES cell comprising the inactivated endogenous Rag1 gene, the inactivated endogenous Rag2 gene, the inactivated endogenous Il2rg gene, the inactivated endogenous Fah gene, the humanized IL6 gene, and the humanized OSM gene prior to step (a). Some such methods comprise implanting and gestating a genetically modified non-human animal one-cell stage embryo comprising an inactivated endogenous Rag1 gene, an inactivated endogenous Rag2 gene, an inactivated endogenous Il2rg gene, an inactivated endogenous Fah gene, a humanized IL6 gene comprising a human IL6 nucleic acid encoding a human IL-6 protein, and a humanized OSM gene comprising a human OSM nucleic acid encoding a human OSM protein in a non-human animal surrogate mother, wherein the non-human animal surrogate mother produces an F0 progeny genetically modified non-human animal comprising the inactivated endogenous Rag1 gene, the inactivated endogenous Rag2 gene, the inactivated endogenous Il2rg gene, the inactivated endogenous Fah gene, the humanized IL6 gene, and the humanized OSM gene. Some such methods further comprise modifying a non-human animal one-cell stage embryo to generate the genetically modified non-human animal one-cell stage embryo comprising the inactivated endogenous Rag1 gene, the inactivated endogenous Rag2 gene, the inactivated endogenous Il2rg gene, the inactivated endogenous Fah gene, the humanized IL6 gene, and the humanized OSM gene prior to gestating the genetically modified non-human animal one-cell stage embryo in the non-human animal surrogate mother.

In another aspect, provided are methods of making a non-human animal with a humanized liver. Some such methods comprise: (a) transplanting human hepatocytes or human hepatocyte progenitors into any of the above genetically modified non-human animals; (b) allowing the human hepatocytes or human hepatocyte progenitors to expand. In some such methods, exogenous urokinase plasminogen activator or an exogenous nucleic acid encoding urokinase plasminogen activator is administered to the genetically modified non-human animal prior to step (a) to prime the liver for improved repopulation by human hepatocytes, optionally wherein the exogenous nucleic acid is an adenovirus or adeno-associated virus (AAV) encoding urokinase plasminogen activator. In some such methods, exogenous herpes simplex virus type 1 thymidine kinase (HSVtk) or an exogenous nucleic acid encoding HSVtk is administered to the genetically modified non-human animal prior to step (a) to prime the liver for improved repopulation by human hepatocytes, optionally wherein the exogenous nucleic acid is an adenovirus or adeno-associated virus (AAV) encoding HSVtk. In some such methods, the human hepatocytes or human hepatocyte progenitors are injected into the genetically modified non-human animal intrasplenically in step (a). In some such methods, at least about ten million human hepatocytes or human hepatocyte progenitors are transplanted into the genetically modified non-human animal in step (a). In some such methods, step (a) is done in the absence of nitisinone or any other compound that ameliorates toxicity caused by Fah deficiency. In some such methods, some or all of step (b) is done in the absence of nitisinone or any other compound that ameliorates toxicity caused by Fah deficiency. In some such methods, nitisinone or any other compound that ameliorates toxicity caused by Fah deficiencies is administered to the non-human animals in step (b) in an on/off cycle to promote human hepatocyte repopulation. In some such methods, the on/off cycle comprises about 5 to about 7 days off and about 3 days on.

In another aspect, provided are methods of preventing, reducing, or ameliorating hepatosteatosis in a non-human animal comprising transplanted human hepatocytes, comprising administering a GP130-activating ligand or a nucleic acid encoding the GP130-activating ligand to the non-human animal, wherein the GP130-activating ligand restores interleukin-6 (IL-6)/interleukin-6 receptor (IL-6R) signaling pathway activity or interleukin-6 receptor subunit beta (GP130) signaling pathway activity in the transplanted human hepatocytes, and provided are methods of preventing, reducing, or ameliorating lipid droplet accumulation in transplanted human hepatocytes in a non-human animal comprising the transplanted human hepatocytes, comprising administering a GP130-activating ligand or a nucleic acid encoding the GP130-activating ligand to the non-human animal, wherein the GP130-activating ligand restores interleukin-6 (IL-6)/interleukin-6 receptor (IL-6R) signaling pathway activity or interleukin-6 receptor subunit beta (GP130) signaling pathway activity in the transplanted human hepatocytes.

In some such methods, the GP130-activating ligand comprises human IL-6 or human-IL-6R-compatible ligand (e.g., human-IL-6R-compatible IL-6). In some such methods, the GP130-activating ligand comprises human IL-6 or human-IL-6R-compatible IL-6. In some such methods, the GP130-activating ligand comprises human OSM or human-OSMR-compatible ligand (e.g., human-OSMR-compatible OSM). In some such methods, the method further comprises administering one or more additional GP130-activating ligands or nucleic acids encoding the one or more additional GP130-activating ligand to the non-human animal. In some such methods, the GP130-activating ligands comprise: (1) human IL-6 or human-IL-6R-compatible ligand (e.g., human-IL-6R-compatible IL-6); and (2) human OSM or human-OSMR-compatible ligand (e.g., human-OSMR-compatible OSM). In some such methods, the method comprises administering the nucleic acid, wherein the nucleic acid comprises a vector comprising an expression construct for the GP130-activating ligand comprising the nucleic acid encoding human IL-6 operably linked to a promoter. In some such methods, the nucleic acid is administered to liver cells or muscle cells. In some such methods, the promoter is a tissue-specific promoter, optionally wherein the tissue-specific promoter is a liver-specific promoter or a muscle-specific promoter, optionally wherein the muscle-specific promoter is a hybrid mouse alpha-myosin heavy-chain (MH) and muscle creatine kinase (CK) promoter (MHCK7). In some such methods, the promoter is a constitutive promoter. In some such methods, the vector is a viral vector. In some such methods, the viral vector is a lentivirus vector or an adeno-associated virus (AAV) vector. In some such methods, the viral vector is the AAV vector, optionally wherein the AAV vector is a recombinant AAV9 vector. In some such methods, the method comprises administering the GP130-activating ligand, optionally wherein the GP130-activating ligand is administered to the liver of the non-human animal. In some such methods, the non-human animal is a male. In some such methods, the non-human animal is a female. In some such methods, the non-human animal is a mammal. In some such methods, the mammal is a rodent. In some such methods, the rodent is a rat or a mouse. In some such methods, the rodent is the rat. In some such methods, the rodent is the mouse.

In some such methods, the non-human animal is immunodeficient. In some such methods, the non-human animal comprises an inactivated endogenous Il2rg gene. In some such methods, the non-human animal comprises an inactivated endogenous Rag1 gene and/or an inactivated endogenous Rag2 gene. In some such methods, the non-human animal comprises an inactivated endogenous Rag2 gene. In some such methods, the non-human animal comprises an inactivated endogenous Rag1 gene and an inactivated endogenous Rag2 gene. In some such methods, the non-human animal comprises an inactivated endogenous Rag2 gene and an inactivated endogenous Il2rg gene. In some such methods, the non-human animal comprises an inactivated endogenous Rag1 gene, an inactivated endogenous Rag2 gene, and an inactivated endogenous Il2rg gene. In some such methods, the non-human animal comprises a severe combined immunodeficiency (SCID) mutation in a Prkdc gene (Prkdc^{scid}). In some such methods, the non-human animal is genetically modified so that endogenous non-human animal hepatocytes in the liver can be selectively and conditionally ablated. In some such methods, the non-human animal comprises a urokinase type plasminogen activator gene operably linked to a liver-specific promoter or a herpes simplex virus type 1 thymidine kinase (HSVtk) gene operably linked to a liver-specific promoter. In some such methods, the non-human animal comprises an inactivated endogenous Fah gene. In some such methods, the non-human animal comprises an inactivated endogenous Rag2 gene, an inactivated endogenous Il2rg gene, and an inactivated endogenous Fah gene. In some such methods, the non-human animal comprises an inactivated endogenous Rag1 gene, an inactivated endogenous Rag2 gene, an inactivated endogenous Il2rg gene, and an inactivated endogenous Fah gene.

In some such methods, the non-human animal comprises a humanized non-human animal SIRPA gene. In some such methods, the humanized non-human animal SIRPA gene comprises a replacement of exons 2-4 of the non-human animal SIRPA gene with exons 2-4 of human SIRPA, wherein the humanized non-human animal SIRPA gene encodes a chimeric SIRPA protein comprising an extracellular portion of a human SIRPA protein and an intracellular portion of a non-human animal SIRPA protein. In some such methods, the humanized non-human animal SIRPA gene is operably linked to an endogenous non-human animal SIRPA promoter. In some such methods, the non-human animal is heterozygous for the humanized SIRPA gene. In some such methods, the non-human animal is homozygous for the humanized SIRPA gene. In some such methods, the non-human animal comprises the humanized SIRPA gene in its germline.

### BRIEF DESCRIPTION OF THE FIGURES

The patent application file contains at least one drawing executed in color. Copies of this patent application with color drawing(s) will be provided by the Office upon request and payment of the necessary fee. FIGS. 1A-1C show abnormal lipid accumulation in humanized livers of mice and rats. H&E, FAH, hASGR1 and HSD17B13 IHC and Oil Red O staining show lipid accumulation in both humanized mouse (FIG. 1A) and rat (FIG. 1B) livers, collected at 12 weeks post-transplant and 7 months post-transplant, respectively. FIG. 1C shows H&E and FAH IHC of rat or human primary hepatocytes engrafted into FSRG mice (left), or mouse or human primary hepatocytes engrafted into FRG rat livers (right). FSRG mouse livers were harvested at 13 weeks post-engraftment, and FRG rat livers were harvested at 15 weeks post-transplant.

FIGS. 2A-2G show that ectopic overexpression of rodent IL-6 receptor or constitutively active GP130 protects human hepatocytes from lipid droplet accumulation in humanized livers in mouse and rat models. FIG. 2A shows western blots of pSTAT3 levels in primary human, rat or mouse hepatocytes treated with human, rat, or mouse IL-6 protein (50 ng/mL for 15 minutes). FIGS. 2B and 2D show western blots of pSTAT3 levels in protein lysates from non-transduced primary human hepatocytes (PHH) or PHH transduced with lentivirus carrying mouse (FIG. 2B) or rat (FIG. 2D) IL-6R for 30 minutes at 5.00E+04 viral genomes (VG)/cell in suspension and treated with mIL-6 (FIG. 2B) or rIL-6 (FIG. 2D). FIGS. 2C and 2E upper panels show H&E, FAH IHC, hASGR1 IHC, and FLAG (IHC or miRNAscope) staining of FSRG mouse (FIG. 2C) or FRG rat (FIG. 2E) livers engrafted with mIL-6R- or rIL-6R-expressing PHH (FLAG+ hepatocytes) or non-transduced PHH (FLAG-hepatocytes). FIG. 2C lower panels show H&E and hASGR1-IHC/FLAG-RNAScope double staining of FSRG mouse livers engrafted with PHH expressing mIL6R, collected after 14 weeks. mIL6R-expressing human hepatocytes (Hu-mIL6R) were detected using a human-specific ASGR1 antibody and FLAG RNAScope probe. Non-transduced human hepatocytes (Hu) were positive for hASGR1, but not FLAG. Non-engrafted regions (Mo) are negative for both hASGR1 and FLAG RNAScope. Experiment was performed twice with 2 different hepatocyte donors, n=6 per cohort. FIG. 2E lower panels show H&E and hASGR1-IHC/FLAG-RNAScope staining of FRG rat livers engrafted with PHH expressing rIL6R and harvested after 22 weeks. rIL6R-expressing human hepatocytes (Hu-rIL6R) were positive for both hASGR1 and FLAG. Non-transduced human hepatocytes (Hu) were hASGR1-positive, but FLAG-negative. Non-engrafted regions (R) are negative for both hASGR1 and FLAG. Experiment performed twice, n=5 per cohort. FIG. 2F shows western blots of pSTAT3 levels in protein lysates from non-transduced PHH or PHH transduced overnight with lentivirus carrying GFP control or mutant GP130^{Y186-Y190del} at 1.00E+05 VG/cell. FIG. 2G upper panels show H&E, hASGR1 IHC, and GFP RNAscope staining of livers engrafted with GP130^{Y186-Y190del} mutant. FIG. 2G lower panels show H&E and hASGR1-IHC/GFP-RNAScope double staining of FSRG mouse livers engrafted with GP130^{Y186-Y190del} expressing PHH (Hu-GP130*), collected after 8 weeks. Hu-GP130* hepatocytes were detected by hASGR1 IHC and GFP-RNAScope. Non-transduced human hepatocytes (Hu) were positive for hASGR1, but not GFP. Non-engrafted regions (Mo) are negative for both. Experiment performed twice with 2 hepatocyte donors, n=3 per cohort.

FIGS. 3A-3B show systemic supplementation with hIL-6 prevents lipid droplet accumulation in humanized livers. FIG. 3A shows serum hIL-6 levels (ELISA) and western blots of STAT3 activation in protein extracts of livers of PHH-engrafted mice treated with AAV9-hIL-6 (5.00E+11 VG/mouse, n=7) versus PBS control (n=5) at the time of hepatocyte transplant. FIG. 3B shows H&E and FAH IHC of mouse livers 8 weeks after AAV9-hIL-6 dosing and PHH transplant. Quantification of the % fatty area and % FAH positivity confirms a decrease in lipid accumulation in hIL-6-treated mice despite similar humanization levels. Data shown as mean±SD (each dot represents one mouse, 2-3 liver lobes/mouse were analyzed).

FIGS. 4A-4B show humanization of the IL-6 gene in recipient mice results in hIL-6 expression in host mice and correction of fatty phenotype of humanized livers. FIG. 4A shows human or mouse IL-6 levels detected in the plasma of IL-6^{m/m}, IL-6^{h/m}, or IL-6^{h/h} mice (referred to elsewhere as FSRG-IL6^{WT}, FSRG-IL6^{HumIn(het)} or FSRG-IL6^{HumIn(homo)} respectively) either before or after LPS (1 mg/kg, intraperitoneal injection) stimulation (20 μg, 2 hours). Blue and pink icons represent male and female mice, respectively. FIG. 4B shows H&E and FAH IHC from PHH-engrafted livers of IL-6^{m/m}, IL-6^{h/m}, or IL-6^{h/h} mice. Quantification shows the % Fatty Area (negative H&E staining) and % FAH+ staining (Mean±SD, 3 liver sections/group; **p<0.01 one-way ANOVA).

FIGS. 5A-5B show human IL-6 over-expression corrects lipid droplet accumulation in humanized liver mice and rats. FIGS. 5A and 5B show H&E, FAH IHC, and Oil Red O staining on livers from AAV9-hIL-6 versus PBS treated humanized liver mice 8 weeks after PHH transplantation (FIG. 5A) or humanized liver rats 12 weeks after PHH transplantation (FIG. 5B), both collected 4 weeks after AAV dosing. For FIG. 5A, quantification of the % fatty area (negative H&E staining), FAH and Oil Red O staining shows mean±SD from one experimental repeat. Each dot represents one mouse (n=4 per group), 2-3 liver lobes/mouse were analyzed. For FIG. 5B, quantification of the % fatty area (negative H&E staining), FAH and Oil Red O staining shows mean±SD (each dot represents one rat (n=4 per group), 2-3 liver lobes/rat were analyzed). *p<0.05, **p<0.01, ***p<0.001; Unpaired t test.

FIGS. 6A-6E show human Kupffer cell engraftment in humanized liver mice leads to correction of fatty phenotype in human hepatocytes. FIG. 6A shows the scheme of dual-engraftment of human immune system and human hepatocytes. FIG. 6B shows hIL-6 and hCD68 RNAscope staining in human immune system (HIS)-engrafted FSRG mice after 2 hours LPS stimulation (1 mg/kg, intraperitoneal injection) shows the presence of human IL-6-producing human Kupffer cells in mouse liver. FIG. 6C shows H&E, FAH, hCD45 and hCD68 IHC staining in mouse livers engrafted with human hepatocytes only (HuHEP), human immune system only (HIS) or dual-engrafted (HIS-HuHEP). Quantification shows a correction of fatty liver phenotype in HIS-HuHEP versus HuHEP mice (each dot represents one mouse, 2-3 liver lobes/mouse were analyzed, *p<0.05, one-way ANOVA). FIG. 6D shows hCD68 IHC confirms depletion of human Kupffer cells in double-humanized HIS-HuHEP mice treated with a control antibody or an anti-CSF1R antibody (20 mg/kg, 2×/week), starting at the time of PHH transplant. Experiment was performed 3 times, with 3 different HSC donors. FIG. 6E shows H&E staining and FAH IHC of liver sections from control or anti-hCSF1R antibody treated HIS-HuHEP mice. Quantification shows mean±SD from one experimental repeat with a single HSC donor (each dot represents one mouse, 2-3 liver lobes/mouse were analyzed, *p<0.05, one-way ANOVA).

FIGS. 7A-7D show restoration of FGF19-FGFR4 or HGF-MET signaling pathways does not correct lipid accumulation in human hepatocytes. FIG. 7A shows AAV9-hFGF19 treatment resulted in expression of hFGF19 protein, inhibition of the FGF19-FGFR4 target gene, Cyp7a1, expression in the liver and reduced bile acid production, as demonstrated by levels of hFGF19 in the serum, human CYP7A1 and mouse Cyp7a1 expression in the liver (levels normalized to human and mouse GAPDH, respectively), and total bile acid (TBA) levels in mice treated with AAV9-FGF19hFGF19 vs. PBS control at the time of hepatocyte transplant. Lines in each graph represent the mean value. FIG. 7B shows H&E and FAH IHC in livers of control versus AAV9-hFGF19 treated mice. FIG. 7C shows human cMET activating antibody mimics hHGF to activate MET, as shown by pMET levels in mouse hepatocytes with the endogenous murine c-Met allele replaced by human MET gene, treated with mouse or human HGF (50 ng/mL), control antibody (REGN1954) or a human-specific cMET activating antibody (REGN6753) for 15 minutes. FIG. 7D shows H&E and FAH IHC in livers of mice treated with REGN1945 or REGN6753 (25 mg/kg, 1×/week).

FIGS. 8A-8B show expression of rodent IL-6R in human hepatocytes eliminates lipid droplet accumulation in humanized livers. Ectopic expression of mouse (FIG. 8A) or rat (FIG. 8B) IL-6R in human hepatocytes eliminates lipid droplet accumulation, as shown by H&E (left), FAH IHC (middle) and FLAG IHC (right) staining of nearby sections of FSRG mouse livers (FIG. 8A) or FRG rat livers (FIG. 8B) engrafted with PHH infected with lentivirus carrying mIL6R or rIL6R, respectively. mIL-6R- or rIL-6R-expressing human hepatocytes are positive for both FAH and FLAG IHC, while non-transduced human hepatocytes are FAH positive, but FLAG negative. Non-engrafted regions (endogenous mouse or rat cells) are negative for both FAH and FLAG IHC.

FIG. 9 shows hIL-6 over-expression by AAV can signal to engrafted human hepatocytes in humanized liver mouse model. RNA levels of human IL-6 target genes, hSOCS3 and hSAA2, in the livers of AAV9-hIL-6 treated mice are shown, as measured by TaqMan qPCR (plotted as mean±SD).

FIGS. 10A-10B show IL-6 expression and signaling in the livers of FSRG-hIL-6 mice. FIG. 10A shows that humanization of IL-6 allele led to a human specific hepatic IL-6 response, shown by hCRP expression in FSRG-IL-6 HumIn(het) and FSRG-IL-6 HumIn(homo) but not FSRG-IL-6^{WT} humanized liver mice (left). Engraftment of human hepatocytes was confirmed by serum hALB (right). FIG. 10B shows human IL-6 was detected (predominately in CD68 positive cells) in the livers of FSRG-IL-6 HumIn(het) but not FSRG-IL-6^{WT} humanized liver mice after 2 hr LPS stimulation, as shown by H&E, FAH IHC, hIL-6, mIL-6, and mCD68 RNAscope staining.

FIGS. 11A-11C show AAV9-hIL-6 dosing activates IL-6 signaling in humanized liver mice. FIG. 11A shows AAV9-hIL-6 dosing resulted in expression of hIL-6 (left) and prototype acute phase reactant hCRP (right) in the serum of humanized liver mice treated with AAV9-hIL-6 or PBS control 8 weeks after PHH transplant and collected 4 weeks after AAV dosing. FIG. 11B shows pSTAT3 levels, and FIG. 11C shows RNA expression of IL-6 target genes, SOCS3 and SAA2, in humanized mouse livers treated with AAV9-hIL-6 versus control PBS. Data plotted as mean±SD.

FIGS. 12A-12C show AAV9-hIL-6 treatment in humanized liver rats. FIG. 12A shows AAV9-hIL-6 dosing resulted in expression of hIL-6 (left) and prototype acute phase reactant hCRP (right) in the serum of humanized liver rats treated with AAV9-hIL-6 or PBS 12 weeks after PHH transplant and collected 4 weeks after AAV dosing. FIG. 12B shows pSTAT3 levels, and FIG. 12C shows RNA expression of IL-6 target genes, SOCS3 and SAA2, in AAV9-hIL-6 versus control PBS treated humanized rat livers. Data plotted as mean±SD.

FIGS. 13A-13B show IL-6 expression and signaling in the livers of FSRG-hIL-6 mice. FIG. 13A shows hCRP and hAlb in the serum of PHH-engrafted FSRG-IL-6^{m/m}, IL-6^{h/m}, or IL-6^{h/h} mice. FIG. 13B shows H&E, FAH IHC, hIL-6, mIL-6, and mCD68 RNAscope staining of livers from either FSRG-IL-6^{h/m} or FSRG-IL-6^{m/m} mice after 2 hours LPS stimulation.

FIGS. 14A-14D show human OSM over-expression also corrects lipid droplet accumulation in engrafted human hepatocytes. Humanized liver mice were treated with AAV9-hOSM or PBS control 12 weeks after PHH transplantation and collected 3 weeks after AAV dosing. FIG. 14A shows hOSM levels in the serum of humanized liver mice at the time of termination. FIG. 14B shows Western blot of liver lysates confirming activation of pSTAT3 in AAV9-hOSM treated mice. FIG. 14C shows RNA expression of human target genes, CRP, SOCS3 and SAA2 in AAV9-hOSM treated versus control mice, confirming activation of downstream gp130 signaling. FIG. 14D shows H&E staining and FAH IHC on liver sections from AAV9-hOSM and PBS treated mice, 3 weeks after AAV dosing. Quantification of the % fatty area (negative H&E staining) and FAH staining shows mean±SD (each dot represents one mouse, 2 liver lobes/mouse were analyzed). *p<0.05, Unpaired t test.

FIGS. 15A-15D show that engraftment of human immune cells led to hIL-6 expression in HIS-HuHEP mice. FIG. 15A shows serum levels of human albumin, human IL-6, and human CRP, and % hCD45+ cells in the blood confirm dual-humanization and intact IL-6 signaling in HIS-HuHEP mice. FIG. 15B shows single and double-IHC for hCD45 and hCD68 in HIS-HuHEP mouse livers. FIG. 15C shows serum ELISAs that demonstrate a complete absence of hCRP in anti-CSF1R treated mice, despite high human albumin levels. FIG. 15D shows RNA expression of human macrophage markers (hCD68, hITGAM, hEMR1), hIL-6, hCD3, hCD20 and mouse CD68 in livers of control antibody or anti-hCSF1R treated HIS-HuHEP mice. Data shown as mean±SD.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous IL6 sequence of an animal refers to a native IL6 sequence that naturally occurs at the IL6 locus in the animal.

“Exogenous” molecules or sequences include molecules or sequences that are not normally present in a cell in that form or that are introduced into a cell from an outside source. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by a heterologous promoter not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a TAR DNA-binding protein 43 (TDP-43) protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, an “IL6 locus” may refer to the specific location of an IL6 gene, IL6 DNA sequence, IL-6-encoding sequence, or IL6 position on a chromosome of the genome of an organism that has been identified as to where such a sequence resides. An “IL6 locus” may comprise a regulatory element of an IL6 gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

A constitutive promoter is one that is active in all tissues or particular tissues at all developing stages. Examples of constitutive promoters include the human cytomegalovirus immediate early (hCMV), mouse cytomegalovirus immediate early (mCMV), human elongation factor 1 alpha (hEF1α), mouse elongation factor 1 alpha (mEF1α), mouse phosphoglycerate kinase (PGK), chicken beta actin hybrid (CAG or CBh), SV40 early, and beta 2 tubulin promoters.

Examples of inducible promoters include, for example, chemically regulated promoters and physically regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

Tissue-specific promoters can be, for example, liver-specific promoters or muscle-specific promoters.

Developmentally regulated promoters include, for example, promoters active only during an embryonic stage of development, or only in an adult cell.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “in vitro” includes artificial environments and to processes or reactions that occur within an artificial environment (e.g., a test tube or an isolated cell or cell line). The term “in vivo” includes natural environments (e.g., a cell, organism, or body) and to processes or reactions that occur within a natural environment. The term “ex vivo” includes cells that have been removed from the body of an individual and processes or reactions that occur within such cells.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

When “at least,” “up to,” “more than,” “no more than,” “less than,” or other similar language modifies a number, it can be understood to modify each number in the series.

As used herein, “no more than” or “less than” is understood as the value adjacent to the phrase and logical lower values or integers, as logical from context, to zero. When “no more than” or “less than” is present before a series of numbers or a range, it is understood that each of the numbers in the series or range is modified.

Unless otherwise apparent from the context, the term “about” encompasses values ±5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

In the event of a conflict between a sequence in the application and an indicated accession number or position in an accession number, the sequence in the application predominates.

### DETAILED DESCRIPTION

### I. Overview

Humanized liver mouse and rat models, in which donor human hepatocytes can repopulate recipient rodent parenchyma, have been used in studying human liver biology, diseases, and therapeutics. However, it has been observed that engrafted human hepatocytes in both humanized liver mice and humanized liver rats show defects, including increased lipid droplet accumulation. Ameliorating such imperfections would improve the accuracy of the models to recapitulate normal human liver biology.

We demonstrate that this abnormality in humanized livers is a result of lack of interleukin-6 (IL-6) receptor signaling in human hepatocytes due to incompatibility between murine or rat IL-6 from host-derived Kupffer cells and human interleukin-6 receptor (IL-6R) expressed on donor hepatocytes. Provided herein are approaches to correct hepatosteatosis in humanized liver animals through restoration of IL-6/GP130/IL-6R pathway signaling in donor human hepatocytes, including: ectopic expression of non-human animal IL-6R in engrafted human hepatocytes, ectopic expression of non-human animal OSMR in engrafted human hepatocytes, expression of constitutively active GP130 in engrafted human hepatocytes, supplementing human IL-6 (or human-IL-6R-compatible ligand (e.g., human-IL-6R-compatible IL-6)) in host animals, supplementing human OSM (or human-OSMR-compatible ligand (e.g., human-OSMR-compatible OSM)) or other human GP130 ligands in host animals, humanizing the IL6 gene in host animals, and humanizing the OSM gene in host animals.

Provided herein are genetically modified non-human animals that are immunodeficient and comprise xenotransplanted hepatocytes such as human hepatocytes, wherein the genetically modified non-human animal and/or the transplanted hepatocytes are modified to restore interleukin-6 (IL-6)/interleukin-6 receptor (IL-6R) signaling pathway activity or interleukin-6 receptor subunit beta (GP130) signaling pathway activity in the transplanted hepatocytes. Also provided are methods of assessing the activity of human-liver-targeting reagents in such non-human animals and methods of making animals with a humanized liver (e.g., with reduced steatosis). Also provided are genetically modified non-human animals comprising an inactivated endogenous Rag2 gene, an inactivated endogenous Il2rg gene, an inactivated endogenous Fah gene, and a humanized IL6 gene and methods of using and making such animals. Also provided are genetically modified non-human animals comprising an inactivated endogenous Rag1 gene, an inactivated endogenous Rag2 gene, an inactivated endogenous Il2rg gene, an inactivated endogenous Fah gene, and a humanized IL6 gene and methods of using and making such animals. Also provided are genetically modified non-human animals comprising an inactivated endogenous Rag2 gene, an inactivated endogenous Il2rg gene, an inactivated endogenous Fah gene, and a humanized OSM gene and methods of using and making such animals. Also provided are genetically modified non-human animals comprising an inactivated endogenous Rag1 gene, an inactivated endogenous Rag2 gene, an inactivated endogenous Il2rg gene, an inactivated endogenous Fah gene, and a humanized OSM gene and methods of using and making such animals. Also provided are genetically modified non-human animals comprising an inactivated endogenous Rag2 gene, an inactivated endogenous Il2rg gene, an inactivated endogenous Fah gene, a humanized IL6 gene, and a humanized OSM gene and methods of using and making such animals. Also provided are genetically modified non-human animals comprising an inactivated endogenous Rag1 gene, an inactivated endogenous Rag2 gene, an inactivated endogenous Il2rg gene, an inactivated endogenous Fah gene, a humanized IL6 gene, and a humanized OSM gene and methods of using and making such animals. Optionally, such non-human animals can also comprise a humanized Growth Hormone gene. Also provided are methods of reducing or ameliorating hepatosteatosis in non-human animals with humanized livers.

### II. Genetically Modified Non-Human Animals Comprising Xenotransplanted Hepatocytes

Provided herein are genetically modified non-human animals (e.g., mice or rats) suitable for xenotransplantation of hepatocytes, such as human hepatocytes, as well as genetically modified non-human animal cells and genomes.

The genetically modified non-human animals can comprise xenotransplanted hepatocytes (e.g., xenotransplanted and expanded hepatocytes). The xenotransplanted hepatocytes can be from any species other than that of the recipient non-human animal. For example, the xenotransplanted hepatocytes can be human hepatocytes. The transplanted hepatocytes can be from a species whose IL-6R is incompatible with the endogenous non-human animal IL-6 (e.g., the transplanted hepatocytes can be human hepatocytes, and the non-human animal can be a mouse or a rat). The transplanted hepatocytes can be normal, healthy cells, or can be diseased or mutant-bearing cells. For example, the transplanted hepatocytes can be wild type hepatocytes, or they can comprise one or more mutations. In a specific example, the transplanted hepatocytes have a wild type Fah gene or an Fah gene that produces a functional protein.

Also provided are modifications to the genetically modified non-human animals and/or the xenotransplanted hepatocytes to restore IL-6/IL-6R signaling pathway or GP130 signaling pathway activity in the xenotransplanted hepatocytes. Such modifications can result in reduced lipid droplet accumulation and reduced steatosis in the xenotransplanted hepatocytes compared to non-human animals in which the genetically modified non-human animals and the xenotransplanted hepatocytes do not have modifications to restore IL-6/IL-6R signaling pathway or GP130 signaling pathway activity in the xenotransplanted hepatocytes.

A. Genetically Modified Non-Human Animals for Xenotransplantation of Hepatocytes

Provided herein are genetically modified non-human animals suitable for xenotransplantation of hepatocytes. Any suitable non-human animal can be used. In some cases, the animals are genetically modified non-human animals in which the non-human animal's immune system has been modified such that it is unable or has reduced ability to mount an immune response to xenografted cells (e.g., human hepatocytes) (e.g., the non-human animal is immunodeficient). In some cases, the animals are genetically modified non-human animals in which (1) non-human (i.e., endogenous) hepatocytes in the liver can be selectively and conditionally ablated; and (2) the non-human animal's immune system has been modified such that it is unable to mount an immune response to xenografted cells (e.g., human hepatocytes) (e.g., the non-human animal is immunodeficient). Also provided are cells and genomes comprising the genetic modifications disclosed herein. The genetically modified non-human animals disclosed herein can be used for in vivo engraftment and expansion of xenotransplanted hepatocytes (e.g., human hepatocytes). Xenotransplantation refers to the transplantation of living cells, tissues, or organs from one species to another. Such cells, tissues, or organs are called xenografts or xenotransplants (e.g., xenotransplanted cells).

Any suitable immunodeficient non-human animal can be used. See, e.g., Weber et al. (2009) Liver Transplantation 15:7-14, herein incorporated by reference in its entirety for all purposes. Such non-human animals can be immunocompromised such that T cells and B cells do not develop. For examples, such immunodeficient non-human animals can lack functional T cells, B cells, and/or natural killer (NK) cells. Immunodeficient non-human animals refer to non-human animals lacking in at least one essential function of the immune system. For example, an immunodeficient non-human animal is one lacking specific components of the immune system or lacking function of specific components of the immune system (such as, for example, B cells, T cells, or NK cells). In some cases, an immunodeficient animal lacks macrophages. In some cases, an immunodeficient animal comprises one or more genetic alterations that prevent or inhibit the development of functional immune cells (such as B cells, T cells, or NK cells). In some examples, the genetic alteration is in the Rag1, Rag2, or Il2rg gene (i.e., the immunodeficient non-human animal is Rag1^{-/-}, Rag2^{-/-}, and/or Il2rg^{-/-}). In some cases, the non-human animal comprises an inactivated endogenous Il2rg gene. In some cases, the non-human animal comprises an inactivated endogenous Rag1 gene and/or an inactivated endogenous Rag2 gene. In some cases, the non-human animal comprises an inactivated endogenous Rag1 gene. In some cases, the non-human animal comprises an inactivated endogenous Rag2 gene. In some cases, the non-human animal comprises an inactivated endogenous Rag1 gene and an inactivated endogenous Rag2 gene. In some cases, the immunodeficient non-human animal comprises an inactivated endogenous Rag2 gene and an inactivated endogenous Il2rg gene (i.e., is Rag2^{-/-} and Il2rg^{-/-}). In some cases, the immunodeficient non-human animal comprises an inactivated endogenous Rag1 gene, an inactivated endogenous Rag2 gene, and an inactivated endogenous Il2rg gene (i.e., is Rag1^{-/-}, Rag2^{-/-}, and Il2rg^{-/-}). In some cases, the non-human animal comprises a severe combined immunodeficiency (SCID) mutation in the Prkdc gene (Prkdc^{scid}). See, e.g., Mercer et al. (2001) Nat. Med. 7(8):927-933, herein incorporated by reference in its entirety for all purposes. Prkdc encodes DNA-dependent protein kinase catalytic subunit and is assigned NCBI GeneID 19090 in mice. The scid mutation is a mutation in the Prkdc gene, protein kinase, DNA activated, catalytic polypeptide. The specific genetic alteration in the Prkd^{scid} allele in mice is a T-to-A transversion point mutation to codon 4046 (codon 4095 in transcript ENSMUST00000023352.8) created a premature stop codon (p.Y4046*). Prkdc is involved in DNA repair via non-homologous end joining (NHEJ). V(D)J recombination, which rearranges the genetic components of antibodies and T cell receptors during B and T cell development, requires NHEJ for proper function. In some cases, the non-human animal comprises a severe combined immunodeficiency (SCID) mutation in the Prkdc gene (Prkdc^{scid}) and an inactivated endogenous Il2rg gene, such as the NOG background. See, e.g., Ito et al. (2002) Blood 100(9):3175-3182, herein incorporated by reference in its entirety for all purposes. NOG mice are immunodeficient mice lacking mature T, B, and NK cells. Other examples of mutations causing immunodeficiency include X-linked SCID characterized by autosomal recessive SCID characterized by JAK3 mutations, ADA mutations, IL7R mutations, CD3 mutations, ARTEMIS (DCLRE1C) mutations, and CD45 (PTPRC) mutations. Although certain non-limiting examples of genetic alterations that result in immunodeficiency are provided above, other known immunodeficient non-human animals can also be used.

Any suitable genetic modification to allow for non-human (i.e., endogenous) hepatocytes in the liver to be selectively and conditionally ablated can be used. In some cases, the non-human animals comprise an inactivated endogenous Fah gene. This inactivation results in a toxic accumulation of tyrosine catabolites within hepatocytes. The compound 2-(2-nitro-4-trifluoro-methylbenzoyl)-1,3-cyclohexanedione (NTBC) can be used to block the enzyme hydroxyphenylpyruvate dioxygenase upstream of FAH and therefore prevents the accumulation of hepatotoxic metabolites. In some cases, the non-human animals comprise a urokinase type plasminogen activator gene (Plau; NCBI GeneID 18792 in mice) operably linked to a liver-specific promoter, such as an albumin promoter. See, e.g., Mercer et al. (2001) Nat. Med. 7(8):927-933, herein incorporated by reference in its entirety for all purposes. Such non-human animals have hepatotoxicity leading to liver failure. In some cases, the non-human animals comprise a herpes simplex virus type 1 thymidine kinase (HSVtk) gene operably linked to a liver-specific promoter, such as an albumin promoter. See, e.g., Hasegawa et al. (2011) Biochem. Biophys. Res. Commun. 405(3):405-410, herein incorporated by reference in its entirety for all purposes. Administration of ganciclovir (GCV), a drug that is not toxic to human or mouse tissues, induces tissue-specific ablation of transgenic liver parenchymal cells. Because HSVtk catalyzes GCV phosphorylation, which is the rate-limiting step that cannot be performed in mammalian cells lacking this transgene, liver cells expressing the transgene are selectively destroyed. Although certain non-limiting examples of genetic modifications to allow for non-human (i.e., endogenous) hepatocytes in the liver to be selectively and conditionally ablated are provided above, any other suitable genetic modifications can also be used.

In one example, genetically modified non-human animals for xenotransplantation of hepatocytes (e.g., transplantation of human hepatocytes) have the following genes inactivated (i.e., knocked out): Fah (encodes fumarylacetoacetase); Rag2 (encodes V(D)J recombination-activating protein 2); and Il2rg (encodes interleukin 2 receptor subunit gamma). Also provided are genetically modified non-human animal cells or genomes having the following genes inactivated (i.e., knocked out): Fah; Rag2; and Il2rg. In one example, genetically modified non-human animals for xenotransplantation of hepatocytes (e.g., transplantation of human hepatocytes) have the following genes inactivated (i.e., knocked out): Fah (encodes fumarylacetoacetase); Rag1 (encodes V(D)J recombination-activating protein 1); Rag2 (encodes V(D)J recombination-activating protein 2); and Il2rg (encodes interleukin 2 receptor subunit gamma). Also provided are genetically modified non-human animal cells or genomes having the following genes inactivated (i.e., knocked out): Fah; Rag1; Rag2; and Il2rg. In one example, the genetically modified non-human animal is a rat. See, e.g., Carbonaro et al. (2022) Sci. Rep. 12(1):14079 and US 2016/0249591, each of which is herein incorporated by reference in its entirety for all purposes. In another example, the genetically modified non-human animal is a mouse. See, e.g., Strom et al. (2010) Methods Mol. Biol. 640:491-509, Azuma et al. (2007) Nat. Biotechnol. 25(8):903-910, and U.S. Pat. No. 8,569,573, each of which is herein incorporated by reference in its entirety for all purposes. In another example, the genetically modified non-human animal is a pig. See, e.g., U.S. Pat. No. 9,000,257, herein incorporated by reference in its entirety for all purposes.

Fah is an essential gene in the tyrosine catabolism pathway. When Fah is mutated in animals, toxic intermediate metabolites accumulate in the liver, causing hepatocyte loss and, ultimately, liver failure and death. This toxicity can be ameliorated by blocking the activity of another tyrosine catabolism enzyme, 4-hydroxyphenylpyruvate dioxygenase, which can be achieved by the administration of the small molecule nitisinone (NTBC). Fah mutant mice are healthy and viable when NTBC is administered. They quickly become moribund and die, however, when NTBC is withdrawn. Thus, NTBC administration and withdrawal allows precise temporal control of hepatocyte toxicity in Fah mutant non-human animals.

Rag1, Rag2, and Il2rg are essential components of the adaptive immune system. When these genes are mutated in animals, T-cells and B-cells do not mature, and the animals are severely compromised. When these animals are challenged with xenotransplanted cells, they are unable to mount an immune response to the foreign cells.

V(D)J recombination-activating protein 1 (also known as RAG1, recombination-activating 1, recombination activating gene 1, and recombination activating protein 1) is encoded by the Rag1 gene (also known as recombination activating 1). RAG1 is a catalytic component of the RAG complex, a multiprotein complex that mediates the DNA cleavage phase during V(D)J recombination. V(D)J recombination assembles a diverse repertoire of immunoglobulin and T-cell receptor genes in developing B and T-lymphocytes through rearrangement of different V (variable), in some cases D (diversity), and J (joining) gene segments. In the RAG complex, RAG1 mediates the DNA-binding to the conserved recombination signal sequences (RSS) and catalyzes the DNA cleavage activities by introducing a double-strand break between the RSS and the adjacent coding segment. RAG2 is not a catalytic component but is required for all known catalytic activities. RAG1 and RAG2 are essential to the generation of mature B cells and T cells, two types of lymphocytes that are crucial components of the adaptive immune system.

Mouse Rag1 maps to 2 E2; 2 53.88 cM on chromosome 2 (NCBI RefSeq Gene ID 19373; Assembly GRCm39 (GCF_000001635.27); location NC_000068.8 (101468597 . . . 101479877, complement). Reference to the mouse Rag1 gene includes the canonical, wild type form as well as all allelic forms and isoforms. The canonical, wild type mouse RAG1 protein has been assigned UniProt accession number P15919 and NCBI Accession No. NP_033045.2. Reference to mouse RAG1 proteins includes wild type forms as well as all allelic forms and isoforms. An mRNA (cDNA) encoding the canonical isoform is assigned NCBI Accession No. NM_009019.2. Reference to the mouse Rag1 mRNA (cDNA) and coding sequence includes the canonical, wild type forms as well as all allelic forms and isoforms.

Rat Rag1 maps to 3q31 on chromosome 3 (NCBI RefSeq Gene ID 84600; Assembly mRatBN7.2 (GCF_015227675.2); location NC_051338.1 (87917061 . . . 87928158, complement). Reference to the rat Rag1 gene includes the canonical, wild type form as well as all allelic forms and isoforms. The canonical, wild type rat RAG1 protein has been assigned UniProt accession number G3V6K9 and NCBI Accession No. NP_445920.1. Reference to rat RAG1 proteins includes canonical, wild type forms as well as all allelic forms and isoforms. An mRNA (cDNA) encoding the canonical isoform is assigned NCBI Accession No. NM_053468.1. Reference to the rat Rag1 mRNA (cDNA) and coding sequence includes the canonical, wild type forms as well as all allelic forms and isoforms.

An inactivated endogenous Rag1 gene is a Rag1 gene that does not produce a RAG1 protein or does not produce a functional RAG1 protein. The non-human animal (or cell or genome) can comprise the inactivated Rag1 gene in its germline. The non-human animal (or cell or genome) can be homozygous for an inactivating mutation in the Rag1 gene. As one example, an inactivated endogenous Rag1 gene can comprise an insertion, a deletion, or one or more point mutations in the endogenous Rag1 gene resulting in loss of expression of functional RAG1 protein. Some inactivated endogenous Rag1 genes can comprise a deletion or disruption of all of the endogenous Rag1 gene or can comprise a deletion or disruption of a fragment of (i.e., a part of or portion of) the endogenous Rag1 gene. For example, some, most, or all of the coding sequence in the endogenous Rag1 gene can be deleted or disrupted. In one example, a 5′ fragment of the Rag1 gene can be deleted or disrupted (e.g., including the start codon). As one example, an inactivated endogenous Rag1 gene can be one in which the start codon of the endogenous Rag1 gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Rag1 gene can be deleted or disrupted (e.g., including the stop codon). In another example, an internal fragment of the Rag1 gene (i.e., a fragment from the middle of the Rag1 gene) can be deleted or disrupted. In another example, all of the coding sequence in the endogenous Rag1 gene is deleted or disrupted.

V(D)J recombination-activating protein 2 (also known as RAG2, recombination-activating 2, recombination activating gene 2, and recombination activating protein 2) is encoded by the Rag2 gene (also known as recombination activating 2). As mentioned above, RAG1 is a catalytic component of the RAG complex, a multiprotein complex that mediates the DNA cleavage phase during V(D)J recombination. RAG2 is not a catalytic component but is required for all known catalytic activities. RAG1 and RAG2 are essential to the generation of mature B cells and T cells, two types of lymphocytes that are crucial components of the adaptive immune system.

Mouse Rag2 maps to 2 E2; 2 53.87 cM on chromosome 2 (NCBI RefSeq Gene ID 19374; Assembly GRCm39 (GCF_000001635.27); location NC_000068.8 (101455057 . . . 101462873). Reference to the mouse Rag2 gene includes the canonical, wild type form as well as all allelic forms and isoforms. The canonical, wild type mouse RAG2 protein has been assigned UniProt accession number P21784 and NCBI Accession No. NP_033046.1. Reference to mouse RAG2 proteins includes canonical, wild type forms as well as all allelic forms and isoforms. An mRNA (cDNA) encoding the canonical isoform is assigned NCBI Accession No. NM_009020.3. Reference to the mouse Rag2 mRNA (cDNA) and coding sequence includes the canonical, wild type forms as well as all allelic forms and isoforms.

Rat Rag2 maps to 3q31 on chromosome 3 (NCBI RefSeq Gene ID 295953; Assembly mRatBN7.2 (GCF_015227675.2); location NC_051338.1 (87902373 . . . 87910227). Reference to the rat Rag2 gene includes the canonical, wild type form as well as all allelic forms and isoforms. The canonical, wild type rat RAG2 protein has been assigned UniProt accession number G3V6K7 and NCBI Accession No. NP_001093998.1. Reference to rat RAG2 proteins includes canonical, wild type forms as well as all allelic forms and isoforms. An mRNA (cDNA) encoding the canonical isoform is assigned NCBI Accession No. NM_001100528.1. Reference to the rat Rag2 mRNA (cDNA) and coding sequence includes the canonical, wild type forms as well as all allelic forms and isoforms.

An inactivated endogenous Rag2 gene is a Rag2 gene that does not produce a RAG2 protein or does not produce a functional RAG2 protein. The non-human animal (or cell or genome) can comprise the inactivated Rag2 gene in its germline. The non-human animal (or cell or genome) can be homozygous for an inactivating mutation in the Rag2 gene. As one example, an inactivated endogenous Rag2 gene can comprise an insertion, a deletion, or one or more point mutations in the endogenous Rag2 gene resulting in loss of expression of functional RAG2 protein. Some inactivated endogenous Rag2 genes can comprise a deletion or disruption of all of the endogenous Rag2 gene or can comprise a deletion or disruption of a fragment of (i.e., a part of or portion of) the endogenous Rag2 gene. For example, some, most, or all of the coding sequence in the endogenous Rag2 gene can be deleted or disrupted. In one example, a 5′ fragment of the Rag2 gene can be deleted or disrupted (e.g., including the start codon). As one example, an inactivated endogenous Rag2 gene can be one in which the start codon of the endogenous Rag2 gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Rag2 gene can be deleted or disrupted (e.g., including the stop codon). In another example, an internal fragment of the Rag2 gene (i.e., a fragment from the middle of the Rag2 gene) can be deleted or disrupted. In another example, all of the coding sequence in the endogenous Rag2 gene is deleted or disrupted.

Interleukin 2 receptor subunit gamma (also known as interleukin 2 receptor, gamma; interleukin 2 receptor, gamma (severe combined immunodeficiency), isoform CRA_a; cytokine receptor common subunit gamma precursor) is encoded by the Il2rg gene (also known as interleukin 2 receptor subunit gamma or IL2RG). IL2RG is a cytokine receptor subunit that is common to receptor complexes for several different interleukin receptors. IL2RG is located on the surface of immature blood-forming cells in bone marrow. IL2RG partners with other proteins to direct blood-forming cells to form lymphocytes. IL2RG also directs the growth and maturation of T cells, B cells, and natural killer cells. Mutations in Il2rg can cause X-linked severe combined immunodeficiency in which lymphocytes cannot develop normally. A lack of functional mature lymphocytes disrupts the immune system's ability to protect the body from infection.

Mouse Il2rg maps to X D; X 43.9 cM on chromosome X (NCBI RefSeq Gene ID 16186; Assembly GRCm39 (GCF_000001635.27); location NC_000086.8 (100307991 . . . 100311861, complement). Reference to the mouse Il2rg gene includes the canonical, wild type form as well as all allelic forms and isoforms. The canonical, wild type mouse IL2rG protein has been assigned UniProt accession number P34902 and NCBI Accession No. NP_038591.1. Reference to mouse IL2RG proteins includes canonical, wild type forms as well as all allelic forms and isoforms. An mRNA (cDNA) encoding the canonical isoform is assigned NCBI Accession No. NM_013563.4. Reference to the mouse Il2rg mRNA (cDNA) and coding sequence includes the canonical, wild type forms as well as all allelic forms and isoforms.

Rat Il2rg maps to Xq22 on chromosome X (NCBI RefSeq Gene ID 140924; Assembly mRatBN7.2 (GCF_015227675.2); location NC_051356.1 (66395330 . . . 66399026, complement). Reference to the rat Il2rg gene includes the canonical, wild type form as well as all allelic forms and isoforms. The canonical, wild type rat IL2rG protein has been assigned UniProt accession number Q68FU6 and NCBI Accession No. NP_543165.1. Reference to rat IL2RG proteins includes canonical, wild type forms as well as all allelic forms and isoforms. An mRNA (cDNA) encoding the canonical isoform is assigned NCBI Accession No. NM_080889.1. Reference to the rat Il2rg mRNA (cDNA) and coding sequence includes the canonical, wild type forms as well as all allelic forms and isoforms.

An inactivated endogenous Il2rg gene is an Il2rg gene that does not produce a IL2RG protein or does not produce a functional IL2RG protein. The non-human animal (or cell or genome) can comprise the inactivated Il2rg gene in its germline. The non-human animal (or cell or genome) can be homozygous for an inactivating mutation in the Il2rg gene. As one example, an inactivated endogenous Il2rg gene can comprise an insertion, a deletion, or one or more point mutations in the endogenous Il2rg gene resulting in loss of expression of functional IL2RG protein. Some inactivated endogenous Il2rg genes can comprise a deletion or disruption of all of the endogenous Il2rg gene or can comprise a deletion or disruption of a fragment of (i.e., a part of or portion of) the endogenous Il2rg gene. For example, some, most, or all of the coding sequence in the endogenous Il2rg gene can be deleted or disrupted. In one example, a 5′ fragment of the Il2rg gene can be deleted or disrupted (e.g., including the start codon). As one example, an inactivated endogenous Il2rg gene can be one in which the start codon of the endogenous Il2rg gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Il2rg gene can be deleted or disrupted (e.g., including the stop codon). In another example, an internal fragment of the Il2rg gene (i.e., a fragment from the middle of the Il2rg gene) can be deleted or disrupted. In another example, all of the coding sequence in the endogenous Il2rg gene is deleted or disrupted.

Fumarylacetoacetase (also known as FAH, FAA, beta-diketonase, or fumarylacetoacetate hydrolase) is encoded by the Fah gene (also known as fumarylacetoacetate hydrolase). FAH is an enzyme required in the last step of the tyrosine catabolic pathway, which hydrolyzes fumarylacetoacetate into fumarate and acetoacetate. A deficiency in Fah leads to the accumulation of toxic metabolites, including fumarylacetoacetate and maleylacetoacetate. 2-(2-Nitro-4-trifluoromethylbenzoyl)-1,3-cyclohexanedione (nitisinone or NTBC) as an inhibitor of 4-hydroxyphenylpyruvate dioxygenase acts by blocking the accumulation of toxic metabolites such as fumarylacetoacetate and maleylacetoacetate and can be effective in ameliorating liver and kidney damage in human patients with Fah deficiency. Several Fah mutations have been found that cause tyrosinemia type I (type 1 hereditary tyrosinemia, or HT) in humans. This condition is characterized by severe liver and kidney disease, neurological problems, and other signs and symptoms that begin in infancy. The altered Fah gene that causes this condition produces an unstable or inactive enzyme, which results in reduced or absent fumarylacetoacetate hydrolase activity. Without sufficient fumarylacetoacetate hydrolase activity, tyrosine and its byproducts are not properly broken down. As a result, fumarylacetoacetate accumulates in the liver and kidneys. Elevated levels of fumarylacetoacetate are thought to be toxic to cells and accumulation of this substance likely causes the liver and kidney problems and other features that are characteristic of tyrosinemia type I.

Mouse Fah maps to 7 D3; 7 48.36 cM on chromosome 7 (NCBI RefSeq Gene ID 14085; Assembly GRCm39 (GCF_000001635.27); location NC_000073.7 (84234367 . . . 84255150, complement). Reference to the mouse Fah gene includes the canonical, wild type form as well as all allelic forms and isoforms. The canonical, wild type mouse FAH protein has been assigned UniProt accession number P35505 and NCBI Accession No. NP_034306.2. Reference to mouse FAH proteins includes canonical, wild type forms as well as all allelic forms and isoforms. An mRNA (cDNA) encoding the canonical isoform is assigned NCBI Accession No. NM_010176.4. Reference to the mouse Fah mRNA (cDNA) and coding sequence includes the canonical, wild type forms as well as all allelic forms and isoforms.

Rat Fah maps to 1q31 on chromosome 1 (NCBI RefSeq Gene ID 29383; Assembly mRatBN7.2 (GCF_015227675.2); location NC_051336.1 (138548830 . . . 138571599, complement). Reference to the rat Fah gene includes the canonical, wild type form as well as all allelic forms and isoforms. The canonical, wild type rat FAH protein has been assigned UniProt accession number P25093 and NCBI Accession No. NP_058877.1. Reference to rat FAH proteins includes canonical, wild type forms as well as all allelic forms and isoforms. An mRNA (cDNA) encoding the canonical isoform is assigned NCBI Accession No. NM_017181.2. Reference to the rat Fah mRNA (cDNA) and coding sequence includes the canonical, wild type forms as well as all allelic forms and isoforms.

An inactivated endogenous Fah gene is a Fah gene that does not produce a FAH protein or does not produce a functional FAH protein. The non-human animal (or cell or genome) can comprise the inactivated Fah gene in its germline. The non-human animal (or cell or genome) can be homozygous for an inactivating mutation in the Fah gene. As one example, an inactivated endogenous Fah gene can comprise an insertion, a deletion, or one or more point mutations in the endogenous Fah gene resulting in loss of expression of functional FAH protein. Some inactivated endogenous Fah genes can comprise a deletion or disruption of all of the endogenous Fah gene or can comprise a deletion or disruption of a fragment of (i.e., a part of or portion of) the endogenous Fah gene. For example, some, most, or all of the coding sequence in the endogenous Fah gene can be deleted or disrupted. In one example, a 5′ fragment of the Fah gene can be deleted or disrupted (e.g., including the start codon). As one example, an inactivated endogenous Fah gene can be one in which the start codon of the endogenous Fah gene has been deleted or has been disrupted or mutated such that the start codon is no longer functional. For example, the start codon can be disrupted by a deletion or insertion within the start codon. Alternatively the start codon can be mutated by, for example, by a substitution of one or more nucleotides. In another example, a 3′ fragment of the Fah gene can be deleted or disrupted (e.g., including the stop codon). In another example, an internal fragment of the Fah gene (i.e., a fragment from the middle of the Fah gene) can be deleted or disrupted. In another example, all of the coding sequence in the endogenous Fah gene is deleted or disrupted.

Some genetically modified non-human animals described herein further comprise a humanized SIRPA gene, although this is not required. For example, such genetically modified non-human animals can comprise inactivated Fah, Rag2, and Il2rg genes and a humanized SIRPA gene. For example, such genetically modified non-human animals can comprise inactivated Fah, Rag1, Rag2, and Il2rg genes and a humanized SIRPA gene. In one example, the non-human animal is a mouse. In another example, the non-human animal is a rat.

SIRPA (also known as BIT, MFR, MYD 1, PTPNS1, SHPS1, SIRPα, and SIRP) encodes tyrosine-protein phosphatase non-receptor type substrate 1 (also known as brain Ig-like molecule with tyrosine-based activation motifs (Bit), CD172 antigen-like family member A, CD172a, inhibitory receptor SHPS-1, macrophage fusion receptor, MyD-1 antigen, SIRPA, SIRPα, and signal-regulatory protein alpha), which is an immunoglobulin-like cell surface receptor for CD47. It acts as docking protein and induces translocation of PTPN6, PTPN11 and other binding partners from the cytosol to the plasma membrane.

Mouse Sirpa maps to 2 F1; 2 63.19 cM on chromosome 2 (NCBI RefSeq Gene ID 19261; Assembly GRCm39 (GCF_000001635.27); location NC_000068.8 (129432962 . . . 129474148). Reference to the mouse Sirpa gene includes the canonical, wild type form as well as all allelic forms and isoforms. The canonical mouse SIRPA protein has been assigned UniProt accession number P97797 and NCBI Accession Nos. NP_001277948.1 and NP_001277949.1. Reference to mouse SIRPA proteins includes canonical forms as well as all allelic forms and isoforms. mRNAs (cDNAs) encoding the canonical isoform are assigned NCBI Accession Nos. NM_001291019.1 and NM_001291020.1. Reference to the mouse Sirpa mRNA (cDNA) and coding sequence includes the canonical forms as well as all allelic forms and isoforms.

Rat Sirpa maps to 3q36 on chromosome 3 (NCBI RefSeq Gene ID 25528; Assembly mRatBN7.2 (GCF_015227675.2); location NC_051338.1 (116819730 . . . 116858099). Reference to the rat Sirpa gene includes the canonical, wild type form as well as all allelic forms and isoforms. The canonical rat SIRPA protein has been assigned UniProt accession number P97710 and NCBI Accession No. NP_037148.2. Reference to rat SIRPA proteins includes canonical forms as well as all allelic forms and isoforms. An mRNA (cDNA) encoding the canonical isoform is assigned NCBI Accession No. NM_013016.2. Reference to the rat Sirpa mRNA (cDNA) and coding sequence includes the canonical forms as well as all allelic forms and isoforms.

Human SIRPA maps to 20p13 on chromosome 20 (NCBI RefSeq Gene ID 140885; Assembly GRCh38.p14 (GCF_000001405.40); location NC_000020.11 (1894167 . . . 1940592). Reference to the human SIRPA gene includes the canonical, wild type form as well as all allelic forms and isoforms. The canonical human SIRPA protein has been assigned UniProt accession number P78324 and NCBI Accession Nos. NP_001035111.1, NP_001035112.1, and NP_542970.1. Reference to human SIRPA proteins includes the canonical form as well as all allelic forms and isoforms. mRNAs (cDNAs) encoding the canonical isoform are assigned NCBI Accession Nos. NM_001040022.1, NM_001040023.1, and NM_080792.2. Reference to the human SIRPA mRNA (cDNA) and coding sequence includes the canonical forms as well as all allelic forms and isoforms.

Examples of non-human animals with humanized SIRPA genes are provided, e.g., in U.S. Pat. No. 9,901,083, herein incorporated by reference in its entirety for all purposes. The non-human animal (or cell or genome) can be, in some cases, homozygous for the humanized SIRPA gene. In other cases, the animal (or cell or genome) can be heterozygous for the humanized SIRPA gene. A diploid organism has two alleles at each genetic locus. Each pair of alleles represents the genotype of a specific genetic locus. Genotypes are described as homozygous if there are two identical alleles at a particular locus and as heterozygous if the two alleles differ. The non-human animal can comprise the humanized SIRPA gene in its germline. The humanized SIRPA gene can comprise a human SIRPA nucleic acid encoding a portion of a human SIRPA protein, such as the extracellular domain of a human SIRPA protein. The human SIRPA nucleic acid can be a genomic nucleic acid, such as a genomic nucleic acid comprising a region of a human SIRPA gene comprising exons 2-4, or can comprise a corresponding part of a human SIRPA complementary DNA (cDNA). Alternatively, the human SIRPA can comprise the entire coding sequence for a human SIRPA protein such that a fully human SIRPA protein is encoded. The human SIRPA nucleic acid can be inserted into the non-human animal Sirpa genomic locus, or it can replace a corresponding region of the non-human animal Sirpa locus (e.g., a region of a human SIRPA gene comprising exons 2-4 can replace exons 2-4 of the non-human animal Sirpa gene). The humanized SIRPA gene (or the human SIRPA nucleic acid) can be operably linked to the endogenous non-human animal Sirpa promoter. In other words, expression of the humanized SIRPA gene can be driven by the endogenous non-human animal Sirpa promoter.

Some genetically modified non-human animals described herein further comprise a humanized IL6 gene. In one example, such genetically modified non-human animals can comprise inactivated Fah, Rag2, and Il2rg genes and a humanized IL6 gene. In another example, such genetically modified non-human animals can comprise inactivated Fah, Rag2, and Il2rg genes and humanized SIRPA and IL6 genes. In one example, such genetically modified non-human animals can comprise inactivated Fah, Rag1, Rag2, and Il2rg genes and a humanized IL6 gene. In another example, such genetically modified non-human animals can comprise inactivated Fah, Rag1, Rag2, and Il2rg genes and humanized SIRPA and IL6 genes. In another example, such genetically modified non-human animals can be immunodeficient non-human animals comprising a humanized IL6 gene. In one example, the non-human animal is a mouse. In another example, the non-human animal is a rat.

IL6 (also known as IL-6 and IFNB2) encodes interleukin-6 (also known as IL-6, IL6, B-cell stimulatory factor 2 (BSF-2), CTL differentiation factor (CDF), hybridoma growth factor, and interferon beta-2 (IFN-beta-2). IL-6 is a cytokine with a wide variety of biological functions in immunity, tissue regeneration, and metabolism. It binds to IL-6R, then the complex associates with the signaling subunit IL6ST/gp130 to trigger the intracellular IL6-signaling pathway.

Mouse Il6 maps to 5 B1; 5 15.7 cM on chromosome 5 (NCBI RefSeq Gene ID 16193; Assembly GRCm39 (GCF_000001635.27); location NC_000071.7 (30218112 . . . 30224973). Reference to the mouse Il6 gene includes the canonical, wild type form as well as all allelic forms and isoforms. The canonical mouse IL-6 protein has been assigned UniProt accession number P08505 and NCBI Accession Nos. NP_001300983.1 and NP_112445.1. Reference to mouse IL-6 proteins includes canonical forms as well as all allelic forms and isoforms. mRNAs (cDNAs) encoding the canonical isoform are assigned NCBI Accession Nos. NM_001314054.1 and NM_031168.2. Reference to the mouse Il6 mRNA (cDNA) and coding sequence includes the canonical forms as well as all allelic forms and isoforms.

Rat Il6 maps to 4q11 on chromosome 4 (NCBI RefSeq Gene ID 24498; Assembly mRatBN7.2 (GCF_015227675.2); location NC_051339.1 (5214602 . . . 5219178, complement). Reference to the rat Il6 gene includes the canonical, wild type form as well as all allelic forms and isoforms. The canonical rat IL-6 protein has been assigned UniProt accession number P20607 and NCBI Accession No. NP_036721.1. Reference to rat IL-6 proteins includes canonical forms as well as all allelic forms and isoforms. An mRNA (cDNA) encoding the canonical isoform is assigned NCBI Accession No. NM_012589.2. Reference to the rat Il6 mRNA (cDNA) and coding sequence includes the canonical forms as well as all allelic forms and isoforms.

Human IL6 maps to 7p15.3 on chromosome 7 (NCBI RefSeq Gene ID 3569; Assembly GRCh38.p14 (GCF_000001405.40); location NC_000007.14 (22727200 . . . 22731998). Reference to the human IL6 gene includes the canonical, wild type form as well as all allelic forms and isoforms. The canonical human IL-6 protein has been assigned UniProt accession number P05231 and NCBI Accession No. NP_000591.1 (SEQ ID NO: 45). Reference to human IL-6 proteins includes the canonical form as well as all allelic forms and isoforms. An mRNA (cDNA) encoding the canonical isoform is assigned NCBI Accession No. NM_000600.4. A coding sequence for the canonical isoform is assigned CCDS Accession No. CCDS5375.1 (SEQ ID NO: 44). Reference to the human IL6 mRNA (cDNA) and coding sequence includes the canonical forms as well as all allelic forms and isoforms.

Examples of non-human animals with humanized IL6 genes are provided, e.g., in U.S. Pat. No. 9,622,460, herein incorporated by reference in its entirety for all purposes. The non-human animal (or cell or genome) can be, in some cases, homozygous for the humanized IL6 gene. In other cases, the animal (or cell or genome) can be heterozygous for the humanized IL6 gene. The non-human animal can comprise the humanized IL6 gene in its germline. The humanized IL6 gene can comprise a human IL6 nucleic acid encoding a human IL-6 protein (e.g., a fully human IL-6 protein). The human IL6 nucleic acid can be a genomic nucleic acid, such as a genomic nucleic acid comprising a region of a human IL6 gene from the start codon to the stop codon, or can comprise a human IL6 complementary DNA (cDNA). The human IL6 nucleic acid can be inserted into the non-human animal 116 genomic locus, or it can replace a corresponding region of the non-human animal 116 locus (e.g., a region of a human IL6 gene from the start codon to the stop codon can replace a region of the non-human animal 116 gene from the start codon to the stop codon). The humanized IL6 gene (or the human IL6 nucleic acid) can be operably linked to the endogenous non-human animal 116 promoter. In other words, expression of the humanized IL6 gene can be driven by the endogenous non-human animal 116 promoter.

Some genetically modified non-human animals described herein further comprise a humanized OSM gene. In one example, such genetically modified non-human animals can comprise inactivated Fah, Rag2, and Il2rg genes and a humanized OSM gene. In another example, such genetically modified non-human animals can comprise inactivated Fah, Rag2, and Il2rg genes and humanized SIRPA and OSM genes. In one example, such genetically modified non-human animals can comprise inactivated Fah, Rag1, Rag2, and Il2rg genes and a humanized OSM gene. In another example, such genetically modified non-human animals can comprise inactivated Fah, Rag1, Rag2, and Il2rg genes and humanized SIRPA and OSM genes. In another example, such genetically modified non-human animals can be immunodeficient non-human animals comprising a humanized OSM gene. In another example, such genetically modified non-human animals can comprise inactivated Fah, Rag2, and Il2rg genes and humanized IL6 and OSM genes. In another example, such genetically modified non-human animals can comprise inactivated Fah, Rag2, and Il2rg genes and humanized SIRPA, IL6, and OSM genes. In another example, such genetically modified non-human animals can comprise inactivated Fah, Rag1, Rag2, and Il2rg genes and humanized IL6 and OSM genes. In another example, such genetically modified non-human animals can comprise inactivated Fah, Rag1, Rag2, and Il2rg genes and humanized SIRPA, IL6, and OSM genes. In another example, such genetically modified non-human animals can be immunodeficient non-human animals comprising humanized IL6 and OSM genes. In one example, the non-human animal is a mouse. In another example, the non-human animal is a rat.

OSM encodes oncostatin-M (also known as OSM). OSM is a pleiotropic cytokine that belongs to the interleukin 6 group of cytokines. Of these cytokines, it most closely resembles leukemia inhibitory factor (LIF) in both structure and function. It is important in liver development, hematopoiesis, inflammation and possibly CNS development. It is also associated with bone formation and destruction. OSM signals through cell surface receptors that contain the protein GP130. The type I receptor is composed of GP130 and LIFR, the type II receptor is composed of GP130 and OSMR.

Mouse Osm maps to 11 A1; 11 2.94 cM on chromosome 11 (NCBI RefSeq Gene ID 18413; Assembly GRCm39 (GCF_000001635.27); location NC_000077.7 (4186785 . . . 4191026). Reference to the mouse Osm gene includes the canonical, wild type form as well as all allelic forms and isoforms. The canonical mouse OSM protein has been assigned UniProt accession number P53347 and NCBI Accession No. NP_001013383.1. Reference to mouse OSM proteins includes canonical forms as well as all allelic forms and isoforms. mRNAs (cDNAs) encoding the canonical isoform are assigned NCBI Accession No. NM_001013365.3. Reference to the mouse Osm mRNA (cDNA) and coding sequence includes the canonical forms as well as all allelic forms and isoforms.

Rat Osm maps to 14q21 on chromosome 14 (NCBI RefSeq Gene ID 289747; Assembly mRatBN7.2 (GCF_015227675.2); location NC_051349.1 (79103638 . . . 79108500). Reference to the rat Osm gene includes the canonical, wild type form as well as all allelic forms and isoforms. The canonical rat OSM protein has been assigned UniProt accession number Q65Z15 and NCBI Accession No. NP_001006962.1. Reference to rat OSM proteins includes canonical forms as well as all allelic forms and isoforms. An mRNA (cDNA) encoding the canonical isoform is assigned NCBI Accession No. NM_001006961.2. Reference to the rat Osm mRNA (cDNA) and coding sequence includes the canonical forms as well as all allelic forms and isoforms.

Human OSM maps to 22q12.2 on chromosome 22 (NCBI RefSeq Gene ID 5008; Assembly GRCh38.p14 (GCF_000001405.40); location NC_000022.11 (30262829 . . . 30266851, complement). Reference to the human OSM gene includes the canonical, wild type form as well as all allelic forms and isoforms. The canonical human OSM protein has been assigned UniProt accession number P13725 and NCBI Accession No. NP_065391.1 (SEQ ID NO: 51). Reference to human OSM proteins includes the canonical form as well as all allelic forms and isoforms. An mRNA (cDNA) encoding the canonical isoform is assigned NCBI Accession No. NM_020530.6. A coding sequence for the canonical isoform is assigned CCDS Accession No. CCDS13873.1 (SEQ ID NO: 50). Reference to the human OSM mRNA (cDNA) and coding sequence includes the canonical forms as well as all allelic forms and isoforms.

Some genetically modified non-human animals described herein further comprise a humanized Growth Hormone gene. Similar to IL6, mGH shows species specificities in receptor binding, and administration of human Growth Hormone (GH) in humanized liver mice could correct fatty liver in humanized liver mice. Tateno et al. (2011) Endocrinology 152:1479-1491, herein incorporated by reference in its entirety for all purposes.

The non-human animals (or cells or genomes) disclosed herein can be male or female. Non-human animal cells disclosed herein can be any type of undifferentiated or differentiated state. For example, a non-human animal cell can be a totipotent cell, a pluripotent cell (e.g., mouse or rat pluripotent cell such as a mouse or rat embryonic stem (ES) cell), or a non-pluripotent cell. Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that are capable of contributing to any tissue of the developing embryo upon introduction into an embryo. ES cells can be derived from the inner cell mass of a blastocyst and are capable of differentiating into cells of any of the three vertebrate germ layers (endoderm, ectoderm, and mesoderm).

The cells provided herein can also be germ cells (e.g., sperm or oocytes). The cells can be mitotically competent cells or mitotically inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells (e.g., hepatocytes).

Suitable cells provided herein also include primary cells. Primary cells include cells or cultures of cells that have been isolated directly from an organism, organ, or tissue. Primary cells include cells that are neither transformed nor immortal. They include any cell obtained from an organism, organ, or tissue which was not previously passed in tissue culture or has been previously passed in tissue culture but is incapable of being indefinitely passed in tissue culture. Such cells can be isolated by conventional techniques and include, for example, liver cells (e.g., hepatocytes).

Other suitable cells provided herein include immortalized cells. Immortalized cells include cells from a multicellular organism that would normally not proliferate indefinitely but, due to mutation or alteration, have evaded normal cellular senescence and instead can keep undergoing division. Such mutations or alterations can occur naturally or be intentionally induced. Numerous types of immortalized cells are well known. Immortalized or primary cells include cells that are typically used for culturing or for expressing recombinant genes or proteins.

The cells provided herein also include one-cell stage embryos (i.e., fertilized oocytes or zygotes). Such one-cell stage embryos can be from any genetic background, can be fresh or frozen, and can be derived from natural breeding or in vitro fertilization. The cells provided herein can be normal, healthy cells, or can be diseased or mutant-bearing cells.

The non-human animal can be a eukaryote, which includes, for example, animals and mammals. The term “animal” includes any member of the animal kingdom, including, for example, mammals, fishes, reptiles, amphibians, and birds. A mammal can be, for example, a pig, a rodent, a rat, or a mouse. Other mammals include, for example, non-human primates, cats, dogs, rabbits, cows, sheep, goats, pigs, and boars, and so forth. Birds include, for example, chickens, turkeys, ostrich, geese, ducks, and so forth. The term “non-human” excludes humans.

The non-human animals can be from any genetic background. For example, suitable mice, mouse cells, or mouse genomes can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 12951/SV, 12951/Svlm), 129S2, 129S4, 129S5, 12959/SvEvH, 129S6 (129/SvEvTac), 129S7, 129S8, 129T1, and 129T2. See, e.g., Festing et al. (1999) Mamm. Genome 10(8):836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/10ScSn, C57BL/10Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 129S6 (129/SvEvTac) strain).

Rats or rat cells or rat genomes can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats, rat cells, or rat genomes can also be obtained from a strain derived from a mix of two or more strains recited above. For example, a suitable rat can be from a DA strain or an ACI strain. The ACI rat strain is characterized as having black agouti, with white belly and feet and an RT1^{av1} haplotype. Such strains are available from a variety of sources including Harlan Laboratories. The Dark Agouti (DA) rat strain is characterized as having an agouti coat and an RT1^{av1} haplotype. Such rats are available from a variety of sources including Charles River and Harlan Laboratories. Some suitable rats, rat cells, and rat genomes can be from an inbred rat strain. See, e.g., US 2014/0235933, herein incorporated by reference in its entirety for all purposes.

The non-human animals described herein can also exhibit decreased liver function, such as phenotypic and biochemical manifestations of human hereditary tyrosinemia type I (HT1). Procedures for testing liver function are well known. See, e.g., Grompe et al. (1993) Genes Dev. 7:2298-2307 and Manning et al. (1999) Proc. Natl. Acad. Sci. U.S.A. 96:11928-11933. As an example, the non-human animals can exhibit one or more of the following (e.g., in the absence of nitisinone or any other compound that ameliorates toxicity caused by Fah deficiency and/or in the absence of transplantation of hepatocytes with a functional copy of Fah): hypertyrosinemia; liver fibrosis; cirrhosis; liver failure; and renal tubular damage or dysfunction. For example, the non-human animals described herein can exhibit hypertyrosinemia, liver failure, and renal tubular damage or dysfunction. Likewise, the non-human animals can exhibit liver fibrosis and cirrhosis. The non-human animals described herein can comprise an accumulation of toxic metabolites such as fumarylacetoacetate and maleylacetoacetate (e.g., in the absence of nitisinone or any other compound that ameliorates toxicity caused by Fah deficiency and/or in the absence of transplantation of hepatocytes with a functional copy of Fah). This accumulation can lead to hepatocyte loss, liver failure, and death (e.g., in the absence of nitisinone or any other compound that ameliorates toxicity caused by Fah deficiency and/or in the absence of transplantation of hepatocytes with a functional copy of Fah).

Some non-human animals described herein can further comprise nitisinone or any other compound that ameliorates toxicity caused by Fah deficiency. Some non-human animals described herein can also comprise transplanted (i.e., xenotransplanted) hepatocytes or transplanted and expanded hepatocytes (e.g., hepatocytes from a different species than the non-human animal, such as human hepatocytes). The transplanted hepatocytes can repopulate the liver of the non-human animals disclosed herein and can restore liver function (e.g., that was lost or decreased due to the inactivated Fah gene). As one example, the non-human animals described herein can comprise xenotransplanted human hepatocytes. The transplanted hepatocytes can be wild type hepatocytes, or they can comprise one or more mutations. In a specific example, the transplanted hepatocytes have a wild type FAH gene or an FAH gene that produces a functional FAH protein.

B. Modifications to Restore IL-6/IL-6R Signaling Pathway Activity or GP130 Signaling Pathway Activity in Xenotransplanted Hepatocytes

The genetically modified non-human animals (e.g., mice or rats) comprising xenotransplanted hepatocytes (e.g., human hepatocytes) disclosed herein can comprise modifications to the genetically modified non-human animals and/or the xenotransplanted hepatocytes to restore IL-6/IL-6R signaling pathway or GP130 signaling pathway activity in the xenotransplanted hepatocytes. In some cases, the genetically modified non-human animal does not comprise (and/or is not modified to comprise) Kupffer cells that are species-matched to the transplanted hepatocytes. In some cases, the genetically modified non-human animal does not comprise (and/or is not modified to comprise) Kupffer cells in the liver that are species-matched to the transplanted hepatocytes. In some cases, the genetically modified non-human animal does not comprise (and/or is not modified to comprise) a reconstituted immune system that is species-matched to the transplanted hepatocytes. In some cases, the genetically modified non-human animal does not comprise (and/or is not modified to comprise) human Kupffer cells. In some cases, the genetically modified non-human animal does not comprise (and/or is not modified to comprise) human Kupffer cells in the liver. In some cases, the genetically modified non-human animal does not comprise (and/or is not modified to comprise) a reconstituted human immune system. In some cases, the genetically modified non-human animal does not comprise (and/or is not modified to comprise) Kupffer cells that are species-compatible to the transplanted hepatocytes (i.e., that produce IL-6 compatible with the IL-6R in the transplanted hepatocytes). In some cases, the genetically modified non-human animal does not comprise (and/or is not modified to comprise) Kupffer cells in the liver that are species-compatible to the transplanted hepatocytes. In some cases, the genetically modified non-human animal does not comprise (and/or is not modified to comprise) a reconstituted immune system that is species-compatible to the transplanted hepatocytes.

The genetically modified non-human animals can comprise xenotransplanted hepatocytes (e.g., xenotransplanted and expanded hepatocytes). The xenotransplanted hepatocytes can be from any species other than that of the recipient non-human animal. For example, the xenotransplanted hepatocytes can be human hepatocytes. In another example, the xenotransplanted hepatocytes can be non-human primate (NHP) hepatocytes, such as cynomolgus hepatocytes. The transplanted hepatocytes can be from a species whose IL-6R is incompatible with the endogenous non-human animal IL-6 (e.g., the transplanted hepatocytes can be human hepatocytes, and the non-human animal can be a mouse or a rat). The cross-species incompatibility between the transplanted hepatocytes and the recipient non-human animals (i.e., the suboptimal interaction or lack of reactivity between the recipient non-human animal IL-6 ligands and the IL-6R on the xenotransplanted hepatocytes) can result in a steatosis-like phenotype and lipid droplet accumulation.

The modifications described herein can result in reduced lipid droplet accumulation and reduced steatosis in the xenotransplanted hepatocytes compared to non-human animals in which the genetically modified non-human animals and the xenotransplanted hepatocytes do not have modifications to restore IL-6/IL-6R signaling pathway or GP130 signaling pathway activity in the xenotransplanted hepatocytes. The modifications can, for example, restore species-matched hepatic IL-6/IL-6R pathway activity or GP130 signaling pathway activity in the xenotransplanted hepatocytes, or can simply restore IL-6/IL-6R signaling pathway activity or GP130 signaling pathway activity in the xenotransplanted hepatocytes. Likewise, the modifications can, for example, restore species-compatible hepatic IL-6/IL-6R pathway activity or GP130 signaling pathway activity in the xenotransplanted hepatocytes.

In some cases, the xenotransplanted hepatocytes are modified to restore IL-6/IL-6R signaling pathway activity or GP130 signaling pathway activity in the xenotransplanted hepatocytes. As one example, the transplanted hepatocytes can be modified to express (i.e., ectopically express) non-human animal IL-6R (i.e., IL-6R from the same species as the recipient non-human animal—in other words, IL-6R species-matched to the recipient non-human animal). As another example, the transplanted hepatocytes can be modified to express (i.e., ectopically express) species-compatible IL-6R (i.e., IL-6R from a species that is compatible with the IL-6 produced by the recipient non-human animal, such that the recipient non-human animal IL-6 can bind to and activate IL-6R signaling). For example, if the hepatocytes are being transplanted into a mouse or a rat, the transplanted hepatocytes can be genetically modified to express mouse IL-6R or rat IL-6R, respectively. In some cases, the transplanted hepatocytes can be modified to comprise a vector comprising an expression construct for the non-human animal IL-6R comprising a nucleic acid encoding the non-human animal IL-6R operably linked to a promoter. Any suitable vector can be used. For example, the vector can be a viral vector, such as a lentiviral vector or an adeno-associated virus (AAV) vector. In a specific example, a lentiviral vector is used. Alternatively, the transplanted hepatocytes can be genetically modified to comprise in their genome a non-human animal IL-6R expression construct comprising a nucleic acid encoding the non-human animal IL-6R operably linked to a promoter. In the case of genomic modification or in the case of a vector, any suitable promoter can be used. In one example, a liver-specific promoter or a promoter active in liver cells (e.g., hepatocytes) can be used. Examples of such promoters include TTR, ALB, and HBV promoters. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

Mouse interleukin-6 receptor subunit alpha (also known as IL-6 receptor subunit alpha, IL-6R subunit alpha, IL-6R-alpha, IL-6RA, IL-6R, IL-6R 1, and CD126) is encoded by the gene Il6ra (also known as Il6r). IL-6R is part of the receptor for interleukin 6. It binds to IL-6 with low affinity but does not transduce a signal. Signal activation necessitate an association with IL6ST. Activation leads to the regulation of the immune response, acute-phase reactions and hematopoiesis. The interaction with membrane-bound IL-6R and IL6ST stimulates classic signaling, and the restricted expression of the IL-6R limits classic IL-6 signaling to only a few tissues such as the liver and some cells of the immune system. Mouse Il6ra maps to 3 F1; 3 39.19 cM on chromosome 3 (NCBI RefSeq Gene ID 16194; Assembly GRCm39 (GCF_000001635.27); location NC_000069.7 (89776631 . . . 89820503, complement). Reference to the mouse Il6ra gene includes the canonical, wild type form as well as all allelic forms and isoforms. The canonical mouse IL-6R protein has been assigned UniProt accession number P22272 and NCBI Accession No. NP_034689.2 (SEQ ID NO: 41). Reference to mouse IL-6R proteins includes canonical forms as well as all allelic forms and isoforms. An mRNA (cDNA) encoding the canonical isoform is assigned NCBI Accession No. NM_010559.3. The coding sequence for the canonical isoform is assigned CCDS Accession No. CCDS38496.1 (SEQ ID NO: 40). Reference to the mouse Il6ra mRNA (cDNA) and coding sequence includes the canonical forms as well as all allelic forms and isoforms.

Rat Il6ra maps to 2q34 on chromosome 2 (NCBI RefSeq Gene ID 24499; Assembly mRatBN7.2 (GCF_015227675.2); location NC_051337.1 (175289157 . . . 175347719, complement). Reference to the rat Il6ra gene includes the canonical, wild type form as well as all allelic forms and isoforms. Rat IL-6R protein has been assigned UniProt accession number P22273. The canonical rat IL-6R protein has been assigned NCBI Accession No. NP_058716.2 (SEQ ID NO: 43). Reference to rat IL-6R proteins includes canonical forms as well as all allelic forms and isoforms. An mRNA (cDNA) encoding the canonical isoform is assigned NCBI Accession No. NM_017020.3. A coding sequence for the canonical isoform is set forth in SEQ ID NO: 42. Reference to the rat Il6ra mRNA (cDNA) and coding sequence includes the canonical forms as well as all allelic forms and isoforms.

As another example, the transplanted hepatocytes can be modified to express (i.e., ectopically express) non-human animal OSMR (i.e., OSMR from the same species as the recipient non-human animal—in other words, OSMR species-matched to the recipient non-human animal). As another example, the transplanted hepatocytes can be modified to express (i.e., ectopically express) species-compatible OSMR (i.e., OSMR from a species that is compatible with the OSM produced by the recipient non-human animal, such that the recipient non-human animal OSM can bind to and activate OSMR signaling). For example, if the hepatocytes are being transplanted into a mouse or a rat, the transplanted hepatocytes can be genetically modified to express mouse OSMR or rat OSMR, respectively. In some cases, the transplanted hepatocytes can be modified to comprise a vector comprising an expression construct for the non-human animal OSMR comprising a nucleic acid encoding the non-human animal OSMR operably linked to a promoter. Any suitable vector can be used. For example, the vector can be a viral vector, such as a lentiviral vector or an adeno-associated virus (AAV) vector. In a specific example, a lentiviral vector is used. Alternatively, the transplanted hepatocytes can be genetically modified to comprise in their genome a non-human animal OSMR expression construct comprising a nucleic acid encoding the non-human animal OSMR operably linked to a promoter. In the case of genomic modification or in the case of a vector, any suitable promoter can be used. In one example, a liver-specific promoter or a promoter active in liver cells (e.g., hepatocytes) can be used. Examples of such promoters include TTR, ALB, and HBV promoters. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

Mouse oncostatin-M-specific receptor subunit beta (also known as oncostatin M receptor or OSMR or OSMRB) is encoded by the gene Osmr (also known as Osmrb or oncostatin M receptor). It is capable of transducing OSM-specific signaling events through association with GP130 and activating STAT3 downstream. Mouse Osmr maps to 15 A1; 15 3.3 cM on chromosome 15 (NCBI RefSeq Gene ID 18414; Assembly GRCm39 (GCF_000001635.27); location NC_000081.7 (6843049 . . . 6904434, complement). Reference to the mouse Osmr gene includes the canonical, wild type form as well as all allelic forms and isoforms. The canonical mouse OSMR protein has been assigned UniProt accession number 070458 and NCBI Accession No. NP_035149.2 (SEQ ID NO: 53). Reference to mouse OSMR proteins includes canonical forms as well as all allelic forms and isoforms. An mRNA (cDNA) encoding the canonical isoform is assigned NCBI Accession No. NM_011019.4. The coding sequence for the canonical isoform is assigned CCDS Accession No. CCDS27368.1 (SEQ ID NO: 52). Reference to the mouse Osmr mRNA (cDNA) and coding sequence includes the canonical forms as well as all allelic forms and isoforms.

Rat Osmr maps to 2q16 on chromosome 2 (NCBI RefSeq Gene ID 310132; Assembly mRatBN7.2 (GCF_015227675.2); location NC_051337.1 (55907119 . . . 55961373, complement). Reference to the rat Osmr gene includes the canonical, wild type form as well as all allelic forms and isoforms. Rat OSMR protein has been assigned UniProt accession number Q65Z14. The canonical rat OSMR protein has been assigned NCBI Accession No. NP_001005384.1 (SEQ ID NO: 55). Reference to rat OSMR proteins includes canonical forms as well as all allelic forms and isoforms. An mRNA (cDNA) encoding the canonical isoform is assigned NCBI Accession No. NM_001005384.1. A coding sequence for the canonical isoform is set forth in SEQ ID NO: 54. Reference to the rat Osmr mRNA (cDNA) and coding sequence includes the canonical forms as well as all allelic forms and isoforms.

As another example, the transplanted hepatocytes can be modified to express (i.e., ectopically express) non-human animal IL-6R and non-human animal OSMR (i.e., IL-6R and OSMR from the same species as the recipient non-human animal—in other words, IL-6R and OSMR species-matched to the recipient non-human animal). As another example, the transplanted hepatocytes can be modified to express (i.e., ectopically express) species-compatible IL-6R and OSMR (i.e., IL-6R and OSMR from a species that is compatible with the IL-6 and OSM, respectively, produced by the recipient non-human animal, such that the recipient non-human animal IL-6 and OSM can bind to and activate IL-6R and OSMR signaling, respectively). As another example, the transplanted hepatocytes can be modified to express (i.e., ectopically express) the non-human animal receptors for IL-6, LIF, OSM, CNTF, IL-11, CTF1, BSF3, or any combination thereof. As another example, the transplanted hepatocytes can be modified to express (i.e., ectopically express) species-compatible receptors for IL-6, LIF, OSM, CNTF, IL-11, CTF1, BSF3, or any combination thereof.

As another example, the transplanted hepatocytes can be modified to express (i.e., ectopically express) non-human animal Growth Hormone Receptor (GHR) (i.e., GHR from the same species as the recipient non-human animal—in other words, GHR species-matched to the recipient non-human animal). As another example, the transplanted hepatocytes can be modified to express (i.e., ectopically express) species-compatible GHR (i.e., GHR from a species that is compatible with the GH produced by the recipient non-human animal, such that the recipient non-human animal GH can bind to and activate GHR signaling). For example, if the hepatocytes are being transplanted into a mouse or a rat, the transplanted hepatocytes can be genetically modified to express mouse GHR or rat GHR, respectively. In some cases, the transplanted hepatocytes can be modified to comprise a vector comprising an expression construct for the non-human animal GHR comprising a nucleic acid encoding the non-human animal GHR operably linked to a promoter. Any suitable vector can be used. For example, the vector can be a viral vector, such as a lentiviral vector or an adeno-associated virus (AAV) vector. In a specific example, a lentiviral vector is used. Alternatively, the transplanted hepatocytes can be genetically modified to comprise in their genome a non-human animal GHR expression construct comprising a nucleic acid encoding the non-human animal GHR operably linked to a promoter. In the case of genomic modification or in the case of a vector, any suitable promoter can be used. In one example, a liver-specific promoter or a promoter active in liver cells (e.g., hepatocytes) can be used. Examples of such promoters include TTR, ALB, and HBV promoters. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

As another example of modifying the transplanted hepatocytes, they can be modified to express (i.e., ectopically express) a ligand-independent, constitutively active form of IL-6R co-receptor glycoprotein 130 (GP130). For example, if the transplanted hepatocytes are human hepatocytes, the constitutively active form of GP130 can be a human GP130. In some cases, the transplanted hepatocytes can be modified to comprise a vector comprising an expression construct for the constitutively active form of GP130 comprising a nucleic acid encoding the constitutively active form of GP130 operably linked to a promoter. Any suitable vector can be used. For example, the vector can be a viral vector, such as a lentiviral vector or an adeno-associated virus (AAV) vector. In a specific example, a lentiviral vector is used. Alternatively, the transplanted hepatocytes can be genetically modified to comprise in their genome a GP130 expression construct comprising a nucleic acid encoding the constitutively active form of GP130 operably linked to a promoter. For example, the expression construct could be at a safe harbor locus in the non-human animal. In the case of genomic modification or in the case of a vector, any suitable promoter can be used. In one example, a liver-specific promoter or a promoter active in liver cells (e.g., hepatocytes) can be used. Examples of such promoters include TTR, ALB, and HBV promoters. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

Human interleukin-6 receptor subunit beta (also known as IL6ST, IL-6 receptor subunit beta, IL-6R subunit beta, IL-6R-beta, IL-6RB, CDw130, interleukin-6 signal transducer, membrane glycoprotein 130 (gp130), CD130, and oncostatin-M receptor subunit alpha) is encoded by the gene IL6ST. The receptor systems for IL-6, LIF, OSM, CNTF, IL-11, CTF1, and BSF3 can utilize IL6ST (GP130) for initiating signal transmission. Binding of IL-6 to IL-6R induces IL6ST (GP130) homodimerization and formation of a high-affinity receptor complex, which activates the intracellular JAK-MAPK and JAK-STAT3 signaling pathways. Human IL6ST maps to 5q11.2 on chromosome 5 (NCBI RefSeq Gene ID 3572; Assembly GRCh38.p14 (GCF_000001405.40); location NC_000005.10 (55935095 . . . 55994963, complement). Reference to the human IL6ST gene includes the canonical, wild type form as well as all allelic forms and isoforms. The canonical human IL6ST (GP130) protein has been assigned UniProt accession number P40189 and NCBI Accession No. NP_002175.2 (SEQ ID NO: 49). Reference to human IL6ST (GP130) proteins includes canonical forms as well as all allelic forms and isoforms. An mRNA (cDNA) encoding the canonical isoform is assigned NCBI Accession No. NM_002184.3. A coding sequence encoding the canonical isoform is assigned CCDS Accession No. CCDS3971.1 (SEQ ID NO: 48). Reference to the human IL6ST mRNA (cDNA) and coding sequence includes the canonical forms as well as all allelic forms and isoforms. In one example, the constitutively active human GP130 comprises a deletion of the region of GP130 from Tyr186 to Tyr190 (GP130^{Y186-Y190del}). An exemplary GP130^{Y186-Y190del} protein is set forth in SEQ ID NO: 47 and is encoded by the coding sequence set forth in SEQ ID NO: 46.

In other cases, the recipient non-human animal is modified to restore IL-6/IL-6R signaling pathway activity or GP130 signaling pathway activity in the xenotransplanted hepatocytes. For example, the non-human animal can comprise IL-6 from the species of the xenotransplanted hepatocytes (e.g., IL-6 that is species-matched with the xenotransplanted hepatocytes). Likewise, the non-human animal can comprise IL-6 compatible with the species of the xenotransplanted hepatocytes (e.g., IL-6 that is species-compatible with the xenotransplanted hepatocytes, such that the IL-6 can activate IL-6R signaling in the xenotransplanted hepatocytes). For example, the non-human animal can comprise human IL-6 (or a human-IL-6R-compatible ligand (e.g., human-IL-6R-compatible IL-6), such as cynomolgus IL-6) if the xenotransplanted hepatocytes are human hepatocytes. For example, the non-human animal can comprise human IL-6 (or a human-IL-6R-compatible IL-6, such as cynomolgus IL-6) if the xenotransplanted hepatocytes are human hepatocytes. Human IL-6 is described above. The IL-6 can be, for example, in the serum of the non-human animal or the liver of the non-human animal. The IL-6 can be expressed by any suitable cell type in the non-human animal. In one example, the IL-6 is expressed in muscle cells in the non-human animal. In one example, the non-human animal comprises a vector comprising an expression construct for species-matched IL-6 (e.g., human IL-6) comprising a nucleic acid encoding the species-matched IL-6 (e.g., human IL-6) operably linked to a promoter. Likewise, the non-human animal can comprise a vector comprising an expression construct for species-compatible IL-6 (e.g., the IL-6 is compatible with the IL-6R expressed by the transplanted hepatocytes such that the IL-6 can activate IL-6R signaling in the hepatocytes) comprising a nucleic acid encoding the species-compatible IL-6 operably linked to a promoter. For example, if the transplanted hepatocytes are human hepatocytes, the non-human animal can comprise a vector comprising an expression construct for human-IL-6R-compatible ligand (e.g., human-IL-6R-compatible IL-6) (e.g., cynomolgus IL-6) comprising a nucleic acid encoding the human-IL-6R-compatible ligand (e.g., human-IL-6R-compatible IL-6) operably linked to a promoter. For example, if the transplanted hepatocytes are human hepatocytes, the non-human animal can comprise a vector comprising an expression construct for human-IL-6R-compatible IL-6 (e.g., cynomolgus IL-6) comprising a nucleic acid encoding the human-IL-6R-compatible IL-6 operably linked to a promoter. For example, the non-human animal can comprise the vector in muscle cells. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Alternatively, the non-human animal can comprise in its genome a species-matched IL-6 (e.g., human IL-6) expression construct comprising a nucleic acid encoding the species-matched IL-6 (e.g., human IL-6) operably linked to a promoter. Likewise, the non-human animal can comprise in its genome a species-compatible IL-6 (e.g., the IL-6 is compatible with the IL-6R expressed by the transplanted hepatocytes such that the IL-6 can activate IL-6R signaling in the hepatocytes) expression construct comprising a nucleic acid encoding the species-compatible IL-6 operably linked to a promoter. For example, if the transplanted hepatocytes are human hepatocytes, the non-human animal can comprise in its genome a human-IL-6R-compatible ligand (e.g., human-IL-6R-compatible IL-6) (e.g., cynomolgus IL-6) expression construct comprising a nucleic acid encoding the human-IL-6R-compatible ligand (e.g., human-IL-6R-compatible IL-6) operably linked to a promoter. For example, if the transplanted hepatocytes are human hepatocytes, the non-human animal can comprise in its genome a human-IL-6R-compatible IL-6 (e.g., cynomolgus IL-6) expression construct comprising a nucleic acid encoding the human-IL-6R-compatible IL-6 operably linked to a promoter. For example, the expression construct could be at a safe harbor locus in the non-human animal. In the case of genomic modification or in the case of a vector, any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MH) and muscle creatine kinase (CK) promoter (MHCK7) as described herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

In another example, the non-human animal can comprise OSM from the species of the xenotransplanted hepatocytes (e.g., OSM that is species-matched with the xenotransplanted hepatocytes). Likewise, the non-human animal can comprise OSM compatible with the species of the xenotransplanted hepatocytes (e.g., OSM that is species-compatible with the xenotransplanted hepatocytes, such that the OSM can activate OSMR signaling in the xenotransplanted hepatocytes). For example, the non-human animal can comprise human OSM (or a human-OSMR-compatible ligand (e.g., human-OSMR-compatible OSM)) if the xenotransplanted hepatocytes are human hepatocytes. Human OSM is described above. The OSM can be, for example, in the serum of the non-human animal or the liver of the non-human animal. The OSM can be expressed by any suitable cell type in the non-human animal. In one example, the OSM is expressed in muscle cells in the non-human animal. In one example, the non-human animal comprises a vector comprising an expression construct for species-matched OSM (e.g., human OSM) comprising a nucleic acid encoding the species-matched OSM (e.g., human OSM) operably linked to a promoter. Likewise, the non-human animal can comprise a vector comprising an expression construct for species-compatible OSM (e.g., the OSM is compatible with the OSMR expressed by the transplanted hepatocytes such that the OSM can activate OSMR signaling in the hepatocytes) comprising a nucleic acid encoding the species-compatible OSM operably linked to a promoter. For example, if the transplanted hepatocytes are human hepatocytes, the non-human animal can comprise a vector comprising an expression construct for human-OSMR-compatible ligand (e.g., human-OSMR-compatible OSM) (e.g., cynomolgus OSM) comprising a nucleic acid encoding the human-OSMR-compatible ligand (e.g., human-OSMR-compatible OSM) operably linked to a promoter. For example, the non-human animal can comprise the vector in muscle cells. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Alternatively, the non-human animal can comprise in its genome a species-matched OSM (e.g., human OSM) expression construct comprising a nucleic acid encoding the species-matched OSM (e.g., human OSM) operably linked to a promoter. Likewise, the non-human animal can comprise in its genome a species-compatible OSM (e.g., the OSM is compatible with the OSMR expressed by the transplanted hepatocytes such that the OSM can activate OSMR signaling in the hepatocytes) expression construct comprising a nucleic acid encoding the species-compatible OSM operably linked to a promoter. For example, if the transplanted hepatocytes are human hepatocytes, the non-human animal can comprise in its genome a human-OSMR-compatible ligand (e.g., human-OSMR-compatible OSM) (e.g., cynomolgus OSM) expression construct comprising a nucleic acid encoding the human-OSMR-compatible ligand (e.g., human-OSMR-compatible OSM) operably linked to a promoter. For example, the expression construct could be at a safe harbor locus in the non-human animal. In the case of genomic modification or in the case of a vector, any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MH) and muscle creatine kinase (CK) promoter (MHCK7) as described herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

In other cases, the recipient non-human animal is modified to restore IL-6/IL-6R signaling pathway activity or GP130 signaling pathway activity in the xenotransplanted hepatocytes. For example, the non-human animal can comprise a GP130 activator (e.g., human GP130 activator), such as a GP130-activating ligand (e.g., human-GP130-activating ligand). In one example, the non-human animal can comprise one or more GP130-activating ligands. In another example, the non-human animal can comprise two or more GP130-activating ligands. In another example, the non-human animal can comprise three or more GP130-activating ligands. In another example, the non-human animal can comprise four or more GP130-activating ligands. Ligands that activate GP130 are known. For example, IL-6, LIF, OSM, CNTF, IL-11, CTF1, and BSF3 are all ligands that can activate GP130. In one example, the non-human animals can comprise a ligand (e.g., IL-6, leukemia inhibitory factor (LIF), oncostatin M (OSM), ciliary neurotrophic factor (CNTF), interleukin-11 (IL-11), cardiotrophin-1 (CTF1), or cardiotrophin-like cytokine factor 1 (BSF3)) from the species of the xenotransplanted hepatocytes (e.g., IL-6, LIF, OSM, CNTF, IL-11, CTF1, or BSF3 that is species-matched or species-compatible with the xenotransplanted hepatocytes). In one example, the non-human animals can comprise IL-6 (e.g., species-matched or species-compatible with the xenotransplanted hepatocytes). In another example, the non-human animals can comprise OSM (e.g., species-matched or species-compatible with the xenotransplanted hepatocytes). In another example, the non-human animals can comprise IL-6 and OSM (e.g., species-matched or species-compatible with the xenotransplanted hepatocytes). In another example, the non-human animals can comprise IL-6, LIF, OSM, CNTF, IL-11, CTF1, BSF3, or any combination thereof (e.g., species-matched or species-compatible with the xenotransplanted hepatocytes). For example, the non-human animal can comprise human IL-6, LIF, OSM, CNTF, IL-11, CTF1, or BSF3 if the xenotransplanted hepatocytes are human hepatocytes. Another example of a GP130 activator is a GP130 agonist antibody or antigen-binding protein (e.g., human GP130 agonist antibody or antigen-binding protein). GP130-activating antibodies are known. See, e.g., Autissier et al. (1997) Eur. J. Immunol. 27(3):794-797, herein incorporated by reference in its entirety for all purposes. Another example of a GP130 activator is a chimeric GP130 ligand, termed IC7Fc, where one GP130 binding site has been removed from IL-6 and replaced with the leukemia inhibitory factor receptor (LIFR) binding site from CNTF and then fused with the fragment crystallizable (Fc) domain of immunoglobulin G (IgG). See, e.g., Findeisen et al. (2019) Nature 574:63-68, herein incorporated by reference in its entirety for all purposes. The GP130 activator (e.g., ligand) can be, for example, in the serum of the non-human animal or the liver of the non-human animal. The GP130 activator (e.g., ligand) can be expressed by any suitable cell type in the non-human animal. In one example, the GP130 activator (e.g., ligand) is expressed in muscle cells in the non-human animal. In one example, the non-human animal comprises a vector comprising an expression construct for the GP130 activator (e.g., ligand) comprising a nucleic acid encoding the GP130 activator operably linked to a promoter. For example, the non-human animal can comprise the vector in muscle cells. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Alternatively, the non-human animal can comprise in its genome a GP130 activator (e.g. ligand) expression construct comprising a nucleic acid encoding the GP130 activator (e.g. ligand) operably linked to a promoter. For example, the expression construct could be at a safe harbor locus in the non-human animal. In the case of genomic modification or in the case of a vector, any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MH) and muscle creatine kinase (CK) promoter (MHCK7) as described herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

In another example, the non-human animal can comprise Growth Hormone (GH) from the species of the xenotransplanted hepatocytes (e.g., GH that is species-matched with the xenotransplanted hepatocytes). Likewise, the non-human animal can comprise GH compatible with the species of the xenotransplanted hepatocytes (e.g., GH that is species-compatible with the xenotransplanted hepatocytes, such that the GH can activate GHR signaling in the xenotransplanted hepatocytes). For example, the non-human animal can comprise human GH (or a human-GHR-compatible GH) if the xenotransplanted hepatocytes are human hepatocytes. The GH can be, for example, in the serum of the non-human animal or the liver of the non-human animal. The GH can be expressed by any suitable cell type in the non-human animal. In one example, the GH is expressed in muscle cells in the non-human animal. In one example, the non-human animal comprises a vector comprising an expression construct for species-matched GH (e.g., human GH) comprising a nucleic acid encoding the species-matched GH (e.g., human GH) operably linked to a promoter. Likewise, the non-human animal can comprise a vector comprising an expression construct for species-compatible GH (e.g., the GH is compatible with the GHR expressed by the transplanted hepatocytes such that the GH can activate GHR signaling in the hepatocytes) comprising a nucleic acid encoding the species-compatible GH operably linked to a promoter. For example, if the transplanted hepatocytes are human hepatocytes, the non-human animal can comprise a vector comprising an expression construct for human-GHR-compatible GH (e.g., cynomolgus GH) comprising a nucleic acid encoding the human-GHR-compatible GH operably linked to a promoter. For example, the non-human animal can comprise the vector in muscle cells. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Alternatively, the non-human animal can comprise in its genome a species-matched GH (e.g., human GH) expression construct comprising a nucleic acid encoding the species-matched GH (e.g., human GH) operably linked to a promoter. Likewise, the non-human animal can comprise in its genome a species-compatible GH (e.g., the GH is compatible with the GHR expressed by the transplanted hepatocytes such that the GH can activate GHR signaling in the hepatocytes) expression construct comprising a nucleic acid encoding the species-compatible GH operably linked to a promoter. For example, if the transplanted hepatocytes are human hepatocytes, the non-human animal can comprise in its genome a human-GHR-compatible GH (e.g., cynomolgus GH) expression construct comprising a nucleic acid encoding the human-GHR-compatible GH operably linked to a promoter. For example, the expression construct could be at a safe harbor locus in the non-human animal. In the case of genomic modification or in the case of a vector, any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MH) and muscle creatine kinase (CK) promoter (MHCK7) as described herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

In another example, the non-human animal can comprise a humanized IL6 locus as described elsewhere herein. Examples of non-human animals with humanized IL6 genes are provided, e.g., in U.S. Pat. No. 9,622,460, herein incorporated by reference in its entirety for all purposes. The non-human animal can be, in some cases, homozygous for the humanized IL6 gene. In other cases, the non-human animal can be heterozygous for the humanized IL6 gene. The non-human animal can comprise the humanized IL6 gene in its germline. The humanized IL6 gene can comprise a human IL6 nucleic acid encoding a human IL-6 protein (e.g., a fully human IL-6 protein). The human IL6 nucleic acid can be a genomic nucleic acid, such as a genomic nucleic acid comprising a region of a human IL6 gene from the start codon to the stop codon, or can comprise a human IL6 complementary DNA (cDNA). The human IL6 nucleic acid can be inserted into the non-human animal 116 genomic locus, or it can replace a corresponding region of the non-human animal 116 locus (e.g., a region of a human IL6 gene from the start codon to the stop codon can replace a region of the non-human animal 116 gene from the start codon to the stop codon). The humanized IL6 gene (or the human IL6 nucleic acid) can be operably linked to the endogenous non-human animal 116 promoter. In other words, expression of the humanized IL6 gene can be driven by the endogenous non-human animal 116 promoter.

In another example, the non-human animal can comprise a humanized OSM locus as described elsewhere herein. The non-human animal can be, in some cases, homozygous for the humanized OSM gene. In other cases, the non-human animal can be heterozygous for the humanized OSM gene. The non-human animal can comprise the humanized OSM gene in its germline. The humanized OSM gene can comprise a human OSM nucleic acid encoding a human OSM protein (e.g., a fully human OSM protein). The human OSM nucleic acid can be a genomic nucleic acid, such as a genomic nucleic acid comprising a region of a human OSM gene from the start codon to the stop codon, or can comprise a human OSM complementary DNA (cDNA). The human OSM nucleic acid can be inserted into the non-human animal Osm genomic locus, or it can replace a corresponding region of the non-human animal Osm locus (e.g., a region of a human OSM gene from the start codon to the stop codon can replace a region of the non-human animal Osm gene from the start codon to the stop codon). The humanized OSM gene (or the human OSM nucleic acid) can be operably linked to the endogenous non-human animal Osm promoter. In other words, expression of the humanized OSM gene can be driven by the endogenous non-human animal Osm promoter.

In another example, the non-human animal can comprise a humanized IL6 locus as described elsewhere herein and a humanized OSM locus as described elsewhere herein.

In another example, the non-human animal can comprise a humanized GH locus. Similar to IL6, mGH shows species specificities in receptor binding, and administration of human Growth Hormone (GH) in humanized liver mice could correct fatty liver in humanized liver mice. Tateno et al. (2011) Endocrinology 152:1479-1491, herein incorporated by reference in its entirety for all purposes.

In another example, the non-human animal can comprise a humanized IL6 locus as described elsewhere herein and a humanized GH locus as described elsewhere herein.

In another example, the non-human animal can comprise a humanized OSM locus as described elsewhere herein and a humanized GH locus as described elsewhere herein.

In another example, the non-human animal can comprise a humanized IL6 locus as described elsewhere herein and a humanized OSM locus as described elsewhere herein and a humanized GH locus as described elsewhere herein.

### III. Methods of Making and Using Genetically Modified Non-Human Animals Comprising Xenotransplanted Hepatocytes

Methods are provided for making non-human animals with humanized livers (e.g., with reduced lipid droplet accumulation and/or reduced hepatosteatosis), methods of preventing, reducing, or ameliorating lipid droplet accumulation and/or hepatosteatosis in non-human animals with xenotransplanted hepatocytes and/or humanized livers, methods of assessing the activity of human-liver-targeting agents, and methods of making the non-human animals disclosed herein.

A. Methods of Making Non-Human Animals with Humanized Livers or Livers from Other Species

Various methods are provided for making a non-human animal (e.g., mouse or rat) comprising xenotransplanted hepatocytes (e.g., transplanted human hepatocytes) and/or a humanized liver or a liver from another species (i.e., a different species from the recipient non-human animal). Such methods can comprise transplanting hepatocytes or hepatocytes progenitors from a species different from the species of the recipient non-human animal (e.g., transplanting human hepatocytes or human hepatocyte progenitors) into a non-human animal described herein and allowing the transplanted hepatocytes or hepatocyte progenitors to expand. Such methods can be used to make non-human animals comprising livers from humans or from any other mammalian species as well (e.g., mouse, dog, pig, or non-human primate, such as cynomolgus) by transplanting the corresponding heterologous hepatocytes or hepatocyte progenitors.

The genetically modified non-human animals (e.g., mice or rats) hepatocytes (e.g., human hepatocytes) into which the hepatocytes or hepatocyte progenitors are transplanted can comprise modifications to the genetically modified non-human animals and/or the xenotransplanted hepatocytes/hepatocyte progenitors to restore IL-6/IL-6R signaling pathway activity or GP130 signaling pathway activity in the xenotransplanted hepatocytes/hepatocyte progenitors. In addition, the methods disclosed herein can further comprise modifying the hepatocytes or hepatocyte progenitors to be transplanted and/or modifying the non-human animal to restore IL-6/IL-6R signaling pathway activity or GP130 signaling pathway activity in the transplanted hepatocytes or hepatocyte progenitors. In some cases, the genetically modified non-human animal does not comprise Kupffer cells that are species-matched to the transplanted hepatocytes/hepatocyte progenitors. In some cases, the genetically modified non-human animal does not comprise Kupffer cells in the liver that are species-matched to the transplanted hepatocytes/hepatocyte progenitors. In some cases, the genetically modified non-human animal does not comprise a reconstituted immune system that is species-matched to the transplanted hepatocytes/hepatocyte progenitors. In some cases, the genetically modified non-human animal does not comprise human Kupffer cells. In some cases, the genetically modified non-human animal does not comprise human Kupffer cells in the liver. In some cases, the genetically modified non-human animal does not comprise a reconstituted human immune system. In some cases, the genetically modified non-human animal does not comprise Kupffer cells that are species-compatible to the transplanted hepatocytes (i.e., that produce IL-6 compatible with the IL-6R in the transplanted hepatocytes). In some cases, the genetically modified non-human animal does not comprise Kupffer cells in the liver that are species-compatible to the transplanted hepatocytes. In some cases, the genetically modified non-human animal does not comprise a reconstituted immune system that is species-compatible to the transplanted hepatocytes.

The xenotransplanted hepatocytes can be from any species other than that of the recipient non-human animal. For example, the xenotransplanted hepatocytes can be human hepatocytes. The transplanted hepatocytes can be from a species whose IL-6R is incompatible with the endogenous non-human animal IL-6 (e.g., the transplanted hepatocytes can be human hepatocytes, and the non-human animal can be a mouse or a rat). The cross-species incompatibility between the transplanted hepatocytes and the recipient non-human animals (i.e., the suboptimal interaction or lack of reactivity between the recipient non-human animal IL-6 ligands and the IL-6R on the xenotransplanted hepatocytes) can result in a steatosis-like phenotype and lipid droplet accumulation.

The modifications described herein can result in reduced lipid droplet accumulation and reduced steatosis in the xenotransplanted hepatocytes compared to non-human animals in which the genetically modified non-human animals and the xenotransplanted hepatocytes do not have modifications to restore IL-6/IL-6R signaling pathway activity or GP130 signaling pathway activity in the xenotransplanted hepatocytes. The modifications can, for example, restore species-matched hepatic IL-6/IL-6R pathway activity or GP130 signaling pathway activity in the xenotransplanted hepatocytes, or can simply restore IL-6/IL-6R signaling pathway activity or GP130 signaling pathway activity in the xenotransplanted hepatocytes. Likewise, the modifications can, for example, restore species-compatible hepatic IL-6/IL-6R pathway activity or GP130 signaling pathway activity in the xenotransplanted hepatocytes.

In some cases, the xenotransplanted hepatocytes/hepatocyte progenitors are modified to restore IL-6/IL-6R signaling pathway activity or GP130 signaling pathway activity in the xenotransplanted hepatocytes/hepatocyte progenitors. Likewise, in some cases, the methods comprise modifying the xenotransplanted hepatocytes/hepatocyte progenitors to restore IL-6/IL-6R signaling pathway activity or GP130 signaling pathway activity in the xenotransplanted hepatocytes/hepatocyte progenitors. As one example, the transplanted hepatocytes/hepatocyte progenitors can be modified to express (i.e., ectopically express) non-human animal IL-6R (i.e., IL-6R from the same species as the recipient non-human animal—in other words, IL-6R species-matched to the recipient non-human animal). Likewise, the transplanted hepatocytes/hepatocyte progenitors can be modified to express (i.e., ectopically express) species-compatible IL-6R (i.e., IL-6R that is compatible with the IL-6 produced by the recipient non-human animal such that the IL-6 can activate IL-6R signaling in the transplanted cells). For example, if the hepatocytes/hepatocyte progenitors are being transplanted into a mouse or a rat, the transplanted hepatocytes/hepatocyte progenitors can be genetically modified to express mouse IL-6R or rat IL-6R, respectively. In some cases, the transplanted hepatocytes/hepatocyte progenitors can be modified to comprise a vector comprising an expression construct for the non-human animal IL-6R comprising a nucleic acid encoding the non-human animal IL-6R operably linked to a promoter. Any suitable vector can be used. For example, the vector can be a viral vector, such as a lentiviral vector or an adeno-associated virus (AAV) vector. In a specific example, a lentiviral vector is used. Alternatively, the transplanted hepatocytes/hepatocyte progenitors can be genetically modified to comprise in their genome a non-human animal IL-6R expression construct comprising a nucleic acid encoding the non-human animal IL-6R operably linked to a promoter. In the case of genomic modification or in the case of a vector, any suitable promoter can be used. In one example, a liver-specific promoter or a promoter active in liver cells (e.g., hepatocytes) can be used. Examples of such promoters include TTR, ALB, and HBV promoters. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

As another example, the transplanted hepatocytes/hepatocyte progenitors can be modified to express (i.e., ectopically express) non-human animal OSMR (i.e., OSMR from the same species as the recipient non-human animal—in other words, OSMR species-matched to the recipient non-human animal). Likewise, the transplanted hepatocytes/hepatocyte progenitors can be modified to express (i.e., ectopically express) species-compatible OSMR (i.e., OSMR that is compatible with the OSM produced by the recipient non-human animal such that the OSM can activate OSMR signaling in the transplanted cells). For example, if the hepatocytes/hepatocyte progenitors are being transplanted into a mouse or a rat, the transplanted hepatocytes/hepatocyte progenitors can be genetically modified to express mouse OSMR or rat OSMR, respectively. In some cases, the transplanted hepatocytes/hepatocyte progenitors can be modified to comprise a vector comprising an expression construct for the non-human animal OSMR comprising a nucleic acid encoding the non-human animal OSMR operably linked to a promoter. Any suitable vector can be used. For example, the vector can be a viral vector, such as a lentiviral vector or an adeno-associated virus (AAV) vector. In a specific example, a lentiviral vector is used. Alternatively, the transplanted hepatocytes/hepatocyte progenitors can be genetically modified to comprise in their genome a non-human animal OSMR expression construct comprising a nucleic acid encoding the non-human animal OSMR operably linked to a promoter. In the case of genomic modification or in the case of a vector, any suitable promoter can be used. In one example, a liver-specific promoter or a promoter active in liver cells (e.g., hepatocytes) can be used. Examples of such promoters include TTR, ALB, and HBV promoters. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

As another example, the transplanted hepatocytes can be modified to express (i.e., ectopically express) non-human animal IL-6R and non-human animal OSMR (i.e., IL-6R and OSMR from the same species as the recipient non-human animal—in other words, IL-6R and OSMR species-matched to the recipient non-human animal). As another example, the transplanted hepatocytes can be modified to express (i.e., ectopically express) species-compatible IL-6R and OSMR (i.e., IL-6R and OSMR from a species that is compatible with the IL-6 and OSM, respectively, produced by the recipient non-human animal, such that the recipient non-human animal IL-6 and OSM can bind to and activate IL-6R and OSMR signaling, respectively). As another example, the transplanted hepatocytes can be modified to express (i.e., ectopically express) the non-human animal receptors for IL-6, LIF, OSM, CNTF, IL-11, CTF1, BSF3, or any combination thereof. As another example, the transplanted hepatocytes can be modified to express (i.e., ectopically express) species-compatible receptors for IL-6, LIF, OSM, CNTF, IL-11, CTF1, BSF3, or any combination thereof.

As another example, the transplanted hepatocytes/hepatocyte progenitors can be modified to express (i.e., ectopically express) non-human animal GHR (i.e., GHR from the same species as the recipient non-human animal—in other words, GHR species-matched to the recipient non-human animal). Likewise, the transplanted hepatocytes/hepatocyte progenitors can be modified to express (i.e., ectopically express) species-compatible GHR (i.e., GHR that is compatible with the GH produced by the recipient non-human animal such that the GH can activate GHR signaling in the transplanted cells). For example, if the hepatocytes/hepatocyte progenitors are being transplanted into a mouse or a rat, the transplanted hepatocytes/hepatocyte progenitors can be genetically modified to express mouse GHR or rat GHR, respectively. In some cases, the transplanted hepatocytes/hepatocyte progenitors can be modified to comprise a vector comprising an expression construct for the non-human animal GHR comprising a nucleic acid encoding the non-human animal GHR operably linked to a promoter. Any suitable vector can be used. For example, the vector can be a viral vector, such as a lentiviral vector or an adeno-associated virus (AAV) vector. In a specific example, a lentiviral vector is used. Alternatively, the transplanted hepatocytes/hepatocyte progenitors can be genetically modified to comprise in their genome a non-human animal GHR expression construct comprising a nucleic acid encoding the non-human animal GHR operably linked to a promoter. In the case of genomic modification or in the case of a vector, any suitable promoter can be used. In one example, a liver-specific promoter or a promoter active in liver cells (e.g., hepatocytes) can be used. Examples of such promoters include TTR, ALB, and HBV promoters. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

As another example of modifying the transplanted hepatocytes/hepatocyte progenitors, they can be modified to express (i.e., ectopically express) a ligand-independent, constitutively active form of IL-6R co-receptor glycoprotein 130 (GP130). In one example, the constitutively active human GP130 comprises a deletion of the region of GP130 from Tyr186 to Tyr190 (GP130^{Y186-Y190del}). For example, if the transplanted hepatocytes/hepatocyte progenitors are human hepatocytes/hepatocyte progenitors, the constitutively active form of GP130 can be a human GP130. In some cases, the transplanted hepatocytes/hepatocyte progenitors can be modified to comprise a vector comprising an expression construct for the constitutively active form of GP130 comprising a nucleic acid encoding the constitutively active form of GP130 operably linked to a promoter. Any suitable vector can be used. For example, the vector can be a viral vector, such as a lentiviral vector or an adeno-associated virus (AAV) vector. In a specific example, a lentiviral vector is used. Alternatively, the transplanted hepatocytes/hepatocyte progenitors can be genetically modified to comprise in their genome a GP130 expression construct comprising a nucleic acid encoding the constitutively active form of GP130 operably linked to a promoter. For example, the expression construct could be at a safe harbor locus in the non-human animal. In the case of genomic modification or in the case of a vector, any suitable promoter can be used. In one example, a liver-specific promoter or a promoter active in liver cells (e.g., hepatocytes) can be used. Examples of such promoters include TTR, ALB, and HBV promoters. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

In other cases, the recipient non-human animal is modified to restore IL-6/IL-6R signaling pathway activity or GP130 signaling pathway activity in the xenotransplanted hepatocytes/hepatocyte progenitors. Likewise, in other cases, the methods can comprise modifying the recipient non-human animal to restore IL-6/IL-6R signaling pathway activity or GP130 signaling pathway activity in the xenotransplanted hepatocytes/hepatocyte progenitors. For example, the non-human animal can comprise IL-6 from the species of the xenotransplanted hepatocytes/hepatocyte progenitors (e.g., IL-6 that is species-matched with the xenotransplanted hepatocytes/hepatocyte progenitors). For example, the non-human animal can comprise human IL-6 if the xenotransplanted hepatocytes/hepatocyte progenitors are human hepatocytes/hepatocyte progenitors. Likewise, the non-human animal can comprise IL-6 species-compatible with the species of the xenotransplanted hepatocytes/hepatocyte progenitors (e.g., IL-6 that can activate IL-6R signaling in the xenotransplanted hepatocytes/hepatocyte progenitors). For example, the non-human animal can comprise human-IL-6R-compatible ligand (e.g., human-IL-6R-compatible IL-6) (e.g., cynomolgus IL-6) if the xenotransplanted hepatocytes/hepatocyte progenitors are human hepatocytes/hepatocyte progenitors. For example, the non-human animal can comprise human-IL-6R-compatible IL-6 (e.g., cynomolgus IL-6) if the xenotransplanted hepatocytes/hepatocyte progenitors are human hepatocytes/hepatocyte progenitors. Human IL-6 is described above. The IL-6 can be, for example, in the serum of the non-human animal or the liver of the non-human animal. The IL-6 can be expressed by any suitable cell type in the non-human animal. In one example, the IL-6 is expressed in muscle cells in the non-human animal. In one example, the non-human animal comprises a vector comprising an expression construct for species-matched IL-6 (e.g., human IL-6) comprising a nucleic acid encoding the species-matched IL-6 (e.g., human IL-6) operably linked to a promoter. Likewise, the non-human animal can comprise a vector comprising an expression construct for species-compatible IL-6 (e.g., the IL-6 is compatible with the IL-6R expressed by the transplanted hepatocytes such that the IL-6 can activate IL-6R signaling in the hepatocytes) comprising a nucleic acid encoding the species-compatible IL-6 operably linked to a promoter. For example, if the transplanted hepatocytes are human hepatocytes, the non-human animal can comprise a vector comprising an expression construct for human-IL-6R-compatible ligand (e.g., human-IL-6R-compatible IL-6) (e.g., cynomolgus IL-6) comprising a nucleic acid encoding the human-IL-6R-compatible ligand (e.g., human-IL-6R-compatible IL-6) operably linked to a promoter. For example, if the transplanted hepatocytes are human hepatocytes, the non-human animal can comprise a vector comprising an expression construct for human-IL-6R-compatible IL-6 (e.g., cynomolgus IL-6) comprising a nucleic acid encoding the human-IL-6R-compatible IL-6 operably linked to a promoter. For example, the non-human animal can comprise the vector in muscle cells. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Alternatively, the non-human animal can comprise in its genome a species-matched IL-6 (e.g., human IL-6) expression construct comprising a nucleic acid encoding the species-matched IL-6 (e.g., human IL-6) operably linked to a promoter. Likewise, the non-human animal can comprise in its genome a species-compatible IL-6 (e.g., the IL-6 is compatible with the IL-6R expressed by the transplanted hepatocytes such that the IL-6 can activate IL-6R signaling in the hepatocytes) expression construct comprising a nucleic acid encoding the species-compatible IL-6 operably linked to a promoter. For example, if the transplanted hepatocytes are human hepatocytes, the non-human animal can comprise in its genome a human-IL-6R-compatible ligand (e.g., human-IL-6R-compatible IL-6) (e.g., cynomolgus IL-6) expression construct comprising a nucleic acid encoding the human-IL-6R-compatible ligand (e.g., human-IL-6R-compatible IL-6) operably linked to a promoter. For example, if the transplanted hepatocytes are human hepatocytes, the non-human animal can comprise in its genome a human-IL-6R-compatible IL-6 (e.g., cynomolgus IL-6) expression construct comprising a nucleic acid encoding the human-IL-6R-compatible IL-6 operably linked to a promoter. For example, the expression construct could be at a safe harbor locus in the non-human animal. In the case of genomic modification or in the case of a vector, any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MH) and muscle creatine kinase (CK) promoter (MHCK7) as described herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

For example, the non-human animal can comprise OSM from the species of the xenotransplanted hepatocytes/hepatocyte progenitors (e.g., OSM that is species-matched with the xenotransplanted hepatocytes/hepatocyte progenitors). For example, the non-human animal can comprise human OSM if the xenotransplanted hepatocytes/hepatocyte progenitors are human hepatocytes/hepatocyte progenitors. Likewise, the non-human animal can comprise OSM species-compatible with the species of the xenotransplanted hepatocytes/hepatocyte progenitors (e.g., OSM that can activate OSMR signaling in the xenotransplanted hepatocytes/hepatocyte progenitors). For example, the non-human animal can comprise human-OSMR-compatible ligand (e.g., human-OSMR-compatible OSM) (e.g., cynomolgus OSM) if the xenotransplanted hepatocytes/hepatocyte progenitors are human hepatocytes/hepatocyte progenitors. Human OSM is described above. The OSM can be, for example, in the serum of the non-human animal or the liver of the non-human animal. The OSM can be expressed by any suitable cell type in the non-human animal. In one example, the OSM is expressed in muscle cells in the non-human animal. In one example, the non-human animal comprises a vector comprising an expression construct for species-matched OSM (e.g., human OSM) comprising a nucleic acid encoding the species-matched OSM (e.g., human OSM) operably linked to a promoter. Likewise, the non-human animal can comprise a vector comprising an expression construct for species-compatible OSM (e.g., the OSM is compatible with the OSMR expressed by the transplanted hepatocytes such that the OSM can activate OSMR signaling in the hepatocytes) comprising a nucleic acid encoding the species-compatible OSM operably linked to a promoter. For example, if the transplanted hepatocytes are human hepatocytes, the non-human animal can comprise a vector comprising an expression construct for human-OSMR-compatible ligand (e.g., human-OSMR-compatible OSM) (e.g., cynomolgus OSM) comprising a nucleic acid encoding the human-OSMR-compatible ligand (e.g., human-OSMR-compatible OSM) operably linked to a promoter. For example, the non-human animal can comprise the vector in muscle cells. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Alternatively, the non-human animal can comprise in its genome a species-matched OSM (e.g., human OSM) expression construct comprising a nucleic acid encoding the species-matched OSM (e.g., human OSM) operably linked to a promoter. Likewise, the non-human animal can comprise in its genome a species-compatible OSM (e.g., the OSM is compatible with the OSMR expressed by the transplanted hepatocytes such that the OSM can activate OSMR signaling in the hepatocytes) expression construct comprising a nucleic acid encoding the species-compatible OSM operably linked to a promoter. For example, if the transplanted hepatocytes are human hepatocytes, the non-human animal can comprise in its genome a human-OSMR-compatible ligand (e.g., human-OSMR-compatible OSM) (e.g., cynomolgus OSM) expression construct comprising a nucleic acid encoding the human-OSMR-compatible ligand (e.g., human-OSMR-compatible OSM) operably linked to a promoter. For example, the expression construct could be at a safe harbor locus in the non-human animal. In the case of genomic modification or in the case of a vector, any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MI-1) and muscle creatine kinase (CK) promoter (MHCK7) as described herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

In other cases, the recipient non-human animal is modified to restore IL-6/IL-6R signaling pathway activity or GP130 signaling pathway activity in the xenotransplanted hepatocytes. For example, the non-human animal can comprise a GP130 activator (e.g., human GP130 activator), such as a GP130-activating ligand (e.g., human-GP130-activating ligand). In one example, the non-human animal can comprise one or more GP130-activating ligands. In another example, the non-human animal can comprise two or more GP130-activating ligands. In another example, the non-human animal can comprise three or more GP130-activating ligands. In another example, the non-human animal can comprise four or more GP130-activating ligands. Ligands that activate GP130 are known. For example, IL-6, LIF, OSM, CNTF, IL-11, CTF1, and BSF3 are all ligands that can activate GP130. In one example, the non-human animals can comprise a ligand (e.g., IL-6, leukemia inhibitory factor (LIF), oncostatin M (OSM), ciliary neurotrophic factor (CNTF), interleukin-11 (IL-11), cardiotrophin-1 (CTF1), or cardiotrophin-like cytokine factor 1 (BSF3)) from the species of the xenotransplanted hepatocytes (e.g., IL-6, LIF, OSM, CNTF, IL-11, CTF1, or BSF3 that is species-matched or species-compatible with the xenotransplanted hepatocytes). In one example, the non-human animals can comprise IL-6 (e.g., species-matched or species-compatible with the xenotransplanted hepatocytes). In another example, the non-human animals can comprise OSM (e.g., species-matched or species-compatible with the xenotransplanted hepatocytes). In another example, the non-human animals can comprise IL-6 and OSM (e.g., species-matched or species-compatible with the xenotransplanted hepatocytes). In another example, the non-human animals can comprise IL-6, LIF, OSM, CNTF, IL-11, CTF1, BSF3, or any combination thereof (e.g., species-matched or species-compatible with the xenotransplanted hepatocytes). For example, the non-human animal can comprise human IL-6, LIF, OSM, CNTF, IL-11, CTF1, or BSF3 if the xenotransplanted hepatocytes are human hepatocytes. Another example of a GP130 activator is a GP130 agonist antibody or antigen-binding protein (e.g., human GP130 agonist antibody or antigen-binding protein). GP130-activating antibodies are known. See, e.g., Autissier et al. (1997) Eur. J. Immunol. 27(3):794-797, herein incorporated by reference in its entirety for all purposes. Another example of a GP130 activator is a chimeric GP130 ligand, termed IC7Fc, where one GP130 binding site has been removed from IL-6 and replaced with the leukemia inhibitory factor receptor (LIFR) binding site from CNTF and then fused with the fragment crystallizable (Fc) domain of immunoglobulin G (IgG). See, e.g., Findeisen et al. (2019) Nature 574:63-68, herein incorporated by reference in its entirety for all purposes. The GP130 activator (e.g., ligand) can be, for example, in the serum of the non-human animal or the liver of the non-human animal. The GP130 activator (e.g., ligand) can be expressed by any suitable cell type in the non-human animal. In one example, the GP130 activator (e.g., ligand) is expressed in muscle cells in the non-human animal. In one example, the non-human animal comprises a vector comprising an expression construct for the GP130 activator (e.g., ligand) comprising a nucleic acid encoding the GP130 activator operably linked to a promoter. For example, the non-human animal can comprise the vector in muscle cells. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Alternatively, the non-human animal can comprise in its genome a GP130 activator (e.g. ligand) expression construct comprising a nucleic acid encoding the GP130 activator (e.g. ligand) operably linked to a promoter. For example, the expression construct could be at a safe harbor locus in the non-human animal. In the case of genomic modification or in the case of a vector, any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MH) and muscle creatine kinase (CK) promoter (MHCK7) as described herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

For example, the non-human animal can comprise GH from the species of the xenotransplanted hepatocytes/hepatocyte progenitors (e.g., GH that is species-matched with the xenotransplanted hepatocytes/hepatocyte progenitors). For example, the non-human animal can comprise human GH if the xenotransplanted hepatocytes/hepatocyte progenitors are human hepatocytes/hepatocyte progenitors. Likewise, the non-human animal can comprise GH species-compatible with the species of the xenotransplanted hepatocytes/hepatocyte progenitors (e.g., GH that can activate GHR signaling in the xenotransplanted hepatocytes/hepatocyte progenitors). For example, the non-human animal can comprise human-GHR-compatible GH (e.g., cynomolgus GH) if the xenotransplanted hepatocytes/hepatocyte progenitors are human hepatocytes/hepatocyte progenitors. The GH can be, for example, in the serum of the non-human animal or the liver of the non-human animal. The GH can be expressed by any suitable cell type in the non-human animal. In one example, the GH is expressed in muscle cells in the non-human animal. In one example, the non-human animal comprises a vector comprising an expression construct for species-matched GH (e.g., human GH) comprising a nucleic acid encoding the species-matched GH (e.g., human GH) operably linked to a promoter. Likewise, the non-human animal can comprise a vector comprising an expression construct for species-compatible GH (e.g., the GH is compatible with the GHR expressed by the transplanted hepatocytes such that the GH can activate GHR signaling in the hepatocytes) comprising a nucleic acid encoding the species-compatible GH operably linked to a promoter. For example, if the transplanted hepatocytes are human hepatocytes, the non-human animal can comprise a vector comprising an expression construct for human-GHR-compatible GH (e.g., cynomolgus GH) comprising a nucleic acid encoding the human-GHR-compatible GH operably linked to a promoter. For example, the non-human animal can comprise the vector in muscle cells. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Alternatively, the non-human animal can comprise in its genome a species-matched GH (e.g., human GH) expression construct comprising a nucleic acid encoding the species-matched GH (e.g., human GH) operably linked to a promoter. Likewise, the non-human animal can comprise in its genome a species-compatible GH (e.g., the GH is compatible with the GHR expressed by the transplanted hepatocytes such that the GH can activate GHR signaling in the hepatocytes) expression construct comprising a nucleic acid encoding the species-compatible GH operably linked to a promoter. For example, if the transplanted hepatocytes are human hepatocytes, the non-human animal can comprise in its genome a human-GHR-compatible GH (e.g., cynomolgus GH) expression construct comprising a nucleic acid encoding the human-GHR-compatible GH operably linked to a promoter. For example, the expression construct could be at a safe harbor locus in the non-human animal. In the case of genomic modification or in the case of a vector, any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MH) and muscle creatine kinase (CK) promoter (MHCK7) as described herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

In another example, the non-human animal can comprise a humanized IL6 locus as described elsewhere herein. Examples of non-human animals with humanized IL6 genes are provided, e.g., in U.S. Pat. No. 9,622,460, herein incorporated by reference in its entirety for all purposes. The non-human animal can be, in some cases, homozygous for the humanized IL6 gene. In other cases, the non-human animal can be heterozygous for the humanized IL6 gene. The non-human animal can comprise the humanized IL6 gene in its germline. The humanized IL6 gene can comprise a human IL6 nucleic acid encoding a human IL-6 protein (e.g., a fully human IL-6 protein). The human IL6 nucleic acid can be a genomic nucleic acid, such as a genomic nucleic acid comprising a region of a human IL6 gene from the start codon to the stop codon, or can comprise a human IL6 complementary DNA (cDNA). The human IL6 nucleic acid can be inserted into the non-human animal 116 genomic locus, or it can replace a corresponding region of the non-human animal 116 locus (e.g., a region of a human IL6 gene from the start codon to the stop codon can replace a region of the non-human animal 116 gene from the start codon to the stop codon). The humanized IL6 gene (or the human IL6 nucleic acid) can be operably linked to the endogenous non-human animal 116 promoter. In other words, expression of the humanized IL6 gene can be driven by the endogenous non-human animal 116 promoter.

In another example, the non-human animal can comprise a humanized OSM locus as described elsewhere herein. The non-human animal can be, in some cases, homozygous for the humanized OSM gene. In other cases, the non-human animal can be heterozygous for the humanized OSM gene. The non-human animal can comprise the humanized OSM gene in its germline. The humanized OSM gene can comprise a human OSM nucleic acid encoding a human OSM protein (e.g., a fully human OSM protein). The human OSM nucleic acid can be a genomic nucleic acid, such as a genomic nucleic acid comprising a region of a human OSM gene from the start codon to the stop codon, or can comprise a human OSM complementary DNA (cDNA). The human OSM nucleic acid can be inserted into the non-human animal Osm genomic locus, or it can replace a corresponding region of the non-human animal Osm locus (e.g., a region of a human OSM gene from the start codon to the stop codon can replace a region of the non-human animal Osm gene from the start codon to the stop codon). The humanized OSM gene (or the human OSM nucleic acid) can be operably linked to the endogenous non-human animal Osm promoter. In other words, expression of the humanized OSM gene can be driven by the endogenous non-human animal Osm promoter.

In another example, the non-human animal can comprise a humanized IL6 locus as described elsewhere herein and a humanized OSM locus as described elsewhere herein.

In another example, the non-human animal can comprise a humanized GH locus. Similar to IL6, mGH shows species specificities in receptor binding, and administration of human Growth Hormone (GH) in humanized liver mice could correct fatty liver in humanized liver mice. Tateno et al. (2011) Endocrinology 152:1479-1491, herein incorporated by reference in its entirety for all purposes.

In another example, the non-human animal can comprise a humanized IL6 locus as described elsewhere herein and a humanized GH locus as described elsewhere herein.

In another example, the non-human animal can comprise a humanized OSM locus as described elsewhere herein and a humanized GH locus as described elsewhere herein.

In another example, the non-human animal can comprise a humanized IL6 locus as described elsewhere herein and a humanized OSM locus as described elsewhere herein and a humanized GH locus as described elsewhere herein.

The hepatocytes or hepatocytes progenitors (e.g., human hepatocytes or human hepatocyte progenitors) can be from any source. If the recipient non-human animals have an inactivated endogenous Fah gene, preferably the hepatocytes or hepatocyte progenitors have a functional Fah gene (e.g., a wild type Fah gene) that produces a functional FAH protein. In that way, for example, the hepatocytes or hepatocyte progenitors can repopulate the liver in the non-human animals and rescue the decreased liver function.

The hepatocytes or hepatocyte progenitors can be transplanted into the non-human animal by any suitable means. For example, the hepatocytes or hepatocyte progenitors can be transplanted by injection into the hepatic artery, the spleen, the portal vein, the peritoneal cavity, hepatic tissue mass, or the lymphatic system, or they can be transplanted as part of a liver tissue graft. In one example, the hepatocytes or hepatocyte progenitors can be transplanted via intrasplenic injection, after which the hepatocytes or hepatocyte progenitors will travel through the vasculature to reach the liver.

Any suitable number of hepatocytes or hepatocyte progenitors can be transplanted. For example, at least about 1 million, at least about 2 million, at least about 3 million, at least about 4 million, at least about 5 million, at least about 6 million, at least about 7 million, at least about 8 million, at least about 9 million, or at least about 10 million hepatocytes or hepatocyte progenitors can be transplanted.

Exogenous urokinase plasminogen activator (uPA; also called urokinase) or an exogenous nucleic acid encoding uPA can be administered to the non-human animal prior to transplantation to prime the liver for improved repopulation by human hepatocytes. Likewise, any other compound that primes the liver for improved repopulation by xenotransplanted hepatocytes (e.g., human hepatocytes) can be used. In a specific example, an adenovirus or an adeno-associated virus (AAV) or adenoviral or AAV vector encoding urokinase plasminogen activator is administered to the non-human animal prior to transplantation. The exogenous uPA or exogenous nucleic acid encoding uPA (or any other priming compound) can be administered at least about 1 day, at least about 2 days, at least about 3 days, at least about 4 days, at least about 5 days, at least about 6 days, or at least about 7 days prior to transplantation. In a specific example, the exogenous uPA or exogenous nucleic acid encoding uPA (or any other priming compound) can be administered about 24 hours to about 48 hours prior to transplantation. The uPA can be human uPA, and it can be a secreted form or can be a non-secreted form.

Nitisinone or any other compound that ameliorates toxicity caused by Fah deficiency can be administered to the non-human animal prior to the transplantation if the non-human animal comprises an inactivated endogenous Fah gene. Optionally, nitisinone or any other compound that ameliorates toxicity caused by Fah deficiency can be administered for a period of time after the transplantation. This can be done, for example, to avoid decreased liver function or liver cell death or death of the recipient non-human animal prior to repopulation with the human hepatocytes or human hepatocyte progenitors. For example, the nitisinone or any other compound that ameliorates toxicity caused by Fah deficiency can be administered for at least about 1 day, at least about 2 days, at least about 3 days, at least about 4 days, at least about 5 days, at least about 6 days, at least about 1 week, at least about 2 weeks, at least about 3 weeks, or at least about 4 weeks after transplantation. Alternatively, the nitisinone or any other compound that ameliorates toxicity caused by Fah deficiency can be withdrawn after no more than about 1 day, no more than about 2 days, no more than about 3 days, no more than about 4 days, no more than about 5 days, no more than about 6 days, no more than about 1 week, no more than about 2 weeks, no more than about 3 weeks, or no more than about 4 weeks after transplantation. The nitisinone dose can be between about 0.2 mg/kg to about 2 mg/kg per day, or about 1 mg/kg per day. Alternatively, the nitisinone dose can be between about 1 mg/kg to about 7 mg/kg, between about 2 mg/kg and about 6 mg/kg, between about 3 mg/kg and about 5 mg/kg, about 4 mg/kg, between about 1 mg/kg and about 4 mg/kg, between about 2 mg/kg and about 4 mg/kg, between about 3 mg/kg and about 4 mg/kg, between about 4 mg/kg and about 7 mg/kg, between about 4 mg/kg and about 6 mg/kg, or between about 4 mg/kg and about 5 mg/kg. The nitisinone or any other compound that ameliorates toxicity caused by Fah deficiency can be administered by any suitable means, such as in drinking water, in food, or by injection. The concentration of nitisinone or any other compound that ameliorates toxicity caused by Fah deficiency in drinking water can be any suitable dose. For example, the dose can be between about 0.5 mg/L to about 30 mg/L, between about 1 mg/L to about 25 mg/L, between about 10 mg/L and about 20 mg/L, or about 20 mg/L.

Alternatively, nitisinone or any other compound that ameliorates toxicity caused by Fah deficiency can be withdrawn (i.e., no longer administered to the non-human animal) prior to the transplantation. For example, nitisinone or any other compound that ameliorates toxicity caused by Fah deficiency can be withdrawn at least about 12 hours, at least about 24 hours, at least about 1 day, at least about 2 days, or at least about 3 days prior to transplantation, or no more than about 12 hours, no more than about 24 hours, no more than about 1 day, no more than about 2 days, or no more than about 3 days prior to transplantation.

Following transplantation, nitisinone or any other compound that ameliorates toxicity caused by Fah deficiency can be cycled off and on to promote repopulation by the transplanted hepatocytes. For example, the cycling can be about 3 days to about 9 days, about 4 days to about 8 days, about 5 days to about 7 days, about 3 days to about 7 days, about 4 days to about 7 days, about 5 days to about 8 days, about 5 days to about 9 days, or about 6 days off, and can be about 1 day to about 5 days, about 2 days to about 4 days, about 1 day to about 4 days, about 1 day to about 3 days, about 3 days to about 4 days, about 3 days to about 5 days, or about 3 days on. In a specific example, the cycling can be about 5 days to about 7 days off and about 3 days on. The cycling off can be for at least about 1 day, at least about 2 days, at least about 3 days, at least about 4 days, at least about 5 days, at least about 6 days, or at least about 7 days, or no more than about 1 day, no more than about 2 days, no more than about 3 days, no more than about 4 days, no more than about 5 days, no more than about 6 days, or no more than about 7 days, and the cycling on can be for at least about 1 day, at least about 2 days, at least about 3 days, at least about 4 days, at least about 5 days, at least about 6 days, or at least about 7 days, or no more than about 1 day, no more than about 2 days, no more than about 3 days, no more than about 4 days, no more than about 5 days, no more than about 6 days, or no more than about 7 days.

The transplanted hepatocytes or hepatocyte progenitors can be allowed to expand for any suitable amount of time. For example, the hepatocytes or hepatocyte progenitors can be allowed to expand for a suitable amount of time to repopulate the liver and rescue the decreased liver function (e.g., to create a humanized liver). For example, the hepatocytes or hepatocyte progenitors can be allowed to expand for at least about 1 day, at least about 2 days, at least about 3 days, at least about 4 days, at least about 5 days, at least about 6 days, at least about 1 week, at least about 2 weeks, at least about 3 weeks, at least about 4 weeks, at least about 5 weeks, at least about 6 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months.

The recipient non-human animals receiving the transplanted hepatocytes or hepatocyte progenitors can be any suitable age. For example, the non-human animals (e.g., mice or rats) can be at least about 1 day old, at least about 2 days old, at least about 1 week old, at least about 2 weeks old, at least about 3 weeks old, at least about 4 weeks old, at least about 1 month old, or at least about 2 months old. Alternatively, the non-human animals can be less than about 1 day old, less than about 2 days old, less than about 1 week old, less than about 2 weeks old, less than about 3 weeks old, less than about 4 weeks old, less than about 1 month old, less than about 2 months old, less than about 3 months old, less than about 4 months old, less than about 5 months old, or less than about 6 months old. In some methods, the recipient non-human animals can be fetuses, and the transplanted hepatocytes or hepatocyte progenitors can be injected, e.g., via the umbilical vein or directly into the fetal liver.

Some methods comprise serial transplantation of the hepatocytes or hepatocyte progenitors. For example, the expanded hepatocytes or hepatocyte progenitors from a first recipient non-human animal can be collected and transplanted and further expanded in a second recipient non-human animal.

The methods can further comprise assessing transplanted hepatocyte repopulation levels in the non-human animals following transplantation and expansion. For example, human hepatocyte repopulation levels can be determined through quantitation of human serum albumin levels and/or immunohistochemistry of liver sections from transplanted non-human animals.

B. Methods of Preventing, Reducing, or Ameliorating Hepatosteatosis in Non-Human Animals Comprising Xenotransplanted Hepatocytes

Various methods are provided for preventing hepatosteatosis in non-human animals (e.g., mice and rats) comprising xenotransplanted hepatocytes (e.g., human hepatocytes and/or a humanized liver). Likewise, various methods are provided for reducing or ameliorating hepatosteatosis in non-human animals (e.g., mice and rats) comprising xenotransplanted hepatocytes (e.g., human hepatocytes and/or a humanized liver). Likewise, methods are provided for preventing lipid droplet accumulation in xenotransplanted hepatocytes in non-human animals (e.g., mice and rats) comprising the xenotransplanted hepatocytes (e.g., human hepatocytes and/or a humanized liver). Likewise, methods are provided for reducing or ameliorating lipid droplet accumulation in xenotransplanted hepatocytes in non-human animals (e.g., mice and rats) comprising the xenotransplanted hepatocytes (e.g., human hepatocytes and/or a humanized liver). Such methods can comprise, for example, administering IL-6 that is species-matched to the xenotransplanted hepatocytes or a nucleic acid encoding the IL-6 (e.g., administering human IL-6 or a nucleic acid encoding the human IL-6) to the non-human animal, wherein the species-matched IL-6 restores interleukin-6 (IL-6)/interleukin-6 receptor (IL-6R) signaling pathway activity or GP130 signaling pathway activity in the transplanted hepatocytes. Likewise, such methods can comprise, for example, administering IL-6 that is species-compatible to the xenotransplanted hepatocytes or a nucleic acid encoding the IL-6 to the non-human animal, wherein the species-compatible IL-6 restores interleukin-6 (IL-6)/interleukin-6 receptor (IL-6R) signaling pathway activity or GP130 signaling pathway activity in the transplanted hepatocytes. For example, if the transplanted hepatocytes are human hepatocytes, such methods can comprise, for example, administering human-IL-6R-compatible ligand (e.g., human-IL-6R-compatible IL-6) (e.g., cynomolgus IL-6) or a nucleic acid encoding the human-IL-6R-compatible ligand (e.g., human-IL-6R-compatible IL-6) (e.g., administering human-IL-6R-compatible ligand (e.g., human-IL-6R-compatible IL-6) or a nucleic acid encoding the human-IL-6R-compatible ligand (e.g., human-IL-6R-compatible IL-6)) to the non-human animal, wherein the human-IL-6R-compatible ligand (e.g., human-IL-6R-compatible IL-6) restores interleukin-6 (IL-6)/interleukin-6 receptor (IL-6R) signaling pathway activity or GP130 signaling pathway activity in the transplanted hepatocytes. For example, if the transplanted hepatocytes are human hepatocytes, such methods can comprise, for example, administering human-IL-6R-compatible IL-6 (e.g., cynomolgus IL-6) or a nucleic acid encoding the human-IL-6R-compatible IL-6 (e.g., administering human-IL-6R-compatible IL-6 or a nucleic acid encoding the human-IL-6R-compatible IL-6) to the non-human animal, wherein the human-IL-6R-compatible IL-6 restores interleukin-6 (IL-6)/interleukin-6 receptor (IL-6R) signaling pathway activity or GP130 signaling pathway activity in the transplanted hepatocytes.

In one example, the nucleic acid is administered, wherein the nucleic acid comprises an expression construct (e.g., a vector comprising an expression construct) for species-matched IL-6 (e.g., human IL-6, or human-IL-6R-compatible ligand (e.g., human-IL-6R-compatible IL-6) if the transplanted hepatocytes are human hepatocytes) comprising a nucleic acid encoding the species-matched IL-6 (e.g., human IL-6, or human-IL-6R-compatible ligand (e.g., human-IL-6R-compatible IL-6) if the transplanted hepatocytes are human hepatocytes) operably linked to a promoter. In one example, the nucleic acid is administered, wherein the nucleic acid comprises an expression construct (e.g., a vector comprising an expression construct) for species-matched IL-6 (e.g., human IL-6, or human-IL-6R-compatible IL-6 if the transplanted hepatocytes are human hepatocytes) comprising a nucleic acid encoding the species-matched IL-6 (e.g., human IL-6, or human-IL-6R-compatible IL-6 if the transplanted hepatocytes are human hepatocytes) operably linked to a promoter. For example, the nucleic acid/vector can be administered to the liver or muscle or such that it reaches the liver or muscle cells. Any suitable administration method can be used, and such methods are disclosed in more detail elsewhere herein. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MI-1) and muscle creatine kinase (CK) promoter (MHCK7) as described herein. Alternatively, a liver-specific promoter or a promoter active in liver cells (e.g., hepatocytes) can be used. Such promoters are described elsewhere herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In another example, the species-matched IL-6 protein (e.g., human IL-6, or human-IL-6R-compatible ligand (e.g., human-IL-6R-compatible IL-6) if the transplanted hepatocytes are human hepatocytes) is administered to the non-human animal. As another example, an inducible promoter can be used. In another example, the species-matched IL-6 protein (e.g., human IL-6, or human-IL-6R-compatible IL-6 if the transplanted hepatocytes are human hepatocytes) is administered to the non-human animal. The IL-6 protein can be administered, e.g., to the liver or such that it reaches the liver of the non-human animal. Suitable methods for administering are described in more detail elsewhere herein.

Such methods can comprise, for example, administering OSM that is species-matched to the xenotransplanted hepatocytes or a nucleic acid encoding the OSM (e.g., administering human OSM or a nucleic acid encoding the human OSM) to the non-human animal, wherein the species-matched OSM restores GP130 signaling pathway activity in the transplanted hepatocytes. Likewise, such methods can comprise, for example, administering OSM that is species-compatible to the xenotransplanted hepatocytes or a nucleic acid encoding the OSM to the non-human animal, wherein the species-compatible OSM restores GP130 signaling pathway activity in the transplanted hepatocytes. For example, if the transplanted hepatocytes are human hepatocytes, such methods can comprise, for example, administering human-OSMR-compatible ligand (e.g., human-OSMR-compatible OSM) (e.g., cynomolgus OSM) or a nucleic acid encoding the human-OSMR-compatible ligand (e.g., human-OSMR-compatible OSM) (e.g., administering human-OSMR-compatible ligand (e.g., human-OSMR-compatible OSM) or a nucleic acid encoding the human-OSMR-compatible ligand (e.g., human-OSMR-compatible OSM)) to the non-human animal, wherein the human-OSMR-compatible ligand (e.g., human-OSMR-compatible OSM) restores GP130 signaling pathway activity in the transplanted hepatocytes.

In one example, the nucleic acid is administered, wherein the nucleic acid comprises an expression construct (e.g., a vector comprising an expression construct) for species-matched OSM (e.g., human OSM, or human-OSMR-compatible ligand (e.g., human-OSMR-compatible OSM) if the transplanted hepatocytes are human hepatocytes) comprising a nucleic acid encoding the species-matched OSM (e.g., human OSM, or human-OSMR-compatible ligand (e.g., human-OSMR-compatible OSM) if the transplanted hepatocytes are human hepatocytes) operably linked to a promoter. For example, the nucleic acid/vector can be administered to the liver or muscle or such that it reaches the liver or muscle cells. Any suitable administration method can be used, and such methods are disclosed in more detail elsewhere herein. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MI-1) and muscle creatine kinase (CK) promoter (MHCK7) as described herein. Alternatively, a liver-specific promoter or a promoter active in liver cells (e.g., hepatocytes) can be used. Such promoters are described elsewhere herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In another example, the species-matched OSM protein (e.g., human OSM, or human-OSMR-compatible ligand (e.g., human-OSMR-compatible OSM) if the transplanted hepatocytes are human hepatocytes) is administered to the non-human animal. The OSM protein can be administered, e.g., to the liver or such that it reaches the liver of the non-human animal. Suitable methods for administering are described in more detail elsewhere herein.

Such methods can comprise, for example, administering GH that is species-matched to the xenotransplanted hepatocytes or a nucleic acid encoding the GH (e.g., administering human GH or a nucleic acid encoding the human GH) to the non-human animal, wherein the species-matched GH restores GH signaling pathway activity in the transplanted hepatocytes. Likewise, such methods can comprise, for example, administering GH that is species-compatible to the xenotransplanted hepatocytes or a nucleic acid encoding the GH to the non-human animal, wherein the species-compatible GH restores GH signaling pathway activity in the transplanted hepatocytes. For example, if the transplanted hepatocytes are human hepatocytes, such methods can comprise, for example, administering human-GHR-compatible GH (e.g., cynomolgus GH) or a nucleic acid encoding the human-GHR-compatible GH (e.g., administering human-GHR-compatible GH or a nucleic acid encoding the human-GHR-compatible GH) to the non-human animal, wherein the human-GHR-compatible GH restores GH signaling pathway activity in the transplanted hepatocytes.

In one example, the nucleic acid is administered, wherein the nucleic acid comprises an expression construct (e.g., a vector comprising an expression construct) for species-matched GH (e.g., human GH, or human-GHR-compatible GH if the transplanted hepatocytes are human hepatocytes) comprising a nucleic acid encoding the species-matched GH (e.g., human GH, or human-GHR-compatible GH if the transplanted hepatocytes are human hepatocytes) operably linked to a promoter. For example, the nucleic acid/vector can be administered to the liver or muscle or such that it reaches the liver or muscle cells. Any suitable administration method can be used, and such methods are disclosed in more detail elsewhere herein. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MH) and muscle creatine kinase (CK) promoter (MHCK7) as described herein. Alternatively, a liver-specific promoter or a promoter active in liver cells (e.g., hepatocytes) can be used. Such promoters are described elsewhere herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In another example, the species-matched GH protein (e.g., human GH, or human-GHR-compatible GH if the transplanted hepatocytes are human hepatocytes) is administered to the non-human animal. The GH protein can be administered, e.g., to the liver or such that it reaches the liver of the non-human animal. Suitable methods for administering are described in more detail elsewhere herein.

Such methods can comprise, for example, administering a GP130 activator (e.g., human GP130 activator), such as a GP130-activating ligand (e.g., human-GP130-activating ligand) to the non-human animal. In one example, one or more GP130-activating ligands are administered. In another example, two or more GP130-activating ligands are administered. In another example, three or more GP130-activating ligands are administered. In another example, four or more GP130-activating ligands are administered. Ligands that activate GP130 are known. For example, IL-6, LIF, OSM, CNTF, IL-11, CTF1, and BSF3 are all ligands that can activate GP130. In one example, a ligand (e.g., IL-6, leukemia inhibitory factor (LIF), oncostatin M (OSM), ciliary neurotrophic factor (CNTF), interleukin-11 (IL-11), cardiotrophin-1 (CTF1), or cardiotrophin-like cytokine factor 1 (BSF3)) from the species of the xenotransplanted hepatocytes (e.g., IL-6, LIF, OSM, CNTF, IL-11, CTF1, or BSF3 that is species-matched or species-compatible with the xenotransplanted hepatocytes) is administered. In one example, the IL-6 (e.g., species-matched or species-compatible with the xenotransplanted hepatocytes) is administered. In another example, OSM (e.g., species-matched or species-compatible with the xenotransplanted hepatocytes) is administered. In another example, IL-6 and OSM (e.g., species-matched or species-compatible with the xenotransplanted hepatocytes) are administered. In another example, IL-6, LIF, OSM, CNTF, IL-11, CTF1, BSF3, or any combination thereof (e.g., species-matched or species-compatible with the xenotransplanted hepatocytes) are administered. For example, human IL-6, LIF, OSM, CNTF, IL-11, CTF1, or BSF3 can be administered if the xenotransplanted hepatocytes are human hepatocytes. Another example of a GP130 activator is a GP130 agonist antibody or antigen-binding protein (e.g., human GP130 agonist antibody or antigen-binding protein). GP130-activating antibodies are known. See, e.g., Autissier et al. (1997) Eur. J. Immunol. 27(3):794-797, herein incorporated by reference in its entirety for all purposes. Another example of a GP130 activator is a chimeric GP130 ligand, termed IC7Fc, where one GP130 binding site has been removed from IL-6 and replaced with the leukemia inhibitory factor receptor (LIFR) binding site from CNTF and then fused with the fragment crystallizable (Fc) domain of immunoglobulin G (IgG). See, e.g., Findeisen et al. (2019) Nature 574:63-68, herein incorporated by reference in its entirety for all purposes. The GP130 activator (e.g., ligand) can be, for example, in the serum of the non-human animal or the liver of the non-human animal. The GP130 activator (e.g., ligand) can be expressed by any suitable cell type in the non-human animal. In one example, the GP130 activator (e.g., ligand) is expressed in muscle cells in the non-human animal. In one example, a vector comprising an expression construct for the GP130 activator (e.g., ligand) comprising a nucleic acid encoding the GP130 activator operably linked to a promoter is administered. For example, the vector is administered to muscle cells. Any suitable vector can be used. For example, the vector can be a viral vector, such as a lentiviral vector or an adeno-associated virus (AAV) vector. In a specific example, an AAV vector is used, such as an AAV serotype for expression in muscle. In a specific example, a recombinant AAV9 vector is used. Alternatively, the non-human animal can comprise in its genome a GP130 activator (e.g. ligand) expression construct comprising a nucleic acid encoding the GP130 activator (e.g. ligand) operably linked to a promoter. For example, the expression construct could be at a safe harbor locus in the non-human animal. In the case of genomic modification or in the case of a vector, any suitable promoter can be used. In one example, a tissue-specific promoter can be used. For example, a muscle-specific promoter or a promoter active in muscle cells can be used. An example of a muscle-specific promoter is a hybrid mouse alpha-myosin heavy-chain (MH) and muscle creatine kinase (CK) promoter (MHCK7) as described herein. In another example, a constitutive promoter can be used. Examples of such promoters include human cytomegalovirus (hCMV), chicken beta-actin/CMV enhancer (CAG), and elongation factor-1 alpha (EF1alpha). As another example, an inducible promoter can be used. In the case of genomic modification, an exogenous promoter can be used or an endogenous promoter at the target genomic locus (e.g., a safe harbor locus) can be used.

The non-human animal can be any of the genetically modified non-human animals for xenotransplantation of hepatocytes as described in more detail elsewhere herein, and the xenotransplanted hepatocytes can be any suitable hepatocytes as disclosed in more detail elsewhere herein.

C. Methods of Assessing Activity of Human-Liver-Targeting Agents

Various methods are provided for assessing activity of human-liver-targeting agents/reagents in vivo using the genetically modified non-human animals comprising human hepatocytes (i.e., xenotransplanted human hepatocytes) or a humanized liver as described elsewhere herein. Such methods can comprise: (a) administering the human-liver-targeting reagent to the non-human animal; and (b) assessing the activity of the human-liver-targeting reagent in the liver of the non-human animal.

A human-liver-targeting reagent can be any reagent that targets a human liver, a cell in a human liver, a protein expressed in a human liver, an RNA expressed in a human liver, a gene expressed in a human liver, or any other target present in a human liver. A non-limiting example of a target gene expressed in the liver is albumin. A human-liver-targeting reagent can be, for example, a known human-liver-targeting reagent, a putative human-liver-targeting reagent (e.g., candidate reagents designed to target a human liver, cells in a human liver, a protein expressed in a human liver, an RNA expressed in a human liver, or a gene expressed in a human liver), or a reagent being screened for human-liver-targeting activity. The human-liver-targeting reagent can be an antibody or antigen-binding protein or any other large molecule or small molecule that targets a protein, an RNA, a gene, or any other target expressed in or present in a human liver. Alternatively, the human-liver-targeting reagent can be any biological or chemical agent that targets a protein, an RNA, a gene, or any other target expressed in or present in a human liver.

For example, a human-liver-targeting reagent can be an antigen-binding protein (e.g., agonist antibody) targeting a protein or antigen expressed by or present in a human liver. The term “antigen-binding protein” includes any protein that binds to an antigen. Examples of antigen-binding proteins include an antibody, an antigen-binding fragment of an antibody, a multispecific antibody (e.g., a bi-specific antibody), an scFv, a bis-scFv, a diabody, a triabody, a tetrabody, a V-NAR, a VHH, a VL, a F(ab), a F(ab)₂, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes). Other human-liver-targeting reagents include small molecules targeting a cell, protein, RNA, gene, or any other target present in or expressed in a human liver.

Other human-liver-targeting reagents can include genome editing reagents such as a nuclease agent (e.g., a Clustered Regularly Interspersed Short Palindromic Repeats (CRISPR)/CRISPR-associated (Cas) (CRISPR/Cas) nuclease, a zinc finger nuclease (ZFN), or a Transcription Activator-Like Effector Nuclease (TALEN)) that cleaves a recognition site within a target gene expressed in a human liver. Likewise, a human-liver-targeting reagent can be an exogenous donor nucleic acid (e.g., a targeting vector or single-stranded oligodeoxynucleotide (ssODN)) designed to recombine with the target gene expressed in a human liver.

Other human-liver-targeting reagents can include RNAi agents designed to target RNAs (e.g., messenger RNAs) expressed in or present in a human liver. An “RNAi agent” is a composition that comprises a small double-stranded RNA or RNA-like (e.g., chemically modified RNA) oligonucleotide molecule capable of facilitating degradation or inhibition of translation of a target RNA, such as messenger RNA (mRNA), in a sequence-specific manner. The oligonucleotide in the RNAi agent is a polymer of linked nucleosides, each of which can be independently modified or unmodified. RNAi agents operate through the RNA interference mechanism (i.e., inducing RNA interference through interaction with the RNA interference pathway machinery (RNA-induced silencing complex or RISC) of mammalian cells). While it is believed that RNAi agents, as that term is used herein, operate primarily through the RNA interference mechanism, the disclosed RNAi agents are not bound by or limited to any particular pathway or mechanism of action. RNAi agents disclosed herein comprise a sense strand and an antisense strand, and include, but are not limited to, short interfering RNAs (siRNAs), double-stranded RNAs (dsRNA), micro RNAs (miRNAs), short hairpin RNAs (shRNA), and dicer substrates. The antisense strand of the RNAi agents described herein is at least partially complementary to a sequence (i.e., a succession or order of nucleobases or nucleotides, described with a succession of letters using standard nomenclature) in the target RNA.

Other human-liver-targeting reagents can include antisense oligonucleotides (ASOs) designed to target a target RNA present in or expressed in a human liver. Single-stranded ASOs and RNA interference (RNAi) share a fundamental principle in that an oligonucleotide binds a target RNA through Watson-Crick base pairing. Without wishing to be bound by theory, during RNAi, a small RNA duplex (RNAi agent) associates with the RNA-induced silencing complex (RISC), one strand (the passenger strand) is lost, and the remaining strand (the guide strand) cooperates with RISC to bind complementary RNA. Argonaute 2 (Ago2), the catalytic component of the RISC, then cleaves the target RNA. The guide strand is always associated with either the complementary sense strand or a protein (RISC). In contrast, an ASO must survive and function as a single strand. ASOs bind to the target RNA and block ribosomes or other factors, such as splicing factors, from binding the RNA or recruit proteins such as nucleases. Different modifications and target regions are chosen for ASOs based on the desired mechanism of action. A gapmer is an ASO oligonucleotide containing 2-5 chemically modified nucleotides (e.g. LNA or 2′-MOE) on each terminus flanking a central 8-10 base gap of DNA. After binding the target RNA, the DNA-RNA hybrid acts substrate for RNase H.

Such human-liver-targeting reagents can be administered by any delivery method (e.g., AAV, LNP, HDD, or injection) and by any route of administration. Means of delivering complexes and molecules and routes of administration are disclosed in more detail elsewhere herein. In particular methods, the reagents can be delivered via AAV-mediated delivery. For example, AAV8 can be used to target the liver. In other particular methods, the reagents can be delivered by hydrodynamic delivery (HDD). The dose can be any suitable dose. In other particular methods, the reagents can be delivered by LNP-mediated delivery. In some method, LNP-mediated delivery of reagents to genetically modified non-human animals in which the genetically modified non-human animal and/or the transplanted hepatocytes are modified to restore interleukin-6 (IL-6)/interleukin-6 receptor (IL-6R) signaling pathway activity or GP130 signaling pathway activity in the transplanted hepatocytes is more efficient than LNP-mediated delivery of reagents to control animals in which the genetically modified non-human animal and the transplanted hepatocytes are not modified to restore interleukin-6 (IL-6)/interleukin-6 receptor (IL-6R) signaling pathway activity or GP130 signaling pathway activity in the transplanted hepatocytes.

Any known methods for assessing activity of the human-liver-targeting reagent can be used. If the human-liver-targeting reagent is a genome editing reagent (e.g., a nuclease agent) designed to target a target genomic locus, such methods can comprise assessing modification of the target genomic locus. Various methods can be used to identify cells having a targeted genetic modification. The screening can comprise a quantitative assay for assessing modification-of-allele (MOA) of a parental chromosome. See, e.g., US 2004/0018626; US 2014/0178879; US 2016/0145646; WO 2016/081923; and Frendewey et al. (2010) Methods Enzymol. 476:295-307, each of which is herein incorporated by reference in its entirety for all purposes. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence. Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, herein incorporated by reference in its entirety for all purposes). Next-generation sequencing (NGS) can also be used for screening. Next-generation sequencing can also be referred to as “NGS” or “massively parallel sequencing” or “high throughput sequencing.” NGS can be used as a screening tool in addition to the MOA assays to define the exact nature of the targeted genetic modification and whether it is consistent across cell types or tissue types or organ types. As one example, the assessing can comprise measuring non-homologous end joining (NHEJ) activity at the targeted genomic locus. This can comprise, for example, measuring the frequency of insertions or deletions within the target genomic locus. For example, the assessing can comprise sequencing the target genomic locus in one or more cells isolated from the non-human animal (e.g., next-generation sequencing). Assessment can comprise isolating a target organ or tissue (e.g., liver) from the non-human animal and assessing modification of target genomic locus in the target organ or tissue. Assessment can also comprise assessing modification of target genomic locus in two or more different cell types within the target organ or tissue. Similarly, assessment can comprise isolating a non-target organ or tissue (e.g., two or more non-target organs or tissues) from the non-human animal and assessing modification of target genomic locus in the non-target organ or tissue.

One example of an assay that can be used are the RNASCOPE™ and BASESCOPE™ RNA in situ hybridization (ISH) assays, which are methods that can quantify cell-specific edited transcripts, including single nucleotide changes, in the context of intact fixed tissue. The BASESCOPE™ RNA ISH assay can complement NGS and qPCR in characterization of gene editing. Whereas NGS/qPCR can provide quantitative average values of wild type and edited sequences, they provide no information on heterogeneity or percentage of edited cells within a tissue. The BASESCOPE™ ISH assay can provide a landscape view of an entire tissue and quantification of wild type versus edited transcripts with single-cell resolution, where the actual number of cells within the target tissue containing the edited mRNA transcript can be quantified. The BASESCOPE™ assay achieves single-molecule RNA detection using paired oligo (“ZZ”) probes to amplify signal without non-specific background. However, the BASESCOPE™ probe design and signal amplification system enables single-molecule RNA detection with a ZZ probe, and it can differentially detect single nucleotide edits and mutations in intact fixed tissue.

Such methods can also comprise measuring expression levels of the RNA (e.g., messenger RNA) produced by a target genomic locus, or by measuring expression levels of the protein encoded by the target genomic locus. For example, protein levels can be measured in liver, or if the target genomic locus encodes a secreted protein, secreted levels can be measured in the serum. For example, the human-liver-targeting reagent can target a target gene expressed in human liver, and the method can comprise measuring expression of an RNA (e.g., a messenger RNA) or a protein encoded by the target gene. Alternatively, the human-liver-targeting reagent can target an RNA (e.g., a messenger RNA) expressed in the human liver, and the method can comprise measuring expression of the RNA or a protein encoded by the target gene. Alternatively, the human-liver-targeting reagent can target a protein expressed in the human liver, and the method can comprise measuring expression of the protein. Methods for assessing expression of mRNA or protein are provided elsewhere herein and are well-known.

Such methods can also comprise measuring activity of an RNA or protein encoded by a target gene, of a protein encoded by a target messenger RNA, or of a targeted protein.

As one specific example, if the human-liver-targeting reagent is a genome editing reagent (e.g., a nuclease agent), percent editing (e.g., total number of insertions or deletions observed over the total number of sequences read in the PCR reaction from a pool of lysed cells) at the target genomic locus can be assessed (e.g., in liver cells).

The various methods provided above for assessing activity in vivo can also be used to assess the activity of human-liver-targeting reagents ex vivo (e.g., in a liver) or in vitro (e.g., in a cell) as described elsewhere herein.

D. Methods of Administering Agents or Compounds to Non-Human Animals or Hepatocytes

The methods disclosed herein can comprise introducing into a non-human animal or cells for transplanting (e.g., hepatocytes or hepatocyte precursors) various agents or compounds (e.g., human-liver-targeting agents, IL-6 or a nucleic acid encoding IL-6, a nucleic acid encoding GP130, a nucleic acid encoding IL-6R, OSM or a nucleic acid encoding OSM, a nucleic acid encoding OSMR, etc.), including nucleic acids, proteins, nucleic-acid-protein complexes, peptide mimetics, antigen-binding proteins, or small molecules. “Introducing” includes presenting to the non-human animal cell or non-human animal the molecule (e.g., nucleic acid or protein or small molecule) in such a manner that it gains access to the interior of the non-human animal cell or to the interior of cells within the non-human animal. The introducing can be accomplished by any means. If multiple components are introduced, they can be introduced simultaneously or sequentially in any combination. In addition, two or more of the components can be introduced into the non-human animal cell or non-human animal by the same delivery method or different delivery methods. Similarly, two or more of the components can be introduced into a non-human animal by the same route of administration or different routes of administration.

Agents or compounds introduced into the cell or non-human animal can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of a human-liver-targeting reagent or other agents or compounds (e.g., IL-6 or a nucleic acid encoding IL-6, a nucleic acid encoding GP130, a nucleic acid encoding IL-6R, OSM or a nucleic acid encoding OSM, a nucleic acid encoding OSMR, etc.) into a cell or non-human animal. Methods for introducing agents into various cell types are known and include, for example, stable transfection methods, transient transfection methods, and virus-mediated methods. Transfection protocols as well as protocols for introducing agents into cells may vary.

Non-limiting transfection methods include chemical-based transfection methods using liposomes; nanoparticles; calcium phosphate (Graham et al. (1973) Virology 52(2):456-467, Bacchetti et al. (1977) Proc. Natl. Acad. Sci. USA 74(4):1590-1594, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Curr. Pharm. Biotechnol. 7(4):277-285). Viral methods can also be used for transfection.

Introduction of human-liver-targeting reagents or other agents or compounds (e.g., IL-6 or a nucleic acid encoding IL-6, a nucleic acid encoding GP130, a nucleic acid encoding IL-6R, OSM or a nucleic acid encoding OSM, a nucleic acid encoding OSMR, etc.) into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA ° NUCLEOFECTOR™ system.

Introduction of human-liver-targeting reagents or other agents or compounds (e.g., IL-6 or a nucleic acid encoding IL-6, a nucleic acid encoding GP130, a nucleic acid encoding IL-6R, OSM or a nucleic acid encoding OSM, a nucleic acid encoding OSMR, etc.) into a cell (e.g., a zygote) can also be accomplished by microinjection. In zygotes (i.e., one-cell stage embryos), microinjection can be into the maternal and/or paternal pronucleus or into the cytoplasm. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. USA 107:15022-15026 and Meyer et al. (2012) Proc. Natl. Acad. Sci. USA 109:9354-9359.

Other methods for introducing human-liver-targeting reagents or other agents or compounds (e.g., IL-6 or a nucleic acid encoding IL-6, a nucleic acid encoding GP130, a nucleic acid encoding IL-6R, OSM or a nucleic acid encoding OSM, a nucleic acid encoding OSMR, etc.) into a cell or non-human animal can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a non-human animal cell or non-human animal in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a non-human animal include hydrodynamic delivery, virus-mediated delivery (e.g., adeno-associated virus (AAV)-mediated delivery), and lipid-nanoparticle-mediated delivery.

Introduction of human-liver-targeting reagents or other agents or compounds (e.g., IL-6 or a nucleic acid encoding IL-6, a nucleic acid encoding GP130, a nucleic acid encoding IL-6R, OSM or a nucleic acid encoding OSM, a nucleic acid encoding OSMR, etc.) into cells or non-human animals can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of human-liver-targeting reagents or other agents or compounds (e.g., IL-6 or a nucleic acid encoding IL-6, a nucleic acid encoding GP130, a nucleic acid encoding IL-6R, OSM or a nucleic acid encoding OSM, a nucleic acid encoding OSMR, etc.) can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression. Exemplary viral titers (e.g., AAV titers) include 10¹², 10¹³, 10¹⁴, 10¹⁵ and 10¹⁶ vector genomes/mL.

Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Serotypes for heart tissue include AAV1, AAV8, and AAV9. Serotypes for kidney tissue include AAV2. Serotypes for lung tissue include AAV4, AAV5, AAV6, and AAV9. Serotypes for pancreas tissue include AAV8. Serotypes for photoreceptor cells include AAV2, AAV5, and AAV8. Serotypes for retinal pigment epithelium tissue include AAV1, AAV2, AAV4, AAV5, and AAV8. Serotypes for skeletal muscle tissue include AAV1, AAV6, AAV7, AAV8, and AAV9. Serotypes for liver tissue include AAV7, AAV8, and AAV9, and particularly AAV8. In a specific example, AAV9 is used.

Introduction of human-liver-targeting reagents or other agents or compounds (e.g., IL-6 or a nucleic acid encoding IL-6, a nucleic acid encoding GP130, a nucleic acid encoding IL-6R, OSM or a nucleic acid encoding OSM, a nucleic acid encoding OSMR, etc.) can also be accomplished by lipid nanoparticle (LNP)-mediated delivery. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyms, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. In a particular example, the route of administration is subcutaneous or intravenous. In another example, the route of administration is intrasplenic injection. In another example, the route of administration is injection into the hepatic artery, the spleen, the portal vein, the peritoneal cavity, hepatic tissue mass, or the lymphatic system, or as part of a liver tissue graft.

Compositions comprising human-liver-targeting reagents or other agents or compounds (e.g., IL-6 or a nucleic acid encoding IL-6, a nucleic acid encoding GP130, a nucleic acid encoding IL-6R, OSM or a nucleic acid encoding OSM, a nucleic acid encoding OSMR, etc.) can be formulated using one or more physiologically and pharmaceutically acceptable carriers, diluents, excipients or auxiliaries. The formulation can depend on the route of administration chosen. The term “pharmaceutically acceptable” means that the carrier, diluent, excipient, or auxiliary is compatible with the other ingredients of the formulation and not substantially deleterious to the recipient thereof.

The frequency of administration and the number of dosages can depend on the half-life of the human-liver-targeting reagents or other agents or compounds (e.g., IL-6 or a nucleic acid encoding IL-6, a nucleic acid encoding GP130, a nucleic acid encoding IL-6R, OSM or a nucleic acid encoding OSM, a nucleic acid encoding OSMR, etc.) and the route of administration among other factors. The introduction of human-liver-targeting reagents or other agents or compounds (e.g., IL-6 or a nucleic acid encoding IL-6, a nucleic acid encoding GP130, a nucleic acid encoding IL-6R, OSM or a nucleic acid encoding OSM, a nucleic acid encoding OSMR, etc.) into the cell or non-human animal can be performed one time or multiple times over a period of time. For example, the introduction can be performed at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time.

III. Methods of Making Non-Human Animals Comprising Inactivated and/or Humanized Genes

Various methods are provided for making non-human animals comprising inactivated genes (e.g., Rag1, Rag2, Il2rg, or Fah genes or combination thereof) and/or humanized genes (e.g., IL6, OSM, GH, or combination thereof). For example, various methods are provided for making a non-human animal comprising inactivated Rag2, Il2rg, and Fah genes and a humanized IL6 gene and optionally a humanized SIRPA gene (or any combination thereof) as disclosed elsewhere herein. For example, various methods are provided for making a non-human animal comprising inactivated Rag1, Rag2, Il2rg, and Fah genes and a humanized IL6 gene and optionally a humanized SIRPA gene (or any combination thereof) as disclosed elsewhere herein. For example, various methods are provided for making a non-human animal comprising inactivated Rag2, Il2rg, and Fah genes and a humanized OSM gene and optionally a humanized SIRPA gene (or any combination thereof) as disclosed elsewhere herein. For example, various methods are provided for making a non-human animal comprising inactivated Rag1, Rag2, Il2rg, and Fah genes and a humanized OSM gene and optionally a humanized SIRPA gene (or any combination thereof) as disclosed elsewhere herein. For example, various methods are provided for making a non-human animal comprising inactivated Rag2, Il2rg, and Fah genes and humanized IL6 and OSM genes and optionally a humanized SIRPA gene (or any combination thereof) as disclosed elsewhere herein. For example, various methods are provided for making a non-human animal comprising inactivated Rag1, Rag2, Il2rg, and Fah genes and humanized IL6 and OSM genes and optionally a humanized SIRPA gene (or any combination thereof) as disclosed elsewhere herein. Likewise, various methods are provided for making inactivated non-human animal Rag2, Il2rg, and Fah genes and a humanized IL6 gene and optionally a humanized SIRPA gene (or any combination thereof) or for making a non-human animal genome or non-human animal cell comprising inactivated Rag2, Il2rg, and Fah genes and a humanized IL6 gene and optionally a humanized SIRPA gene (or any combination thereof) as disclosed elsewhere herein. Likewise, various methods are provided for making inactivated non-human animal Rag1, Rag2, Il2rg, and Fah genes and a humanized IL6 gene and optionally a humanized SIRPA gene (or any combination thereof) or for making a non-human animal genome or non-human animal cell comprising inactivated Rag1, Rag2, Il2rg, and Fah genes and a humanized IL6 gene and optionally a humanized SIRPA gene (or any combination thereof) as disclosed elsewhere herein. Likewise, various methods are provided for making inactivated non-human animal Rag2, Il2rg, and Fah genes and a humanized OSM gene and optionally a humanized SIRPA gene (or any combination thereof) or for making a non-human animal genome or non-human animal cell comprising inactivated Rag2, Il2rg, and Fah genes and a humanized OSM gene and optionally a humanized SIRPA gene (or any combination thereof) as disclosed elsewhere herein. Likewise, various methods are provided for making inactivated non-human animal Rag1, Rag2, Il2rg, and Fah genes and a humanized OSM gene and optionally a humanized SIRPA gene (or any combination thereof) or for making a non-human animal genome or non-human animal cell comprising inactivated Rag1, Rag2, Il2rg, and Fah genes and a humanized OSM gene and optionally a humanized SIRPA gene (or any combination thereof) as disclosed elsewhere herein. Likewise, various methods are provided for making inactivated non-human animal Rag2, Il2rg, and Fah genes and humanized IL6 and OSM genes and optionally a humanized SIRPA gene (or any combination thereof) or for making a non-human animal genome or non-human animal cell comprising inactivated Rag2, Il2rg, and Fah genes and humanized IL6 and OSM genes and optionally a humanized SIRPA gene (or any combination thereof) as disclosed elsewhere herein. Likewise, various methods are provided for making inactivated non-human animal Rag1, Rag2, Il2rg, and Fah genes and humanized IL6 and OSM genes and optionally a humanized SIRPA gene (or any combination thereof) or for making a non-human animal genome or non-human animal cell comprising inactivated Rag1, Rag2, Il2rg, and Fah genes and humanized IL6 and OSM genes and optionally a humanized SIRPA gene (or any combination thereof) as disclosed elsewhere herein. Any convenient method or protocol for producing a genetically modified non-human animal (e.g., mouse or rat) is suitable for producing such a genetically modified rat. Any convenient method or protocol for producing a genetically modified organism is suitable for producing such a genetically modified non-human animal. See, e.g., Poueymirou et al. (2007) Nat. Biotechnol. 25(1):91-99; U.S. Pat. Nos. 7,294,754; 7,576,259; 7,659,442; 8,816,150; 9,414,575; 9,730,434; and 10,039,269, each of which is herein incorporated by reference in its entirety for all purposes (describing mouse ES cells and the VELOCIMOUSE® method for making a genetically modified mouse). See also US 2014/0235933 A1, US 2014/0310828 A1, each of which is herein incorporated by reference in its entirety for all purposes (describing rat ES cells and methods for making a genetically modified rat). See also Cho et al. (2009) Curr. Protoc. Cell. Biol. 42:19.11.1-19.11.22 (doi: 10.1002/0471143030.cb1911s42) and Gama Sosa et al. (2010) Brain Struct. Funct. 214(2-3):91-109, each of which is herein incorporated by reference in its entirety for all purposes. Such genetically modified non-human animals can be generated, for example, through gene knock-out at targeted Rag2, Il2rg, and Fah genes and gene humanization at a targeted IL6 gene and optionally a gene humanization at a targeted SIRPA gene. Such genetically modified non-human animals can be generated, for example, through gene knock-out at targeted Rag1, Rag2, Il2rg, and Fah genes and gene humanization at a targeted IL6 gene and optionally a gene humanization at a targeted SIRPA gene.

For example, a method of producing a non-human animal comprising inactivated Rag2, Il2rg, and Fah genes and a humanized IL6 gene and optionally a humanized SIRPA gene can comprise: (1) providing a pluripotent non-human animal cell (e.g., a non-human animal embryonic stem (ES) cell) comprising inactivated Rag2, Il2rg, and Fah genes and a humanized IL6 gene and optionally a humanized SIRPA gene; (2) introducing the genetically modified pluripotent non-human animal cell into a non-human animal host embryo; and (3) gestating (e.g., implanting and gestating) the non-human animal host embryo in a surrogate non-human animal mother. For example, a method of producing a non-human animal comprising inactivated Rag1, Rag2, Il2rg, and Fah genes and a humanized IL6 gene and optionally a humanized SIRPA gene can comprise: (1) providing a pluripotent non-human animal cell (e.g., a non-human animal embryonic stem (ES) cell) comprising inactivated Rag1, Rag2, Il2rg, and Fah genes and a humanized IL6 gene and optionally a humanized SIRPA gene; (2) introducing the genetically modified pluripotent non-human animal cell into a non-human animal host embryo; and (3) gestating (e.g., implanting and gestating) the non-human animal host embryo in a surrogate non-human animal mother. For example, a method of producing a non-human animal comprising inactivated Rag2, Il2rg, and Fah genes and a humanized OSM gene and optionally a humanized SIRPA gene can comprise: (1) providing a pluripotent non-human animal cell (e.g., a non-human animal embryonic stem (ES) cell) comprising inactivated Rag2, Il2rg, and Fah genes and a humanized OSM gene and optionally a humanized SIRPA gene; (2) introducing the genetically modified pluripotent non-human animal cell into a non-human animal host embryo; and (3) gestating (e.g., implanting and gestating) the non-human animal host embryo in a surrogate non-human animal mother. For example, a method of producing a non-human animal comprising inactivated Rag1, Rag2, Il2rg, and Fah genes and a humanized OSM gene and optionally a humanized SIRPA gene can comprise: (1) providing a pluripotent non-human animal cell (e.g., a non-human animal embryonic stem (ES) cell) comprising inactivated Rag1, Rag2, Il2rg, and Fah genes and a humanized OSM gene and optionally a humanized SIRPA gene; (2) introducing the genetically modified pluripotent non-human animal cell into a non-human animal host embryo; and (3) gestating (e.g., implanting and gestating) the non-human animal host embryo in a surrogate non-human animal mother. For example, a method of producing a non-human animal comprising inactivated Rag2, Il2rg, and Fah genes and humanized IL6 and OSM genes and optionally a humanized SIRPA gene can comprise: (1) providing a pluripotent non-human animal cell (e.g., a non-human animal embryonic stem (ES) cell) comprising inactivated Rag2, Il2rg, and Fah genes and humanized IL6 and OSM genes and optionally a humanized SIRPA gene; (2) introducing the genetically modified pluripotent non-human animal cell into a non-human animal host embryo; and (3) gestating (e.g., implanting and gestating) the non-human animal host embryo in a surrogate non-human animal mother. For example, a method of producing a non-human animal comprising inactivated Rag1, Rag2, Il2rg, and Fah genes and humanized IL6 and OSM genes and optionally a humanized SIRPA gene can comprise: (1) providing a pluripotent non-human animal cell (e.g., a non-human animal embryonic stem (ES) cell) comprising inactivated Rag1, Rag2, Il2rg, and Fah genes and humanized IL6 and OSM genes and optionally a humanized SIRPA gene; (2) introducing the genetically modified pluripotent non-human animal cell into a non-human animal host embryo; and (3) gestating (e.g., implanting and gestating) the non-human animal host embryo in a surrogate non-human animal mother.

Alternatively, a method of producing the non-human animals described elsewhere herein can comprise gestating (e.g., implanting and gestating) a non-human animal one-cell stage embryo comprising the inactivated Rag2, Il2rg, and Fah genes and a humanized IL6 gene and optionally a humanized SIRPA gene in a non-human animal surrogate mother. Alternatively, a method of producing the non-human animals described elsewhere herein can comprise gestating (e.g., implanting and gestating) a non-human animal one-cell stage embryo comprising the inactivated Rag2, Il2rg, and Fah genes and a humanized OSM gene and optionally a humanized SIRPA gene in a non-human animal surrogate mother. Alternatively, a method of producing the non-human animals described elsewhere herein can comprise gestating (e.g., implanting and gestating) a non-human animal one-cell stage embryo comprising the inactivated Rag1, Rag2, Il2rg, and Fah genes and a humanized IL6 gene and optionally a humanized SIRPA gene in a non-human animal surrogate mother. Alternatively, a method of producing the non-human animals described elsewhere herein can comprise gestating (e.g., implanting and gestating) a non-human animal one-cell stage embryo comprising the inactivated Rag1, Rag2, Il2rg, and Fah genes and a humanized OSM gene and optionally a humanized SIRPA gene in a non-human animal surrogate mother. Alternatively, a method of producing the non-human animals described elsewhere herein can comprise gestating (e.g., implanting and gestating) a non-human animal one-cell stage embryo comprising the inactivated Rag2, Il2rg, and Fah genes and humanized IL6 and OSM genes and optionally a humanized SIRPA gene in a non-human animal surrogate mother. Alternatively, a method of producing the non-human animals described elsewhere herein can comprise gestating (e.g., implanting and gestating) a non-human animal one-cell stage embryo comprising the inactivated Rag1, Rag2, Il2rg, and Fah genes and humanized IL6 and OSM genes and optionally a humanized SIRPA gene in a non-human animal surrogate mother.

As another example, the method of producing a non-human animal comprising inactivated Rag2, Il2rg, and Fah genes and a humanized IL6 gene and optionally a humanized SIRPA gene (or any combination thereof) can comprise: (1) modifying the genome of a non-human animal pluripotent cell (e.g., a non-human animal ES cell) to comprise the inactivated Rag2, Il2rg, and Fah genes and a humanized IL6 gene and optionally a humanized SIRPA gene; (2) identifying or selecting the genetically modified non-human animal pluripotent cell comprising the inactivated Rag2, Il2rg, and Fah genes and a humanized IL6 gene and optionally a humanized SIRPA gene; (3) introducing the genetically modified non-human animal pluripotent cell into a non-human animal host embryo; and (4) gestating (e.g., implanting and gestating) the non-human animal host embryo in a non-human animal surrogate mother. Optionally, the host embryo comprising modified non-human animal pluripotent cell (e.g., a non-human animal ES cell) can be incubated until the blastocyst stage before being implanted into and gestated in the non-human animal surrogate mother to produce an F0 non-human animal. The non-human animal surrogate mother can then produce an F0 generation non-human animal comprising the inactivated Rag2, Il2rg, and Fah genes and a humanized IL6 gene and optionally a humanized SIRPA gene (and capable of transmitting the genetic modifications through the germline). The step of modifying the genome to comprise the inactivated Rag2, Il2rg, and Fah genes and a humanized IL6 gene and optionally a humanized SIRPA gene can be done in any background. For example, the modifying step can be done in a non-human animal pluripotent cell in which none of the Rag2, Il2rg, and Fah genes have already been inactivated and the IL6 gene has not been humanized and the SIRPA gene has not been humanized. Alternatively, the modifying step for any one of the genes can be done in a non-human animal pluripotent cell in which one or more of Rag2, Il2rg, and Fah loci or genes have already been inactivated (e.g., one, two, or all three of the other loci or genes have already been inactivated) and/or the IL6 gene has already been humanized (and optionally the SIRPA gene has already been humanized). If two or more of the genes are inactivated in non-human animal pluripotent cells in which none of the other genes have already been inactivated/humanized, non-human animals comprising the separate inactivated/humanized genes can be crossed to produce non-human animals having the two or more inactivated/humanized genes. As another example, the method of producing a non-human animal comprising inactivated Rag1, Rag2, Il2rg, and Fah genes and a humanized IL6 gene and optionally a humanized SIRPA gene (or any combination thereof) can comprise: (1) modifying the genome of a non-human animal pluripotent cell (e.g., a non-human animal ES cell) to comprise the inactivated Rag1, Rag2, Il2rg, and Fah genes and a humanized IL6 gene and optionally a humanized SIRPA gene; (2) identifying or selecting the genetically modified non-human animal pluripotent cell comprising the inactivated Rag1, Rag2, Il2rg, and Fah genes and a humanized IL6 gene and optionally a humanized SIRPA gene; (3) introducing the genetically modified non-human animal pluripotent cell into a non-human animal host embryo; and (4) gestating (e.g., implanting and gestating) the non-human animal host embryo in a non-human animal surrogate mother. Optionally, the host embryo comprising modified non-human animal pluripotent cell (e.g., a non-human animal ES cell) can be incubated until the blastocyst stage before being implanted into and gestated in the non-human animal surrogate mother to produce an F0 non-human animal. The non-human animal surrogate mother can then produce an F0 generation non-human animal comprising the inactivated Rag1, Rag2, Il2rg, and Fah genes and a humanized IL6 gene and optionally a humanized SIRPA gene (and capable of transmitting the genetic modifications through the germline). The step of modifying the genome to comprise the inactivated Rag1, Rag2, Il2rg, and Fah genes and a humanized IL6 gene and optionally a humanized SIRPA gene can be done in any background. For example, the modifying step can be done in a non-human animal pluripotent cell in which none of the Rag1, Rag2, Il2rg, and Fah genes have already been inactivated and the IL6 gene has not been humanized and the SIRPA gene has not been humanized. Alternatively, the modifying step for any one of the genes can be done in a non-human animal pluripotent cell in which one or more of Rag1, Rag2, Il2rg, and Fah loci or genes have already been inactivated (e.g., one, two, or all three of the other loci or genes have already been inactivated) and/or the IL6 gene has already been humanized (and optionally the SIRPA gene has already been humanized). If two or more of the genes are inactivated in non-human animal pluripotent cells in which none of the other genes have already been inactivated/humanized, non-human animals comprising the separate inactivated/humanized genes can be crossed to produce non-human animals having the two or more inactivated/humanized genes.

As another example, the method of producing a non-human animal comprising inactivated Rag2, Il2rg, and Fah genes and a humanized OSM gene and optionally a humanized SIRPA gene (or any combination thereof) can comprise: (1) modifying the genome of a non-human animal pluripotent cell (e.g., a non-human animal ES cell) to comprise the inactivated Rag2, Il2rg, and Fah genes and a humanized OSM gene and optionally a humanized SIRPA gene; (2) identifying or selecting the genetically modified non-human animal pluripotent cell comprising the inactivated Rag2, Il2rg, and Fah genes and a humanized OSM gene and optionally a humanized SIRPA gene; (3) introducing the genetically modified non-human animal pluripotent cell into a non-human animal host embryo; and (4) gestating (e.g., implanting and gestating) the non-human animal host embryo in a non-human animal surrogate mother. Optionally, the host embryo comprising modified non-human animal pluripotent cell (e.g., a non-human animal ES cell) can be incubated until the blastocyst stage before being implanted into and gestated in the non-human animal surrogate mother to produce an F0 non-human animal. The non-human animal surrogate mother can then produce an F0 generation non-human animal comprising the inactivated Rag2, Il2rg, and Fah genes and a humanized OSM gene and optionally a humanized SIRPA gene (and capable of transmitting the genetic modifications through the germline). The step of modifying the genome to comprise the inactivated Rag2, Il2rg, and Fah genes and a humanized OSM gene and optionally a humanized SIRPA gene can be done in any background. For example, the modifying step can be done in a non-human animal pluripotent cell in which none of the Rag2, Il2rg, and Fah genes have already been inactivated and the OSM gene has not been humanized and the SIRPA gene has not been humanized. Alternatively, the modifying step for any one of the genes can be done in a non-human animal pluripotent cell in which one or more of Rag2, Il2rg, and Fah loci or genes have already been inactivated (e.g., one, two, or all three of the other loci or genes have already been inactivated) and/or the OSM gene has already been humanized (and optionally the SIRPA gene has already been humanized). If two or more of the genes are inactivated in non-human animal pluripotent cells in which none of the other genes have already been inactivated/humanized, non-human animals comprising the separate inactivated/humanized genes can be crossed to produce non-human animals having the two or more inactivated/humanized genes. As another example, the method of producing a non-human animal comprising inactivated Rag1, Rag2, Il2rg, and Fah genes and a humanized OSM gene and optionally a humanized SIRPA gene (or any combination thereof) can comprise: (1) modifying the genome of a non-human animal pluripotent cell (e.g., a non-human animal ES cell) to comprise the inactivated Rag1, Rag2, Il2rg, and Fah genes and a humanized OSM gene and optionally a humanized SIRPA gene; (2) identifying or selecting the genetically modified non-human animal pluripotent cell comprising the inactivated Rag1, Rag2, Il2rg, and Fah genes and a humanized OSM gene and optionally a humanized SIRPA gene; (3) introducing the genetically modified non-human animal pluripotent cell into a non-human animal host embryo; and (4) gestating (e.g., implanting and gestating) the non-human animal host embryo in a non-human animal surrogate mother. Optionally, the host embryo comprising modified non-human animal pluripotent cell (e.g., a non-human animal ES cell) can be incubated until the blastocyst stage before being implanted into and gestated in the non-human animal surrogate mother to produce an F0 non-human animal. The non-human animal surrogate mother can then produce an F0 generation non-human animal comprising the inactivated Rag1, Rag2, Il2rg, and Fah genes and a humanized OSM gene and optionally a humanized SIRPA gene (and capable of transmitting the genetic modifications through the germline). The step of modifying the genome to comprise the inactivated Rag1, Rag2, Il2rg, and Fah genes and a humanized OSM gene and optionally a humanized SIRPA gene can be done in any background. For example, the modifying step can be done in a non-human animal pluripotent cell in which none of the Rag1, Rag2, Il2rg, and Fah genes have already been inactivated and the OSM gene has not been humanized and the SIRPA gene has not been humanized. Alternatively, the modifying step for any one of the genes can be done in a non-human animal pluripotent cell in which one or more of Rag1, Rag2, Il2rg, and Fah loci or genes have already been inactivated (e.g., one, two, or all three of the other loci or genes have already been inactivated) and/or the OSM gene has already been humanized (and optionally the SIRPA gene has already been humanized). If two or more of the genes are inactivated in non-human animal pluripotent cells in which none of the other genes have already been inactivated/humanized, non-human animals comprising the separate inactivated/humanized genes can be crossed to produce non-human animals having the two or more inactivated/humanized genes.

As another example, the method of producing a non-human animal comprising inactivated Rag2, Il2rg, and Fah genes and humanized IL6 and OSM genes and optionally a humanized SIRPA gene (or any combination thereof) can comprise: (1) modifying the genome of a non-human animal pluripotent cell (e.g., a non-human animal ES cell) to comprise the inactivated Rag2, Il2rg, and Fah genes and humanized IL6 and OSM genes and optionally a humanized SIRPA gene; (2) identifying or selecting the genetically modified non-human animal pluripotent cell comprising the inactivated Rag2, Il2rg, and Fah genes and humanized IL6 and OSM genes and optionally a humanized SIRPA gene; (3) introducing the genetically modified non-human animal pluripotent cell into a non-human animal host embryo; and (4) gestating (e.g., implanting and gestating) the non-human animal host embryo in a non-human animal surrogate mother. Optionally, the host embryo comprising modified non-human animal pluripotent cell (e.g., a non-human animal ES cell) can be incubated until the blastocyst stage before being implanted into and gestated in the non-human animal surrogate mother to produce an F0 non-human animal. The non-human animal surrogate mother can then produce an F0 generation non-human animal comprising the inactivated Rag2, Il2rg, and Fah genes and humanized IL6 and OSM genes and optionally a humanized SIRPA gene (and capable of transmitting the genetic modifications through the germline). The step of modifying the genome to comprise the inactivated Rag2, Il2rg, and Fah genes and humanized IL6 and OSM genes and optionally a humanized SIRPA gene can be done in any background. For example, the modifying step can be done in a non-human animal pluripotent cell in which none of the Rag2, Il2rg, and Fah genes have already been inactivated and the IL6 and OSM genes have not been humanized and the SIRPA gene has not been humanized. Alternatively, the modifying step for any one of the genes can be done in a non-human animal pluripotent cell in which one or more of Rag2, Il2rg, and Fah loci or genes have already been inactivated (e.g., one, two, or all three of the other loci or genes have already been inactivated) and/or one or both the IL6 gene and OSM gene have already been humanized (and optionally the SIRPA gene has already been humanized). If two or more of the genes are inactivated in non-human animal pluripotent cells in which none of the other genes have already been inactivated/humanized, non-human animals comprising the separate inactivated/humanized genes can be crossed to produce non-human animals having the two or more inactivated/humanized genes. As another example, the method of producing a non-human animal comprising inactivated Rag1, Rag2, Il2rg, and Fah genes and humanized IL6 and OSM genes and optionally a humanized SIRPA gene (or any combination thereof) can comprise: (1) modifying the genome of a non-human animal pluripotent cell (e.g., a non-human animal ES cell) to comprise the inactivated Rag1, Rag2, Il2rg, and Fah genes and humanized IL6 and OSM genes and optionally a humanized SIRPA gene; (2) identifying or selecting the genetically modified non-human animal pluripotent cell comprising the inactivated Rag1, Rag2, Il2rg, and Fah genes and humanized IL6 and OSM genes and optionally a humanized SIRPA gene; (3) introducing the genetically modified non-human animal pluripotent cell into a non-human animal host embryo; and (4) gestating (e.g., implanting and gestating) the non-human animal host embryo in a non-human animal surrogate mother. Optionally, the host embryo comprising modified non-human animal pluripotent cell (e.g., a non-human animal ES cell) can be incubated until the blastocyst stage before being implanted into and gestated in the non-human animal surrogate mother to produce an F0 non-human animal. The non-human animal surrogate mother can then produce an F0 generation non-human animal comprising the inactivated Rag1, Rag2, Il2rg, and Fah genes and humanized IL6 and OSM genes and optionally a humanized SIRPA gene (and capable of transmitting the genetic modifications through the germline). The step of modifying the genome to comprise the inactivated Rag1, Rag2, Il2rg, and Fah genes and humanized IL6 and OSM genes and optionally a humanized SIRPA gene can be done in any background. For example, the modifying step can be done in a non-human animal pluripotent cell in which none of the Rag1, Rag2, Il2rg, and Fah genes have already been inactivated and the IL6 and OSM genes have not been humanized and the SIRPA gene has not been humanized. Alternatively, the modifying step for any one of the genes can be done in a non-human animal pluripotent cell in which one or more of Rag1, Rag2, Il2rg, and Fah loci or genes have already been inactivated (e.g., one, two, or all three of the other loci or genes have already been inactivated) and/or one or both the IL6 gene and OSM gene have already been humanized (and optionally the SIRPA gene has already been humanized). If two or more of the genes are inactivated in non-human animal pluripotent cells in which none of the other genes have already been inactivated/humanized, non-human animals comprising the separate inactivated/humanized genes can be crossed to produce non-human animals having the two or more inactivated/humanized genes.

Alternatively, the method of producing the non-human animals described elsewhere herein can comprise: (1) modifying the genome of a non-human animal one-cell stage embryo to comprise the inactivated Rag2, Il2rg, and Fah genes and a humanized IL6 gene and optionally a humanized SIRPA gene; (2) selecting the genetically modified non-human animal embryo; and (3) gestating (e.g., implanting and gestating) the genetically modified non-human animal embryo in a surrogate non-human animal mother. Progeny that are capable of transmitting the genetic modifications though the germline are generated. The step of modifying the genome to comprise the inactivated Rag2, Il2rg, and Fah genes and a humanized IL6 gene and optionally a humanized SIRPA gene can be done in any background. For example, the modifying step can be done in a non-human animal one-cell stage embryo in which none of the Rag2, Il2rg, and Fah genes have already been inactivated and the IL6 gene has not been humanized (and optionally the SIRPA gene has not been humanized). Alternatively, the modifying step for any one of the genes can be done in a non-human animal one-cell stage embryo in which one or more of Rag2, Il2rg, and Fah loci or genes have already been inactivated (e.g., one, two, or all three of the other loci or genes have already been inactivated) and/or the IL6 gene has already been humanized (and optionally the SIRPA gene has already been humanized). If two or more of the genes are inactivated in non-human animal one-cell stage embryos in which none of the other genes have already been inactivated/humanized, non-human animals comprising the separate inactivated/humanized genes can be crossed to produce non-human animals having the two or more inactivated/humanized genes. Alternatively, the method of producing the non-human animals described elsewhere herein can comprise: (1) modifying the genome of a non-human animal one-cell stage embryo to comprise the inactivated Rag1, Rag2, Il2rg, and Fah genes and a humanized IL6 gene and optionally a humanized SIRPA gene; (2) selecting the genetically modified non-human animal embryo; and (3) gestating (e.g., implanting and gestating) the genetically modified non-human animal embryo in a surrogate non-human animal mother. Progeny that are capable of transmitting the genetic modifications though the germline are generated. The step of modifying the genome to comprise the inactivated Rag1, Rag2, Il2rg, and Fah genes and a humanized IL6 gene and optionally a humanized SIRPA gene can be done in any background. For example, the modifying step can be done in a non-human animal one-cell stage embryo in which none of the Rag1, Rag2, Il2rg, and Fah genes have already been inactivated and the IL6 gene has not been humanized (and optionally the SIRPA gene has not been humanized). Alternatively, the modifying step for any one of the genes can be done in a non-human animal one-cell stage embryo in which one or more of Rag1, Rag2, Il2rg, and Fah loci or genes have already been inactivated (e.g., one, two, or all three of the other loci or genes have already been inactivated) and/or the IL6 gene has already been humanized (and optionally the SIRPA gene has already been humanized). If two or more of the genes are inactivated in non-human animal one-cell stage embryos in which none of the other genes have already been inactivated/humanized, non-human animals comprising the separate inactivated/humanized genes can be crossed to produce non-human animals having the two or more inactivated/humanized genes.

Alternatively, the method of producing the non-human animals described elsewhere herein can comprise: (1) modifying the genome of a non-human animal one-cell stage embryo to comprise the inactivated Rag2, Il2rg, and Fah genes and a humanized OSM gene and optionally a humanized SIRPA gene; (2) selecting the genetically modified non-human animal embryo; and (3) gestating (e.g., implanting and gestating) the genetically modified non-human animal embryo in a surrogate non-human animal mother. Progeny that are capable of transmitting the genetic modifications though the germline are generated. The step of modifying the genome to comprise the inactivated Rag2, Il2rg, and Fah genes and a humanized OSM gene and optionally a humanized SIRPA gene can be done in any background. For example, the modifying step can be done in a non-human animal one-cell stage embryo in which none of the Rag2, Il2rg, and Fah genes have already been inactivated and the OSM gene has not been humanized (and optionally the SIRPA gene has not been humanized). Alternatively, the modifying step for any one of the genes can be done in a non-human animal one-cell stage embryo in which one or more of Rag2, Il2rg, and Fah loci or genes have already been inactivated (e.g., one, two, or all three of the other loci or genes have already been inactivated) and/or the OSM gene has already been humanized (and optionally the SIRPA gene has already been humanized). If two or more of the genes are inactivated in non-human animal one-cell stage embryos in which none of the other genes have already been inactivated/humanized, non-human animals comprising the separate inactivated/humanized genes can be crossed to produce non-human animals having the two or more inactivated/humanized genes. Alternatively, the method of producing the non-human animals described elsewhere herein can comprise: (1) modifying the genome of a non-human animal one-cell stage embryo to comprise the inactivated Rag1, Rag2, Il2rg, and Fah genes and a humanized OSM gene and optionally a humanized SIRPA gene; (2) selecting the genetically modified non-human animal embryo; and (3) gestating (e.g., implanting and gestating) the genetically modified non-human animal embryo in a surrogate non-human animal mother. Progeny that are capable of transmitting the genetic modifications though the germline are generated. The step of modifying the genome to comprise the inactivated Rag1, Rag2, Il2rg, and Fah genes and a humanized OSM gene and optionally a humanized SIRPA gene can be done in any background. For example, the modifying step can be done in a non-human animal one-cell stage embryo in which none of the Rag1, Rag2, Il2rg, and Fah genes have already been inactivated and the OSM gene has not been humanized (and optionally the SIRPA gene has not been humanized). Alternatively, the modifying step for any one of the genes can be done in a non-human animal one-cell stage embryo in which one or more of Rag1, Rag2, Il2rg, and Fah loci or genes have already been inactivated (e.g., one, two, or all three of the other loci or genes have already been inactivated) and/or the OSM gene has already been humanized (and optionally the SIRPA gene has already been humanized). If two or more of the genes are inactivated in non-human animal one-cell stage embryos in which none of the other genes have already been inactivated/humanized, non-human animals comprising the separate inactivated/humanized genes can be crossed to produce non-human animals having the two or more inactivated/humanized genes.

Alternatively, the method of producing the non-human animals described elsewhere herein can comprise: (1) modifying the genome of a non-human animal one-cell stage embryo to comprise the inactivated Rag2, Il2rg, and Fah genes and humanized IL6 and OSM genes and optionally a humanized SIRPA gene; (2) selecting the genetically modified non-human animal embryo; and (3) gestating (e.g., implanting and gestating) the genetically modified non-human animal embryo in a surrogate non-human animal mother. Progeny that are capable of transmitting the genetic modifications though the germline are generated. The step of modifying the genome to comprise the inactivated Rag2, Il2rg, and Fah genes and humanized IL6 and OSM genes and optionally a humanized SIRPA gene can be done in any background. For example, the modifying step can be done in a non-human animal one-cell stage embryo in which none of the Rag2, Il2rg, and Fah genes have already been inactivated and the IL6 and OSM genes have not been humanized (and optionally the SIRPA gene has not been humanized). Alternatively, the modifying step for any one of the genes can be done in a non-human animal one-cell stage embryo in which one or more of Rag2, Il2rg, and Fah loci or genes have already been inactivated (e.g., one, two, or all three of the other loci or genes have already been inactivated) and/or one or both of the IL6 gene and the OSM gene has already been humanized (and optionally the SIRPA gene has already been humanized). If two or more of the genes are inactivated in non-human animal one-cell stage embryos in which none of the other genes have already been inactivated/humanized, non-human animals comprising the separate inactivated/humanized genes can be crossed to produce non-human animals having the two or more inactivated/humanized genes. Alternatively, the method of producing the non-human animals described elsewhere herein can comprise: (1) modifying the genome of a non-human animal one-cell stage embryo to comprise the inactivated Rag1, Rag2, Il2rg, and Fah genes and humanized IL6 and OSM genes and optionally a humanized SIRPA gene; (2) selecting the genetically modified non-human animal embryo; and (3) gestating (e.g., implanting and gestating) the genetically modified non-human animal embryo in a surrogate non-human animal mother. Progeny that are capable of transmitting the genetic modifications though the germline are generated. The step of modifying the genome to comprise the inactivated Rag1, Rag2, Il2rg, and Fah genes and humanized IL6 and OSM genes and optionally a humanized SIRPA gene can be done in any background. For example, the modifying step can be done in a non-human animal one-cell stage embryo in which none of the Rag1, Rag2, Il2rg, and Fah genes have already been inactivated and the IL6 and OSM genes have not been humanized (and optionally the SIRPA gene has not been humanized). Alternatively, the modifying step for any one of the genes can be done in a non-human animal one-cell stage embryo in which one or more of Rag1, Rag2, Il2rg, and Fah loci or genes have already been inactivated (e.g., one, two, or all three of the other loci or genes have already been inactivated) and/or one or both of the IL6 gene and the OSM gene has already been humanized (and optionally the SIRPA gene has already been humanized). If two or more of the genes are inactivated in non-human animal one-cell stage embryos in which none of the other genes have already been inactivated/humanized, non-human animals comprising the separate inactivated/humanized genes can be crossed to produce non-human animals having the two or more inactivated/humanized genes.

The genes can be inactivated/humanized simultaneously or sequentially in the same cell to produce a single non-human animal with the inactivated/humanized genes or in different cells to produce different non-human animals with different inactivated/humanized genes that are then crossed with each other, or the genes can be inactivated/humanized sequentially by producing a first non-human animal with one or more of the inactivated/humanized genes and then isolating a non-human animal pluripotent cell or one-cell stage embryo to inactivate/humanize one or more additional genes and produce a non-human animal with the initial one or more inactivated/humanized genes and the one or more additional inactivated/humanized genes. The genes can be inactivated/humanized by any known means (e.g., through use of a nuclease agent, through use of an exogenous donor nucleic acid, or through use of both a nuclease agent and an exogenous donor nucleic acid). For example, two or more or all of the Rag2, Il2rg, and Fah genes can be inactivated simultaneously via multiplex genome editing. Alternatively, two or more or all of the Rag2, Il2rg, and Fah genes can be inactivated and the IL6 gene and optionally the SIRPA gene can be humanized simultaneously or sequentially in different cells to produce different non-human animals that are subsequently crossed to produce non-human animals with all of the inactivated/humanized genes (e.g., Rag2 can be inactivated in a first cell to produce a first non-human animal, and Il2rg can be inactivated in a second cell to produce a second non-human animal that is crossed to the first non-human animal). Alternatively, two or more or all of the Rag2, Il2rg, and Fah genes can be inactivated and the IL6 gene and optionally the SIRPA gene can be humanized sequentially in the same cell. For example, two or more or all of the Rag1, Rag2, Il2rg, and Fah genes can be inactivated simultaneously via multiplex genome editing (e.g., Rag1 and Rag2 can be inactivated simultaneously in the same cell). Alternatively, two or more or all of the Rag1, Rag2, Il2rg, and Fah genes can be inactivated and the IL6 gene and optionally the SIRPA gene can be humanized simultaneously or sequentially in different cells to produce different non-human animals that are subsequently crossed to produce non-human animals with all of the inactivated/humanized genes (e.g., Rag1 and Rag2 can be inactivated in a first cell to produce a first non-human animal, and Il2rg can be inactivated in a second cell to produce a second non-human animal that is crossed to the first non-human animal). Alternatively, two or more or all of the Rag1, Rag2, Il2rg, and Fah genes can be inactivated and the IL6 gene and optionally the SIRPA gene can be humanized sequentially in the same cell.

The genes can be inactivated/humanized simultaneously or sequentially in the same cell to produce a single non-human animal with the inactivated/humanized genes or in different cells to produce different non-human animals with different inactivated/humanized genes that are then crossed with each other, or the genes can be inactivated/humanized sequentially by producing a first non-human animal with one or more of the inactivated/humanized genes and then isolating a non-human animal pluripotent cell or one-cell stage embryo to inactivate/humanize one or more additional genes and produce a non-human animal with the initial one or more inactivated/humanized genes and the one or more additional inactivated/humanized genes. The genes can be inactivated/humanized by any known means (e.g., through use of a nuclease agent, through use of an exogenous donor nucleic acid, or through use of both a nuclease agent and an exogenous donor nucleic acid). For example, two or more or all of the Rag2, Il2rg, and Fah genes can be inactivated simultaneously via multiplex genome editing. Alternatively, two or more or all of the Rag2, Il2rg, and Fah genes can be inactivated and the OSM gene and optionally the SIRPA gene can be humanized simultaneously or sequentially in different cells to produce different non-human animals that are subsequently crossed to produce non-human animals with all of the inactivated/humanized genes (e.g., Rag2 can be inactivated in a first cell to produce a first non-human animal, and Il2rg can be inactivated in a second cell to produce a second non-human animal that is crossed to the first non-human animal). Alternatively, two or more or all of the Rag2, Il2rg, and Fah genes can be inactivated and the OSM gene and optionally the SIRPA gene can be humanized sequentially in the same cell. For example, two or more or all of the Rag1, Rag2, Il2rg, and Fah genes can be inactivated simultaneously via multiplex genome editing (e.g., Rag1 and Rag2 can be inactivated simultaneously in the same cell). Alternatively, two or more or all of the Rag1, Rag2, Il2rg, and Fah genes can be inactivated and the OSM gene and optionally the SIRPA gene can be humanized simultaneously or sequentially in different cells to produce different non-human animals that are subsequently crossed to produce non-human animals with all of the inactivated/humanized genes (e.g., Rag1 and Rag2 can be inactivated in a first cell to produce a first non-human animal, and Il2rg can be inactivated in a second cell to produce a second non-human animal that is crossed to the first non-human animal). Alternatively, two or more or all of the Rag1, Rag2, Il2rg, and Fah genes can be inactivated and the OSM gene and optionally the SIRPA gene can be humanized sequentially in the same cell.

The genes can be inactivated/humanized simultaneously or sequentially in the same cell to produce a single non-human animal with the inactivated/humanized genes or in different cells to produce different non-human animals with different inactivated/humanized genes that are then crossed with each other, or the genes can be inactivated/humanized sequentially by producing a first non-human animal with one or more of the inactivated/humanized genes and then isolating a non-human animal pluripotent cell or one-cell stage embryo to inactivate/humanize one or more additional genes and produce a non-human animal with the initial one or more inactivated/humanized genes and the one or more additional inactivated/humanized genes. The genes can be inactivated/humanized by any known means (e.g., through use of a nuclease agent, through use of an exogenous donor nucleic acid, or through use of both a nuclease agent and an exogenous donor nucleic acid). For example, two or more or all of the Rag2, Il2rg, and Fah genes can be inactivated simultaneously via multiplex genome editing. Alternatively, two or more or all of the Rag2, Il2rg, and Fah genes can be inactivated and the IL6 gene and the OSM gene and optionally the SIRPA gene can be humanized simultaneously or sequentially in different cells to produce different non-human animals that are subsequently crossed to produce non-human animals with all of the inactivated/humanized genes (e.g., Rag2 can be inactivated in a first cell to produce a first non-human animal, and Il2rg can be inactivated in a second cell to produce a second non-human animal that is crossed to the first non-human animal). Alternatively, two or more or all of the Rag2, Il2rg, and Fah genes can be inactivated and the IL6 and OSM genes and optionally the SIRPA gene can be humanized sequentially in the same cell. For example, two or more or all of the Rag1, Rag2, Il2rg, and Fah genes can be inactivated simultaneously via multiplex genome editing (e.g., Rag1 and Rag2 can be inactivated simultaneously in the same cell). Alternatively, two or more or all of the Rag1, Rag2, Il2rg, and Fah genes can be inactivated and the IL6 gene and the OSM gene and optionally the SIRPA gene can be humanized simultaneously or sequentially in different cells to produce different non-human animals that are subsequently crossed to produce non-human animals with all of the inactivated/humanized genes (e.g., Rag1 and Rag2 can be inactivated in a first cell to produce a first non-human animal, and Il2rg can be inactivated in a second cell to produce a second non-human animal that is crossed to the first non-human animal). Alternatively, two or more or all of the Rag1, Rag2, Il2rg, and Fah genes can be inactivated and the IL6 and OSM genes and optionally the SIRPA gene can be humanized sequentially in the same cell.

Nuclear transfer techniques can also be used to generate the non-human animals. Briefly, methods for nuclear transfer can include the steps of: (1) enucleating an oocyte or providing an enucleated oocyte; (2) isolating or providing a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods, oocytes are generally retrieved from deceased animals, although they may be isolated also from either oviducts and/or ovaries of live animals. Oocytes can be matured in a variety of well-known media prior to enucleation. Enucleation of the oocyte can be performed in a number of well-known manners. Insertion of the donor cell or nucleus into the enucleated oocyte to form a reconstituted cell can be by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell can be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, can be cultured in well-known media and then transferred to the womb of an animal. See, e.g., US 2008/0092249, WO 1999/005266, US 2004/0177390, WO 2008/017234, and U.S. Pat. No. 7,612,250, each of which is herein incorporated by reference in its entirety for all purposes.

The modified cell or one-cell stage embryo (or any other modified non-human animal cell) can be generated, for example, through recombination by: (a) introducing into the cell one or more exogenous donor nucleic acids (e.g., targeting vectors) comprising 5′ and 3′ homology arms corresponding to (e.g., capable of hybridizing to or that hybridize to) 5′ and 3′ target sites (e.g., target sites flanking the endogenous sequences intended for deletion and/or replacement with an insert nucleic acid (e.g., comprising a reporter gene such as lacZ or eGFP) flanked by the 5′ and 3′ homology arms), wherein the exogenous donor nucleic acids are designed for inactivation of the endogenous Rag1 gene, Rag2 gene, Rag1 and Rag2 genes, Il2rg gene, or Fah gene or humanization of the endogenous IL6 gene and optionally humanization of the endogenous SIRPA gene; and (b) identifying at least one cell comprising in its genome the inactivated endogenous Rag1 gene, Rag2 gene, Rag1 and Rag2 genes, Il2rg gene, or Fah gene or the humanization of the endogenous IL6 gene (or optionally the humanization of the endogenous SIRPA gene). The modified cell or one-cell stage embryo (or any other modified non-human animal cell) can be generated, for example, through recombination by: (a) introducing into the cell one or more exogenous donor nucleic acids (e.g., targeting vectors) comprising 5′ and 3′ homology arms corresponding to (e.g., capable of hybridizing to or that hybridize to) 5′ and 3′ target sites (e.g., target sites flanking the endogenous sequences intended for deletion and/or replacement with an insert nucleic acid (e.g., comprising a reporter gene such as lacZ or eGFP) flanked by the 5′ and 3′ homology arms), wherein the exogenous donor nucleic acids are designed for inactivation of the endogenous Rag1 gene, Rag2 gene, Rag1 and Rag2 genes, Il2rg gene, or Fah gene or humanization of the endogenous OSM gene and optionally humanization of the endogenous SIRPA gene; and (b) identifying at least one cell comprising in its genome the inactivated endogenous Rag1 gene, Rag2 gene, Rag1 and Rag2 genes, Il2rg gene, or Fah gene or the humanization of the endogenous OSM gene (or optionally the humanization of the endogenous SIRPA gene). The modified cell or one-cell stage embryo (or any other modified non-human animal cell) can be generated, for example, through recombination by: (a) introducing into the cell one or more exogenous donor nucleic acids (e.g., targeting vectors) comprising 5′ and 3′ homology arms corresponding to (e.g., capable of hybridizing to or that hybridize to) 5′ and 3′ target sites (e.g., target sites flanking the endogenous sequences intended for deletion and/or replacement with an insert nucleic acid (e.g., comprising a reporter gene such as lacZ or eGFP) flanked by the 5′ and 3′ homology arms), wherein the exogenous donor nucleic acids are designed for inactivation of the endogenous Rag1 gene, Rag2 gene, Rag1 and Rag2 genes, Il2rg gene, or Fah gene or humanization of the endogenous IL6 gene or OSM gene and optionally humanization of the endogenous SIRPA gene; and (b) identifying at least one cell comprising in its genome the inactivated endogenous Rag1 gene, Rag2 gene, Rag1 and Rag2 genes, Il2rg gene, or Fah gene or the humanization of the endogenous IL6 gene or OSM gene (or optionally the humanization of the endogenous SIRPA gene).

Alternatively, the modified pluripotent cell or one-cell stage embryo (or any other modified non-human animal cell) can be generated by (a) introducing into the cell: (i) a nuclease agent (or a nucleic acid encoding the nuclease agent), wherein the nuclease agent induces a nick or double-strand break at a target site within the endogenous Rag1 gene, Rag2 gene, Rag1 and Rag2 genes, Il2rg gene, or Fah gene or the endogenous IL6 gene (or optionally the endogenous SIRPA gene); and (ii) one or more exogenous donor nucleic acids (e.g., targeting vectors) optionally comprising an insert nucleic acid flanked by, for example, 5′ and 3′ homology arms corresponding to 5′ and 3′ target sites (e.g., target sites flanking the endogenous sequences intended for deletion and/or replacement with the insert nucleic acid), wherein the exogenous donor nucleic acids are designed for inactivation of the endogenous Rag1 gene, Rag2 gene, Rag1 and Rag2 genes, Il2rg gene, or Fah gene or humanization of the endogenous IL6 gene (or optionally humanization of the endogenous SIRPA gene); and (c) identifying at least one cell comprising the inactivated endogenous Rag1 gene, Rag2 gene, Rag1 and Rag2 genes, Il2rg gene, or Fah gene or the humanized IL6 gene (or optionally the humanized SIRPA gene). Alternatively, the modified pluripotent cell or one-cell stage embryo (or any other modified non-human animal cell) can be generated by (a) introducing into the cell a nuclease agent (or a nucleic acid encoding the nuclease agent), wherein the nuclease agent induces a nick or double-strand break at a target site within the endogenous Rag1 gene, Rag2 gene, Rag1 and Rag2 genes, Il2rg gene, or Fah gene to inactivate the endogenous Rag1 gene, Rag2 gene, Rag1 and Rag2 genes, Il2rg gene, or Fah gene; and (b) identifying at least one cell comprising the inactivated endogenous Rag1 gene, Rag2 gene, Rag1 and Rag2 genes, Il2rg gene, or Fah gene. Any nuclease agent that induces a nick or double-strand break into a desired recognition site to facilitate recombination of the exogenous donor nucleic acid with the target genomic locus or insertion of the exogenous donor nucleic acid into the target genomic locus can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems (e.g., CRISPR/Cas9 systems) or components of such systems (e.g., CRISPR/Cas9). See, e.g., US 2013/0309670 and US 2015/0159175, each of which is herein incorporated by reference in its entirety for all purposes. In one example, the nuclease comprises a Cas9 protein and a guide RNA.

Alternatively, the modified pluripotent cell or one-cell stage embryo (or any other modified non-human animal cell) can be generated by (a) introducing into the cell: (i) a nuclease agent (or a nucleic acid encoding the nuclease agent), wherein the nuclease agent induces a nick or double-strand break at a target site within the endogenous Rag1 gene, Rag2 gene, Rag1 and Rag2 genes, Il2rg gene, or Fah gene or the endogenous OSM gene (or optionally the endogenous SIRPA gene); and (ii) one or more exogenous donor nucleic acids (e.g., targeting vectors) optionally comprising an insert nucleic acid flanked by, for example, 5′ and 3′ homology arms corresponding to 5′ and 3′ target sites (e.g., target sites flanking the endogenous sequences intended for deletion and/or replacement with the insert nucleic acid), wherein the exogenous donor nucleic acids are designed for inactivation of the endogenous Rag1 gene, Rag2 gene, Rag1 and Rag2 genes, Il2rg gene, or Fah gene or humanization of the endogenous OSM gene (or optionally humanization of the endogenous SIRPA gene); and (c) identifying at least one cell comprising the inactivated endogenous Rag1 gene, Rag2 gene, Rag1 and Rag2 genes, Il2rg gene, or Fah gene or the humanized OSM gene (or optionally the humanized SIRPA gene). Alternatively, the modified pluripotent cell or one-cell stage embryo (or any other modified non-human animal cell) can be generated by (a) introducing into the cell a nuclease agent (or a nucleic acid encoding the nuclease agent), wherein the nuclease agent induces a nick or double-strand break at a target site within the endogenous Rag1 gene, Rag2 gene, Rag1 and Rag2 genes, Il2rg gene, or Fah gene to inactivate the endogenous Rag1 gene, Rag2 gene, Rag1 and Rag2 genes, Il2rg gene, or Fah gene; and (b) identifying at least one cell comprising the inactivated endogenous Rag1 gene, Rag2 gene, Rag1 and Rag2 genes, Il2rg gene, or Fah gene. Any nuclease agent that induces a nick or double-strand break into a desired recognition site to facilitate recombination of the exogenous donor nucleic acid with the target genomic locus or insertion of the exogenous donor nucleic acid into the target genomic locus can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems (e.g., CRISPR/Cas9 systems) or components of such systems (e.g., CRISPR/Cas9). See, e.g., US 2013/0309670 and US 2015/0159175, each of which is herein incorporated by reference in its entirety for all purposes. In one example, the nuclease comprises a Cas9 protein and a guide RNA.

Alternatively, the modified pluripotent cell or one-cell stage embryo (or any other modified non-human animal cell) can be generated by (a) introducing into the cell: (i) a nuclease agent (or a nucleic acid encoding the nuclease agent), wherein the nuclease agent induces a nick or double-strand break at a target site within the endogenous Rag1 gene, Rag2 gene, Rag1 and Rag2 genes, Il2rg gene, or Fah gene or the endogenous IL6 gene or the endogenous OSM gene (or optionally the endogenous SIRPA gene); and (ii) one or more exogenous donor nucleic acids (e.g., targeting vectors) optionally comprising an insert nucleic acid flanked by, for example, 5′ and 3′ homology arms corresponding to 5′ and 3′ target sites (e.g., target sites flanking the endogenous sequences intended for deletion and/or replacement with the insert nucleic acid), wherein the exogenous donor nucleic acids are designed for inactivation of the endogenous Rag1 gene, Rag2 gene, Rag1 and Rag2 genes, Il2rg gene, or Fah gene or humanization of the endogenous IL6 gene or humanization of the endogenous OSM gene (or optionally humanization of the endogenous SIRPA gene); and (c) identifying at least one cell comprising the inactivated endogenous Rag1 gene, Rag2 gene, Rag1 and Rag2 genes, Il2rg gene, or Fah gene or the humanized IL6 gene or the humanized OSM gene (or optionally the humanized SIRPA gene). Alternatively, the modified pluripotent cell or one-cell stage embryo (or any other modified non-human animal cell) can be generated by (a) introducing into the cell a nuclease agent (or a nucleic acid encoding the nuclease agent), wherein the nuclease agent induces a nick or double-strand break at a target site within the endogenous Rag1 gene, Rag2 gene, Rag1 and Rag2 genes, Il2rg gene, or Fah gene to inactivate the endogenous Rag1 gene, Rag2 gene, Rag1 and Rag2 genes, Il2rg gene, or Fah gene; and (b) identifying at least one cell comprising the inactivated endogenous Rag1 gene, Rag2 gene, Rag1 and Rag2 genes, Il2rg gene, or Fah gene. Any nuclease agent that induces a nick or double-strand break into a desired recognition site to facilitate recombination of the exogenous donor nucleic acid with the target genomic locus or insertion of the exogenous donor nucleic acid into the target genomic locus can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems (e.g., CRISPR/Cas9 systems) or components of such systems (e.g., CRISPR/Cas9). See, e.g., US 2013/0309670 and US 2015/0159175, each of which is herein incorporated by reference in its entirety for all purposes. In one example, the nuclease comprises a Cas9 protein and a guide RNA.

The step of modifying the genome can, for example, utilize exogenous repair templates (e.g., targeting vectors) to modify an endogenous Rag1 gene, Rag2 gene, Rag1 and Rag2 genes, Il2rg gene, or Fah gene to comprise an inactivated endogenous Rag1 gene, Rag2 gene, Rag1 and Rag2 genes, Il2rg gene, or Fah gene disclosed herein or to modify an endogenous IL6 gene to comprise a humanized IL6 gene as disclosed herein (or optionally to modify an endogenous SIRPA gene to comprise a humanized SIRPA gene as disclosed herein). As one example, the targeting vector can comprise a nucleic acid insert comprising a sequence (e.g., a reporter gene such as lacZ or eGFP) to be integrated in the endogenous Rag1 gene, Rag2 gene, Rag1 and Rag2 genes, Il2rg gene, Fah gene, or IL6 gene (or optionally SIRPA gene) flanked by a 5′ homology arm targeting a 5′ target sequence in the endogenous Rag1 gene, Rag2 gene, Rag1 and Rag2 genes, Il2rg gene, Fah gene, or IL6 gene (or optionally SIRPA gene) and a 3′ homology arm targeting a 3′ target sequence in the endogenous Rag1 gene, Rag2 gene, Rag1 and Rag2 genes, Il2rg gene, Fah gene, or IL6 gene (or optionally SIRPA gene). Integration of a nucleic acid insert in the endogenous Rag1 gene, Rag2 gene, Rag1 and Rag2 genes, Il2rg gene, Fah gene, or IL6 gene (or optionally SIRPA gene) can result in addition of a nucleic acid sequence of interest in the endogenous Rag1 gene, Rag2 gene, Rag1 and Rag2 genes, Il2rg gene, Fah gene, or IL6 gene (or optionally SIRPA gene), deletion of a nucleic acid sequence of interest in the endogenous Rag1 gene, Rag2 gene, Rag1 and Rag2 genes, Il2rg gene, Fah gene, or IL6 gene (or optionally SIRPA gene), or replacement of a nucleic acid sequence of interest in the endogenous Rag1 gene, Rag2 gene, Rag1 and Rag2 genes, Il2rg gene, Fah gene, or IL6 gene (or optionally SIRPA gene) (i.e., deleting a segment of the endogenous Rag1 gene, Rag2 gene, Rag1 and Rag2 genes, Il2rg gene, Fah gene, or IL6 gene (or optionally SIRPA gene) and replacing with a reporter gene or a human IL6 gene sequence in the case of IL6 humanization (or optionally a human SIRPA gene in the case of SIRPA humanization)). As another example, the targeting vector can comprise a 5′ homology arm targeting a 5′ target sequence in the endogenous Rag1 gene, Rag2 gene, Rag1 and Rag2 genes, Il2rg gene, or Fah gene and a 3′ homology arm targeting a 3′ target sequence in the endogenous Rag1 gene, Rag2 gene, Rag1 and Rag2 genes, Il2rg gene, or Fah gene, wherein the 5′ target sequence and the 3′ target sequence flank a sequence at the endogenous Rag1 gene, Rag2 gene, Rag1 and Rag2 genes, Il2rg gene, or Fah gene intended to be deleted without any corresponding insert nucleic acid.

The step of modifying the genome can, for example, utilize exogenous repair templates (e.g., targeting vectors) to modify an endogenous Rag1 gene, Rag2 gene, Rag1 and Rag2 genes, Il2rg gene, or Fah gene to comprise an inactivated endogenous Rag1 gene, Rag2 gene, Rag1 and Rag2 genes, Il2rg gene, or Fah gene disclosed herein or to modify an endogenous OSM gene to comprise a humanized OSM gene as disclosed herein (or optionally to modify an endogenous SIRPA gene to comprise a humanized SIRPA gene as disclosed herein). As one example, the targeting vector can comprise a nucleic acid insert comprising a sequence (e.g., a reporter gene such as lacZ or eGFP) to be integrated in the endogenous Rag1 gene, Rag2 gene, Rag1 and Rag2 genes, Il2rg gene, Fah gene, or OSM gene (or optionally SIRPA gene) flanked by a 5′ homology arm targeting a 5′ target sequence in the endogenous Rag1 gene, Rag2 gene, Rag1 and Rag2 genes, Il2rg gene, Fah gene, or OSM gene (or optionally SIRPA gene) and a 3′ homology arm targeting a 3′ target sequence in the endogenous Rag1 gene, Rag2 gene, Rag1 and Rag2 genes, Il2rg gene, Fah gene, or OSM gene (or optionally SIRPA gene). Integration of a nucleic acid insert in the endogenous Rag1 gene, Rag2 gene, Rag1 and Rag2 genes, Il2rg gene, Fah gene, or OSM gene (or optionally SIRPA gene) can result in addition of a nucleic acid sequence of interest in the endogenous Rag1 gene, Rag2 gene, Rag1 and Rag2 genes, Il2rg gene, Fah gene, or OSM gene (or optionally SIRPA gene), deletion of a nucleic acid sequence of interest in the endogenous Rag1 gene, Rag2 gene, Rag1 and Rag2 genes, Il2rg gene, Fah gene, or OSM gene (or optionally SIRPA gene), or replacement of a nucleic acid sequence of interest in the endogenous Rag1 gene, Rag2 gene, Rag1 and Rag2 genes, Il2rg gene, Fah gene, or OSM gene (or optionally SIRPA gene) (i.e., deleting a segment of the endogenous Rag1 gene, Rag2 gene, Rag1 and Rag2 genes, Il2rg gene, Fah gene, or OSM gene (or optionally SIRPA gene) and replacing with a reporter gene or a human OSM gene sequence in the case of OSM humanization (or optionally a human SIRPA gene in the case of SIRPA humanization)). As another example, the targeting vector can comprise a 5′ homology arm targeting a 5′ target sequence in the endogenous Rag1 gene, Rag2 gene, Rag1 and Rag2 genes, Il2rg gene, or Fah gene and a 3′ homology arm targeting a 3′ target sequence in the endogenous Rag1 gene, Rag2 gene, Rag1 and Rag2 genes, Il2rg gene, or Fah gene, wherein the 5′ target sequence and the 3′ target sequence flank a sequence at the endogenous Rag1 gene, Rag2 gene, Rag1 and Rag2 genes, Il2rg gene, or Fah gene intended to be deleted without any corresponding insert nucleic acid.

The step of modifying the genome can, for example, utilize exogenous repair templates (e.g., targeting vectors) to modify an endogenous Rag1 gene, Rag2 gene, Rag1 and Rag2 genes, Il2rg gene, or Fah gene to comprise an inactivated endogenous Rag1 gene, Rag2 gene, Rag1 and Rag2 genes, Il2rg gene, or Fah gene disclosed herein or to modify an endogenous IL6 gene to comprise a humanized IL6 gene as disclosed herein or to modify an endogenous OSM gene to comprise a humanized OSM gene as disclosed herein (or optionally to modify an endogenous SIRPA gene to comprise a humanized SIRPA gene as disclosed herein). As one example, the targeting vector can comprise a nucleic acid insert comprising a sequence (e.g., a reporter gene such as lacZ or eGFP) to be integrated in the endogenous Rag1 gene, Rag2 gene, Rag1 and Rag2 genes, Il2rg gene, Fah gene, IL6 gene, or OSM gene (or optionally SIRPA gene) flanked by a 5′ homology arm targeting a 5′ target sequence in the endogenous Rag1 gene, Rag2 gene, Rag1 and Rag2 genes, Il2rg gene, Fah gene, IL6 gene, or OSM gene (or optionally SIRPA gene) and a 3′ homology arm targeting a 3′ target sequence in the endogenous Rag1 gene, Rag2 gene, Rag1 and Rag2 genes, Il2rg gene, Fah gene, IL6 gene, or OSM gene (or optionally SIRPA gene). Integration of a nucleic acid insert in the endogenous Rag1 gene, Rag2 gene, Rag1 and Rag2 genes, Il2rg gene, Fah gene, IL6 gene, or OSM gene (or optionally SIRPA gene) can result in addition of a nucleic acid sequence of interest in the endogenous Rag1 gene, Rag2 gene, Rag1 and Rag2 genes, Il2rg gene, Fah gene, IL6 gene, or OSM gene (or optionally SIRPA gene), deletion of a nucleic acid sequence of interest in the endogenous Rag1 gene, Rag2 gene, Rag1 and Rag2 genes, Il2rg gene, Fah gene, IL6 gene, or OSM gene (or optionally SIRPA gene), or replacement of a nucleic acid sequence of interest in the endogenous Rag1 gene, Rag2 gene, Rag1 and Rag2 genes, Il2rg gene, Fah gene, IL6 gene, or OSM gene (or optionally SIRPA gene) (i.e., deleting a segment of the endogenous Rag1 gene, Rag2 gene, Rag1 and Rag2 genes, Il2rg gene, Fah gene, IL6 gene, or OSM gene (or optionally SIRPA gene) and replacing with a reporter gene or a human IL6 gene sequence in the case of IL6 humanization and replacing with a reporter gene or a human OSM gene sequence in the case of OSM humanization (or optionally a human SIRPA gene in the case of SIRPA humanization)). As another example, the targeting vector can comprise a 5′ homology arm targeting a 5′ target sequence in the endogenous Rag1 gene, Rag2 gene, Rag1 and Rag2 genes, Il2rg gene, or Fah gene and a 3′ homology arm targeting a 3′ target sequence in the endogenous Rag1 gene, Rag2 gene, Rag1 and Rag2 genes, Il2rg gene, or Fah gene, wherein the 5′ target sequence and the 3′ target sequence flank a sequence at the endogenous Rag1 gene, Rag2 gene, Rag1 and Rag2 genes, Il2rg gene, or Fah gene intended to be deleted without any corresponding insert nucleic acid.

The exogenous repair templates can be for non-homologous-end-joining-mediated insertion or homologous recombination. Exogenous repair templates can comprise deoxyribonucleic acid (DNA) or ribonucleic acid (RNA), they can be single-stranded or double-stranded, and they can be in linear or circular form. For example, a repair template can be a single-stranded oligodeoxynucleotide (ssODN). Exogenous repair templates can also comprise a heterologous sequence that is not present at an untargeted endogenous Rag1 gene, Rag2 gene, Rag1 and Rag2 genes, Il2rg gene, or Fah gene. For example, an exogenous repair template can comprise a selection cassette, such as a selection cassette flanked by recombinase recognition sites.

In cells other than one-cell stage embryos, the exogenous repair template can be a “large targeting vector” or “LTVEC,” which includes targeting vectors that comprise homology arms that correspond to and are derived from nucleic acid sequences larger than those typically used by other approaches intended to perform homologous recombination in cells. See, e.g., US 2004/0018626; WO 2013/163394; U.S. Pat. Nos. 9,834,786; 10,301,646; WO 2015/088643; U.S. Pat. Nos. 9,228,208; 9,546,384; 10,208,317; and US 2019-0112619, each of which is herein incorporated by reference in its entirety for all purposes. LTVECs also include targeting vectors comprising nucleic acid inserts having nucleic acid sequences larger than those typically used by other approaches intended to perform homologous recombination in cells. For example, LTVECs make possible the modification of large loci that cannot be accommodated by traditional plasmid-based targeting vectors because of their size limitations. For example, the targeted locus can be (i.e., the 5′ and 3′ homology arms can correspond to) a locus of the cell that is not targetable using a conventional method or that can be targeted only incorrectly or only with significantly low efficiency in the absence of a nick or double-strand break induced by a nuclease agent (e.g., a Cas protein). LTVECs can be of any length and are typically at least 10 kb in length. The sum total of the 5′ homology arm and the 3′ homology arm in an LTVEC is typically at least 10 kb. Generation and use of large targeting vectors (LTVECs) derived from bacterial artificial chromosome (BAC) DNA through bacterial homologous recombination (BHR) reactions using VELOCIGENE® genetic engineering technology is described, e.g., in U.S. Pat. No. 6,586,251 and Valenzuela et al. (2003) Nat. Biotechnol. 21(6):652-659, each of which is herein incorporated by reference in its entirety for all purposes. Generation of LTVECs through in vitro assembly methods is described, e.g., in US 2015/0376628 and WO 2015/200334, each of which is herein incorporated by reference in its entirety for all purposes.

The methods can further comprise identifying a cell or non-human animal having a modified target genomic locus. Various methods can be used to identify cells and non-human animals having a targeted genetic modification. The screening step can comprise, for example, a quantitative assay for assessing modification-of-allele (MOA) of a parental chromosome. See, e.g., US 2004/0018626; US 2014/0178879; US 2016/0145646; WO 2016/081923; and Frendewey et al. (2010) Methods Enzymol. 476:295-307, each of which is herein incorporated by reference in its entirety for all purposes. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence. Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, incorporated herein by reference in its entirety for all purposes).

The various methods provided herein allow for the generation of a genetically modified F0 non-human animal wherein the cells of the genetically modified F0 non-human animal comprise the inactivated Rag2, Il2rg, and Fah genes and humanized IL6 gene and optionally humanized SIRPA gene (or any combination thereof). The cells of the genetically modified F0 non-human animal can be heterozygous for the inactivated Rag2, Il2rg, and Fah genes and humanized IL6 gene and optionally humanized SIRPA gene (or any combination thereof) or can be homozygous for the inactivated Rag2, Il2rg, and Fah genes and humanized IL6 gene and optionally humanized SIRPA gene (or any combination thereof). The various methods provided herein allow for the generation of a genetically modified F0 non-human animal wherein the cells of the genetically modified F0 non-human animal comprise the inactivated Rag2, Il2rg, and Fah genes and humanized OSM gene and optionally humanized SIRPA gene (or any combination thereof). The cells of the genetically modified F0 non-human animal can be heterozygous for the inactivated Rag2, Il2rg, and Fah genes and humanized OSM gene and optionally humanized SIRPA gene (or any combination thereof) or can be homozygous for the inactivated Rag2, Il2rg, and Fah genes and humanized OSM gene and optionally humanized SIRPA gene (or any combination thereof). The various methods provided herein allow for the generation of a genetically modified F0 non-human animal wherein the cells of the genetically modified F0 non-human animal comprise the inactivated Rag2, Il2rg, and Fah genes and humanized IL6 and OSM genes and optionally humanized SIRPA gene (or any combination thereof). The cells of the genetically modified F0 non-human animal can be heterozygous for the inactivated Rag2, Il2rg, and Fah genes and humanized IL6 and OSM genes and optionally humanized SIRPA gene (or any combination thereof) or can be homozygous for the inactivated Rag2, Il2rg, and Fah genes and humanized IL6 and OSM genes and optionally humanized SIRPA gene (or any combination thereof). The various methods provided herein allow for the generation of a genetically modified F0 non-human animal wherein the cells of the genetically modified F0 non-human animal comprise the inactivated Rag1, Rag2, Il2rg, and Fah genes and humanized IL6 gene and optionally humanized SIRPA gene (or any combination thereof). The cells of the genetically modified F0 non-human animal can be heterozygous for the inactivated Rag1, Rag2, Il2rg, and Fah genes and humanized IL6 gene and optionally humanized SIRPA gene (or any combination thereof) or can be homozygous for the inactivated Rag1, Rag2, Il2rg, and Fah genes and humanized IL6 gene and optionally humanized SIRPA gene (or any combination thereof). The various methods provided herein allow for the generation of a genetically modified F0 non-human animal wherein the cells of the genetically modified F0 non-human animal comprise the inactivated Rag1, Rag2, Il2rg, and Fah genes and humanized OSM gene and optionally humanized SIRPA gene (or any combination thereof). The cells of the genetically modified F0 non-human animal can be heterozygous for the inactivated Rag1, Rag2, Il2rg, and Fah genes and humanized OSM gene and optionally humanized SIRPA gene (or any combination thereof) or can be homozygous for the inactivated Rag1, Rag2, Il2rg, and Fah genes and humanized OSM gene and optionally humanized SIRPA gene (or any combination thereof). The various methods provided herein allow for the generation of a genetically modified F0 non-human animal wherein the cells of the genetically modified F0 non-human animal comprise the inactivated Rag1, Rag2, Il2rg, and Fah genes and humanized IL6 and OSM genes and optionally humanized SIRPA gene (or any combination thereof). The cells of the genetically modified F0 non-human animal can be heterozygous for the inactivated Rag1, Rag2, Il2rg, and Fah genes and humanized IL6 and OSM genes and optionally humanized SIRPA gene (or any combination thereof) or can be homozygous for the inactivated Rag1, Rag2, Il2rg, and Fah genes and humanized IL6 and OSM genes and optionally humanized SIRPA gene (or any combination thereof).

Any of the methods described herein can also be used, for example, for making non-human animals comprising a humanized Growth Hormone gene or optionally further comprising a humanized Growth Hormone gene in combination with any of the other inactivated genes and/or humanized genes disclosed herein.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 1
Description of Sequences.

| SEQ ID NO | Type | Description |
|---|---|---|
| 1-13 | DNA | Probe Sequences |
| 14-39 | DNA | Primer Sequences |
| 40 | DNA | mIL-6R Coding Sequence |
| 41 | Protein | mIL-6R Protein Sequence |
| 42 | DNA | rIL-6R Coding Sequence |
| 43 | Protein | rIL-6R Protein Sequence |
| 44 | DNA | hIL-6 Coding Sequence |
| 45 | Protein | hIL-6 Protein Sequence |
| 46 | DNA | GP130 (Tyr186-Tyr190 Del) Coding Sequence |
| 47 | Protein | GP130 (Tyr186-Tyr190 Del) Protein Sequence |
| 48 | DNA | GP130 (WT) Coding Sequence |
| 49 | Protein | GP130 (WT) Protein Sequence |
| 50 | DNA | hOSM Coding Sequence |
| 51 | Protein | hOSM Protein Sequence |
| 52 | DNA | mOSMR Coding Sequence |
| 53 | Protein | mOSMR Protein Sequence |
| 54 | DNA | rOSMR Coding Sequence |
| 55 | Protein | rOSMR Protein Sequence |

### EXAMPLES

### Example 1. Human, but not Rodent, Hepatocytes Engrafted in Host Mice or Rats Showed Hepatic Steatosis

We generated humanized liver mouse and rat models by engrafting human hepatocytes in the livers of FSRG (FAH^{-/-}, SIRPa^{hu/hu}, RAG2^{-/-}, IL2Rg^{-/-}) mice or FRG (FAH^{-/-}, Rag1/2^{-/-}, IL2rg^{-/-}) rats, respectively. In both models, humanized livers showed a steatosis-like phenotype marked by existence of positive Oil-Red-O staining (FIGS. 1A and 1B). Further, extensive distribution of eosin-negative vacuoles occurred in fumarylacetoacetate hydrolase (FAH)+ areas of humanized mouse or rat livers, suggesting an accumulation of lipid droplets within human engraftments (FIGS. 1A and 1B). Accordingly, immunohistological analysis showed positive cytoplasmic staining of lipid binding protein 17β-hydroxysteroid dehydrogenase type 13 (HSD17B13) in all human asialoglycoprotein receptor 1 (hASGR1)-positive regions (FIGS. 1A and 1B), confirming a human-specific hepato-steatosis phenotype in humanized liver rodents. Interestingly, this abnormality did not occur in rat or mouse hepatocytes engrafted in either FSRG mice or FRG rats (FIG. 1C), suggesting that it is not a result of the engraftment process. Such a phenomenon raises the possibility that lipid over-accumulation in engrafted human hepatocytes could be a result of impaired signaling in donor cells due to cross-species incompatibility between recipient rodents and human hepatocytes.

### Example 2. Restoration of IL-6-IL-6R Signaling Through Ectopic Expression of Rodent IL-6R in Human Hepatocytes Eliminates Lipid Droplet Accumulation in Humanized Livers

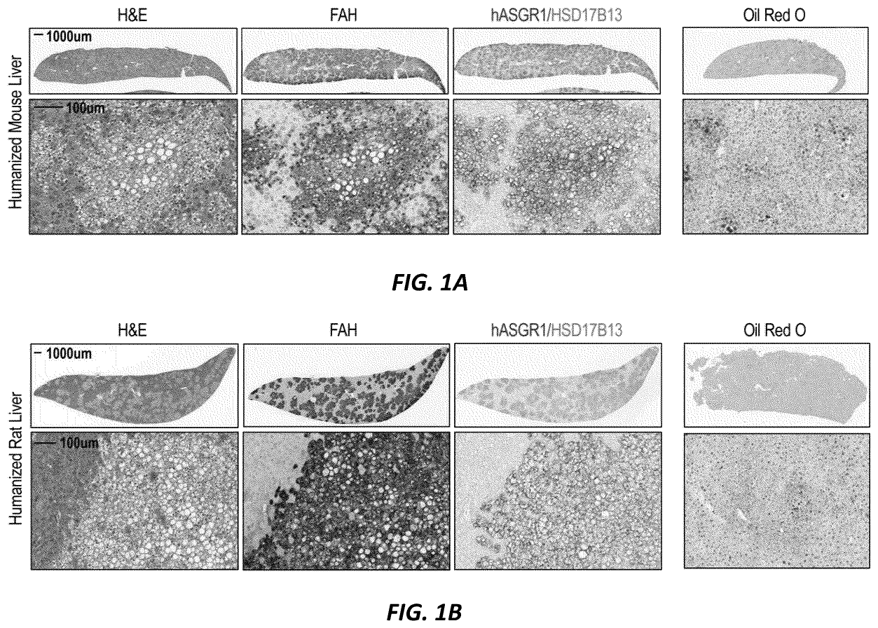

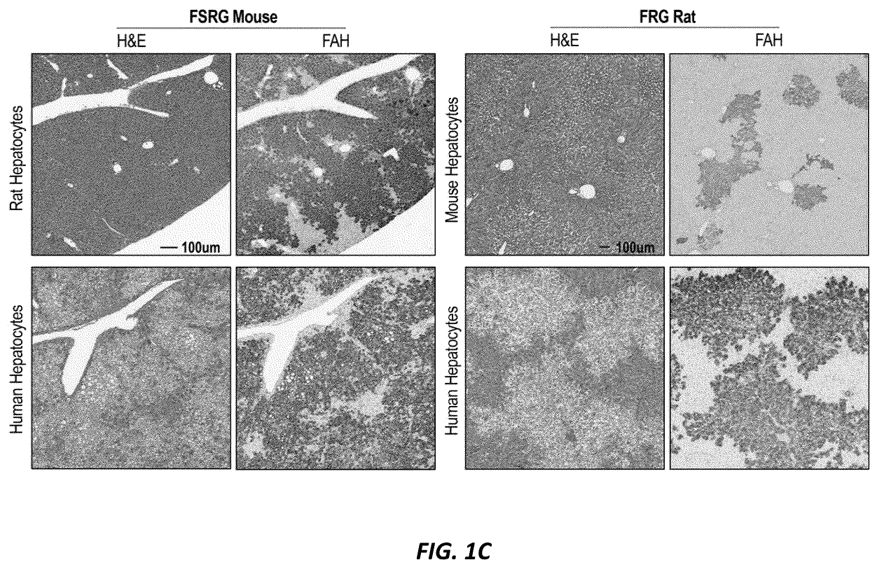

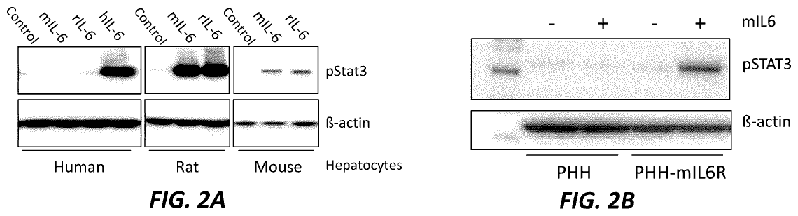

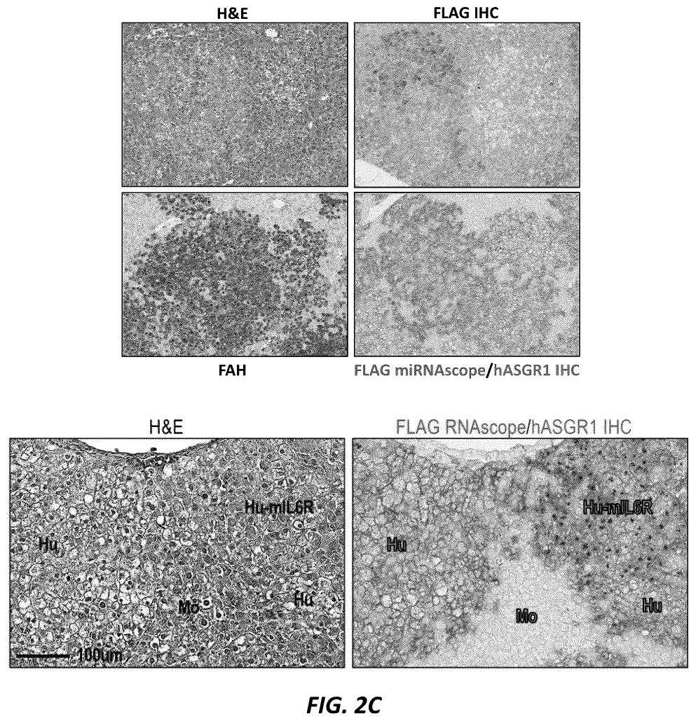

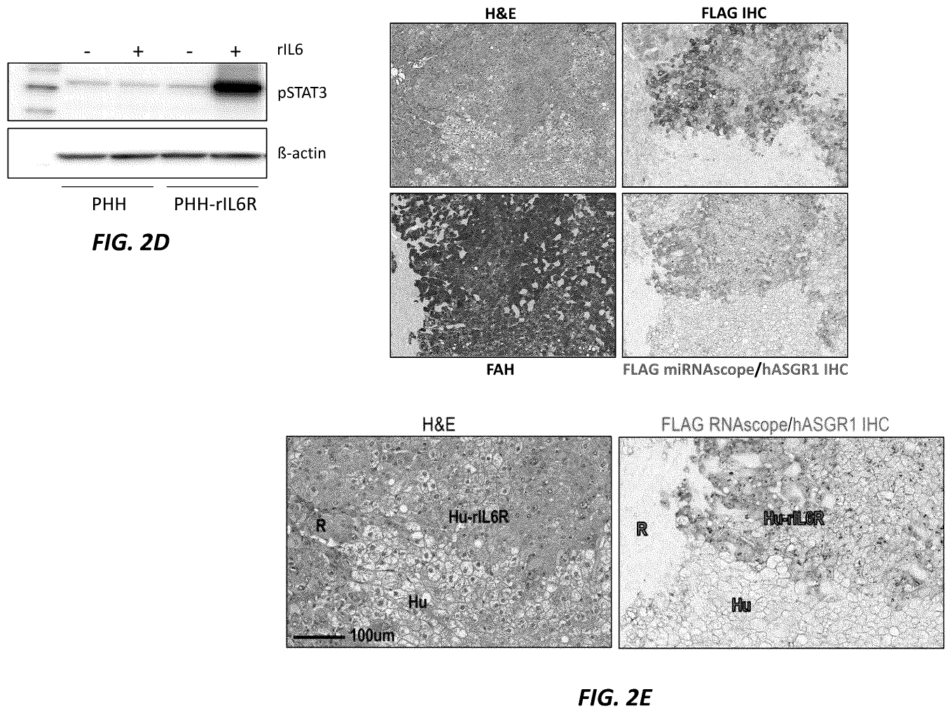

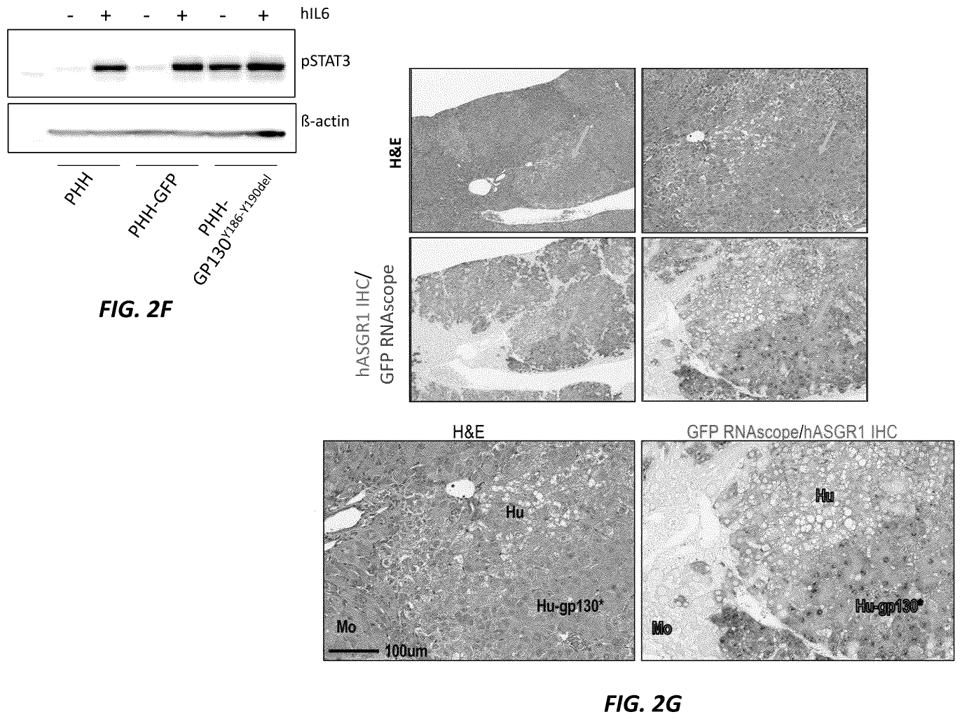

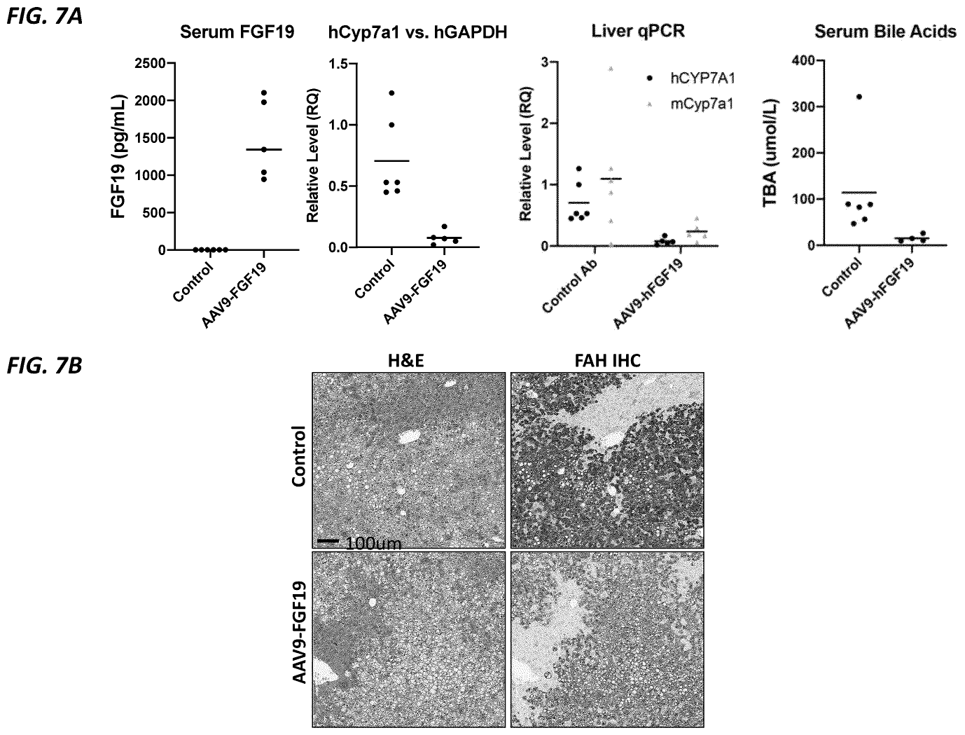

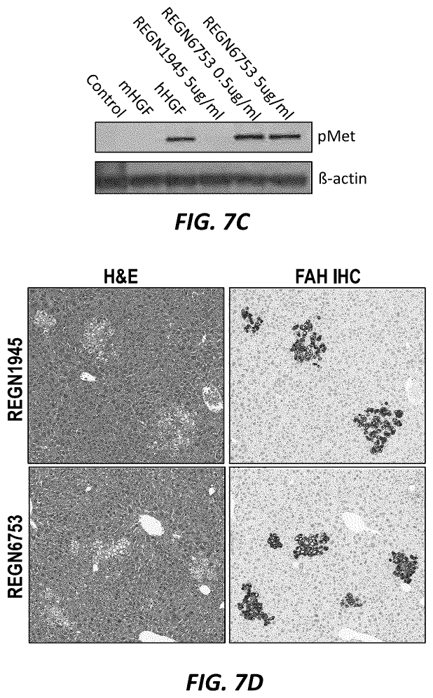

Incompatibilities of ligand-receptor interactions across species have been widely observed, especially in inter-species transplantation studies. In the liver, a number of receptors expressed on the surface of hepatocytes need to interact with ligands produced by other cell types to transduce downstream signals, some of which show cross-species incompatibility between murine ligands and human receptors. A lack of reactivity between murine ligands and their human receptors might contribute to the fatty phenotype in humanized liver mouse models. Such ligand-receptor pairs include FGF19-FGFR4, HGF-MET, GH-GHR, OSM-OSMR, and IL-6-IL-6R. We tested this concept by over-expressing human FGF19 or applying activating antibodies to MET receptor in humanized liver mice. We found that neither restoration of FGF19-FGFR4 or MET signaling in humanized livers lead to correction of this phenotype (FIGS. 7A-7D) Cyp7a1 encodes a bile acid synthesis enzyme that is expressed by pericentral hepatocytes in the livers. This gene is regulated by FGF19 in human and its homologue FGF15 in mouse. In this system, we used Cyp7a1 expression and bile acid levels as readouts of FGF19 activity. Separately, another study demonstrated that administration of human growth hormone in humanized liver mice could correct fatty liver in humanized liver mice. However, genetic defects in GH-GHR pathway have not been found in patients with fatty liver disease. Rather, observations both in patients and experimental animal models show dysregulation of multiple cytokines in development of hepatic steatosis. In particular, IL-6 is among the cytokines frequently dysregulated in patients with hepatic steatosis, including nonalcoholic fatty liver disease (NAFLD) and nonalcoholic steatohepatitis (NASH). We therefore sought to investigate the role of IL-6-IL-6R signaling in hepatic lipid droplet accumulation. As shown in FIG. 2A, treatment with mouse or rat IL-6 can induce Signal Transducer and Activator of Transcription 3 (STAT3) phosphorylation in both primary murine and rat hepatocytes, but not in human hepatocytes, consistent with suboptimal interaction between mouse or rat IL-6 ligand and human IL-6R. We therefore hypothesized that cross-species incompatibility between mouse or rat IL-6 and hIL-6R might play a role in lipid droplet accumulation in humanized liver mice and rats.

To test this hypothesis, we sought to restore the IL-6R signaling by ectopic expression of rodent IL-6R in humanized livers. To this end, primary human hepatocytes were transduced with lentivirus carrying mIL-6R or rIL-6R expression vectors before transplantation into recipient mice or rats, respectively (mIL-6R rIL-6R coding and protein sequence set forth in SEQ ID NOS: 40-41 and 42-43, respectively). As shown in FIG. 2B, unlike untreated cells, lentivirus-treated primary hepatocytes responded robustly to mIL-6 treatment by elevation of STAT3 phosphorylation ex vivo.

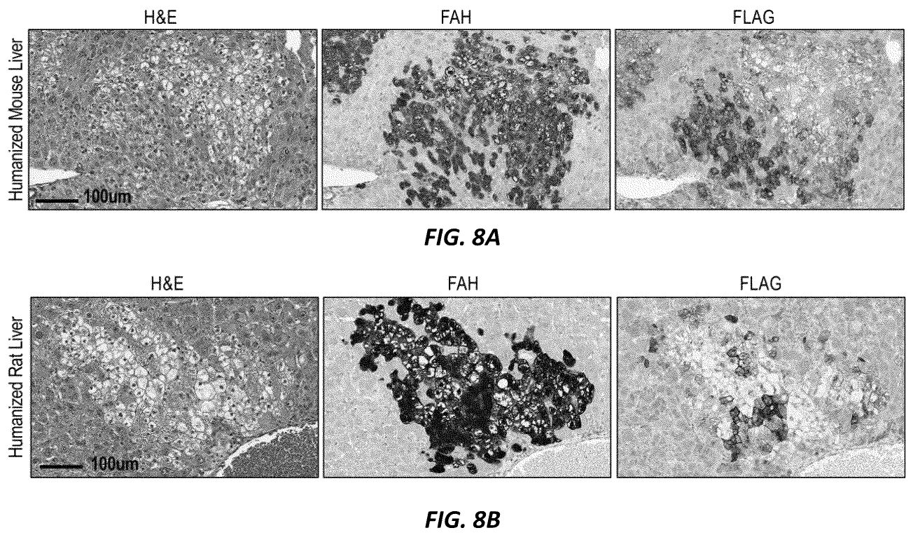

In vivo, mIL-6R expression in humanized livers was detected by immunoreactivity to an antibody (FIG. 8A) or in situ hybridization to a FLAG-specific miRNAscope probe that recognizes a FLAG-tag sequence fused to the mIL-6R transgene (FIG. 2C). Since no selection procedure was applied before transplantation, humanized livers consisted of both mIL-6R expressing (FLAG-positive) and non-expressing (FLAG-negative) regions of FAH-expressing (hASGR1-expressing) human hepatocytes. As expected, contrary to the evident steatosis morphology of FLAG-negative human hepatocytes, FLAG-positive, mIL-6R-expressing areas showed a significant reduction, if not complete disappearance of lipid droplet accumulation (FIG. 2C and FIG. 8A). Similarly, human hepatocytes expressing rat IL-6R also showed efficient phospho-STAT3 activation upon rIL-6 treatment ex vivo (FIG. 2D) and lack of lipid droplet accumulation in humanized liver rats (FIG. 2E and FIG. 8B). Thus, restoration of IL-6R signaling in human hepatocytes through ectopic expression of rodent IL-6R reactive to host-derived IL-6 eliminated lipid droplet accumulation in humanized livers.

### Example 3. Restoration of GP130 Signaling Through Ectopic Expression of Constitutive Activated GP130 in Human Hepatocytes Eliminates Lipid Droplet Accumulation in Humanized Livers

Upon IL-6 binding, IL-6R requires interaction with the GP130 co-receptor to activate downstream pathways. We asked whether hepatic activation of GP130 is sufficient to prevent lipid droplet accumulation. To this end, we expressed in human hepatocytes a ligand-independent, constitutively activated form of GP130 with a deletion from Tyr186 to Tyr190 (GP130^{Y186-Y190del}), through lentiviral transduction (coding and protein sequence set forth in SEQ ID NOS: 46-47, respectively). Expression of this mutant resulted in an elevation of pSTAT3 level in primary human hepatocytes ex vivo, even without treatment with IL-6 (FIG. 2F).

Upon engraftment of the transduced hepatocytes in recipient mouse livers, GP130^{(186-Y190del} expression was detected by in situ hybridization to a RNAscope probe specific to a GFP tag sequence in the transgene. Humanized livers consisted of both mIL-6R expressing (GFP-positive) and non-expressing (GFP-negative) regions of hASGR1-expressing human hepatocytes (FIG. 2G). Further, in humanized murine livers, contrary to the evident steatosis morphology of GFP-negative human hepatocytes, GFP-positive, GP130^{Y186-Y190del} expressing areas showed a complete disappearance of lipid droplet accumulation (FIG. 2G). Therefore, GP130 activation is sufficient to protect humanized engraftments from hepatic steatosis in humanized liver mice.

### Example 4. Human IL-6 Prevents Lipid Droplet Accumulation in Human Hepatocytes

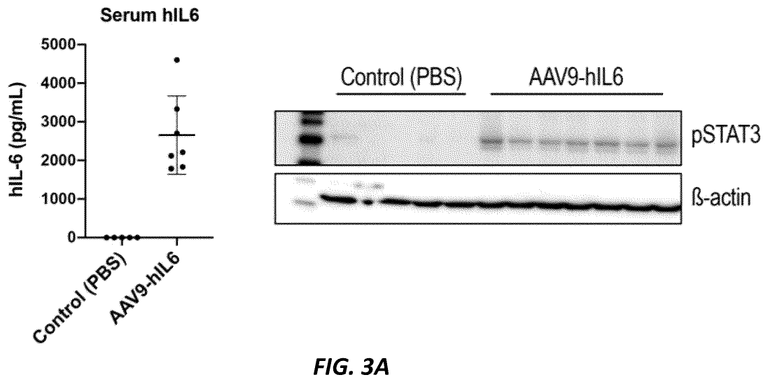

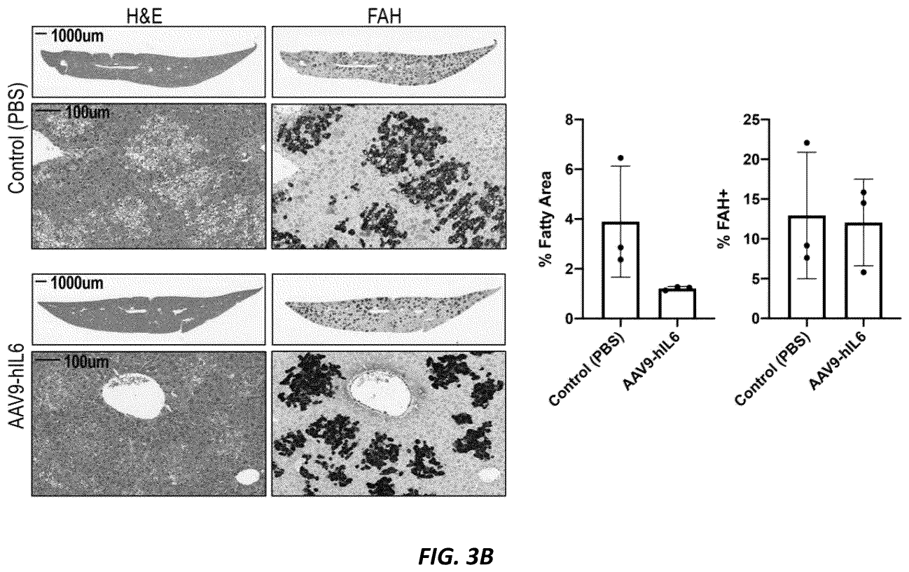

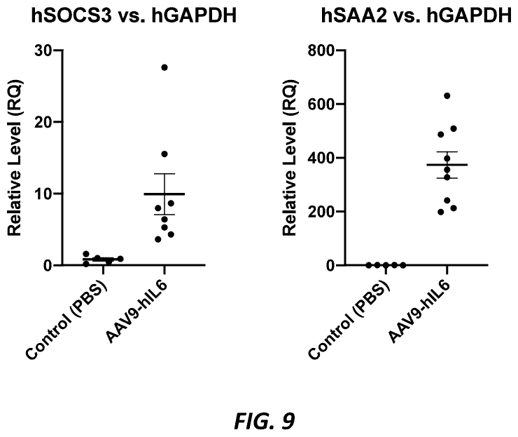

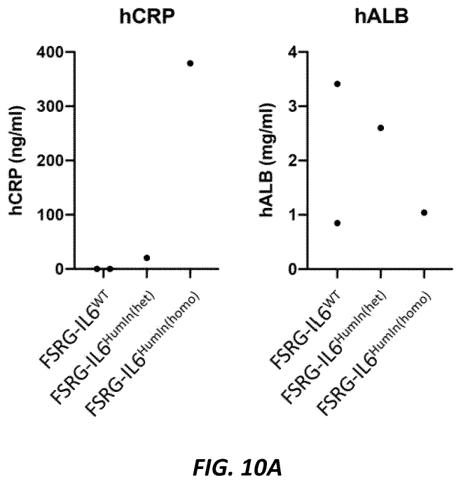

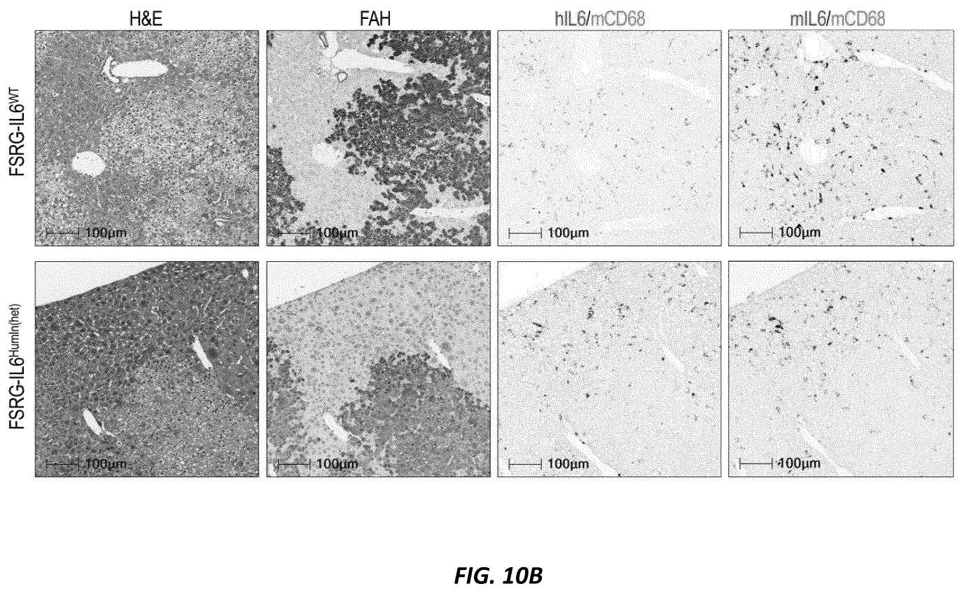

Our observations on correction of liver fattiness through restoration of hepatic IL-6-GP130 signaling suggested that supplementation with huIL-6 in host animals would protect humanized livers from hepatic steatosis. To test this hypothesis, immediately before human hepatocyte transplantation, FSRG mice were dosed with AAV9 encoding an expression vector in which the huIL-6 coding region was placed under control of the muscle-specific promoter, MHCK7, a hybrid promoter with the enhancer/promoter regions of mouse muscle creatine kinase (CK) and alpha-myosin heavy-chain genes (hIL-6 coding and protein sequences set forth in SEQ ID NOS: 44-45, respectively). As a result, huIL-6 expression was maintained in the serum of humanized liver mice until 8 weeks post-transplantation, when tissues were harvested. At 8 weeks post-transplantation of human hepatocytes, AAV-huIL-6 dosed FSRG mice expressed 1.74.8 ng/mL of huIL-6 in the serum and exhibited robust phosphorylation of STAT3 in the liver (FIG. 3A). In contrast, neither serum hIL-6, nor liver STAT3 phosphorylation, were detectable in control humanized liver mice. In addition, we also observed increased expression of IL-6 target genes, including human SOCS3 and SAA2, in the livers (FIG. 9). Significantly, compared to control animals with similar amounts of human engraftment, humanized livers of mice expressing with huIL-6 showed a substantial decrease of eosin-negative area (˜2-6-fold, almost to baseline level), mostly in FAH+ regions, indicative of a reduction of lipid droplet accumulation (FIG. 3B) and supporting a protective role of huIL-6 against hepatic steatosis in this model.

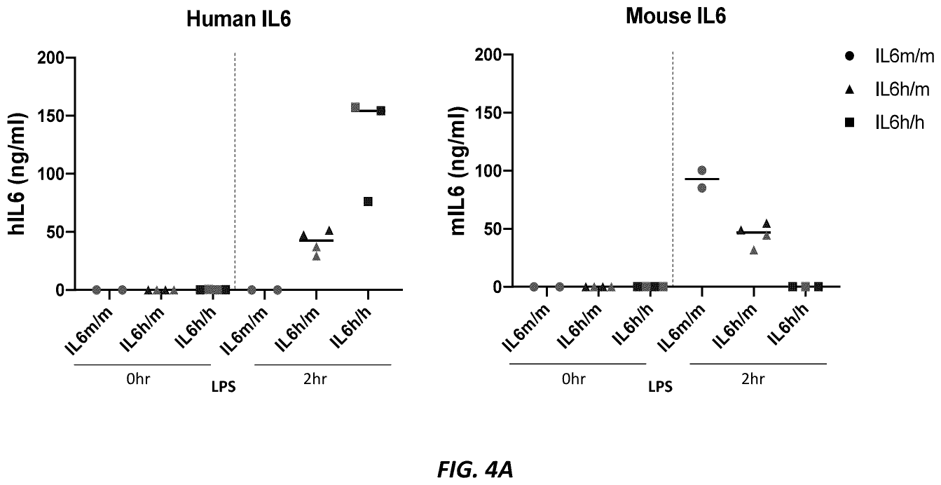

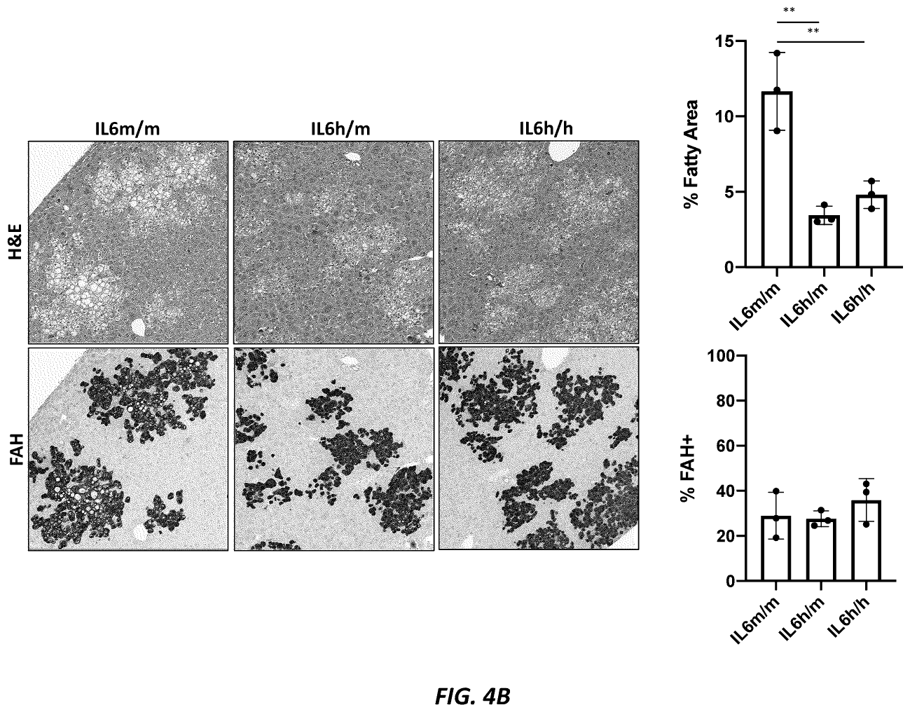

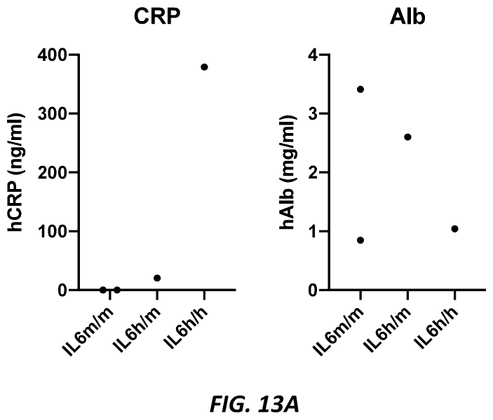

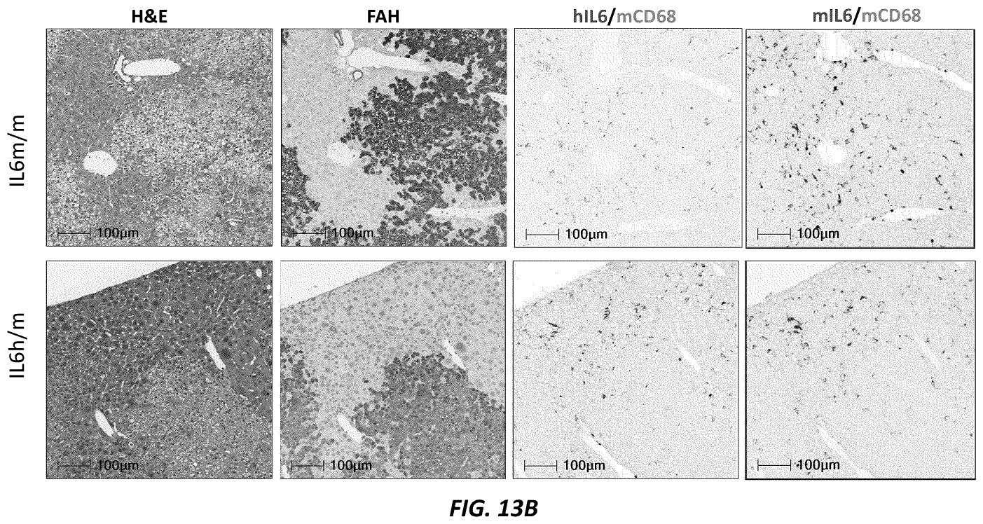

Systemic supplementation of hIL-6 through AAV dosing resulted in serum levels of the cytokine which were higher than the normal range, which could cause non-physiological and potentially detrimental effects. To address these concerns, we generated a recipient mouse with an FSRG background in which the coding region of murine IL-6 allele was replaced with its human homologue. The humanized IL6 locus is described, e.g., in U.S. Pat. No. 9,622,460, herein incorporated by reference in its entirety for all purposes. In such an animal, human IL-6 gene expression is controlled by the endogenous murine IL-6 promoter. Lipopolysaccharide (LPS) treatment resulted in induction of huIL-6 expression in the serum of mice with homozygous humanized IL-6 allele (FIG. 4A). In comparison, only murine IL-6 was detected in mice with wild type murine IL-6 alleles (FIG. 4A). In heterozygous mice, in which the IL-6 locus has one allele each of human and murine IL-6 genes, both human and murine IL-6 protein were detected in the serum (FIG. 4A). After liver humanization, human C-reactive protein (CRP) was detected in the serum of both FSRG-IL-6^{HumIn(het)} and FSRG-IL-6^{HumIn(homo)} mice, but not FSRG-IL-6^{WT} mice (FIG. 13A), confirming a human-specific IL-6 response in humanized liver mice with humanized IL-6 allele. Consistently, human hepatocytes engrafted in the livers of FSRG-IL-6^{WT} mice showed abundant lipid droplet accumulation, whereas humanized livers in both FSRG-IL-6^{HumIn(het)} or FSRG-IL-6^{HumIn(homo)} mice showed significant alleviation of the fatty phenotype, with ˜2-3-fold reduction of eosin-negative vacuoles area in FAH-positive regions (FIG. 4B), demonstrating a role of endogenous IL-6 in protecting human hepatocytes from lipid droplet accumulation in this model.

### Example 5. Human IL-6 Reverses Lipid Droplet Accumulation in Human Hepatocytes

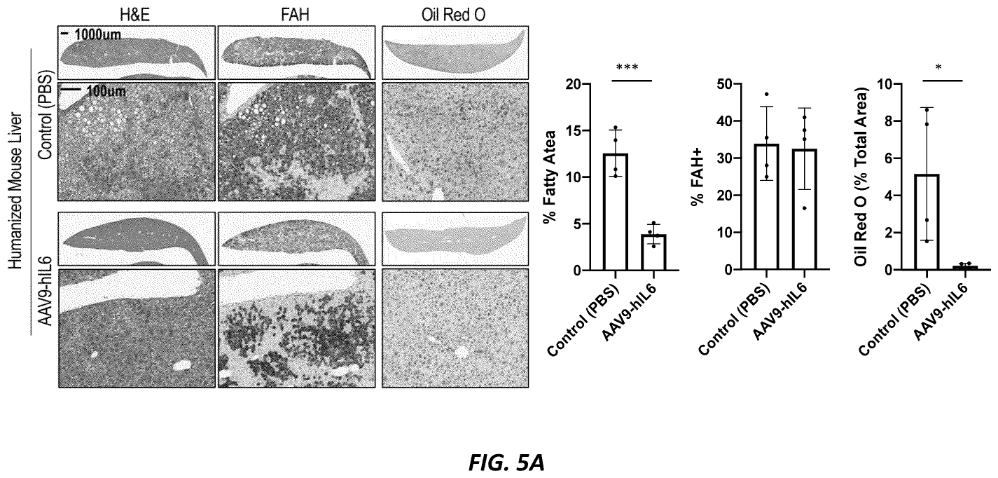

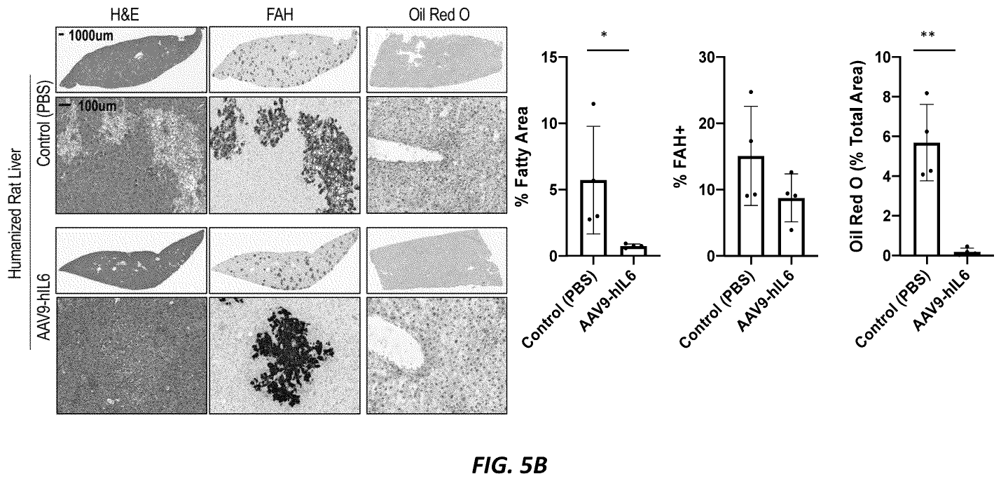

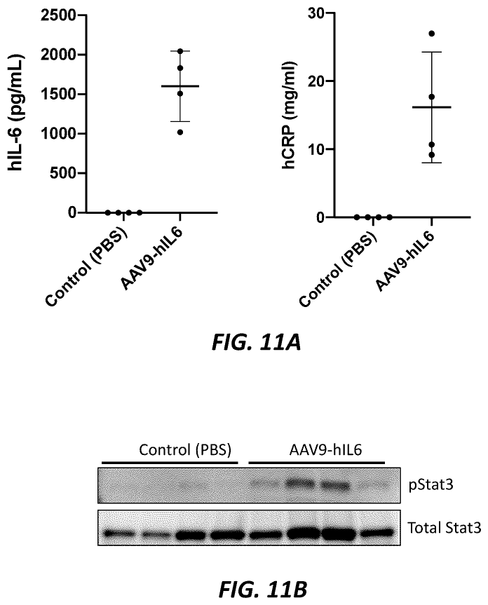

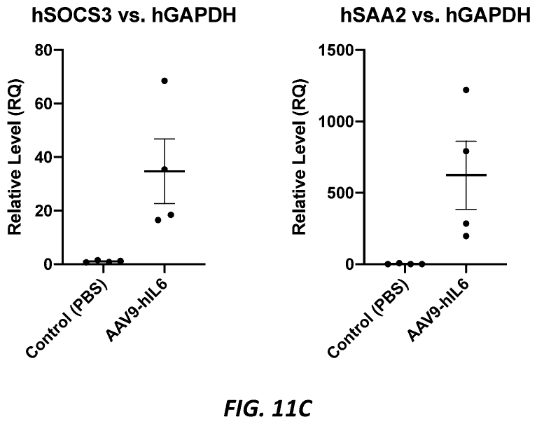

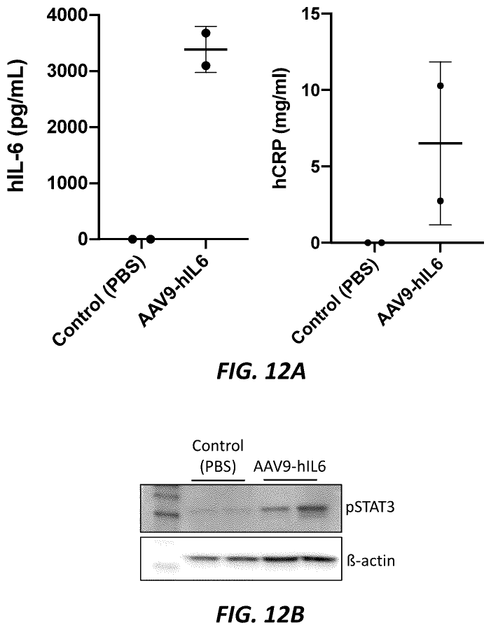

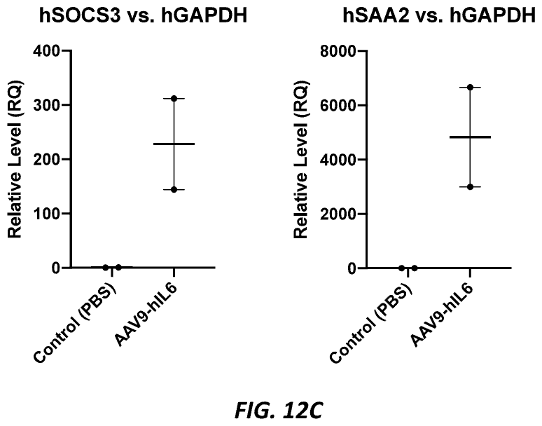

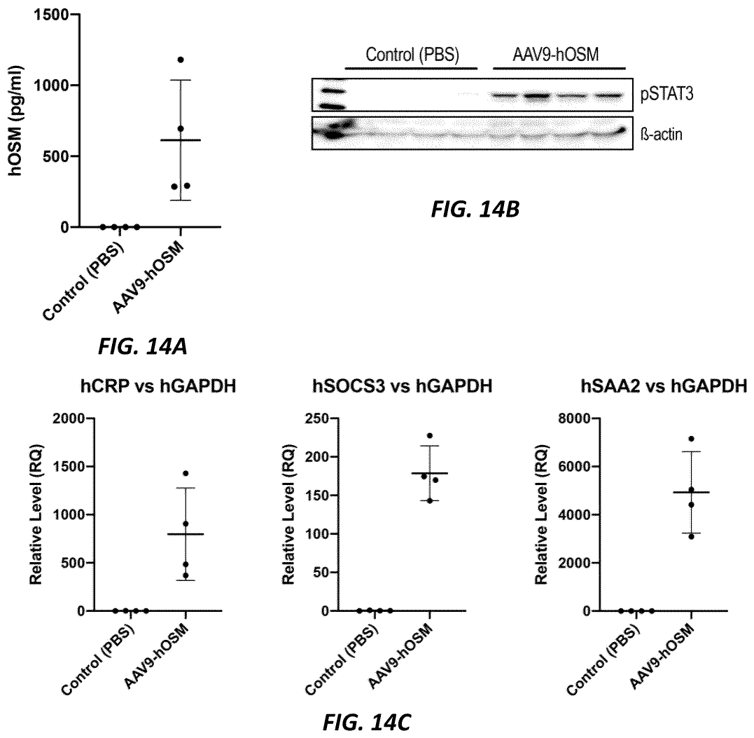

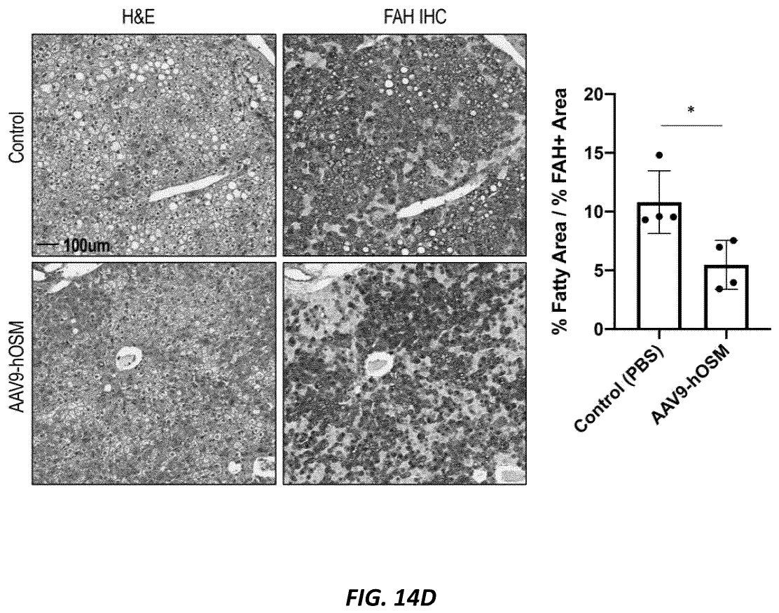

Next, we asked whether huIL-6 treatment can reverse the steatosis phenotype in humanized livers. This question was addressed in both humanized liver mouse and rat models. Rather than administering AAV9-huIL-6 before hepatocyte transplantation, viral dosing was performed in FSRG mice or FRG rats that were already engrafted with human hepatocytes. At 4 weeks after AAV9-huIL-6 dosing, tissues were collected. Again, huIL-6 expression and hepatic IL-6 pathway activation was confirmed by serum huIL-6 and human CRP, liver pSTAT3, and qRT-PCR for IL-6 target genes (FIGS. 11A-11C and FIGS. 12A-12C). In both mouse (FIG. 5A) and rat (FIG. 5B) models, huIL-6 dosing led to a nearly complete elimination of lipid droplet accumulation, marked by robust reduction of eosin-negative vacuoles in FAH+ regions and disappearance of Oil-Red-O staining in humanized livers. Thus, activated IL-6R signaling in human hepatocytes not only prevents, but also reverses, hepatic steatosis formation in humanized liver rodents. In addition, dosing with hOSM, another hepatic GP130/STAT3 pathway activating ligand, also led to correction of liver fattiness in humanized liver mice (FIGS. 14A-14D), supporting a protective role of GP130 pathway against lipid droplet accumulation in livers.

### Example 6. IL-6-Producing Human Kupffer Cells Protect Human Hepatocytes from Lipid Droplet Accumulation in Humanized Liver Mice

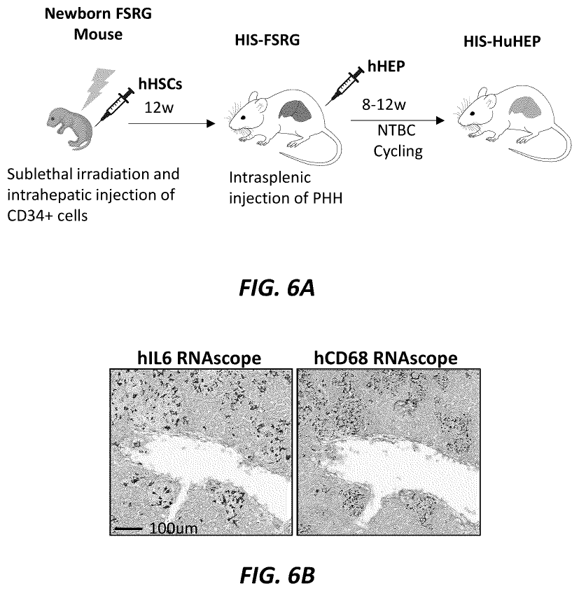

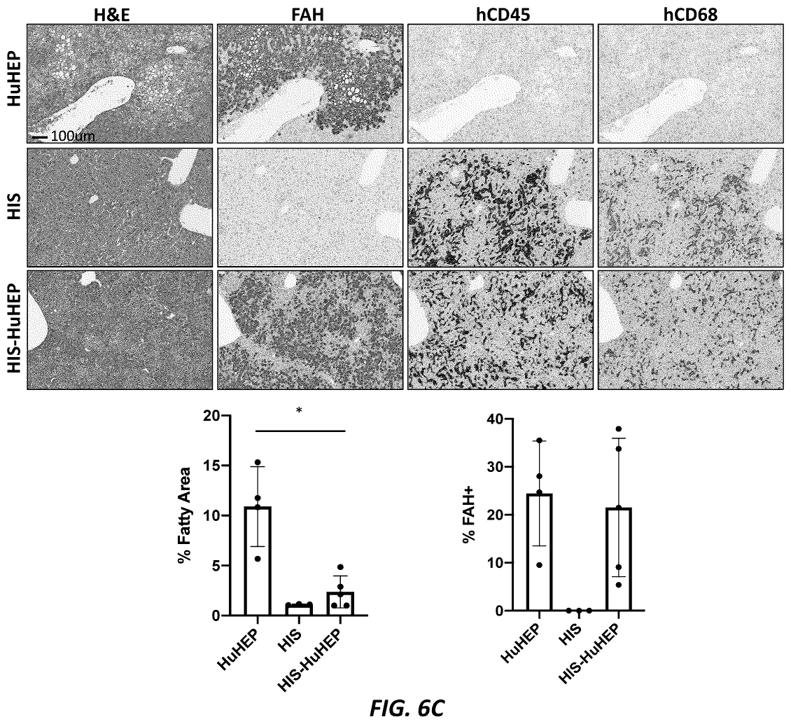

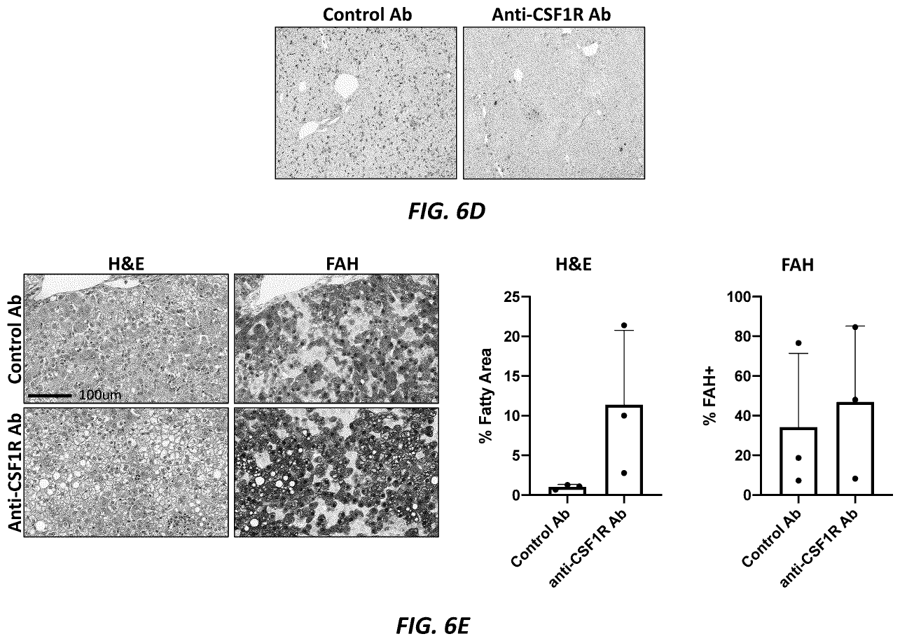

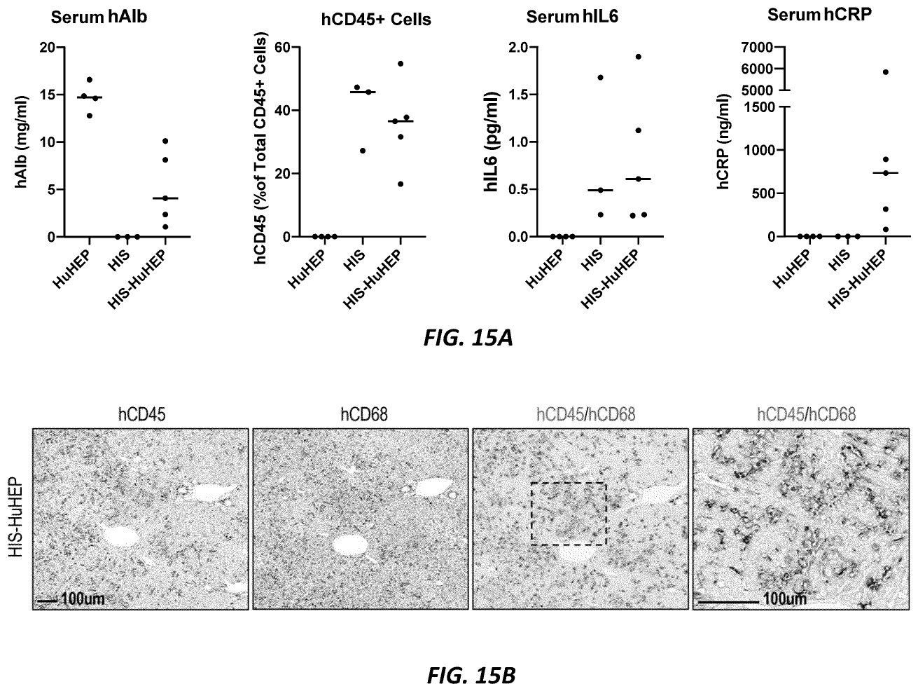

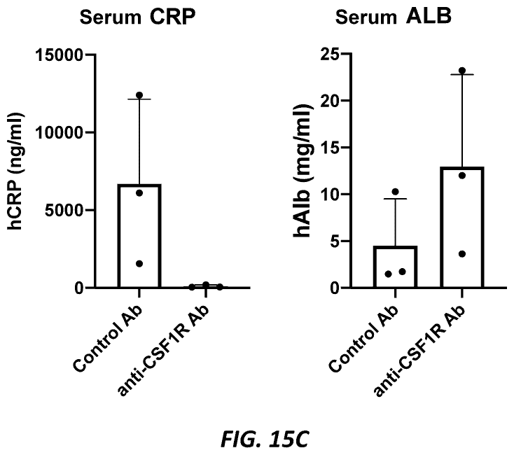

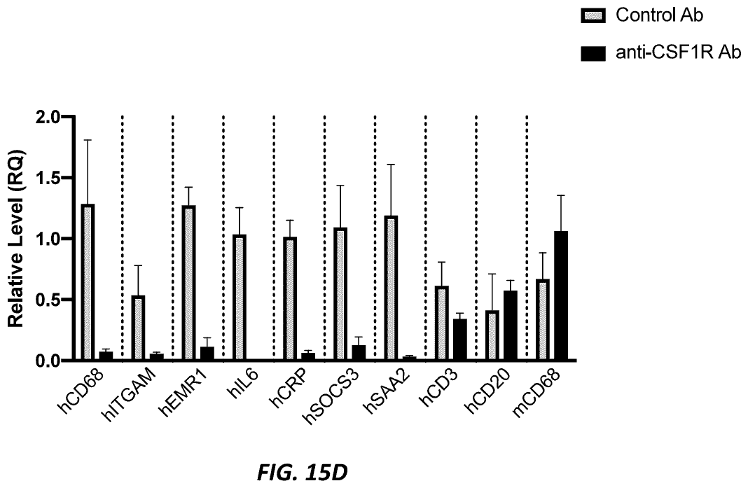

Kupffer cells are one of the major sources of IL-6 in livers. These cells are located adjacent to hepatocytes where Kupffer cell-derived IL-6 could support IL-6R pathway activation in nearby hepatocytes without elevating circulating IL-6 to harmful levels. We therefore attempted to address the role of Kupffer cells in protecting human hepatocytes from lipid droplet accumulation. To this end, we performed human immune cell reconstitution in FSRG mice prior to human hepatocyte transplantation (FIG. 6A). Human fetal liver cells containing hematopoietic stem cells were engrafted into irradiated neonatal FSRG pups. By week 12, about 25-50% of total leukocytes in peripheral blood of these mice were human immune cells expressing hCD45 (FIG. 15A). Impressively, abundant human CD68-positive cells that also express human IL-6 were detected in the livers of these animals, suggesting the engraftment of human Kupffer cells (FIG. 6B). Following human immune cell reconstitution, human hepatocytes were then transplanted to repopulate the liver parenchyma of the host mice to generate double humanized mice engrafted with both human immune system (HIS) and human Hepatocytes (HuHEP) (FIG. 6A). As shown in FIG. 6C, CD45-positive human immune cells intermingled with FAH positive human hepatocytes in the livers of double humanized mice. Indeed, the majority of the hCD45-positive cells in the liver were also hCD68-positive (FIG. 15B). We confirmed expression of huIL-6 in the serum of these mice at physiological levels, as well as expression of human CRP produced by engrafted human hepatocytes, suggesting that the IL-6 signaling pathway was restored in the double-humanized mice (FIG. 15A). Significantly, in contrast to the evident hepatic steatosis in humanized liver mice without human immune cells (FIG. 6C, upper panel), humanized livers engrafted with human immune cells showed very low, if any, lipid droplet accumulation (FIG. 6C, lower panel), indicating a role of engrafted human Kupffer cells in protection of human hepatocytes from steatosis.

To validate the role of engrafted human Kupffer cells, we pharmacologically depleted this cell population in the livers of double humanized mice and assessed whether such treatment would lead to lipid droplet accumulation in human hepatocytes. The colony-stimulating factor 1 receptor (CSF1R) pathway has been shown to serve as a critical survival signal for macrophages including Kupffer cells. In our study, we specifically depleted human Kupffer cells by blocking the interaction between hCSF1R, but not mCSF1R, and its ligands with a human-specific CSF1R antibody. As shown in FIG. 6D, hCD68-expressing cells in humanized livers of HIS-HuHEP mice were almost completely depleted upon hCSF1R antibody treatment. However, CD3+ T-cells and other non-macrophage cell types (e.g., hCD3+ and hCD20+) showed only moderate changes (FIG. 15D), implying that the anti-hCSF1R antibody specifically ablated human CD68-positive cells in livers of HIS-HuHEP mice. Impressively, RNA expression of human IL-6 and its target genes hSOC3, hSAA2, and hCRP in the liver, and human CRP in the serum, were dramatically decreased (FIGS. 15C and 15D), and extensive steatosis re-occurred in anti-hCSF1R antibody-treated HIS-HuHEP mice (FIG. 6E). Immunohistochemical analysis confirmed a more than 3-fold increase in lipid droplet accumulation, marked by eosin-negative vacuoles in FAH-positive, humanized regions of the liver (FIG. 6E). Therefore, Kupffer cells producing human IL-6 play a major role in controlling lipid droplet accumulation in hepatocytes in this model.

Hepatic steatosis in humanized liver rodent models, resulting from incompatibility between human hepatocyte-expressed receptors and host-derived ligands, provides an opportunity to identify important signaling pathways involved in fatty liver disease. In this report, we addressed the role of IL6-IL6R/GP130 signaling in human hepatosteatosis. First, we demonstrated that excessive lipid droplet accumulation in humanized livers is associated with incompatibility between rodent IL6 ligand expressed by non-parenchymal cells of host animals and human IL6R expressed on donor hepatocytes. We showed that lipid accumulation could be corrected by ectopic expression of rodent IL6R or constitutive activation of GP130 in donor hepatocytes. Further, supplementation of huIL6 systemically, either via ectopic expression or genetic humanization of the murine IL6 allele, again corrected the hepatosteatosis. Finally, engraftment of human Kupffer cells into host animals also corrected the fatty phenotype. Our results in humanized rodent liver models reveal a role for hepatic GP130 signaling, maintained by IL6 that is normally provided by non-parenchymal liver cells, in protecting hepatocytes from excessive lipid accumulation. In these examples, we used multiple approaches to demonstrate the importance of the hepatic IL6-GP130 signaling pathway in regulating liver fattiness. Lipid droplet accumulation in human engraftment represents a major defect in humanized liver models that greatly compromises their accuracy in recapitulating normal human liver biology. Our observations show an important role of both Kupffer cell-derived IL6 and its downstream IL6R/GP130 signaling in regulating lipid accumulation in hepatocytes and provide methods to improve humanized liver animal models.

### Methods

Animals. The FSRG mouse strain was developed with Regeneron VELOCIGENE technology and was rendered immune-deficient by deletion of Rag2 and IL-2Rg genes. The SIRPa gene was also humanized to allow for superior engraftment of human tissue (prevents phagocytosis of incoming human cells by facilitating “don't-eat-me-signal” to murine phagocytes). Lastly, FAH was deleted to induce murine liver ablation and allow for human hepatocyte engraftment. The FRG rat strain is described in Carbonaro et al., “Efficient engraftment and viral transduction of human hepatocytes in an FRG rat liver humanization model,” Sci. Rep. 12(1):14079, (2022), herein incorporated by reference in its entirety for all purposes. All studies were done in accordance with the Institutional Animal Care and Use Committee (IACUC) guidelines at Regeneron Pharmaceuticals, Inc. Animals were maintained with 2-(2-Nitro-4-trifluoromethylbenzoyl)-1,3-cyclohexanedione (NTBC)-containing drinking water at a concentration of 8 mg/L. NTBC was purchased from Medinoah (90-1596). The drinking water also contained antibiotics, Sulfamethoxazole (640m/mL, RPI 547000) and Trimethoprim (128m/mL, RPI T59000), in 3% dextrose water.

Hepatocyte Transplantation. Cryopreserved rat and mouse hepatocytes were purchased from ThermoFisher (RTCP10) and Yecuris (20-0019), respectively. Human cryoplatable primary hepatocytes were purchased from BioIVT (F00995-P and M00995-P). An adenoviral vector expressing human urokinase (uPA) was purchased from Yecuris (CuRx™ uPA Liver Tx Enhancer, 20-0029), and given by tail vein I.V. injection at 1.25×10⁹ PFU (plaque forming units) per 25 g of body weight in 100 μL sterile phosphate-buffered saline (PBS), 24 hours prior to hepatocyte transplant. Hepatocyte number and viability was determined by ViaStain AOPI Staining Solution (Nexcelom CS2-0106), using the Nexcelom Cellometer Auto 2000. 0.5-1 million live hepatocytes in 100 μL Roswell Park Memorial Institute (RPMI) 1640 Medium were injected into the spleen via a 32 gauge needle. NTBC was withdrawn from the drinking water the day before transplant and mice were put into an NTBC on/off cycling schedule, as follows: 7 days off, 3 days on for the first 2.5-3 weeks, then gradually increasing the NTBC off times to 8, 10, 14, then 28 days between 3-day on cycles.

HSC Engraftment and Immune Check. Single cell suspension of human fetal liver tissue (FL) was prepared by Collagenase D digestion (100 ng/mL; Roche) for 25 minutes at 37° C. Human CD34+ hematopoietic stem cells (HSCs) were isolated from the cell suspension by positive immunomagnetic selection using anti-human CD34 microbeads according to the manufacturer's instructions (Miltenyi Biotec). Newborn pups were sublethally irradiated (360 cGy; X-RAD 320 irradiator) 4-24 hours prior to an intrahepatic injection of 1×10⁵ human FL-derived CD34+ cells. Engraftment with human immune system was checked by retro-orbital bleed of mice 12 weeks post-HSC injection and red blood cell lysed blood cells were analyzed by FACS for human CD45 and mouse CD45. Gating was performed on hCD45+/mCD45− cells and human CD45+ population was analyzed for CD3 (T cells), CD19 (B cells), NKp46 (NK cells), and CD14 (monocytes/macrophages). Upon engraftment check, mice were subjected to human hepatocyte transplantation as further described.

ELISA Assays. Blood was collected from the submandibular vein and spun in MiniCollect serum separator tubes (#450472). Engraftment of human hepatocytes was monitored using the Human Albumin ELISA Kit (abcam, ab108788). Human and mouse IL-6 and human CRP were measured using SimpleStep ELISA kits ab178013, ab222503, and ab260058, respectively. FGF19 was detected using the Human FGF19 ELISA kit (ab230943).

Hepatocyte Culture and Western Blotting. Primary human, mouse, or rat hepatocytes were thawed in Cryopreserved Hepatocyte Recovery Medium (CHRM, CM7000, ThermoFisher) and plated in Williams E Media, with no phenol red (A1217601, ThermoFisher) with Primary Hepatocyte Thawing/Plating supplements (CM3000, ThermoFisher). 24 hours post-plating, media is changed and Primary Hepatocyte Maintenance Supplements (CM4000, ThermoFisher), plus 1% FBS are added. Hepatocytes were treated with recombinant human IL-6 (ab119444), mouse IL-6 (ab238300) or rat IL-6 (Cell Applications, RP3009), human HGF (R&D 294-HG-025), or mouse HGF (R&D 2207-HG-025) for 15 minutes at 50 ng/mL. Cells were lysed using RIPA buffer (ThermoFisher, 89900), containing protease and phosphatase inhibitors (ThermoFisher, A32965 & 88667), run by SDS-PAGE and probed using antibodies against pSTAT3, total STAT3 (Cell Signaling 9145, 4904), and B-actin (Sigma A5316).

Histology, Immunohistochemistry, and RNAscope. Livers were fixed in 10% normal buffered formalin for 24 hours, washed and stored in 70% ethanol until paraffin embedding and sectioning. Sections were stained by hematoxylin and eosin (H&E), according to standard protocols (staining performed by Histoserv, Inc.). Engrafted hepatocytes were detected by IHC staining for FAH (ab151998) and human ASGR1 (ab254261), HSD17B13 (Sigma HPA029125) and FLAG (ab205606) and human immune cells were detected using antibodies against hCD45 (Agilent/Dako #IS751) and hCD68 (Agilent/Dako #IS609). RNAscope probes for hIL-6 (310371), mIL-6 (315891), hCD68 (818681), mCD68 (316611) and FLAG (1139121-S1) were purchased from Advanced Cell Diagnostics (ACD). Oil Red O staining was performed on livers fresh frozen in Tissue-Tek O.C.T. Compound (ThermoFisher 14-373-65). All histology slides were scanned using a Leica Aperio AT2 scanner. FAH IHC, H&E and Oil Red O staining were quantified using ImageJ.

Adeno-associated Virus (AAV) Production and Delivery. Recombinant AAV was produced by transient transfection of HEK 293T cells. Briefly, cells were transfected with AAV Rep-Cap, Adenovirus Helper, and AAV genome plasmids using PEI-Max (Polysciences). Virus containing supernatant were concentrated by tangential flow filtration and cells were lysed by sequential freeze and thaw (3×). Lysates were treated with Benzonase (Millipore Sigma) for one hour at 37° C. and clarified by centrifugation and filtration (0.2 μm PES). AAV was purified from clarified cell lysates and concentrated supernatant by iodixanol gradient ultracentrifugation. Virus fractions were concentrated and buffer-exchanged to 1×PBS+0.001% Pluronic F68 (Thermo Fisher Scientific) using Amicon 100 kDa MWCO Ultra Centrifugal filters (Millipore Sigma). AAV genomes were quantified by qPCR using TaqMan primers and probes specific for inverted terminal repeats. A standard curve was generated using serial dilutions of virus with a known concentration. Adeno-associated viruses (AAVs) were delivered intravenously by tail-vein injection at 5.00E+11 viral genomes (VG)/mouse.

Lentiviral Vector Production, Titration and Infection. Lentiviral particles were produced following standard lipofectamine-mediated co-transfection of HEK 293T cells with the transfer plasmid encoding mIL-6R and rIL-6R under the CMV promoter (pLVX-CMV-, subcloned from pLVX-EF1a-IRES-puro plasmid, Takara), a second generation packaging plasmid encoding the gag, pol and rev genes (psPAX2, obtained from the Tronolab at Ecole Polytechnique Fëdërale de Lausanne, Switzerland) and a plasmid encoding the vesicular stomatitis virus envelope glycoprotein G (VSV-G) as envelope plasmid (pMD2-G, Tronolab). The day before transfection cells were washed with phosphate buffered saline solution (PBS) once then detached from vessel with TrypLE™ Express (Life Technologies). After neutralization of TrypLE Express with cell medium containing FBS, cells were centrifuged at 1200 rpm for 5 minutes at 25° C., then resuspended in complete DMEM medium, counted and seeded in 150 mm cell culture dishes at a density of 10×10⁶ cells/plate. On the day of transfection, the cell culture medium was replaced by fresh Opti-MEM medium (Gibco/Life Technologies) supplemented with 25 nM chloroquine (Sigma-Aldrich). The DNA mix was prepared by mixing 20 μg of transfer plasmid DNA, 20 μg of packaging plasmid and 10 μg of envelope plasmid, 1.5 mL of Opti-MEM with 60 μL of PLUS' Reagent (Life Technologies). In parallel 100 mL of Lipofectamine® TLX (Life Technologies) was diluted in 1.5 mL of OptiMEM medium. DNA mix was then added to the lipofectamine mix and the new combined solution was incubated at room temperature for 20 minutes before being added directly to the cells dropwise. The culture medium was changed 6 to 8 hours after transfection and the cells were then incubated for 48 hours at 37° C. in an incubator with 5% CO₂ atmosphere. At day 2 post-transfection, cell media containing the lentiviral particles were centrifuged for 10 minutes at 3000 rpm to remove the debris, then passed through a 0.45 μm pore size filter. The filtered supernatants were then treated with 1 pg/mL DNAse and 1 mM MgCl₂ for 15 minutes at 37° C. to remove residual DNA. For concentrating the lentiviral vectors batch, the supernatants were then ultracentrifuged at 27,100 rpm for 90 minutes. After ultracentrifugation, pellets were resuspended in a suitable volume of PBS (50 to 100 mL) overnight. The resuspended virus was finally processed through a series of short centrifugations (30 seconds at 13500 rpm) to clarify the lentiviral solution of remaining debris. The batches of lentiviral particles were titrated by RT-qPCR using a SYBR® technology-based kit from Clontech/Takara then stocked at −80° C. until use for transduction. Lentivirus infection of human hepatocytes was done ex-vivo prior to engraftment, at an MOI of 5.00E+04 VG/cell. Cells were infected for 30 minutes in suspension immediately upon thawing cryopreserved cells, washed with PBS, and prepared for engraftment as above.

Antibody Dosing. The human-specific cMET antibody (Regeneron, REGN6753) was dosed 1×/week at 25 mg/kg. The anti-CSF1R antibody (Regeneron, H4H32090P) was given 2 times per week at a concentration of 20 mg/kg. An isotype control antibody (Regeneron, REGN1945), which does not bind any human or mouse proteins, was used as control. Antibodies were diluted in PBS and given by intraperitoneal (IP) injection.

TaqMan real-time polymerase chain reaction (PCR). Tissues were homogenized in RNALater and purified using MagMAX-96 for Microarrays Total RNA Isolation Kit (Ambion by Life Technologies). Genomic DNA was removed using RNase-Free DNase Set (Qiagen). mRNA was reverse-transcribed into cDNA using SuperScript VILO Master Mix (Invitrogen Life Technologies) and Veriti 96-well PCR Thermal Cycler (Thermo Fisher Scientific). cDNA was amplified with SensiFAST Probe Hi-ROX (Meridian Life Science) using 12k Flex System (Applied Biosystems). PCR reactions were done in triplicate. GAPDH was used to normalize cDNA input differences. Data reported as comparative CT method using delta CT. Probe sequences are listed in Table 2. Primer sequences are listed in Table 3.

TABLE 2
Real-time PCR probe sequences.

| | | SEQ |
|---|---|---|
| | | ID |
| Gene | Probe Sequence | NO |
| hIL6 | CGGCATCTCAGCCCTGAGAAAGGA | 1 |
| hSOCS3 | TCCAAGAGCGAGTACCAGCTGG | 2 |
| hSAA2 | AATATCCAGAGACTCACAGGCCGTGG | 3 |
| hCRP | TGTCTGGTCTGGGAGCTCGTTAAC | 4 |
| hCD68 | AACAGCACTGCCACCAGCCCAG | 5 |
| hITGAM | CCTCTCACTCCGACTTTCTGGCTGA | 6 |
| hEMR1 | CCACACGGAAACCAAACACAAAGGG | 7 |
| hCD3 | TCCAGAGACAACGCCAAGGATTCC | 8 |
| hCD20 | TGCTATGCAATCTGGTCCAAAACCAC | 9 |
| mCD68 | CTTCCCACAGGCAGCACAGTGG | 10 |
| hCYP7A1 | CCCTCAACATCCGGACAGCTAAGGA | 11 |
| hGAPDH | TCAACAGCGACACCCACTCCTC | 12 |
| mGAPDH | ATCCACTGGTGCTGCCAAGGCTG | 13 |

TABLE 3
Real-time PCR primer sequences.

| | | | SEQ ID |
|---|---|---|---|
| | Forward | Reverse | NOS |
| Gene | Primer (F) | Primer (R) | (F, R) |
| hIL6 | TGACAAACAA | GTGCCTCTTT | 14, 15 |
| | ATTCGGTACA | GCTGCTTTCA | |
| | TCCTC | C | |
| hSOCS3 | TGCGCCTCAA | TCACTGCGCT | 16, 17 |
| | GACCTTCAG | CCAGTAGAAG | |
| hSAA2 | GGCTGCAGAA | ATCGGCCAGC | 18, 19 |
| | GTGATCAGCA | GAGTCCTC | |
| | ATG | | |
| hCRP | AGCGCCTGAG | TGGACCGTTT | 20, 21 |
| | AATGGAGGTA | CCCAGCATA | |
| | A | | |
| hCD68 | GCCACGGTTC | GGTGGGCAGA | 22, 23 |
| | ATCCAACAAG | ACTGGTGAAT | |
| | | C | |
| hITGAM | TGCCACACCA | GTTGCCTTTG | 24, 25 |
| | AGGAGCG | AGGGTAGCAT | |
| | | TG | |
| hEMR1 | AGCTGGGAAG | GCTGGGCACA | 26, 27 |
| | GGCACATAAG | AGGTACTG | |
| | AC | | |
| hCD3 | GAAGGGCCGA | TGCAGCTCTC | 28, 29 |
| | TTCACCATC | AGACTGTCCA | |
| | | T | |
| hCD20 | CCGGCAGAGC | GGCCCACCAG | 30, 31 |
| | CAATGAAAGG | TGAAGACATC | |
| mCD68 | GGCGGTGGAA | GGAGCTCTCG | 32, 33 |
| | TACAATGTGT | AAGAGATGAA | |
| | C | TTCTG | |
| hCYP7A1 | GAATCGCTGA | ACCGTCCTCA | 34, 35 |
| | GGCTTTCCAG | AGGTGCAAAG | |
| | TG | TG | |
| hGAPDH | CCAGGTGGTC | GCTTGACAAA | 36, 37 |
| | TCCTCTGACT | GTGGTCGTTG | |
| | | A | |
| mGAPDH | TGCCCAGAAC | GGAAGGCCAT | 38, 39 |
| | ATCATCCCT | GCCAGTGAG | |

1. A genetically modified non-human animal,
wherein the genetically modified non-human animal comprises transplanted hepatocytes from a different species than the non-human animal, optionally wherein the hepatocyte are human hepatocytes, and
wherein the genetically modified non-human animal and/or the transplanted hepatocytes are modified to restore interleukin-6 (IL-6)/interleukin-6 receptor (IL-6R) signaling pathway activity or to restore interleukin-6 receptor subunit beta (GP130) signaling pathway activity in the transplanted hepatocytes.

2.-11. (canceled)

12. A genetically modified non-human animal comprising an inactivated endogenous Rag2 gene, an inactivated endogenous Il2rg gene, and an inactivated endogenous Fah gene, optionally wherein the genetically modified non-human animal comprises an inactivated endogenous Rag1 gene
wherein the genetically modified non-human animal comprises transplanted hepatocytes, wherein the transplanted hepatocytes are human hepatocytes, and
wherein the genetically modified non-human animal and/or the transplanted hepatocytes are modified to restore interleukin-6 (IL-6)/interleukin-6 receptor (IL-6R) signaling pathway activity or interleukin-6 receptor subunit beta (GP130) signaling pathway activity in the transplanted hepatocytes.

13.-93. (canceled)

94. A method of assessing the activity of a human-liver-targeting reagent in vivo, comprising:
(a) administering the human-liver-targeting reagent to the genetically modified non-human animal of claim 1; and
(b) assessing the activity of the human-liver-targeting reagent in the liver of the genetically modified non-human animal.

95.-105. (canceled)

106. A method of making a non-human animal with a humanized liver, comprising:
(a) transplanting human hepatocytes or human hepatocyte progenitors into a genetically modified non-human animal; and
(b) allowing the human hepatocytes or human hepatocyte progenitors to expand, wherein the genetically modified non-human animal and/or the transplanted human hepatocytes or human hepatocyte progenitors are modified to restore interleukin-6 (IL-6)/interleukin-6 receptor (IL-6R) signaling pathway activity or interleukin-6 receptor subunit beta (GP130) signaling pathway activity in the transplanted human hepatocytes or human hepatocyte progenitors.

107.-115. (canceled)

116. A method of making a non-human animal with a humanized liver, comprising:
(a) transplanting human hepatocytes or human hepatocyte progenitors into a genetically modified non-human animal comprising an inactivated endogenous Rag2 gene, an inactivated endogenous Il2rg gene, and an inactivated endogenous Fah gene, optionally wherein the genetically modified non-human animal comprises an inactivated endogenous Rag1 gene; and
(b) allowing the human hepatocytes or human hepatocyte progenitors to expand, wherein the genetically modified non-human animal and/or the transplanted human hepatocytes or human hepatocyte progenitors are modified to restore interleukin-6 (IL-6)/interleukin-6 receptor (IL-6R) signaling pathway activity or interleukin-6 receptor subunit beta (GP130) signaling pathway activity in the transplanted human hepatocytes or human hepatocyte progenitors.

117.-253. (canceled)

254. A genetically modified non-human animal comprising an inactivated endogenous Rag2 gene, an inactivated endogenous Il2rg gene, an inactivated endogenous Fah gene, and a humanized IL6 gene comprising a human IL6 nucleic acid encoding a human IL-6 protein, optionally wherein the genetically modified non-human animal comprises an inactivated endogenous Rag1 gene.

255. A genetically modified non-human animal comprising an inactivated endogenous Rag2 gene, an inactivated endogenous Il2rg gene, an inactivated endogenous Fah gene, and a humanized OSM gene comprising a human OSM nucleic acid encoding a human OSM protein, optionally wherein the genetically modified non-human animal comprises an inactivated endogenous Rag1 gene.

256. A genetically modified non-human animal comprising an inactivated endogenous Rag2 gene, an inactivated endogenous Il2rg gene, an inactivated endogenous Fah gene, a humanized IL6 gene comprising a human IL6 nucleic acid encoding a human IL-6 protein, and a humanized OSM gene comprising a human OSM nucleic acid encoding a human OSM protein, optionally wherein the genetically modified non-human animal comprises an inactivated endogenous Rag1 gene.

257.-285. (canceled)

286. A genetically modified non-human animal cell comprising an inactivated endogenous Rag2 gene, an inactivated endogenous Il2rg gene, an inactivated endogenous Fah gene, and a humanized IL6 gene comprising a human IL6 nucleic acid encoding a human IL-6 protein, optionally wherein the genetically modified non-human animal cell comprises an inactivated endogenous Rag1 gene.

287. A genetically modified non-human animal cell comprising an inactivated endogenous Rag2 gene, an inactivated endogenous Il2rg gene, an inactivated endogenous Fah gene, and a humanized OSM gene comprising a human OSM nucleic acid encoding a human OSM protein, optionally wherein the genetically modified non-human animal cell comprises an inactivated endogenous Rag1 gene.

288. A genetically modified non-human animal cell comprising an inactivated endogenous Rag2 gene, an inactivated endogenous Il2rg gene, an inactivated endogenous Fah gene, a humanized IL6 gene comprising a human IL6 nucleic acid encoding a human IL-6 protein, and a humanized OSM gene comprising a human OSM nucleic acid encoding a human OSM protein, optionally wherein the genetically modified non-human animal cell comprises an inactivated endogenous Rag1 gene.

289. A genetically modified non-human animal genome comprising an inactivated endogenous Rag2 gene, an inactivated endogenous Il2rg gene, an inactivated endogenous Fah gene, and a humanized IL6 gene comprising a human IL6 nucleic acid encoding a human IL-6 protein, optionally wherein the genetically modified non-human animal genome comprises an inactivated endogenous Rag1 gene.

290. A genetically modified non-human animal genome comprising an inactivated endogenous Rag2 gene, an inactivated endogenous Il2rg gene, an inactivated endogenous Fah gene, and a humanized OSM gene comprising a human OSM nucleic acid encoding a human OSM protein, optionally wherein the genetically modified non-human animal genome comprises an inactivated endogenous Rag1 gene.

291. A genetically modified non-human animal genome comprising an inactivated endogenous Rag2 gene, an inactivated endogenous Il2rg gene, an inactivated endogenous Fah gene, a humanized IL6 gene comprising a human IL6 nucleic acid encoding a human IL-6 protein, and a humanized OSM gene comprising a human OSM nucleic acid encoding a human OSM protein, optionally wherein the genetically modified non-human animal genome comprises an inactivated endogenous Rag1 gene.

292. A method of making the genetically modified non-human animal of claim 254, comprising:
(a) introducing a genetically modified non-human animal embryonic stem (ES) cell comprising an inactivated endogenous Rag2 gene, an inactivated endogenous Il2rg gene, an inactivated endogenous Fah gene, a humanized IL6 gene comprising a human IL6 nucleic acid encoding a human IL-6 protein and/or a humanized OSM gene comprising a human OSM nucleic acid encoding a human OSM protein, and optionally an inactivated endogenous Rag1 gene into a non-human animal host embryo; and
(b) implanting and gestating the non-human animal host embryo in a non-human animal surrogate mother, wherein the non-human animal surrogate mother produces an F0 progeny genetically modified non-human animal comprising the inactivated endogenous Rag2 gene, the inactivated endogenous Il2rg gene, the inactivated endogenous Fah gene, the humanized IL6 gene and/or the humanized OSM gene, and optionally the inactivated endogenous Rag1 gene.

293. (canceled)

294. A method of making the genetically modified rat of claim 254, comprising implanting and gestating a genetically modified non-human animal one-cell stage embryo comprising an inactivated endogenous Rag2 gene, an inactivated endogenous Il2rg gene, an inactivated endogenous Fah gene, a humanized IL6 gene comprising a human IL6 nucleic acid encoding a human IL-6 protein and/or a humanized OSM gene comprising a human OSM nucleic acid encoding a human OSM protein, and optionally an inactivated endogenous Rag1 gene in a non-human animal surrogate mother, wherein the non-human animal surrogate mother produces an F0 progeny genetically modified non-human animal comprising the inactivated endogenous Rag1 gene, the inactivated endogenous Rag2 gene, the inactivated endogenous Il2rg gene, the inactivated endogenous Fah gene, and the humanized IL6 gene and/or the humanized OSM gene.

295. (canceled)

296. A method of making a non-human animal with a humanized liver, comprising:
(a) transplanting human hepatocytes or human hepatocyte progenitors into the genetically modified non-human animal of claim 254;
(b) allowing the human hepatocytes or human hepatocyte progenitors to expand.

297.-303. (canceled)

304. A method of preventing, reducing, or ameliorating hepatosteatosis in a non-human animal comprising transplanted human hepatocytes, comprising administering a GP130-activating ligand or a nucleic acid encoding the GP130-activating ligand to the non-human animal, wherein the GP130-activating ligand restores interleukin-6 (IL-6)/interleukin-6 receptor (IL-6R) signaling pathway activity or interleukin-6 receptor subunit beta (GP130) signaling pathway activity in the transplanted human hepatocytes.

305. A method of preventing, reducing, or ameliorating lipid droplet accumulation in transplanted human hepatocytes in a non-human animal comprising the transplanted human hepatocytes, comprising administering a GP130-activating ligand or a nucleic acid encoding the GP130-activating ligand to the non-human animal, wherein the GP130-activating ligand restores interleukin-6 (IL-6)/interleukin-6 receptor (IL-6R) signaling pathway activity or interleukin-6 receptor subunit beta (GP130) signaling pathway activity in the transplanted human hepatocytes.

306.-340. (canceled)

341. A method of assessing the activity of a human-liver-targeting reagent in vivo, comprising:
(a) administering the human-liver-targeting reagent to the genetically modified non-human animal of claim 12; and
(b) assessing the activity of the human-liver-targeting reagent in the liver of the genetically modified non-human animal.

342. A method of making the genetically modified non-human animal of claim 255, comprising:
(a) introducing a genetically modified non-human animal embryonic stem (ES) cell comprising an inactivated endogenous Rag2 gene, an inactivated endogenous Il2rg gene, an inactivated endogenous Fah gene, a humanized IL6 gene comprising a human IL6 nucleic acid encoding a human IL-6 protein and/or a humanized OSM gene comprising a human OSM nucleic acid encoding a human OSM protein, and optionally an inactivated endogenous Rag1 gene into a non-human animal host embryo; and
(b) implanting and gestating the non-human animal host embryo in a non-human animal surrogate mother, wherein the non-human animal surrogate mother produces an F0 progeny genetically modified non-human animal comprising the inactivated endogenous Rag2 gene, the inactivated endogenous Il2rg gene, the inactivated endogenous Fah gene, the humanized IL6 gene and/or the humanized OSM gene, and optionally the inactivated endogenous Rag1 gene.

343. A method of making the genetically modified non-human animal of claim 256, comprising:
(a) introducing a genetically modified non-human animal embryonic stem (ES) cell comprising an inactivated endogenous Rag2 gene, an inactivated endogenous Il2rg gene, an inactivated endogenous Fah gene, a humanized IL6 gene comprising a human IL6 nucleic acid encoding a human IL-6 protein and/or a humanized OSM gene comprising a human OSM nucleic acid encoding a human OSM protein, and optionally an inactivated endogenous Rag1 gene into a non-human animal host embryo; and
(b) implanting and gestating the non-human animal host embryo in a non-human animal surrogate mother, wherein the non-human animal surrogate mother produces an F0 progeny genetically modified non-human animal comprising the inactivated endogenous Rag2 gene, the inactivated endogenous Il2rg gene, the inactivated endogenous Fah gene, the humanized IL6 gene and/or the humanized OSM gene, and optionally the inactivated endogenous Rag1 gene.

344. A method of making the genetically modified rat of claim 255, comprising implanting and gestating a genetically modified non-human animal one-cell stage embryo comprising an inactivated endogenous Rag2 gene, an inactivated endogenous Il2rg gene, an inactivated endogenous Fah gene, a humanized IL6 gene comprising a human IL6 nucleic acid encoding a human IL-6 protein and/or a humanized OSM gene comprising a human OSM nucleic acid encoding a human OSM protein, and optionally an inactivated endogenous Rag1 gene in a non-human animal surrogate mother, wherein the non-human animal surrogate mother produces an F0 progeny genetically modified non-human animal comprising the inactivated endogenous Rag1 gene, the inactivated endogenous Rag2 gene, the inactivated endogenous Il2rg gene, the inactivated endogenous Fah gene, and the humanized IL6 gene and/or the humanized OSM gene.

345. A method of making the genetically modified rat of claim 256, comprising implanting and gestating a genetically modified non-human animal one-cell stage embryo comprising an inactivated endogenous Rag2 gene, an inactivated endogenous Il2rg gene, an inactivated endogenous Fah gene, a humanized IL6 gene comprising a human IL6 nucleic acid encoding a human IL-6 protein and/or a humanized OSM gene comprising a human OSM nucleic acid encoding a human OSM protein, and optionally an inactivated endogenous Rag1 gene in a non-human animal surrogate mother, wherein the non-human animal surrogate mother produces an F0 progeny genetically modified non-human animal comprising the inactivated endogenous Rag1 gene, the inactivated endogenous Rag2 gene, the inactivated endogenous Il2rg gene, the inactivated endogenous Fah gene, and the humanized IL6 gene and/or the humanized OSM gene.

346. A method of making a non-human animal with a humanized liver, comprising:
(a) transplanting human hepatocytes or human hepatocyte progenitors into the genetically modified non-human animal of claim 255;
(b) allowing the human hepatocytes or human hepatocyte progenitors to expand.

347. A method of making a non-human animal with a humanized liver, comprising:
(a) transplanting human hepatocytes or human hepatocyte progenitors into the genetically modified non-human animal of claim 256;
(b) allowing the human hepatocytes or human hepatocyte progenitors to expand.


SEQUENCE LISTING (55 sequences)

SEQ ID NO: 1 - DNA, 24 bp, synthetic construct
  source 1..24: mol_type = other DNA
cggcatctcagccctgagaaagga

SEQ ID NO: 2 - DNA, 22 bp, synthetic construct
  source 1..22: mol_type = other DNA
tccaagagcgagtaccagctgg

SEQ ID NO: 3 - DNA, 26 bp, synthetic construct
  source 1..26: mol_type = other DNA
aatatccagagactcacaggccgtgg

SEQ ID NO: 4 - DNA, 24 bp, synthetic construct
  source 1..24: mol_type = other DNA
tgtctggtctgggagctcgttaac

SEQ ID NO: 5 - DNA, 22 bp, synthetic construct
  source 1..22: mol_type = other DNA
aacagcactgccaccagcccag

SEQ ID NO: 6 - DNA, 25 bp, synthetic construct
  source 1..25: mol_type = other DNA
cctctcactccgactttctggctga

SEQ ID NO: 7 - DNA, 25 bp, synthetic construct
  source 1..25: mol_type = other DNA
ccacacggaaaccaaacacaaaggg

SEQ ID NO: 8 - DNA, 24 bp, synthetic construct
  source 1..24: mol_type = other DNA
tccagagacaacgccaaggattcc

SEQ ID NO: 9 - DNA, 26 bp, synthetic construct
  source 1..26: mol_type = other DNA
tgctatgcaatctggtccaaaaccac

SEQ ID NO: 10 - DNA, 22 bp, synthetic construct
  source 1..22: mol_type = other DNA
cttcccacaggcagcacagtgg

SEQ ID NO: 11 - DNA, 25 bp, synthetic construct
  source 1..25: mol_type = other DNA
ccctcaacatccggacagctaagga

SEQ ID NO: 12 - DNA, 22 bp, synthetic construct
  source 1..22: mol_type = other DNA
tcaacagcgacacccactcctc

SEQ ID NO: 13 - DNA, 23 bp, synthetic construct
  source 1..23: mol_type = other DNA
atccactggtgctgccaaggctg

SEQ ID NO: 14 - DNA, 25 bp, synthetic construct
  source 1..25: mol_type = other DNA
tgacaaacaaattcggtacatcctc

SEQ ID NO: 15 - DNA, 21 bp, synthetic construct
  source 1..21: mol_type = other DNA
gtgcctctttgctgctttcac

SEQ ID NO: 16 - DNA, 19 bp, synthetic construct
  source 1..19: mol_type = other DNA
tgcgcctcaagaccttcag

SEQ ID NO: 17 - DNA, 20 bp, synthetic construct
  source 1..20: mol_type = other DNA
tcactgcgctccagtagaag

SEQ ID NO: 18 - DNA, 23 bp, synthetic construct
  source 1..23: mol_type = other DNA
ggctgcagaagtgatcagcaatg

SEQ ID NO: 19 - DNA, 18 bp, synthetic construct
  source 1..18: mol_type = other DNA
atcggccagcgagtcctc

SEQ ID NO: 20 - DNA, 21 bp, synthetic construct
  source 1..21: mol_type = other DNA
agcgcctgagaatggaggtaa

SEQ ID NO: 21 - DNA, 19 bp, synthetic construct
  source 1..19: mol_type = other DNA
tggaccgtttcccagcata

SEQ ID NO: 22 - DNA, 20 bp, synthetic construct
  source 1..20: mol_type = other DNA
gccacggttcatccaacaag

SEQ ID NO: 23 - DNA, 21 bp, synthetic construct
  source 1..21: mol_type = other DNA
ggtgggcagaactggtgaatc

SEQ ID NO: 24 - DNA, 17 bp, synthetic construct
  source 1..17: mol_type = other DNA
tgccacaccaaggagcg

SEQ ID NO: 25 - DNA, 22 bp, synthetic construct
  source 1..22: mol_type = other DNA
gttgcctttgagggtagcattg

SEQ ID NO: 26 - DNA, 22 bp, synthetic construct
  source 1..22: mol_type = other DNA
agctgggaagggcacataagac

SEQ ID NO: 27 - DNA, 18 bp, synthetic construct
  source 1..18: mol_type = other DNA
gctgggcacaaggtactg

SEQ ID NO: 28 - DNA, 19 bp, synthetic construct
  source 1..19: mol_type = other DNA
gaagggccgattcaccatc

SEQ ID NO: 29 - DNA, 21 bp, synthetic construct
  source 1..21: mol_type = other DNA
tgcagctctcagactgtccat

SEQ ID NO: 30 - DNA, 20 bp, synthetic construct
  source 1..20: mol_type = other DNA
ccggcagagccaatgaaagg

SEQ ID NO: 31 - DNA, 20 bp, synthetic construct
  source 1..20: mol_type = other DNA
ggcccaccagtgaagacatc

SEQ ID NO: 32 - DNA, 21 bp, synthetic construct
  source 1..21: mol_type = other DNA
ggcggtggaatacaatgtgtc

SEQ ID NO: 33 - DNA, 25 bp, synthetic construct
  source 1..25: mol_type = other DNA
ggagctctcgaagagatgaattctg

SEQ ID NO: 34 - DNA, 22 bp, synthetic construct
  source 1..22: mol_type = other DNA
gaatcgctgaggctttccagtg

SEQ ID NO: 35 - DNA, 22 bp, synthetic construct
  source 1..22: mol_type = other DNA
accgtcctcaaggtgcaaagtg

SEQ ID NO: 36 - DNA, 20 bp, synthetic construct
  source 1..20: mol_type = other DNA
ccaggtggtctcctctgact

SEQ ID NO: 37 - DNA, 21 bp, synthetic construct
  source 1..21: mol_type = other DNA
gcttgacaaagtggtcgttga

SEQ ID NO: 38 - DNA, 19 bp, synthetic construct
  source 1..19: mol_type = other DNA
tgcccagaacatcatccct

SEQ ID NO: 39 - DNA, 19 bp, synthetic construct
  source 1..19: mol_type = other DNA
ggaaggccatgccagtgag

SEQ ID NO: 40 - DNA, 1383 bp, Mus musculus
  source 1..1383: mol_type = other DNA
atgctgaccgtcggctgcacgctgttggtcgccctgctggccgcgcccgcggtcgcgctggtcctcgggagctgccgcgcgctggaggtggcaaatggcacagtgacaagcctgccaggggccaccgttaccctgatttgccccgggaaggaagcagcaggcaatgttaccattcactgggtgtactctggctcacaaaacagagaatggactaccacaggaaacacactggttctgagggacgtgcagctcagcgacactggggactatttatgctccctgaatgatcacctggtggggactgtgcccttgctggtggatgttcccccagaggagcccaagctctcctgcttccggaagaacccccttgtcaacgccatctgtgagtggcgtccgagcagcaccccctctccaaccacgaaggctgtgctgtttgcaaagaaaatcaacaccaccaacgggaagagtgacttccaggtgccctgtcagtattctcagcagctgaaaagcttctcctgccaggtggagatcctggagggtgacaaagtataccacatagtgtcactgtgcgttgcaaacagtgtgggaagcaagtccagccacaacgaagcgtttcacagcttaaaaatggtgcagccggatccacctgccaaccttgtggtatcagccatacctggaaggccgcgctggctcaaagtcagctggcagcaccctgagacctgggacccgagttactacttgctgcagttccagcttcgataccgacctgtatggtcaaaggagttcacggtgttgctgctcccggtggcccagtaccaatgcgtcatccatgatgccttgcgaggagtgaagcacgtggtccaggtccgtgggaaggaggagcttgaccttggccagtggagcgaatggtccccagaggtcacgggcactccttggatagcagagcccaggaccaccccggcaggaatcctctggaaccccacacaggtctctgttgaagactctgccaaccacgaggatcagtacgaaagttctacagaagcaacgagtgtcctcgccccagtgcaagaatcctcgtccatgtccctgcccacattcctggtagctggaggaagcttggcgtttgggttgcttctctgtgtcttcatcatcctgagactcaagcagaaatggaagtcagaggctgagaaggaaagcaagacgacctctcctccacccccaccgtattccttgggcccactgaagccgaccttccttctggttcctctcctcaccccacacagctctgggtctgacaataccgtaaaccacagctgcctgggtgtcagggacgcacagagcccttatgacaacagcaacagagactacttattccccagataa

SEQ ID NO: 41 - protein, 460 aa, Mus musculus
MLTVGCTLLVALLAAPAVALVLGSCRALEVANGTVTSLPGATVTLICPGKEAAGNVTIHWVYSGSQNREWTTTGNTLVLRDVQLSDTGDYLCSLNDHLVGTVPLLVDVPPEEPKLSCFRKNPLVNAICEWRPSSTPSPTTKAVLFAKKINTTNGKSDFQVPCQYSQQLKSFSCQVEILEGDKVYHIVSLCVANSVGSKSSHNEAFHSLKMVQPDPPANLVVSAIPGRPRWLKVSWQHPETWDPSYYLLQFQLRYRPVWSKEFTVLLLPVAQYQCVIHDALRGVKHVVQVRGKEELDLGQWSEWSPEVTGTPWIAEPRTTPAGILWNPTQVSVEDSANHEDQYESSTEATSVLAPVQESSSMSLPTFLVAGGSLAFGLLLCVFIILRLKQKWKSEAEKESKTTSPPPPPYSLGPLKPTFLLVPLLTPHSSGSDNTVNHSCLGVRDAQSPYDNSNRDYLFPR

SEQ ID NO: 42 - DNA, 1389 bp, Rattus norvegicus
  source 1..1389: mol_type = other DNA
atgctggccgtcggctgcaccctgctggtcgccctgctggccgcgcccgcagtcgcgctggtccttgggagctgccgcgcgctggaggtggcaaatggtacggtgacgagcctgccaggggccactgttaccctgatctgccctgggaaggaagcagcaggcaatgctaccattcactgggtgtactcaggctcacagagcagagaatggactaccacgggaaacacactggttctgagggccgtgcaggtcaacgacactgggcactatttgtgcttcctggatgatcatctggttgggactgtgcccttgctggtggatgttcccccagaggagcccaagctctcctgcttccggaagaacccccttgtaaatgccttttgtgagtggcatccaagcagcactccctctccaaccacgaaggctgtgatgtttgcaaagaaaatcaacaccaccaatgggaagagtgacttccaggtgccttgccagtattctcagcagctgaaaagcttctcctgcgaggtggagatcctggagggtgacaaagtgtaccacatagtgtcactgtgcgttgcaaacagtgtcggaagcaggtccagccacaatgtagtatttcagagtttaaaaatggtgcagccggatccacctgccaaccttgtggtatcagccatacctggaaggcctcgttggctcaaagtcagttggcaagaccctgagtcctgggacccaagttactacttgttgcaattcgagcttcgataccgacctgtatggtcaaagacgttcacggtgtggccgctccaggtggcccagcatcaatgtgtcatccatgatgccttgcgaggagtaaagcatgtggtgcaggtccgagggaaggaggagtttgacattggccagtggagcaaatggtccccggaggtcacaggcactccttggctagcagagcccaggaccactccggcagggatcccggggaaccccacacaggtctctgttgaagactatgacaaccacgaggatcagtacggaagttctacagaagcaacgagtgtcctcgccccagtgcaaggatcctcgcctatacccctgcccacattcctggtagctggaggaagcctggcgtttggattgcttctctgtgtcttcatcatcttgagactcaagaagaaatggaagtcacaggctgagaaggaaagcaagacgacttctcccccaccgtatcccttgggaccgctgaagccgaccttcctcctggttcctctcctcaccccatcagggtcccataacagctctgggactgacaacaccggaagccacagctgcctgggtgtcagggacccacagtgccctaatgacaacagcaacagagactacttattccccagataa

SEQ ID NO: 43 - protein, 462 aa, Rattus norvegicus
MLAVGCTLLVALLAAPAVALVLGSCRALEVANGTVTSLPGATVTLICPGKEAAGNATIHWVYSGSQSREWTTTGNTLVLRAVQVNDTGHYLCFLDDHLVGTVPLLVDVPPEEPKLSCFRKNPLVNAFCEWHPSSTPSPTTKAVMFAKKINTTNGKSDFQVPCQYSQQLKSFSCEVEILEGDKVYHIVSLCVANSVGSRSSHNVVFQSLKMVQPDPPANLVVSAIPGRPRWLKVSWQDPESWDPSYYLLQFELRYRPVWSKTFTVWPLQVAQHQCVIHDALRGVKHVVQVRGKEEFDIGQWSKWSPEVTGTPWLAEPRTTPAGIPGNPTQVSVEDYDNHEDQYGSSTEATSVLAPVQGSSPIPLPTFLVAGGSLAFGLLLCVFIILRLKKKWKSQAEKESKTTSPPPYPLGPLKPTFLLVPLLTPSGSHNSSGTDNTGSHSCLGVRDPQCPNDNSNRDYLFPR

SEQ ID NO: 44 - DNA, 639 bp, Homo sapiens
  source 1..639: mol_type = other DNA
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 45 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 46 - DNA, 2742 bp, synthetic construct
  source 1..2742: mol_type = other DNA
atgttgacgttgcagacttggctagtgcaagccttgtttattttcctcaccactgaatctacaggtgaacttctagatccatgtggttatatcagtcctgaatctccagttgtacaacttcattctaatttcactgcagtttgtgtgctaaaggaaaaatgtatggattattttcatgtaaatgctaattacattgtctggaaaacaaaccattttactattcctaaggagcaatatactatcataaacagaacagcatccagtgtcacctttacagatatagcttcattaaatattcagctcacttgcaacattcttacattcggacagcttgaacagaatgtttatggaatcacaataatttcaggcttgcctccagaaaaacctaaaaatttgagttgcattgtgaacgaggggaagaaaatgaggtgtgagtgggatggtggaagggaaacacacttggagacaaacttcactttaaaatctgaatgggcaacacacaagtttgctgattgcaaagcaaaacgtgacacccccacctcatgcactgttgattttgtcaacattgaagtctgggtagaagcagagaatgcccttgggaaggttacatcagatcatatcaattttgatcctgtatataaagtgaagcccaatccgccacataatttatcagtgatcaactcagaggaactgtctagtatcttaaaattgacatggaccaacccaagtattaagagtgttataatactaaaatataacattcaatataggaccaaagatgcctcaacttggagccagattcctcctgaagacacagcatccacccgatcttcattcactgtccaagaccttaaaccttttacagaatatgtgtttaggattcgctgtatgaaggaagatggtaagggatactggagtgactggagtgaagaagcaagtgggatcacctatgaagatagaccatctaaagcaccaagtttctggtataaaatagatccatcccatactcaaggctacagaactgtacaactcgtgtggaagacattgcctccttttgaagccaatggaaaaatcttggattatgaagtgactctcacaagatggaaatcacatttacaaaattacacagttaatgccacaaaactgacagtaaatctcacaaatgatcgctatctagcaaccctaacagtaagaaatcttgttggcaaatcagatgcagctgttttaactatccctgcctgtgactttcaagctactcaccctgtaatggatcttaaagcattccccaaagataacatgctttgggtggaatggactactccaagggaatctgtaaagaaatatatacttgagtggtgtgtgttatcagataaagcaccctgtatcacagactggcaacaagaagatggtaccgtgcatcgcacctatttaagagggaacttagcagagagcaaatgctatttgataacagttactccagtatatgctgatggaccaggaagccctgaatccataaaggcataccttaaacaagctccaccttccaaaggacctactgttcggacaaaaaaagtagggaaaaacgaagctgtcttagagtgggaccaacttcctgttgatgttcagaatggatttatcagaaattatactatattttatagaaccatcattggaaatgaaactgctgtgaatgtggattcttcccacacagaatatacattgtcctctttgactagtgacacattgtacatggtacgaatggcagcatacacagatgaaggtgggaaggatggtccagaattcacttttactaccccaaagtttgctcaaggagaaattgaagccatagtcgtgcctgtttgcttagcattcctattgacaactcttctgggagtgctgttctgctttaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccagtattctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtga

SEQ ID NO: 47 - protein, 913 aa, synthetic construct
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 48 - DNA, 2757 bp, Homo sapiens
  source 1..2757: mol_type = other DNA
atgttgacgttgcagacttggctagtgcaagccttgtttattttcctcaccactgaatctacaggtgaacttctagatccatgtggttatatcagtcctgaatctccagttgtacaacttcattctaatttcactgcagtttgtgtgctaaaggaaaaatgtatggattattttcatgtaaatgctaattacattgtctggaaaacaaaccattttactattcctaaggagcaatatactatcataaacagaacagcatccagtgtcacctttacagatatagcttcattaaatattcagctcacttgcaacattcttacattcggacagcttgaacagaatgtttatggaatcacaataatttcaggcttgcctccagaaaaacctaaaaatttgagttgcattgtgaacgaggggaagaaaatgaggtgtgagtgggatggtggaagggaaacacacttggagacaaacttcactttaaaatctgaatgggcaacacacaagtttgctgattgcaaagcaaaacgtgacacccccacctcatgcactgttgattattctactgtgtattttgtcaacattgaagtctgggtagaagcagagaatgcccttgggaaggttacatcagatcatatcaattttgatcctgtatataaagtgaagcccaatccgccacataatttatcagtgatcaactcagaggaactgtctagtatcttaaaattgacatggaccaacccaagtattaagagtgttataatactaaaatataacattcaatataggaccaaagatgcctcaacttggagccagattcctcctgaagacacagcatccacccgatcttcattcactgtccaagaccttaaaccttttacagaatatgtgtttaggattcgctgtatgaaggaagatggtaagggatactggagtgactggagtgaagaagcaagtgggatcacctatgaagatagaccatctaaagcaccaagtttctggtataaaatagatccatcccatactcaaggctacagaactgtacaactcgtgtggaagacattgcctccttttgaagccaatggaaaaatcttggattatgaagtgactctcacaagatggaaatcacatttacaaaattacacagttaatgccacaaaactgacagtaaatctcacaaatgatcgctatctagcaaccctaacagtaagaaatcttgttggcaaatcagatgcagctgttttaactatccctgcctgtgactttcaagctactcaccctgtaatggatcttaaagcattccccaaagataacatgctttgggtggaatggactactccaagggaatctgtaaagaaatatatacttgagtggtgtgtgttatcagataaagcaccctgtatcacagactggcaacaagaagatggtaccgtgcatcgcacctatttaagagggaacttagcagagagcaaatgctatttgataacagttactccagtatatgctgatggaccaggaagccctgaatccataaaggcataccttaaacaagctccaccttccaaaggacctactgttcggacaaaaaaagtagggaaaaacgaagctgtcttagagtgggaccaacttcctgttgatgttcagaatggatttatcagaaattatactatattttatagaaccatcattggaaatgaaactgctgtgaatgtggattcttcccacacagaatatacattgtcctctttgactagtgacacattgtacatggtacgaatggcagcatacacagatgaaggtgggaaggatggtccagaattcacttttactaccccaaagtttgctcaaggagaaattgaagccatagtcgtgcctgtttgcttagcattcctattgacaactcttctgggagtgctgttctgctttaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccagtattctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtga

SEQ ID NO: 49 - protein, 918 aa, synthetic construct
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 50 - DNA, 759 bp, Homo sapiens
  source 1..759: mol_type = other DNA
atgggggtactgctcacacagaggacgctgctcagtctggtccttgcactcctgtttccaagcatggcgagcatggcggctataggcagctgctcgaaagagtaccgcgtgctccttggccagctccagaagcagacagatctcatgcaggacaccagcagactcctggacccctatatacgtatccaaggcctggatgttcctaaactgagagagcactgcagggagcgccccggggccttccccagtgaggagaccctgagggggctgggcaggcggggcttcctgcagaccctcaatgccacactgggctgcgtcctgcacagactggccgacttagagcagcgcctccccaaggcccaggatttggagaggtctgggctgaacatcgaggacttggagaagctgcagatggcgaggccgaacatcctcgggctcaggaacaacatctactgcatggcccagctgctggacaactcagacacggctgagcccacgaaggctggccggggggcctctcagccgcccacccccacccctgcctcggatgcttttcagcgcaagctggagggctgcaggttcctgcatggctaccatcgcttcatgcactcagtggggcgggtcttcagcaagtggggggagagcccgaaccggagccggagacacagcccccaccaggccctgaggaagggggtgcgcaggaccagaccctccaggaaaggcaagagactcatgaccaggggacagctgccccggtag

SEQ ID NO: 51 - protein, 252 aa, Homo sapiens
MGVLLTQRTLLSLVLALLFPSMASMAAIGSCSKEYRVLLGQLQKQTDLMQDTSRLLDPYIRIQGLDVPKLREHCRERPGAFPSEETLRGLGRRGFLQTLNATLGCVLHRLADLEQRLPKAQDLERSGLNIEDLEKLQMARPNILGLRNNIYCMAQLLDNSDTAEPTKAGRGASQPPTPTPASDAFQRKLEGCRFLHGYHRFMHSVGRVFSKWGESPNRSRRHSPHQALRKGVRRTRPSRKGKRLMTRGQLPR

SEQ ID NO: 52 - DNA, 2916 bp, Mus musculus
  source 1..2916: mol_type = other DNA
atggctttctctgtggtccttcatccagccttcctcctggcagtgctgtccctgagggcatcccgaagcgaagtcttggaggagcctttaccattgactcctgagatacataaagtttcttttcaattgaaacttcaagaagtgaatttagaatggactgtcccagcccttactcatgaagaattaaacatgatatttcagatagagatcagtagactgaacatatccaacaccatctgggtggagaattatagcaccactgtgaagcgtgaagaagctgtgcgttggaactggacgtctgatatccctttggagtgtgtcaaacatttcataagaatcagggctctggtagatgacaccaagtcccttccacagagttcctggggcaactggagttcctggaaagaagttaatgcaaaggtttccgttgaacctgataaatcattaatatttcctaaagacaaagtgttggaagaaggctccaatgtcaccatctgtctgatgtatgggcagaatgtatataatgtatcctgtaagttgcaagatgagccaatccatggagaacaacttgattcccacgtgtcattattaaaattgaacaatgtagttttccttagtgacacagggacaaacatcaattgtcaagccacgaagggtcctaaaagaatatttggtactgttctctttgtctcgaaagtgctcgaggaacctaagaatgtttcctgtgaaacccgagactttaagactttggactgttcatgggaacctggggtagatacgactttgacttggcgtaaacaaagattccaaaactacactttatgtgaatcgttctctaagagatgtgaggtttctaactacaggaactcctatacctggcaaatcactgaaggctcacaggaaatgtataactttactctcacagctgaaaaccaactaaggaaaagaagtgtcaacattaattttaacctgacccatagagttcatccaaaggctccgcaggacgtcacccttaaaattataggtgctacaaaagccaacatgacttggaaggttcactcccatggaaacaactacacacttttgtgtcaggttaaactccaatatggagaagtgattcatgagcacaatgtttctgtccacatgagcgcaaactacctcttcagtgatctggatccagacacaaagtacaaggcttttgtgcgctgtgcaagtgccaaccacttctggaaatggagcgactggacccaaaaagagttcagcacacccgagactgctccctcacaggctcttgatgtatggagacaagtgtggtcggagaatggaagacgcattgtgactttattctggaagccactattaaaatcacaggccaatggcaaaatcatatcctataatatagttgtagaaaatgaagccaaaccaactgagtcagaacactactgtgtctgggcaccagccctcagcacaaacctgagccttgacctgcaaccttacaagattcgcatcacagccaacaacagcatgggggcatctcctgagtccttgatggtcctttctaatgattctggacacgaagaggtcaaggaaaagacaattaaaggtataaaggatgcattcaatatttcttgggagcccgtatctggagacacgatgggctatgttgtggactggtgtgcacattcccaggaccaacgctgtgatttgcagtggaagaaccttggtcccaataccacaagcaccaccatcacctcagatgattttaaaccaggcgtccgttacaacttcagaatttttgaaaggtctgtggaacacaaagctcggttagtagagaaacaaagaggatacacccaggaactggctcctttggtgaatccaaaagtggagattccttactcgacccctaactccttcgttctaagatggccagattatgacagcgacttccaggctggttttataaaagggtacctcgtgtatgtgaaatccaaggagatgcagtgcaaccaaccctgggaaaggaccctccttccagataattcagtcctctgtaaatacgacatcaatggctcagagacaaagacactcaccgtggaaaaccttcagccagagtccctctatgagtttttcgtcactccgtacaccagcgctggcccaggacccaatgaaacgttcacaaaggtcacaactccagatgcacgctcccacatgctgctgcagatcatactacccatgaccctctgcgtcttgctcagcatcattgtctgctactggaaaagtcagtgggtgaaggagaagtgctaccctgacattcccaatccgtacaagagcagcattctgtcactcataaaatccaagaagaatcctcacttaataatgaatgtcaaagactgcattccagatgtccttgaagtgataaacaaagcagaaggcagcaagacacagtgtgtaggctctgggaaacttcacattgaagatgtacccactaagccgccaatcgtgccaacagaaaaggattcctcagggcctgtgccctgcatcttctttgagaattttacttacgatcagtcagcttttgactctggttcccatggcctcattccaggtcccctaaaagacacagcacaccaacttggactattggctccacctaacaagttccagaacgtattaaaaaatgactacatgaagcccctggtcgaaagtccaactgaagaaactagcttgatttatgtgtcacagctggcttcacccatgtgcggagacaaggacacgcttgccacagaaccacccgtgccagtgcatggttcagagtataaaaggcaaatggtagttcccgggagcctcgcatcaccttctctgaaggaggataacagcttgacctcaacggtcctcttaggccaaggtgaacagtaa

SEQ ID NO: 53 - protein, 971 aa, Mus musculus
MAFSVVLHPAFLLAVLSLRASRSEVLEEPLPLTPEIHKVSFQLKLQEVNLEWTVPALTHEELNMIFQIEISRLNISNTIWVENYSTTVKREEAVRWNWTSDIPLECVKHFIRIRALVDDTKSLPQSSWGNWSSWKEVNAKVSVEPDKSLIFPKDKVLEEGSNVTICLMYGQNVYNVSCKLQDEPIHGEQLDSHVSLLKLNNVVFLSDTGTNINCQATKGPKRIFGTVLFVSKVLEEPKNVSCETRDFKTLDCSWEPGVDTTLTWRKQRFQNYTLCESFSKRCEVSNYRNSYTWQITEGSQEMYNFTLTAENQLRKRSVNINFNLTHRVHPKAPQDVTLKIIGATKANMTWKVHSHGNNYTLLCQVKLQYGEVIHEHNVSVHMSANYLFSDLDPDTKYKAFVRCASANHFWKWSDWTQKEFSTPETAPSQALDVWRQVWSENGRRIVTLFWKPLLKSQANGKIISYNIVVENEAKPTESEHYCVWAPALSTNLSLDLQPYKIRITANNSMGASPESLMVLSNDSGHEEVKEKTIKGIKDAFNISWEPVSGDTMGYVVDWCAHSQDQRCDLQWKNLGPNTTSTTITSDDFKPGVRYNFRIFERSVEHKARLVEKQRGYTQELAPLVNPKVEIPYSTPNSFVLRWPDYDSDFQAGFIKGYLVYVKSKEMQCNQPWERTLLPDNSVLCKYDINGSETKTLTVENLQPESLYEFFVTPYTSAGPGPNETFTKVTTPDARSHMLLQIILPMTLCVLLSIIVCYWKSQWVKEKCYPDIPNPYKSSILSLIKSKKNPHLIMNVKDCIPDVLEVINKAEGSKTQCVGSGKLHIEDVPTKPPIVPTEKDSSGPVPCIFFENFTYDQSAFDSGSHGLIPGPLKDTAHQLGLLAPPNKFQNVLKNDYMKPLVESPTEETSLIYVSQLASPMCGDKDTLATEPPVPVHGSEYKRQMVVPGSLASPSLKEDNSLTSTVLLGQGEQ

SEQ ID NO: 54 - DNA, 2889 bp, Rattus norvegicus
  source 1..2889: mol_type = other DNA
atggctttctctgtggtccttcatcaagtgaccttccttctggcagtgctgtccctgaggacttcccaaagtaaagtcttgggggagcctttacagttgactcctgagatacacacagtttctcttcaatctgcacttcaagaagcgaatttagaatggactgtcccaacctttagtcatcaagaattaaacattgtatttcagatagagatcagtcggatgagaacatccaacaccatctgggtggggaattacagcaccactgtgaagcatgaagaaactgtgcattggaaatggacatctgatatccccttggaatgtgccacacatttcataagaataagggctatggtagatgacgcccagtaccctccacagagttcctggagcaactggagttcctggaaagaagttaatgcacaagtttcagttccacctgatacattattaatatttcctgaagacagactgttggaagaaggttccaatgtcaccatctgtttgatgattgggcagaatctatataatgtatcctgtaagttgcaagaagagccaatccgtggagagcaacttgattcacacctgtcactaataaaattgaataatgtagtgttccttaacaacgcagggacaaacatcaattgtaaagccatgaatggtactaaaaatacatttggtactgttctctttgtctcaaaagtgcttgaggaacctaagaatttttcctgtgaaacccgagactttaagactttgaactgtttgtgggaacctgggatagacacgactttgagttggcataaacaacgttcccaacactacactttatatgaatcgttctctgggagacgtgaggtttctaaccacagaaactcgcatacctggcaaatcactgaagactcacaggaaacatataacttcactctcacagctgaaaacaacctaaggaaaagaagtgtcagcatcagttttaacctgacccacagagttcatccaaaggctccccatgatgtcactcttaaaactgtaggtgctacaaaagcccacatgacttggaaggttccctcccgtggagactacacacttttgtgtcaggttgaactccagtgtgaaggagaagtgatacatgagcacaatgtttctgtccacacgagcgccaactacctcttcagtgatctggagccagacacagagtacaaggcttgtgtgcgctgtgcgagtgccaaccacttctggaaatggagtgactggatgcaaaaaaagttcagaacacccgaggctgccccctcagaagctcttgatgtgtggagagacgtgcggacagagaatggaagacatgttgtgactttattctggaagccactattaaaatcacaggccaatggcaaaatcatatcctataatatagttgtagaaaatgaagccaatccaacggagtcagaacagtactctgttcgggcgccagcccttggcacaaacctgagcctggacctgcatccctacaagattcacatctcagccaacaacagcgcgggggcatctcctgagtccttggtggtcctttctagtcattctggacatgaagaggtccatgaaaaaacaattaaaggtataaagaatggattcaatatttcctgggagccggtatctggagacgcgataggctatgttgtggactggtgtgcacattctcagacccaacgctgtgatttgcagtggaagaacgtaggtcccaatatcacaagcaccatcatcacctcagacgcttttgagccaggggtccgttacaacttcagaatttttgaaagatctgtggaagagaatgttcggttagtagagaaacaaagaggatacacccaggaattggctccgtcggtgaatccaggagtgacgattcataacttgaccccaaactccttcagtctaaaatggcaggattatgccagcgacttccagtctggctttataaaagggtacctcgtatatttgaaatccaaggagctgcagtgcaacccaaactgggagaggactgttctgtcagataagtcagtgctctgtaaatatgacgtcgatgacccagagacaaagacactcactgtggaaaaccttcggccagagtccctctatgagtttctcgtcactccatataccagcgctggccagggacccaatgaaacatacacaaaggtcacgactccagatgtacgctcccacatgctgctgcagatcatattacccatgaccctcggcgtcttcctcagcatcattgtgtgttactggaaaagtcagtgggtgaaggagaagtgctatcctgacattcccaatccatacaagagcagcatcctatcactcataaaatccaagaagaatcctcacttaataatgaatgtcaaagactgcattccagatgtccttgaagttataaacaaagcagaaggcagcaagacacagtgtgtaggctctgggaaacttcacactgaagatgtacccactaagcctccattggtgccaacagaaaaggattcctctggccccatgcccttcgtcttccttgagaattttacttacgatcagtcagcttttgattctggttctcatggcttcattccaggacccctaaaaaacacaccgcaccaactcggactattggctccgcctaacaagttacagaatgtattagaaaacgattacatgaagtccctggtcgaaagtccaactgaagaaactagtttgatttatgtgtcccagctggcttcacctatatgcggagacaaggacagtcttgtcacgaacccacccatgccagtgcacggttcagagtataaaaagcaaatggcacttccagggagcctcacatcagcttctctgaaggagaacaacttgacttcttag

SEQ ID NO: 55 - protein, 962 aa, Rattus norvegicus
MAFSVVLHQVTFLLAVLSLRTSQSKVLGEPLQLTPEIHTVSLQSALQEANLEWTVPTFSHQELNIVFQIEISRMRTSNTIWVGNYSTTVKHEETVHWKWTSDIPLECATHFIRIRAMVDDAQYPPQSSWSNWSSWKEVNAQVSVPPDTLLIFPEDRLLEEGSNVTICLMIGQNLYNVSCKLQEEPIRGEQLDSHLSLIKLNNVVFLNNAGTNINCKAMNGTKNTFGTVLFVSKVLEEPKNFSCETRDFKTLNCLWEPGIDTTLSWHKQRSQHYTLYESFSGRREVSNHRNSHTWQITEDSQETYNFTLTAENNLRKRSVSISFNLTHRVHPKAPHDVTLKTVGATKAHMTWKVPSRGDYTLLCQVELQCEGEVIHEHNVSVHTSANYLFSDLEPDTEYKACVRCASANHFWKWSDWMQKKFRTPEAAPSEALDVWRDVRTENGRHVVTLFWKPLLKSQANGKIISYNIVVENEANPTESEQYSVRAPALGTNLSLDLHPYKIHISANNSAGASPESLVVLSSHSGHEEVHEKTIKGIKNGFNISWEPVSGDAIGYVVDWCAHSQTQRCDLQWKNVGPNITSTIITSDAFEPGVRYNFRIFERSVEENVRLVEKQRGYTQELAPSVNPGVTIHNLTPNSFSLKWQDYASDFQSGFIKGYLVYLKSKELQCNPNWERTVLSDKSVLCKYDVDDPETKTLTVENLRPESLYEFLVTPYTSAGQGPNETYTKVTTPDVRSHMLLQIILPMTLGVFLSIIVCYWKSQWVKEKCYPDIPNPYKSSILSLIKSKKNPHLIMNVKDCIPDVLEVINKAEGSKTQCVGSGKLHTEDVPTKPPLVPTEKDSSGPMPFVFLENFTYDQSAFDSGSHGFIPGPLKNTPHQLGLLAPPNKLQNVLENDYMKSLVESPTEETSLIYVSQLASPICGDKDSLVTNPPMPVHGSEYKKQMALPGSLTSASLKENNLTS